SEQUENCE LISTING (168 sequences)

SEQ ID NO: 1 - DNA, 5199 bp, Homo sapiens
atgtctggagaagtgcgtttgaggcagttggagcagtttattttggacgggcccgctcagaccaatgggcagtgcttcagtgtggagacattactggatatactcatctgcctttatgatgaatgcaataattctccattgagaagagagaagaacattctcgaatacctagaatgggctaaaccatttacttctaaagtgaaacaaatgcgattacatagagaagactttgaaatattaaaggtgattggtcgaggagcttttggggaggttgctgtagtaaaactaaaaaatgcagataaagtgtttgccatgaaaatattgaataaatgggaaatgctgaaaagagctgagacagcatgttttcgtgaagaaagggatgtattagtgaatggagacaataaatggattacaaccttgcactatgctttccaggatgacaataacttatacctggttatggattattatgttggtggggatttgcttactctactcagcaaatttgaagatagattgcctgaagatatggctagattttacttggctgagatggtgatagcaattgactcagttcatcagctacattatgtacacagagacattaaacctgacaatatactgatggatatgaatggacatattcggttagcagattttggttcttgtctgaagctgatggaagatggaacggttcagtcctcagtggctgtaggaactccagattatatctctcctgaaatccttcaagccatggaagatggaaaagggagatatggacctgaatgtgactggtggtctttgggggtctgtatgtatgaaatgctttacggagaaacaccattttatgcagaatcgctggtggagacatacggaaaaatcatgaaccacaaagagaggtttcagtttccagcccaagtgactgatgtgtctgaaaatgctaaggatcttattcgaaggctcatttgtagcagagaacatcgacttggtcaaaatggaatagaagactttaagaaacacccatttttcagtggaattgattgggataatattcggaactgtgaagcaccttatattccagaagttagtagcccaacagatacatcgaattttgatgtagatgatgattgtttaaaaaattctgaaacgatgcccccaccaacacatactgcattttctggccaccatctgccatttgttggttttacatatactagtagctgtgtactttctgatcggagctgtttaagagttacggctggtcccacctcactggatcttgatgttaatgttcagaggactctagacaacaacttagcaactgaagcttatgaaagaagaattaagcgccttgagcaagaaaaacttgaactcagtagaaaacttcaagagtcaacacagactgtccaagctctgcagtattcaactgttgatggtccactaacagcaagcaaagatttagaaataaaaaacttaaaagaagaaattgaaaaactaagaaaacaagtaacagaatcaagtcatttggaacagcaacttgaagaagctaatgctgtgaggcaagaactagatgatgcttttagacaaatcaaggcttatgaaaaacaaatcaaaacgttacaacaagaaagagaagatctaaataaggaactagtccaggctagtgagcgattaaaaaaccaatccaaagagctgaaagacgcacactgtcagaggaaactggccatgcaggaattcatggagatcaatgagcggctaacagaattgcacacccaaaaacagaaacttgctcgccatgtccgagataaggaagaagaggtggacctggtgatgcaaaaagttgaaagcttaaggcaagaactgcgcagaacagaaagagccaaaaaagaggttagtattcatacagaagctctagctgctgaagcatctaaagacaggaagctacgtgaacagagtgagcactattctaagcaactggaaaatgaattggagggactgaagcaaaaacaaattagttactcaccaggagtatgcagcatagaacatcagcaagagataaccaaactaaagactgatttggaaaagaaaagtatcttttatgaagaagaattatctaaaagagaaggaatacatgcaaatgaaataaaaaatcttaagaaagaactgcatgattcagaaggtcagcaacttgctctcaacaaagaaattatgattttaaaagacaaattggaaaaaaccagaagagaaagtcaaagtgaaagggaggaatttgaaagtgagttcaaacaacaatatgaacgagaaaaagtgttgttaactgaagaaaataaaaagctgacgagtgaacttgataagcttactactttgtatgagaacttaagtatacacaaccagcagttagaagaagaggttaaagatctagcagacaagaaagaatcagttgcacattgggaagcccaaatcacagaaataattcagtgggtcagcgatgaaaaggatgcacgagggtatcttcaggccttagcttctaaaatgactgaagaattggaggcattaagaaattccagcttgggtacacgagcaacagatatgccctggaaaatgcgtcgttttgcgaaactggatatgtcagctagactggagttgcagtcggctctggatgcagaaataagagccaaacaggccatccaagaagagttgaataaagttaaagcatctaatatcataacagaatgtaaactaaaagattcagagaagaagaacttggaactactctcagaaatcgaacagctgataaaggacactgaagagcttagatctgaaaagggtatagagcaccaagactcacagcattctttcttggcatttttgaatacgcctaccgatgctctggatcaatttgaacgctctccatcctgtactccagctagcaaaggcagacgtactgtagactccactccactttcagttcacacaccaaccttaaggaaaaaaggatgtcctggttcaactggctttccacctaagcgcaagactcaccagttttttgtaaaatcttttactactcctaccaagtgtcatcagtgtacctccttgatggtgggtttaataagacagggctgttcatgtgaagtgtgtggattctcatgccatataacttgtgtaaacaaagctccaaccacttgtccagttcctcctgaacagacaaaaggtcccctgggtatagatcctcagaaaggaataggaacagcatatgaaggtcatgtcaggattcctaagccagctggagtgaagaaagggtggcagagagcactggctatagtgtgtgacttcaaactctttctgtacgatattgctgaaggaaaagcatctcagcccagtgttgtcattagtcaagtgattgacatgagggatgaagaattttctgtgagttcagtcttggcttctgatgttatccatgcaagtcggaaagatataccctgtatatttagggtcacagcttcccagctctcagcatctaataacaaatgttcaatcctgatgctagcagacactgagaatgagaagaataagtgggtgggagtgctgagtgaattgcacaagattttgaagaaaaacaaattcagagaccgctcagtctatgttcccaaagaggcttatgacagcactctacccctcattaaaacaacccaggcagccgcaatcatagatcatgaaagaattgctttgggaaacgaagaagggttatttgttgtacatgtcaccaaagatgaaattattagagttggtgacaataagaagattcatcagattgaactcattccaaatgatcagcttgttgctgtgatctcaggacgaaatcgtcatgtacgactttttcctatgtcagcattggatgggcgagagaccgatttttacaagctgtcagaaactaaagggtgtcaaaccgtaacttctggaaaggtgcgccatggagctctcacatgcctgtgtgtggctatgaaaaggcaggtcctctgttatgaactatttcagagcaagacccgtcacagaaaatttaaagaaattcaagtcccatataatgtccagtggatggcaatcttcagtgaacaactctgtgtgggattccagtcaggatttctaagataccccttgaatggagaaggaaatccatacagtatgctccattcaaatgaccatacactatcatttattgcacatcaaccaatggatgctatctgcgcagttgagatctccagtaaagaatatctgctgtgttttaacagcattgggatatacactgactgccagggccgaagatctagacaacaggaattgatgtggccagcaaatccttcctcttgttgttacaatgcaccatatctctcggtgtacagtgaaaatgcagttgatatctttgatgtgaactccatggaatggattcagactcttcctctcaaaaaggttcgacccttaaacaatgaaggatcattaaatcttttagggttggagaccattagattaatatatttcaaaaataagatggcagaaggggacgaactggtagtacctgaaacatcagataatagtcggaaacaaatggttagaaacattaacaataagcggcgttattccttcagagtcccagaagaggaaaggatgcagcagaggagggaaatgctacgagatccagaaatgagaaataaattaatttctaatccaactaattttaatcacatagcacacatgggtcctggagatggaatacagatcctgaaagatctgcccatgaaccctcggcctcaggaaagtcggacagtattcagtggctcagtcagtattccatctatcaccaaatcccgccctgagccaggccgctccatgagtgctagcagtggcttgtcagcaaggtcatccgcacagaatggcagcgcattaaagagggaattctctggaggaagctacagtgccaagcggcagcccatgccctccccgtcagagggctctttgtcctctggaggcatggaccaaggaagtgatgccccagcgagggactttgacggagaggactctgactctccgaggcattccacagcttccaacagttccaacctaagcagccccccaagcccagtttcaccccgaaaaaccaagagcctctccctggagagcactgaccgcgggagctgggacccgtga

SEQ ID NO: 2 - protein, 1732 aa, Homo sapiens
MSGEVRLRQLEQFILDGPAQTNGQCFSVETLLDILICLYDECNNSPLRREKNILEYLEWAKPFTSKVKQMRLHREDFEILKVIGRGAFGEVAVVKLKNADKVFAMKILNKWEMLKRAETACFREERDVLVNGDNKWITTLHYAFQDDNNLYLVMDYYVGGDLLTLLSKFEDRLPEDMARFYLAEMVIAIDSVHQLHYVHRDIKPDNILMDMNGHIRLADFGSCLKLMEDGTVQSSVAVGTPDYISPEILQAMEDGKGRYGPECDWWSLGVCMYEMLYGETPFYAESLVETYGKIMNHKERFQFPAQVTDVSENAKDLIRRLICSREHRLGQNGIEDFKKHPFFSGIDWDNIRNCEAPYIPEVSSPTDTSNFDVDDDCLKNSETMPPPTHTAFSGHHLPFVGFTYTSSCVLSDRSCLRVTAGPTSLDLDVNVQRTLDNNLATEAYERRIKRLEQEKLELSRKLQESTQTVQALQYSTVDGPLTASKDLEIKNLKEEIEKLRKQVTESSHLEQQLEEANAVRQELDDAFRQIKAYEKQIKTLQQEREDLNKELVQASERLKNQSKELKDAHCQRKLAMQEFMEINERLTELHTQKQKLARHVRDKEEEVDLVMQKVESLRQELRRTERAKKEVSIHTEALAAEASKDRKLREQSEHYSKQLENELEGLKQKQISYSPGVCSIEHQQEITKLKTDLEKKSIFYEEELSKREGIHANEIKNLKKELHDSEGQQLALNKEIMILKDKLEKTRRESQSEREEFESEFKQQYEREKVLLTEENKKLTSELDKLTTLYENLSIHNQQLEEEVKDLADKKESVAHWEAQITEIIQWVSDEKDARGYLQALASKMTEELEALRNSSLGTRATDMPWKMRRFAKLDMSARLELQSALDAEIRAKQAIQEELNKVKASNIITECKLKDSEKKNLELLSEIEQLIKDTEELRSEKGIEHQDSQHSFLAFLNTPTDALDQFERSPSCTPASKGRRTVDSTPLSVHTPTLRKKGCPGSTGFPPKRKTHQFFVKSFTTPTKCHQCTSLMVGLIRQGCSCEVCGFSCHITCVNKAPTTCPVPPEQTKGPLGIDPQKGIGTAYEGHVRIPKPAGVKKGWQRALAIVCDFKLFLYDIAEGKASQPSVVISQVIDMRDEEFSVSSVLASDVIHASRKDIPCIFRVTASQLSASNNKCSILMLADTENEKNKWVGVLSELHKILKKNKFRDRSVYVPKEAYDSTLPLIKTTQAAAIIDHERIALGNEEGLFVVHVTKDEIIRVGDNKKIHQIELIPNDQLVAVISGRNRHVRLFPMSALDGRETDFYKLSETKGCQTVTSGKVRHGALTCLCVAMKRQVLCYELFQSKTRHRKFKEIQVPYNVQWMAIFSEQLCVGFQSGFLRYPLNGEGNPYSMLHSNDHTLSFIAHQPMDAICAVEISSKEYLLCFNSIGIYTDCQGRRSRQQELMWPANPSSCCYNAPYLSVYSENAVDIFDVNSMEWIQTLPLKKVRPLNNEGSLNLLGLETIRLIYFKNKMAEGDELVVPETSDNSRKQMVRNINNKRRYSFRVPEEERMQQRREMLRDPEMRNKLISNPTNFNHIAHMGPGDGIQILKDLPMNPRPQESRTVFSGSVSIPSITKSRPEPGRSMSASSGLSARSSAQNGSALKREFSGGSYSAKRQPMPSPSEGSLSSGGMDQGSDAPARDFDGEDSDSPRHSTASNSSNLSSPPSPVSPRKTKSLSLESTDRGSWDP

SEQ ID NO: 3 - DNA, 322885 bp, Homo sapiens
  misc_feature (41338)..(41520): N can be any one of A, T, G and C
atgtctggagaagtgcgtttgaggcagttggagcagtttattttggacgggcccgctcagaccaatgggcagtgcttcagtgtggagacattactggatatactcatctgcctttatgatgaatgcaataattctccattgagaagagagaagaacattctcgaatacctagaatggggtaagtttgtaatttttaaagaaactgttaatctttagctggtcattatgatttaattggtatagagagttatgagtaaactctactccgttgaaagaaaaaatacaataaaaaaacagacactagttattcaagattttccgctagtttttggtaaatattttcccaggagtagaattgtgctgctcaattgttgccttgatttaaggcctagctaatgtggaacttggctatgaagaacttatgtttttaggtcaacagtaagagattttaaatcaattctgtgatgggcaaaagtggattattggttacagttgcctaatacatggcactataatagtccttcatttgaaaggtatttattggtggaaacattcagactatggaaattattctcaaaataggaggagatattaaaatatctgtgtttgccttaggtcaacagaaagctgaaggtggtgatcagtgggttttatttcatctctacatagtacaaggaaaggaaactgctaaacatggactttccaaaaattatgtttgtaagattaaatgacatagtttaaaaatcttgtgatagtagttagagaatgtttgggggcagcttccacttgagagttagattttactcgaaagttaatttaaatctaaactggttaggtttttggcttcattcttctaaatatagatgtgatttagaagacctactgtagtggaatagatcaatgatgtgagcaaacgctccatgaccttcaagcttgaaggcttggctcaagttcttcagcgtctgctgttacatacagggcacattctaaggtccagaagttcactgttgcaataagcacgcatcgtcacccagatctgtctgtagtagtggtacgttggcttttgaaggattttggccaaatatgattttgtacctttggaatactaatcattttagtttgttcttatgctaagttcttatgctaaaatatctgttcccaagtaaaactttattttcctgaagtaaaagtcaagtgctgattgtgtctttttttttttttttttttggatttgaaatttattcaaagttagtttctcaggtaaaccactgatacaagaatgtctacacgtaataactttaattattcaatttgccagtataagaaggtttctagcaatgtgatgtcataaggtttaaaaaaatatgtaaatgtgaatagaataacttttcaaaacagactgccagactcatcctgggaatgtaagaaatctaaaccatctttcaagtaacttctcacttaagagtgagacaattttggctttggtactggttttgtgagtatacgtcttttatttgttgaataaagtattttgtaaagcatatgtagttttacatcccatgtttcacaaattgagaaattctgtagtgttaatttttttttttaagacttaaagggttttggtacttttagagttggaacttgagttcttaagttatggttccttaatgacactctagtctgtgatttttgtcatttagtattctttcctatctgcgaagctatgggagaactctatgtaccagtagtaaatataattttaaatgatttatgataaagagaaaactgtatcctccattgccttctgattattagagtttaggtaagtagttttatgttatgagatatttcgctgtattatcagaataatttatatatattttccattaaatatgttttcttggatgttgtttcttatatatgtataagaacacctcatttatgtatatttttatataagccaaatatcaaataagtatttggctgagtattatttttattagtatggaagtctggataaagagtaaaaagaaagttaagaatggaaaaggctgtcaggagttgcgtagtgagtagtggtatttgctgtctttaagttatttgagaatttaggttaagagtgccagtctatttaagaattgaaatagatcagatgttcttcatatgaattgataactgcattttggggaaatgtcctattttattttaattacttaacaatatgtgaaaatcagttaagactaagaatatttccaaattgtgaattgacctcagttcagtgttctaaacattattttttgggactctggtggaaatttacagggattgttgttggggttaaatttggtaatttagatttgccttaaatggtgtaattttaagtttaatttttaataattgaaaaaatattgtcttttatttaaatctttaaaagactatttgcaacaggaacgcagtattaataacacaagttgttacatatttgtgttcgttatttatattaatgaaagtgtcagctagatgctacttatctttatgctgatgatagattactcttaaagtacagtcagccaagattcttaccggctgtattcagcttcatttctatcaatttcattttattcctagttttagtccattttatttttatattggtacagttaatgttttgttcatagcagtatcaaaacaataagaaaactgacaattcaaagtcagatatgttgtttttctttgacagcctttcttgcttttggtgctgatggcttgagggaggaaataagtggaaagagaaagagaggattaagccaatgatcactagagcaagggaataaaagtgccttaagactatagatcacatttaaatttaataagatgtattatttttcatttatagtaaatagtatttgaaagactatgttttttgtctgcttagttttaagttgttgaaatgattatggtttgtatgtttgtgtatacagtaatagtcgatgtcagcataactctagaataaattacagtggagttgtagaagtaaaaataacttccacattgtaaactaatttgctagtaatgactgaattagtatatcattcgttaaatcggccaccccctactttgaaatgggaaattggaattttggggaattatttaccaatttacttaataaatgtaagataatattacttaatacaaaataaatatgcaaggctgtctgtacatggataactattttgatgctttttgaattttggactttgtaacatacagaaattatcactaaataactaattttatagcttattcagattttgtatatacatagaaaatagtctgtttaataaatttgtacctagagcatatcagcaggtactatgctttaagcttttctttttattagtatagttttaaagctgttcactttttttttatgacacgagtgttaattaagtgaggtgttgatggatatgtatcttttctaggacacttttgttcttcacatatgctgttcctcattgttagcttttcctgagtagaatttattaattaatcagtccgaattatcaaaatgttcacttaatacagatgattacatttattttatttaagttctcaactagaataatcagcaatattggttgggtacttgtttcctttatactagccacctaatattctattctaggaatgataatgagtaattacaattattttatctagtacaatgctggcacatggtagaagtgtgttcaatagatgtaatggctattattataacatttctgtttgtctctctctttttaagagaaattacacttagaggtagttgccagatgatactggttactggcattcatgatggaatagcataggagtgatgctgttgataaaattacatctaggaaaaattccctataaagtagagtgaacttaggtataatgaactagtgctgttaacccctaaagtctttgaaatacttgatgttcattagtaatttttttgtttttaggaacatcacccttttatattatgtattgatttattttagagacagggttgtattataattgtttttaggaacattactctttttattttatgtattgctttattttagagacagggttttgttagtctgttctcgcattgctataaagaactacctaagactgagtaatttatagagaaaagaggtttgattggctcacagttccgcaggctacaaggaggcatgcaggggaggcctcaggaaacttgcaatcatggtaaaaggcaaaggagaagccgaccccgtcctgcatggctggagtgggaggaagagagagaagggggagatgctacacgcttttaaacaaccagatcttgtgagaactcactcactatcacaacacaaaggggaagtctgcccccatgatccagtcacctcccgccaggtccctcctccaacactgggattacagtttgacatgagatttgggtgacggcacaaatccaaaccatgtcaagggtcttactgtgttacctaggctggtcttgaactcctgggctcaagagatcctcccacctcagcctcttaaagagctgggattacaggcatgcaccaccacacccagcttttttaaaaaaggaatgtattgcttcatgagtgaaaacaggatgtcaggattaacactttaggaattttagacatatatttattttatatggaaatgtaaggcataccttagtatagcagtatataaaaacaaatagtgcagtatgttggaaattttatgaaatgtcccataattcctaggacttgaatactattacacagtaagctcaggctattttaggataaaataatttagctcaaataaatattttattgtaccactcttttatggcagtcaaaaatttattctctgacctttttctcatttggtacatttattgacagtatgttacgtatgtagctcattgtttttctcctgctggttgtgataggttttaaaaccaattactaagtctaaccagcatttaaaaaaaattaaagactagaatagaaaatataaagtacatcaaatatagaaggaataagtattgttgtgcaaaacattgtgttttcctgtagtccttattcccatatttcttactataggttgagataaaaatagtttgaaatacttcagtgtaggtgcagctatgctatgttgactgttaaacagtgaaaatatagacagttcattttttcaaaaaggacacacgatagattagggacttactcaagaacctagtgaagggaaagagtgtaaggtatgggcaagatacagacttggaaatagatgggaagaggagacatgggggagggagaaaaagagaatgagtagggagagggagacagagaatgagaatgaatactgtgttcctgagttgctacttcagtctgacttgttgaatgatagggtattttttttttttatgattgggtatttttaatttaaaagaacttaatgggcaggggagttaatttcactcttgtatttctttcaatggtttattgtctcttcctttgtttttttccgtttctgagtttgggctaatgggcattgactttgattgattgtcagaggtatatacgtttttgagtttgagtgataaccatatgacctataaacaaaggtagtagtttcttggttttagcttctcatcacttcagctgaactctagggtagatgtaggtgaggatttgctagttctatcagattttttctcatcatttgttgctcaggaaactgccagttttaggtatcgaggaccaagaattgcttagctccttttgccctgtcttgatgagtcaagaagtctttgcataataattgttatgagagaccatcatggctcactacagcctcaacctcctgggctcaggatcctcctgtctcagcttaccaagtagctggagctacaagcatgtgccaccatgcccagctaattaaaaaatttttgtgtgtgtggagatgggggggtctcattatgttgtcctgactggtctcaaactcctgccctcaagtgatccgcctgccttgggcttccaaagtgttggaattgcaagtgtgatcatctgcacttcgtgtgatttttgtttttgaattacccacactgtaagtatggattatcccagacacaagttttttgttgtatataaaaactccttggccgggtgcggtggtttacgcctgtaatcccagcactttgggaggccgaggtgggcggatcacgaggtcaggagattgagaccatcctggctaacatggtgaaaccgcgtctctactaaaaattcaaaaaaaaaaattagccgggcgcggtgacgggtgcctgtagtcccagctactcaggaggctgaggtgggagaatggcgtgaacccaggagtcagagcttgcagtgagccgagatcgcgccactgcactccagtctgggcgacagagcaagactctgtctcaaaaaaaaaaacaaaaaacaaaaaaaaaaactcctttccaaatctgggggacttccttttcttctccattaatggtgagtagaactccaggattattctttctagctagcttctacacacttccttcctatgactgccatctctttttctaatcagtacgtagggaatggctaatcttctattggctagtgtttctttatctatcactgtatgagagcagcactttagctttcatgttaacggggcacaaagacctggctccccctttccaatcccactgtttgcctttactcttcctcatggaactgtttgcttctcttttgggattagggcctcttcactctaacctccctccctagtttcaaagtgtgttctggaaatccatgggatatttaatagatgcttgagcaaggggaaaggtttatatgaataaatcagtttgggaaaagattttagacaacatttaatgttaaaaatacagcagattatcctagaatttatgaagtttatctgactatggaataggctttttttcagagcgtctggaggatggctggcaaccagtgtttcacagaatacgctttgggaaatgttcttccagtataaggtcatggttttccttgtaacttcctgagtagcagggttagaaagaaattttgagagaaatttgggatatcgtgtggattggagagagaatccaaatctgaataggttatattttacttagtggctgtgaacttcattttctgtgtgaaagtcatgtgcactgaaagataaatttaatcatggtgaaataacatgagtgatgctgttggtaaaattacatctaagaaaagcaccctaaaaagtagagtgaaatttgacatcataatgaaccactgctgtcaacccctaaagtccttgaaattactgagagatctcttaattatgaccttttcctgctctaattaagcttacttttctttatgtgtaggcatttacattaaatacagaaattgtcactttattttttacagctttcattactgtagatgttgcaaaactattgtctatctgaagagattacattcattactttgtgatttttaagaggaaaatatcatggactcatagttttaaaatcctgttacagtgttaagtattgttttttgcagtgtaacacattttggctaggccattggtttcagacttttggactagtgtgggaactttactactatttattacatccatgaagcgctgtgtttctagtgccagcaactatttatatatactgttaaggaaactagaaaatatatacttttattaaggagaaaacataattggtgctattttggcattgtgtgaacaattaaagctgtggttactattaacttttgttcagagtttttgggaggccagataataatcatagaggacaatctagtttcattgtttaaagacagacagcatataggggaataaaattagttttctcgcttattttttttttttttttttggtagagatagggtctcactatgttgtccacgctggtcttgaactcctagcctcaagcaattctctgaccttggcctccccaaagtgctgggattacagatgtgagcagccgcacctggccttttttcttttttctgatttagatttatttttctctttaactcttcctcaccagtgccattcttcccactcctgatctaattatactgcaaagtactacttatatggatgaaaacaatcttttgaaaagattgtgaattatttcagtcatgtgcaatcatgtgttaactttcagttttatttcagattgcttaaacgtcactgtaggcttaatcaaagacaaaaacaccttctgtttaatttggtctattcatagtgataacacttaatccagtttccttagagtttccatggtgaatatctacaagtagaacacctaacctgaaatgtttctgttctaaaccaatatactggaaaagagcaaggggttttggattaagactgactgctttgattattaactgggtgatttagttatttaacctccctatgtctctttatgaaaaatggacataagactaatagtttatttttctaaaaagttaaaagttggtttaggattcttccttttctggtgatttatggttgtattttgtcagcttgcttcccattctggtttttttttttttttttttgagatacagtctcactgtgttgcccaggctggagtgcagtggcgtgatcttagctcactgcagcctctacctcccgaggtcaggcaattcttctgcctcagcctcccaaggagctgggactacaggtgcacgctgccatgcctggctaatttttttcttgtattttagtagacacaaggtttcaccatgttgcccaggttcatcttgaactcctgaactcaggcaatccaccgggctaggtctcccaaagtgctgggattttaggcatgagccaccatgcccgtcctcccattttgctttttaaaattttgataaaggaataattaaaatgccatttattggtaaataataactgaaatgcatagctatactaatataaggatgtaaataactattttactattaacatacatttgtatcttactgatagtattttattatattgggattgaagatgccaatatttctgtgtgactatgcaaaaaaatcttacaggataagctttttgcattggaacattttgttgttatataattattcttcagtaagtatcaagttaatgctcattcatccaccaacaacaacaaatatgagatgactaattacattttatttttatatttatatatctccaatctgcacatcagtggtagagttctggacctcaacgcagagggttaaaatcatcatgaaaaattattgatgtgttgttcatttaggactttttatgtattgaagattatgcagcacccgtaaatgtatttgtgacctgacagtacgacacaaacgtgtggtgtggtatgtagaaacttaatgtacactagtgtttcctaaattatatttctagagatataaatgaatattctgaaaattaagagttcagtggctgtgtaagattggaaaatagtgctttttgtatgcctctttggatattacaaagcacattatcatagaaaaagtttcaaaaagtttgctgtagaaaccatttaaactttgttttacctagaatttgcctgagtaatttaacattggaactccttatgaagaacagctgacagagtttgggaaatgctggtatatacttaaccgtaggtagtgtgagagcctttaaactggttgtgtgacagaaagtggttaataaagtcttctaacacataaattgtcacaatttatatcaatttccaaacaaaggcaaaggtttgaaggtttgcctttatcctttccatattttttagcgtatctgtttatgtcatctaagcaatattaagttggtgtggagttttgcatggggtgtatatatctttacaaagaaaggacagtgagacttgatacatgacttcgaggagtttatatgtgcacttcttggtttgcaaagatgcacctgctaagctagtgattttgtttttttccagtaaagctttattatgttctaatccagttacctctctaaccattggtacagaatttgggatcgatattgtcatggctgtgaaatagaacaatgctaattagattttatgaaagttttgtaaaagcagtatagttatcttgctacagcaagaatcccatccttaagtatcatcgtttacttagagtggctaatgcaatgccccaagttgactcagatactattaaaagcactcaagcatctacttagaagtggtatgccctgagatttaaaaaatacttgtttgaaaagattaggaaaagcccccgcaatgaaacagtgactaaagcccctttttgagattgtattgctagctacctgtgatctggccctgagctaaaagcataactaacaaacatttaacaacagactaagttcgggtatgtagtgaagctgacatatgatatcataaagcttttccatactctctacttatagagattctcctagagtttatggttaaaactacaataatattttcaggtctactgtagtatctgtttctgcctttttgttgcatgcgttcgtaatgtgttacccatttctgactaatacatgctaagttgacagtttcttctaagagtcctttgctgtgttatgttgcttgaggctgtgattaagtgtgatcatcatatttgtctcttttgcctatgtacctctggtaatcccagttaagtgtagtagctcatgagttctggttttattcaacctctgtgtgcatctggctcagttgacttctctgtattaaacatgtacaaatgggttacgttcctggatcttactgatcttattaatcatgtttgtttttccattaaatactaagcaataacaacttccttattacatgtacctctaacttgaaaatactgtgaattctacacatccaagacctttgtagaaggaagcaaaaaaaaaaaaaaaaccacttagaacatgatcctttgatcatgtcaacatcctgcttaatatacttcagtgactactgaaagctttcagaatgacatttgaactcctctttagcatgccatacagaatttttaatatgatcctgtttacttctctttttcttcactttatttatttatttagttttttgagatggagtctcgctctgtcacccaggctggagtgcaatggcacgatcttggctcactgcagcctccgccttctgggttcaagtgattctcccgcctcagcctcccgagtagctgggattataggcatccaccaccatgcctggccaatttttgtatttttagtagagatggggtttcactatgttggccaggctggtctcaaactcctgacctcgggttatccacccaccttggcctcccaaagtgctgagattacaggcgtgagccaccactcccggcagatgctatgatattttaatggctgaaaaagtggtgtcaaatttgatgatggtgatgaagaaaataattttcataatccttgaagtatatgaacttcgttgaaagagaataaagtcagcttaataagaatgacatctaaaggtatgagaggacctagtaatctttgaaagaccaaccttaagagacttctctgacagcaagaaaaatgaaagagtaaacaacaagaaatgaagacgaggtttcttgagacaacaaaccacaatgcagacattatacaaatgtgagttacttttatcctatccattttgggattaatgtaaagaataagagtaatctatacagagtaatggatttaagataaaaaaatgtttgcctcagcttcccaaagtgctgggattacaggcatgagccaccatgcctggctttttcttcacttaaaaaaattttgaaataatctcgagtttacagaaacattgcaagtatagtataatttttttcctctggaaccatttgagagtaagtagcctacctaatgctctgtcatccccatatattttctgcattcttctacctaaccacaatacaactattaaaatgaagaaattaacattggtgtatattgtcatctaatctttagggctgaattgtttaggaaggagaagggaatggccacttattatgtatgtatgtgtgtgtgtgcaattttatatttattttccactgtcatcttaaaaattatttaaagttctttaagaaatttacctatagtaaaattcacttattttggtgtgtatattgtgagttttaacaaatgtaaagtcatgtgaccactaccatagtcaaggtacagagcagttccatcatccctaaaattctcttgtgctattcttttgtcccacactcatcttttactccagctataaatgaatttgactcttttactaattaaaccataactttgattattcatgacttttcacatattggttaaattgtttggaaactcctctttcactgcaccccataggagacatttggtaatgtaggttgaatgaattatgccgttatacttgttatatgcagggctagaagcagtagctctaatcttgaaacattgtatgaaactcatatttttaatgccagggacttgtttcttctttgatttaccctattttatttcaaagtaattttttccttttaaaattatttaataattagaaatagtgataataaaaattcaaaagtggtttttgtgtttttctttttttgttttttttttgagatagaatcttgctttgttgcccaggctggagtgcagtggtgtgatctgggctcactgcaacctctacctcctgggtttaagcaattcttctgcctcagcctcctgagtagctgggactgcagacatgtgctactatgcctggctgatttttgtatttttttgtagagggggtatcgccatgttgaccaggctggcctggaactcctggcctcaagtgatgcgcccacctcggcttcccaaagtgccaggattaccggcgtgattacaggcgccaccacatgtggtcaaaagtgatttcttatatgagtagtcatttattatcccctagcagttggcaatttgtaaacaagtacttcatggtggtgaggtaaggagaagagggtgctgctatttaaaagaatgaggtaaaatgcagataatctcaaatttttcaaggtaaaagttcttagattaactctgcatttaactttatttgcacaatgtccatcataaactttagagtctggtgatgatggattgagcatacaaatataacttcatttccttctgcaattcactaacactataataaaataatttttttaaagagtcataaacctttaaggatgataagagtggtagaagagactatagaaacaaaattttggaagctgaaaagcaggtagatgagtggtaatgacttaggaggtctgagaaagtcagattccaaattagcagtggtgaaagctgaggacctgttcagtttacttggcagaatcttcagaagttttagaaattgctatctttaggaacctctggaagttgaggtatgggtagggctaaagtacagaggattgattgaggctggtttagaagcagtagaaactctagagcaattcccctgttgagtgccattgggttactttatccctctcctcaaacaaatgttggatgttcttagtacaacagaggttttctggattgggcaacataggcatagtttatggtggacatttcatattgaaaataaggagattaagtaaatttgtatttacttctgctcccagaatgctggcaactgggattaggacaggagattagaagattagaagagtcttgtctggagaatctgactctccccaagaagaaagaacttagagatgcagatatcagaggcttctcagtaaagggctcaggcagtcacactacagtgagatttgttatcagggggtttcacccaaggatttggagcttcacatcatgttttagttctcgtaattaaatatgaatggaaaatagaattgccagacatctgaggatcctcaaagcctctatagggtacagtcccaaacaaagaaagcaacccacatgaaacatacttttcagggaaagcataacttcgaaaaaaaaaaaaaacccaaaaaaaccaaatcacataaaactatcagtattttccagagatgagagaaaatcctgcaatcatgaagtcaggatgccataaaaaggagcaaaagagaatgaaaaaagttctttgaaattattatgatagctaaaatggaaacctcagtacaagattggaaagtagaaaagtggagcaaaaagccacagagagatagaaaagaggagatagaggagaaaattagaggaccaattgaggaggcctgaataataatctgatgaatgataggaattctaaaagagcactttgaaagaacagagggggaaaaaaatcatcaatacaagaaaatgttgtagaaccaaaggacacgagttcctagatagaaggggcctaccaagtacctaggtcagtggatgaaaatagattcgcatcaaggttcagaatcaatatattttgcaattcctaaaactctggggacaaacaagagaccccaaaaggttccaaagagtgtgaatacaaaggataagaatcagaaactctttggccttttcagcagcaactctagaagctagaagacagtgttcatattttgaaggaaaacagtttctaaactgaaattctatatccagacaaattcttaagtatgagggcgggataaggacatttttaggcatgaaggtgttaaaaattctgtaacctgtgggctacaaatggtttacttaggataaggagcttcccaggatgtgggacttctactaaaaccagcacagtcttaggcaaactggaatgagttgattgccttagctgcaactaggtaaaggagtgaatgaagatgaggaagtaacaggatataggaagcaggagatacaatgcaggagagagtagaaaggatttcctaagactgtaatgaagggagaatacaaaatgacaactttgcaccaagcatagaggtgaaccataacagacaggatgcagttcagaaggtttcaggacagacttaagtatctgatatggatgaacatcttgagaggagattagaaaatctggggagggtttaaggttgcttaggaaatgaagcaaatgagaaaacaataagaaaaatagaaattaggaaaggaaaagtaataacttatatggttatagtaatggaaacacttaatatttatctaactaaaattatgctgtaaccatattgggaggatggtatggaaagggaattttttttttggtggaagtgatagtaggtacggggcgtaatgtttttcctaaatcttaagtgatgattgaatgtgtatttatgaataactgataaaaatgaaaattttaaaaacagctgtagtttttacagaattaaaaagtctaaatccggctgggcacagtggctcatgcctgtaatcccagcattttgggaggccgaggtgggcagatcacgaggtcaagagattgagaccatcctagccaacatagtgaaaccctgtctgtactaaaaatacaaaaattagctgggcatggtggcacatgcctgtagtcccagctaccctggaggctgaggcaggagaatcgcttgaacccaggaggcagaggttgcagtaagccgagatcgcgccaccgcactctagcttggcgacagagtgagacttcgtctcaaaaaaaaaaaaaaaatgtctacatcctttactcctacttcactgatcttattttcaaacttgtgtttttctttttgaggtatttctaagtcctgtaatcgaggagctcaaaactggatagagggaatgggaaatttgtgttagaatttatgagtatattctttgagcagatcagtctttttcttcttcagttatttctctgttcttatgatggcaatttatagtctttgtcttaggagtttagttgattcctaaaaaacctatgtaggttaaattcgttagtgatacacagatacttctgtgaacacacagcaaaagaacactaacagagcagtgaagacaaatcacacaaatgatgtaaatgacgcaaaggatggatatgattattatttcttaaaactaaggagctgccaggtgctatgaggaggcatcagaatacgtgtcaactatacaactgcaaaggtagctttggtttagcagcgcttacccgctcttctgatgttcaagttgatacttgtaaatgcaaagaaatgccgtgtgtgcgtgtgtgtaaattctcatagcttttggagtacaagaggtttttggttacatggatgaattgtatagtggtgaagtccaagattttagtacactcgtcacctgagtactgtacattttacccaatatatagttttttatcccttaccccctttcttccttctcccttctgagtctccacctccattatactactctgtctgcctttgtgtacccatagcttagctcccacttataagtgagaacatacggtatttggttttccattcctgagttacttcacttagaataatggcctccagctccatccaagttgttgcaaaagacattattttgttctttttatggctgagtagtattccatggtatgtgtgtatatatataccatatattctttatccctacattggttgatgggcacttaggttggttccatatctttacaattgtgaattgtgttgcaataaacatatgtgtgcaggtgtgtttttgatgtaatgacttattttcctttgagtagatacccagcagtgggattgctggatcaaatggtagatatacttttagttctttaaaaaatctccatacagttttccatagaggttgtactaacttacgttcccagtagcagtctataagtgttcgcttttcaccacatccacagcaacatctgttgtttttgactttttaataatgagaggtgccttatattaaatgaaattcctcttgtaatgaatttcaacttctcctagagaaaatcccttgaaaatctaaagaactgaccagctcttccctacatgtctgttttagggcaaggttaagagaagtttgtttcaggggattgctgaactgtaggactctgagaaaactggtccgctttctaactactcctgagtcaggttttcagaggacgagaatgacgtctttatttactttaacattatgactctttacattcagatgcatattgaaaaacacaccccaaaagaaattattattgaattatgtaatactttgcttccttgtatgagaaatcattctgtttagtttgttgactacttctgttgctcaaggtcgtttttattttccagactgtgggtcatttattttgttcatgattgcatacctaaaaatttatcttgcattttatctaaagggaaaaacatcaaaatagtagcaaagccataaaaaatttacataataaatctgtaatcattttagtttctaaatttaatgtctttgctgagaatcaatagaatttaaagcagatattaatcagtgtatctagataacattatatatttacagcttaaaatagggttaggatgaaatatttatgacagtttagtatattttgttttcaattaggtgtattactaaaaaacatgtaaaccattccatttaaaaaaagaagtaaaaggagatgatgtaatataactagtatttggaaatgacaaaggaattttccctccagctgtagaagtatttcctttgcaacagtgtttctcatttatgtttcagttctcagatatacaaagatgattgctgtgagagggcattagttttttaacagacaattaaattcagtttttgaaaaccctaattgtttagtgttcgtcaaccctaagaatttttacataaagctgcttaaatattagttaaaaagtcaagtttcattttctttccaaaattctctgcacatggctcaaactgtatttgagtctgaatttcttttgaaaatccagtaagctttatgaagaaatgccaagaaaatgtttagttcaaatttactcttctttcatagaatgactttcactttaactgtttcacttgatttctatttatgctactctacagctgtccatttgtgaaagttaccagcctctagaatgtaaagagctatatatgattctattctagcatattgaggtgaagttatagatgagcagtgtgttttgttcagcttagttttgattaattttccttttttatggatattgaattatctttataattcagtgaaaaggcagtgtttgagatatttcatgtgctttcttctctgtaaagttctcagtttttgtcactgaaacctataagccactgagcggctgctgtctgtatctgcttatgccagatagtccttggctttcatttgccagaatgcagcaagctttttgttatatagcttcaaatggcttttttccttaaaagaagcatgtttttgctggagaagtatgaatctatattaacagttgattataaaggacaaaactctgggacagccaaatggaagtgatgcgtagggcaaggtttagggtaagggagtagagggggaggatttccatgccctccttaattttcttctgagtttttaggttatagcaccaacatactaatgacactagcttgtttatatacatgtttagccaaattatgaaaagtaaaagaaactatttacctgaggatattattacaaaaaaaatgcctcaagttgagtaagggcaattataatagtggttgaatttctggtactattttgtcataatttcacttgttattttttatgtgtttgtgtatatttcttggatgtgctttaactgttgcttgtcttcagtaccttggcaatatatatgaatttggaattgtggcacgactctggggagggaggtattctggaaaatgccatatgtgtgaaatttttacctactacactgagaaagagagattttttttgagacagataaaaaaaaacattgtatatagtcacactgctttttctcagtctgctttcttttggtgtatgtatatatttatgtatgtatatattatgtatatatatgtatatatatgtatatatatgagtgtgtgtgtatatgtatatatacacacacacacacataaaacaaaatttgccattttagcccctttaaagtgtattatttggtggtgtcaattacatttcaaaatgtataacatcaccactatttcaaaaaccttttcatcacctcaagcagaagctctgtatccattaaccaacaactctacattctctcctttccccagtttgtggtaacttctgatctactttctgtctctctgaatttctacatattgtagatattttgtataagtggaatcatttaatattattttgtgtctggcatatttcatttagcatgctattcgtaaggttcattcatgtggcattatgtgtcagaacttcattcctgtttatggctgaataatattccattgtatggctataccacattctgtttatctgttcatttgttgatggatacttgcgttgtttccaccttttggttattatgaataatgctgcagtgaacattggcatacaagtatctgtttgagtccctatttttaactcttttgcatatatgcttaggagtgatatttctgggtcatatagtaattctatgtttaactttttgaggaactgccaaactgtttttcaaagcagtggtaccgtcatacattcccaccagcaatgtatgaaggttgagttttgctacatgcttgctaacatttggtactttctgttttttgattagtcattttagtaggtatgaagtggtaggtggttcattgtggctttgatctacttccttaataattactgataacgagcagcttttcatgtgcttattggcaatttgtatatcttctttggagaaatgtctgattaaccctttgcccactttaaaagatagactatttttagaaaagttttaggttcacagtgaaattgagcagaaggtatagaaatttcttagatttccagcctccccatatgcatagtctcgcctattctttgtctatttaaaaattgcatttatcttttgttgttagagttcttcatacattctggatattctttgtcatgtatattatttacacatattttctcccattctgtaggttgctttttcactttcttgataatgtgcattaatgatatacaagtttttcattttgatgaaatccaatttgtccctttttttttttcttgtgcttttggtgtgatatttaagaatccattgccaaatccaatgtcatattggtttaccctttgcttttttccaaaagtttttttttttttttttttgatgtggggagagtctctctctgtttcccaggctggagtgcagtggcacaatgtcggctctctgcatcctccgcctcctgagttcaagcgattttcctgcttcagcctcccaagtagctgggattacaggcatgtgacaccatgcccagctaatttttgtatttttagtagatgcggggtttcaccattatggccaggctggtctcgaactcctgacctcaagtgatctgcccacctcggcctcccaaagtgctgggattacaagtgtgagccactatgcccggcctatttttttcccaaaagttttgtgtttcacttcttatatttgttatgaatccattttgagttaatttttatatatgatttgaagtaagtatccaacttcattcttttccatgtggaaattccaattgttccagatttgccatatagaaaagagattattctttctgtattgaatggactcagcatcgttgtggaagatcaactggccttagatgtttggatttttttctagactctcagttctattccattgatctatatgtttatccttctgccagtaccatacagcctggattgctatagttttgtagtaagttttgaaattgaaaagggtaagtcctctaaactgtttttccaggttattttggcttttctgggctccttgcagttccatattgtgatgattggcttttccatttctgcagaaaaggctgttggaattttgttagggctcttctttattaacaaaacccgattgactgtgttttctagctttatttccagttaggtgtggtcacgtcagtagttctggctgattgatgtacaaagtgatgtgtgatgcttaccagaagcttcattaaaaggaaggctttccccactttattcccgtctgctgcacagaacacatgtgatgttttcagagcttggactgtgatgatgagggcatgagggccatactccaaggatggcccagcagtgagctggaagcatcctggattcctgacagctttgtgaaactgccataccagcattggaccttcaaatgtcttttacttgaaaggaaaatatattttcattttgtttaaattggggttgagttttttgttagagctaaacttaatttatacacttttcctactacattaacttggttatctcttcattgtgtgtactgtgtgagggagtgagataatatattaccagaattgctggaactatacttaatttaaattttttttaatttcatgctgggcatggtgtttcatgcctgtaatcccagcactttgagaggctgaggtgggtggatcacttgaacctaggagtttgagaccagccgggcaacatggtgaaaccccatctctacaaaaatacaaaaattagccagatgtggtggtgtgcgtctatagtcccagttgctctggaggctaaggtgggaggatcgcttgagcctgggcagtgtaggttgtaataagctgagatcacaccactgtaccctagcctgggtgagaacctgtatcaaaaagaaaaaattatatttcatatatatgtattgtgaatttgactttaaaattatttttccttagtatgtaatactaaaacgttctttaacttaaatgtagcctcaacatttttttaaaaagcagaaaaaaattttcactgctgtaacatagcagatttgtcatttttgtgctaacccgttgaatattttttatatacatacttagttttctacataggaacatttctggtatttttataccttgcttttttttttcacttaatgtgataaagtgatctagtgcaagttaattaacctcctttttttttttttttttttttttttttttgagacggagtctcgctctgtcgcccaggctggagtgcagtggcgcgatctcggctcactgcaagctccgcctcccgggttcacgccattctcctgcctcagcctcccgagtagctgggactacaggcgcccgctaccacgcccggctaattttttgtatttttagtagagacggggtttcaccttgttagccaggatggtctcgatctcctgacctcgtgatccgcccgcctcggcctcccaaagtgctgggattacaggcgtgagccaccgcgcccggccaattaacctctttttgcttcagtttctttctctctgaaaatgggaatagttattacacatgcttcgtggggttgttttgagcattaaataaaatattggagtaaaatgctagaccagtgcccaacatgtagtgaattcctggccctcaatgaatgttagttatcattaccattattaatatcgtgtgatcttaagctcttcttctatattattgtaaaatcttgataattacgctcttcaaagtctgcttattatttcattaaataaatacacagttatttattagatactaaatttttttttttttgagatgaagtctagctcagtcgcccaggctggagtgcagtggcgcgatctcagctcactgcaagctctgcctcccgggttcatgccattctcccgcctcagcctcccgagtagctgggactacaggtgcctgccaccacgcccagctaatttttttgtatttttagtagagacggggtttcactgtgttagccaggatggtcttgatcttctgaccttgtgatccacccgcctcagcctcccaaagtgctgggattacaggtgtgagccaccgtgcctggcctaaaatttttttaaacatttaatttacttccatttatttggtgttacaaataatgctattataaactgatctatgattaaatgtcattttattacaagctagagacacttttaaaagcagcatttatcccttattttataaggtagtgaagaaaatgtgaaatagattttgtttttctaaggcggagttttgttcttgttgcccaggctggagtgcaatggcgccatctcggctcactgcaacctccacctcccaggttcaggcgattcttctgcctcaggccaacctcccgagtagccaccatgcccggctgattttgtatttttagtagagatggggtttctccatattggttaggctggtctcaaactcccgacctcaggtgatccgtccacctaggcctccccaaagtgctgggattacaggcgtgagccactgcacccagcaatagatctttttttttttttttaatgactttaccatagtagctttgtatatgaatctgatggcaattgggatgtagatatcttttgcaataaataataatagtcacagattgtataaccagttctcataacacattataagttctgtaagcttgaggatttagtttggtttaattctctttttgtggtaatttttagaaactgtaaacatttaagtgttaaaaggtgattaagaatatgccatagtcactattttctggcatttatcactgtttttaattatatatatttgcaattatttgattattattgatcttccttactagagcagggactgtgtctgttttgcttaccagctcttccccaatacctaacataagtgcttggtgtattgtaggcactcaataaatatttatttgattaatgaaagaattggtgagtgaacaacggagttaagcaaataaatagggaataaaatgttttgatacagttaaatctcaacattgagtcagtagctatgatgaagaaataacatctaaaactccaactttagatacaattttgcagagatttggggggaatatagtaaaatataacaaggagggcaatttagagatgaagtatttaactctagggttttggttttgtttgaatgatagtggaaacatccaggtagaatgttcaggagagagaaatatgggccaaaggtcaaagggtagtcattcaaataatgacagtaattaagttcaataaattattgaatgtctaccttgtgctagctattgtatagttgtggaaggtaggtgagtaagacatattcctcataataataattcaattaaaatacctcttaccctgataattctcttcacaagtaaagttttaacaaagttagaattttgattttagaatttagtggaaagcaatgattatgaccaaattatcttaataccttgttgaattttcactgtatcttctaagtgataaagatgattttaattcagtgaactttgtagttgagacttttgttaataaaatatttttaaaaagttttactgccattctgtgtagaaataattggagcaagatttttaaaaataatcattatgatacctatcttttatctttctgtggaaaaaatatggaaacgcttatgtctctagaggtaaatatttagtgaaatgtggttttcatttgaaattaaaattaatcatttttgatttcatattttttctttaacactatgatactttaaaaattattggctgggcttggtggctcacgcctgtaatcccagcactttgggaggccaagacaggcggatcactaggtcaggagtttgagaccagcccggccaatatggtgacaccccatctctactaagaatacaaaaataagccgggcatggtggcgtgtgcctgtagtcccagctactcgggaggctgaggcagaagaatcacttgaacctgggaggcagaagttgaaatgagccgagatcgcaccactgcactccagcctgggcaacagagcgagactccgtctgaaaaaaaaaaaaattatctgttatagacctcagggctatttgttatttttaaattgtctttaaaatgaacttgaattttgtgaacattgtactttatttattgaatttatgattctgtagaagaaacaggtcaatttctggtggtacatgtcttaacttcagtattggaataaaaaaagtttaaatgatagtacagtaataagaggtagctgattacacaatacaaatgttgagggttaatagtgtgcttttggttggaacagaaaacctgatgcaaactggtttgaagaatatggaaagttttactagttaacctatcaagagcaaaagtggggtagacttcttggtttagtggctctatagtattatcaaatatccaaagtctttcagtctcactgttgttgtagtatagatttgggtgttttttttgtttgtttttgtttttaatttttgagacagagtctcactctgtcacccaggctggaatgcagtggtgtgactttggctcactacaacctccgcctcctggattcaagcgattctcctgcctcagcctcctgagtagctgggatgacaggcgcgtgccaccatgcctgggtaatttttgtatttttagtagagacggggtttcaccatgttagacaggctggttttgaactcctgacctcaagtgatccgcctgcttcggcctcccaaactgctgggattacaggcatgagccaccgcgcccagcctagacttcttttttaacactgttttccctcagggttatcagatggcaccggagcaacatgattttttgttcaggtggggaggagggaggaaggctacctgtagccctcttatttcatggaagcacctagaaagtcttccctccattctcaatgaccaaaattaggtctcctctctattcctgaaccaatataattggtattggtgccaataagattattgaggtatatgggctgtgtatgggaagtgcagtgatcttaacaaagttaggattctgtttaggaagacggaatggattctgtctagacaacaaacagtctcccctatagttggcattgtggcctacagtttatttgtatcattttcgaaaggaatttttagtgctatagtaaagctttatgctatactttgtgtttccccttcccaccccaccacctgtaactaacaccttccctaataatgtgtttttgatttagggattctgggccagtgttactgtgtgtaagagaggaaaggatgcattatagatatgtataattaaattttaattttaaaattataaatagtcattaaaatgatacctttctcagccatgccatcctttgttggcatatccctcctctgaatgcctataaaatttgtttatagtgctcatttggaaaccaatatgaatcttggattatttattgtctttttatgtatgtcataccaggatcatttcttgtatatctaggacaaataaattctattatatctgtgttactggtatctgcagaaggtgtcattaagagaagatttaaagtcaattcaataaattttttaaatgtacagtaaagtattatgtctataatggagtgtagcagcaatgaagatgaacaacatgtggttccttccctaaaggaattaaacagaattaaatgtaataaagggggcagtagagagagattactttggtgtggggagtagaaaggctcaatcatggaagtgacattttgagcttactcttaaggaattaatgttttgtgaaacagagaaaagggggcattttagatggaaaggaagctcaaaggcatgtgcttggagcatagtacttgggtgcagttgaggcatagtgggaaataaagttgaaatggtggattggggccatttcagaaggcttactacttaatttgaatttgactctatggacagtgggaagccattgaatatatatatttgtttttatttttagaggtagagtcttgctgtcactcacccaagctagagtgtagtgttgcagtcatagctcactgcagctttaaacttctgggctcaagtgagcctcccagtagctgggactataggtgcacaccaccatgcctagcttttttttttttttttttgtagagacagggtctcaccatgttgcccaggctggtctggaactcctagccccagtagtcttcccactttggcatcccatgtatttctttttaaagcaagaagacttggaaattattttccgccgaaacataagtacccttaagattcgttgttttttgccatgatacagtgtttgtggagtacacacattaattcatttattttaacagatatttattgcatacctcctatgtgacagacataaaaatctggagatacaacagtgaacgagacaggcagggtttctgctctcatggagcttacattataatttggggggaggtcaggaaatagacatgtaaataagattatttcagataatggtaagagctataaagaaaataagaggatgctgtgacagagtgttttgggtggtaatttagatgagttggttagtgaagacctctgaggatttgatgtttgaactgatgttaaatgagaaggtgccagccatgtgactatcttgggtaagagcaccctaggtagagggaacagtaagtgctgggtccttaagatggaaaaaggaacagaaggaagaccagtgtggctggattagagtgagtgaaggggagagtggtagggtatgaggtttctcctggaaaggagtttatattttcctctaagggttaaagccagtgcagagttttgagcagaagggtgatatctagatttatgctttaaaagggtaactgtggttgtcatgtgaataaaatcatgccagttaggacctttgtagcagtacacgtgagagagagagtagtgccttgaagttgggctgtagtaggggagatggaggtttgtgatatcattttatttaatttttttattttgaattattattattattatttttgaaacaagagttttgctctttttgcccaggctggagtacaatgacatgatctctccccactgcagcctccacctctggggttcaagtgattctcctgcctcagcctcccaaatagctgggattacaggcacctgccaccacacctggccaattttgtatttttagtagagacggggtttcaccacgttggccaggctggtctcgaactcctgacctcaggtgatctgcccgcctcagccttccaaagtgttgggattgcaggcatgagccaccacacctggccttaaaaattttttttttgagaccgagtcttggtctgtcacccaggctggagtgaaatggtgcagtcttggctcactgcaacccccgcctcccgggttcaagcaattctcctgcctcagcctccccaatagctgggaccacaggtgtgcaccacgcccggctaatttttgtatttttagtagagatggggttttgccatgttggctaggctggtcttgaactcccggccttaaggaatccacttgccttggcctcccaaagttctaggtttacaggcatgagccactgcacctggccgatatcattttaattttaagatttttcatttttttattttttgaggcaggatctccctctgttgcccaggctggagtatagtggcacgatcacagctcactgcagcttagacctcctgggctcaagtgatcctcccacctctgcctcccattcacttctgattgcttttattatttcatgaagttgataaaatgtaatcagctgaggatattgggaaataatgggtgattagacatttagtgagagaagagaagatttgtgtatgtttgggatcccacagaccatccccatgttaggtgctttactaggaggactgatgggacaagcatgtaatgatactcatagattaatgtttattacagtgaaaagctacaaagcaaaatcaacaaagggaaaaggctaatggagcaaagccggaggaaaccaagcacatacttccaaggatcctctcccattggaatcatacaggatgtgtttaatcccctcagtaatgaattttaatgtatagtgtgaagtattgtctgccagagaaactcattgccctacagttttgaatagctagaaggacgatgttgaacattaccaacacaaataaatgataaacgtttgagatgatggatattgtaattaccctgatctgatcaccatacgtcttatgtattgaaacatcactgtgtactccatgaatatgtataattatttgtcaattaaaaaccctgggcactgagtttatttttttattggtggctagtcacgtaggaaccctctgcctagcctctgcctagcacatactgaaattcaacaaggttgaagattcaacaaggtattaagtaatttggtcataatcatattgctttccattgaattctaaaatcaaaattctaactttgttaaaactttacttgtgaagagaattatcagggtaagaggtattttaattgaattattaatattaattgaattattatcattccttctggacttccagaaggatagtagattttcagcataaattatattatttgcagaaatagttcaggtacagtgagccattcttaccagttaggattgtgagaacactcccaaaatctaagttcccagatgccagccatagaccaaccttgcaaataggccttgctaagaaatagcagtctcaggtctgtgatgtaactcttttccgcacaatacgaaacagtacttttggagcattggagagagaccatcctaggagagagtattgagattgtcaggaatgtgtgttcatgtaaaatgtgtgatgatgacttttatcagcaagcattgatgtagtttggaagtttttccccaccaaatctcatgttgaaatgtgacccccaatgttgaaggtggggtctagtgggagatatttgggttatggagacagattttcatgaatggcttggtgtccttcctgtggttaatgtgtgagttctcgctgtgtttgttctcccaatatctgattgttaaaaagagcctgatacctccccactccccattgctcccgctcttgccacgtgacatgccagcttcttttctccatctaaaaccgcaattacttttgcatcaacctaatactatgaatgaaagcttcttgaagccctcaccataaacagatgctggtgccatgcttttttgtacagcttgcagaactgtgagccaaaataaatctctttgtaaattacccagccattcagatattcctttatagcagttcaaaatggactaatacactaatacagaaaattggtaccgggagtggaatgttgctataaagataactgaaaatgtggaagtggctttggaactggataacgtacggaggttggaagagtttggagggctaagaagatgataggaagacagggcaagtttggaacttactaaagactgattaagtggctctgataaaaatgctgatagaagaatggacaacaaagcccaagctgacgaggaatttactgggaactctggagcaaagttcacccttgttacaccttagcaaagaacttgactgcattgtgtccatgttctaggactttgtgaaaggctgatcttaagagtgatgacctagactaactggcagaagaaatttctaagcaccaaagcattcgagaatgatatgactgcttctaacagcctgtgatcaaatatgggagcaaatgaatgacttaaagttggagcttataataaaaagggaaacagcataaaattttggaaaaatctcagtctggtgcagtagtaaagaaggaagaagtgttttcaggagaggaatccaagggggctgtggagcaaccacttgctagagagagtagcatgatgaaaaggtaggtaagtgccgatattaagacagtgggaaaaaggccctgaaggcatttcagaaatcttccaggctgtccttcccattataggctcagaggcttaggaggacagaatggtttcaaggggccaggcctagggcttcaactgccttgtgccgactcaggatgctgttcttctctctgactccagctggattaataaaagcatgattgtgattttttttttctttacctatatccaagtactcaggagtaggcagagttacagttttaactagggtttgctgacataatttgaattgttcagaaatgaggtggctcctttttttacactagcgtgagacagagaaacaggtggagattttgtggattttattctcattcgataatcccatggtaaaccctaaatgatcctctttctagtgacaggaccatctaggactcacagaggatacaatttgtatggaataacctgtcaagataagcagagtatcagatactgacagtccttcattgtgacctcttatgggacgccattacctggcttggttttggcctagtaatttcctggctagatggatggaatttgggtctgttatttcaggggcatatggatagaaccaataatgtaaatttttgtttgcttttcataggcaatttgtggttattaaataattacgtaagatgtaaagtcctcagaagaagtggtgcttaacttgatatatgcatccaaataacttagtggctttttcaaaaaatatatgattgctactcacttctagagattatgactcagttaactcaggatggaagtcagtgtatgtgagtttttaaaatgcttccctgttaattccaatgtgttcttctgattaagaatcatagttccagaccatttaatgttagcttagtccttttataagttttggactctccattaatgaaactgtgtagatagtttgtattgctacctgttttgcacaagaaacaagaattttgtcaggtggggtgtgtgtctggctgggtagactggagaacgttggaccatgtttctctatccagaggtatgtaccagagaatgctttgatccagaacaaaggggatttttttttttttttttttgagatggggtctcactctgtcacccagtgacagattgcagtggcacaaccttgggtaactgcagccttctcctcctggaatcaagcgatcttcccacctcagcctcctgagtagctgtgatgacagacgtgtaccatcatgcccggctaattttttctattttgggtagagacagggttttgccatgttgcccaggctggtctcgaagtcctgagctcaagtgatccacccaccttggcctcccaacatgctgggattacagatgtaattaatcctggcctagaacaaagtggatttctaacagttaggggaacttgggtttccagcaagagaaaaatgcaatcaaatgttgctctgaagtttgggcttagggaccaacttagtatttctttaggaatcatagattaagaaaatgtgtacattctctccagtgctattagtattaaggttaggccctctcctggggatggtggtgggtagaatacttgataatcacctgctgggcctcagatttaaagcttacggacttacatgtggttcactggaaaaccaaccagtatttataacataatctttgagataatgtattccaaattccaaagtagttatttgtaactaaatttctagattatgactagttcataagttgtgaattgctttaagttcagcttttcatttcaaggagaataaaatataaaaaattaaatcattactttttaggaaatgcaaattcttatatttttagttacaaaatatcaaaatatcctatttaatactcatcaaaccataccatcatgacatttgatgtgtcaagacaaagcattaatagaagatagtttatggattattcattagttatttaaaacaatctagtaaagcacttaattaaaaagtatatatattttttgagatatagtctgtgttgcccagactggagtgcagtggtgtgatcttgtctcactgcagcccactccacctcttgggttcaagcagttcttgtgccccagcctcccgagtgctgggattacagtcatgcgccaccatgcccagctaatttgggcaaaatcccggccaacatggcaaaatcccatctcaaactcctggcctcaagtgaactgcctgccctggcctcccaaagtgctgagattacaggcgtgagccaccatgcctggcctaataaagcattaacaaaaaatttagatgcaagggtacatatgcttatttattacatggatattacatgcataatggtggggggttgggcttccgtactcatcacccaaatactgggcattgtatccattggtaatttttccaccctccgtacttctgtcctctttcctttggagtctcgagtttttttcctccatttttatctccatatgtaccctttgtttagctcccacttataaagcattttgaaaataaatataattatagcataaacattttcattacaaatagaatcttcctacctggttttcatttgctttttctatttttaggtggagagagtagtaaatgattatgataaaattttataaactattagtaaatattatcatagtattttcataggatatgaagagtaagcctgccatttttttggtgggtcaatacttttcactcatttatatttctctcgtttatgtgtatatagaaagcttatataaaactttctgtagcatttaatagcaacagatttataaatgtttgcttacaaaaatatgtacaaaaagtacaaatgagactatatgtacttttccaggaacatactgttgtatcaatatgtgtattttaagtcatggtccttagagtagtgatgttctgccactaaaatagaataatagaaatctttaatttttaggaaagattaagtcaatgggtagaattttctcaatttaaagtttttcatcccatgaattataaaaagttatttaaaaaattgtgttaaaatatatataacataaaatttactattttaaatcttttttaagtgtatagttcagtgacatcaagtacatacacattgttgtgcaaccatcctgatcatccatctcagaacattttgtgttttgctaaactgaaactgtactcattaaaaatgactttactcccactttccataccttgccctctggctactgtcattctacttcttgtctatgaatttgactactcttaagtacctcatgagtagaatcataacagtatttgtccttttttgactagcttatttcactggttctgcacttgaataaatgtaagaaccactagcaaagagtaaggtagagctgtgcttctttgctgtttgtgttttacctgctcctaagcaaacagtattactgttttaaatacttgaaaaattatttatctgcatttaaaaaataacatattaatcaagatcttagataactgccggtaataccagagagcctgttgctgctgcagtgtgtgctagaggaattctgctgtctttttgacttagctgctatcacagtcctcacatcccacatgtgtaacagtaaacactggaagttattttattttctatgcagacattttagagtattatataaacaaccgcttaattaattttggtttaaactcctaggaagtgatgaaatcacattttaacaggactgaataggaaccataaaataatttttaggtaatctctgttctcaccctaagaatattaattctgccattctaggtaatgtttactctaattcacttaaaatttttcttttccctttttttttttttttttgagacggagttttgcttttgttgcccaggctggagtgcaatggcgcaatctcgactcaccacaacctctgcctcccgggttcaagtgattctcctgcctcagctcccaagtagctgggattacaggcatgtgtcaccacgcccggctaattttgtatttttagtagagacggggtttctccatgttggtcaggctggtcgcgaactcccgacctcaagtgatccacccgcctcagcctcccaaagtgctgggattacaggcgtgagccaacatgcctggcttctcttccttttaattattagaactcttttttcctgattgactgctctggttttacccagagccaggtaggttttccccttcccattttgttattaagggacatgtgcagaggttcaggactcctgagtctttgggcagtgctgatctcactcctctgctcagcctcatgctttgtcttaaaacttaattgtctgagttggagcacttgcaaaagctctaactcaggctagtggttgggagggttctcagagacttaattgctgcctggtgctcagggcctccaggtcccactacctctggctcagtacaggcatggggaaccagccacaggagactgaccagctggagactggcaagcgtgggggctctgagctgcttccaggggccctgggacaactgcttttgagggcctcatgtctactggaccatgggatccaagagaagagcccttgcctgaaatcacaggacctggaatggggctggcagttgtacaggttggagaactgggcctggtaatccctttaggagctgtgctctgaggagctttggagtttccctggcatgtctggggtgagagtgggggaaaaaagtgactctgagggttagggcaatgacctactcacaccccacctggttttgtgtaaaaccagagcagccaatcacgagaaagcgttctaatgattgattaaaaggactgcctttttctttctttaaaccacagcaataaagggttttgctaggctaactttctcagtttggcttggcctagattttcacatggctcagtattctccacattcacatgggccatggtgtttgtggaagtgaaggattggaatttgggagaatgtggtgaggagagagacagctgacttggtgcagtatctgggctatggaacaggcactgcataactgctgatggagtcttggctaccagtagcagccggtctatcccagagaggcagaggccctgagaggcagttctgaccccctgagctgttgtgctgggatccctggcttggcatgcttaccttagaagagaagcaggctaagaaactgatctctgtctcagctaaaacaatccttcttgcatactgcagacgatccctattaggaaacagaacttccagaatgcagtccatttgtaagtttttatcattcatttataccggagagtcaattaagatcagattttttttgttaaaatataaattggcaattttagtttttaaaagatctactaaagctcaggagataatttagtgtaatagtcttcaaactgtggtatctgtatccttggtgcgtgaagactttccaagggatatgcggatgcatatagttttaagggaatcaattttcaggtctttagctttcctaattagtcttctaaaatccatctgtttgagaacagtctgaactctgtggattttctttccactcccatttcacaatgaaccatctcccatttttataaaataaaggcatatttctcacccatctcatatgttcccatggtactgtcttgtgttgtgaagatctcttaggtgtaaaacaaaagggactgtttgagaatgtgttgctcttgaaagagggcaaaagaaaaagagcttctataaaaagtattgaagcaggtattttatttcactagcttacaaaccagtggcaaaattcttatccgtcttctttcagagaactctgaagcaaaatggttgataccaggctatgtaataacacattcatttgaattaaaaattaaagtcagacttattctaatagaaaataagtctttttttatacaaggttttaattccatttagtacagtactttgtatctttttttcttttttttgagatggagtcttgctctgtggcccaggctggattacagtggcacaatctcggttcactgcaacctccacctcccaggttcaagttattctcctgcctcagccttccaggtagctgggattacaggcacacaccaccacatccggctaattttttgtatttttattagagatggggtttcaccatgttggccaggctgatcttgaactcctaacctcaagtgatctgcccaccttggcttcccaaagtgctgggattacaagtgtgagccaccgttcccggccttagtacagtatgttaaattttgtgtgtgtgtgaatcaaattattttcaagtctgcaaaaaactgtatctccacaaagaggcttaaattgctgacaaatttctggggtgataaggcagcatatttaattaaataaaagtaatgtctccaaatataaagccttcagttaaatatatcttttggaattacaaagaattaaaccacttaatttataatataaattatattactttaatatgtgaagtaagaaaatgatagttaaatatctcaactttaattttctggtatggtcagacctttacagatatttccatgctattttaactttaggatataaaacagtagcagtctactaagttttctattattaaaattagtatagaaattatcaaaatactgaaaaaactgttttatgaaagcagtacctccatcactgtaacttatttcctttcttttagaatacatcttcaaaagagaccagaatttatttaaagatagtagtttgaattttaaatttccagtcaatttttcattatctcaaaattgagctaatgaagagttgatggagttaggagaacacaggtttccaactaatgccatcaaggcagcaggatcctcacatccatgaagaccaaaagaaagatgtaatggaagagagaactacggatctgtcttccatcctaatttaaccttcaggaaataccttctgcacaaaacacttcattaaataaaaaaaattacaagacagtaagcttaaaaaaaaaaaaggctaggaccacaacttgcccagttaagtctgaatggtgatgttattggttttaagcctgaagaaaagaatgtaacttctgcttctctaaaatcacatcttcatattgggctaatttaaatttaaatcacttaatgccttaacagtgctaaaaattccaagaatgtcagacatactgatattcaaaataaagtacaaaaataagtcaaatataaatatgtttgcctgtcatgctcctctttaatttaccctgatgagctaaagttgattataattataataatatatgttatttcaagtactactattctttttaagaactcatgacctaggaccccaaaacgcaattagggatatacaatttggagtatggtttacttctcagtaaagtttaatactaattgaagtactaaattgaacaaggtcaatattttgtagtactaatcccaataacttgatcccatgcaagaaacaaaagggaattttaaaggctgttttggaatcccagaacattggggttctaccttaacttcctagtattcccttcaagtctcagttgagtgttagagaacaagtcttacttttgtcattttgacagtgaggactggctttgccaattagattatgttgtggacattttccataaattttcataaattccaggttttgatagaattctatttaaagcacatacgcaatctgaaataggttagaaattacaccatctgcatccatttaaacctataattaaacatttgagttataatttaaaagtgttcaaggcagtacattgcttttcaaaagctttgtagagacagtgtaacaacaacagaaaaatttcacaaccactgctgtagtgtagtggagaaagcctgggctctaaagtcagtcttagtggcctagtttgaatcttttctctggcaatattggcttagaagttaagttaaaaactactaagggtggctgggtgcggtggctcacgcctgtaatcccagcacttaggaaggccgaggtggctggatcacgaggtcaggagattgagaccatcctggctaatatggtgaaaccccgtctctactaaaaatacaaaaaattagccgggcgtcgtggcgggtgcctgtagtcctagctactcgggaggctgaggcaggagaatgatgtgaaccatggaggtggagcttgcagtgagccgagattgcgccactgcactccagcctgggcgacagagtgagactgtctgaaaagaaacaaacaaacaaaagttttatccagtatatctattcctagttttctaagagattttaaatttttagtaaaaactgttacagaattttatcaattgtttgcatgcattgagaggatcttaatgatttttcagtttttaaattccattatgtggtgattaacattgaagtttgaatattaaatcagttttaaattcctggattaaactcatcttgattgtgatggataacgtatttctggatttgatttactaatgctttgattaggatattttttggattatgtttgccagagagattagtctgtagtcttgtaatactgtccttgtaagttttaagtattaacattatgctgtcctcataaaaaaggatgagatgtagtccctcaaataatggtgccatttctacatttaatattgagacgtttggaaaaatttagtgataaatccatctcattctggagttttcttgaaagattttaaattatggattcaatttctttattagagataggattgtttaagttctctgttggtcatttggcaagttgagtttttctaggaatttgcccacttttaattttaaagtttattggcgtacattttttcacaatgattgtttttctgtttactctcttaatgtccataattgtacacattttttctttttctttttttttttttttttagtatttattgatcattcttgggtgtttctcggagagggggatttggcagggtcataggacaatagtggagggaaggttagcagataaacatgtgaacaagggtctctggttttcctaggcagaggaccctgcggccttccgcagtgtttgtgtccctgggtacttgagattagggagtggtgatgactcttaacgagcatgctgccttcaagcatctgtttaacaaagcacatcttgcaccacccttaatccgtttaaccctgagtggacacagcacatgtttcagagagcacagggttgggggtaaggttatagattaacagcatcccaaggcagaagaatttttcttagtatagaacaaaatggagtctcctatgtccacttctttctacacagacacagcaacaatctgatctctctttcctttccccacatttcccccctttctattagacaaaaccgccatcgtcatcatggcccgctctcaaggagctgttgggcacacctcccagacggggcggcggctgggcagaggcgccccgcacctcccggacggggtgactgccgggagggctgccccccacctcccgaacggggcggctggccgagcgggggctgccccccgacctccctcccggacggggcagctgccaggcggagacgctcctcacttcccagacggggcggctgctgggcggaggggctcctcacttctcagacggggcggccgggcagagacgctcctcacctcccagacacgctcctcacctcccagacgggttggcagtcgggcagagacgctcctcagatcccagacggggtcgcggccgggcagaggcgctcctcacatcccagacggggcggcggggcagaggcgctccccacatctcagacgatgggcggcagggcagagacgctcctcacttcctagacgtgatggcggccgggaagaggcgctcctcacttcccagactgggcggccgggcagaggggctcctcacatcccagacgatgggcagccaggcagagacgctcctcacttcccagagggggtggcggctgggcagaggctgccatctcggcactttgggaggccaaggcaggtggctgggaggtggaggttgtagcgagccgagatcaggccactgcactccagcctgggcaacattgagcactgagtgagcgagactccgtctgcaatcccggcacctcggggaggcccagnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnggagggggagggggaacattttttcatttctaatattgtttatttgtgtcatctgtcttttttccagcactccttttagaggtttatcaatcttgtgaatttttggtaatgttgattttctctatttaatagttgttttaattttattaacatctactctcatctgtattgtctttcttttacttttctcttttccatcttttaaagttctatgattagcttgttaatttttagcattattgttttctaatgtaagtattaagactttagatttctaaataccaccttagttgcatctcatatgttttgatatgttgtctttttattattattccattctaaatgccctctgctttctaatttttatttttgacctgtgaattgtttagagttatgtcttaatttccaatgcttttttaaaagagccattagtttaaaatattttttatttgtatcatactccagtatttaagatttaaaaatgttctgtatggttttcaaaatagtatttcttcttgtagttgtattgcatttgtgtctactagattcaggttgttacttagattgttcaaacttttgtatgcttcacttatcaattactgagacaggtgttaaaattacctagcttaatggcaagtttgccaatttttctgtgtaactcttgtcagtttttgcccattttatacattgaggctaggtcagttaggtatataaagtgtagaatttctgtgttttcctgatgaattgcacctttttctaatgacagtcattatctgtagtgaagctttttgccttaaagtttcccctttctcttcatattaatataaataatatacctttatttggatattatttacctggtttatctttgtccatacttttattttcattctttctgtattcttgtgtttggatagtttttggaaacagcatataactggatttttctcctgtggtgtgaaatctttgtattttaatggagcagggaggccatttagatttattataaatacagatttatttggatatatttctaccatttaaaaagctttttctatatttatttttccgtatgttctcactgtcttttagattaattcagtatatttgccagatttaattttcccctctagttgtttggaagacataacacttcatttctcttcggtttgagactgcccagttgaaatttccacatttaagttatgaaagtaatcactttcttttatgctcttcctaaggatcctctcctgtgacttctagtctctgtttcttttaatttagaaaaagataatctgattggaatagtttgggtcaggtgcctacttttggatcatagctattataaggtgtggaaggtggagaatttttgtggagatgtggccactggggggttcctttccttcactccttgcatctctactaccctattcatgaactgtatattgttaacttttatcagccattttaggagatatatactacggaattgctgcaaacccaaatatatgtaattccatagtctttctgtaagactatttctctcaaatttacagggtgggggaaatactcttccttttttttttttttttttttttttttggtgtatacgaatatttgaaaactatcaaaccaggaaacacctccccccaccaccacctcccaatcccaaacctgtcatctctcattggctaagctgttccaagagctttgggttatttgtgtttttttttttttttccttgtttcttccattcttttcctttggtgcttatctgagaattcctagggaactttgaggaagtgtcatcagtatttcacagttagaatgaactattgtcctaacatttcatggaggttcaggaccatggacagatcacatgtgctacctgtctcagtcccacccacatattttagcaggttaaaatattcttctgaagtctccactggcatcattcttttccttataagttgaactcctccctatttttatcagtcatcttacagccaggtcttgcctctttaaagttgttttgttgttgttgttggtttttttttttttgagacagagtttcgctcttgtcgcccaggctggagtgcagtggtgccatctcggctcaccgcagcctcagcctcctgagtgtctgggattacaggcatgcgccaccacgcccggctaattttgtatttttttagtagagatggggtttctccatgttgtcaggctggtctcgaattcccaatcctcaggtgatctgcccgccttggaggcgtgagccaccatgcccggccttttttttgagacagagtctcattctgtcacccaggctggagtgcagtggtgcaatctcgactcactgcaacctctgcctcccgggttcaagcagttctcctgcctcaacctcccgagtagctgagattacaggcatgcgccaccatgcctggctaatttttgtatttttggtagagatggggtttcaccatgtttgccaggctggtctcaaactcctgatctcaccttaggtgatccacccaccttggcctcccaaagtgctgggattacaggcgtgagccaccgtgcccaacccctctttgaagtttttatctcacttatgtataaaatacttatcttgccaatacgtttgagttagtaaatagtttagtctctgcaaagaaatggagaattagataggttaagtaacttatttaatgatggagccaggttttgcagtcaactttttctgactctgaaaccatatctctcaattacttgatatacttcattaaaaaaataattattctgttcattatactcttggtttatgactttaaaaatatgttttaaaatcaacttataatttatttttgaaattacagggccaggagcagtagctcacatctgtaatactagctattagggaggctgaggcagagggtcacttgagaccaggagtttgacaccagcctgggcaacatagtgagatcccaaatctaaaatcaaaaacaccttacacatttgcccaattttctattatagtgttcttttttttcttactatattgtaagagctccttgaattttagggatagcaaccctctgaccatcatagattacatatatttcccccatttgtattttttctttttaacttagagtatttttaaaaataaaacttcagtacataataaaactatcttttatttagttaaatcttttcctttagggtttttcttttggcattctgtttagaaaacttcttccccatccaaagaacataaaaatatccaattctgatatatctgtggtatatgtaggttacttttagattgtctatattggaactgataattttagaacacactttaagagcagtagggtaaggaccttaatatttctttttaaatgttttcttggcttttctcctacatttattcttataggtgaccttaaaattatctttacattctctttctttttccaaaatctaatttggaagaacttttaaaaagataaacaaagtggccaaactttgcttttcacttttgtcatattttaaacatatgaaagagttaaacattaatttataatagtgttcttaccagctggatagaaatacatttacttagtacctatatttattttttctttctttgtaatttatcttctaggaagctattatgtaaaatttatggtgaaccagttgttgatttatacattattagtggtgtgataactgaggatgctcgaaagaaggcatgtaatgagattgttgctatgggaagattgagtaggtaactacccagaatgtttcccatttctagtacaatatctggtattgaataggtgctcagtaaagagttaaatacatgaagagttaaatgaataatgaagactttttttgggtgaatttactaagctcgtaatttcagccttattgatcagaatcagagatacaagtatattcagtagtcttatttataggaggcaaagtagatgaactagaatgccagttaggaatttccaataaagaatatatctatcagccatcagagtcatttgtatcgttctccccactgcttttttttttcctacaggaaaaattttatcagatgtttacttgaagaatgaaactggaataaggatagctactttagtgaatttaagttctgatagcatgataggaatgatgtgccaagtggattaatatttttgcccagtgtttaacatatccctctactgtgcacaaaagactgctacctaaatttctcactcacagattttctctactgataatatctgcctcctggccacgtactttctttagctgtgtcattagctcaccattagatcttgctctgatttagtcataattatttaaaagaaaatacacaagttgtacatgaatatatgtttatttaaaaaacagtataattaacatttattgagccattttatgcaccagatatcacattaagtattatatattcttatttaattaaaaacccatttttaacccattgagggactcaaacaggtacttacatgccagtgttcatagcactggcatgtaatagccactcacaatagccaaaaaatgtaaacaccccaagtcttcattaacttgtagttggataaaatgactgtagcataagcatacagtggagtattatttagccataagaagagtgaatttctgacacatgctgtgcaacatcactgaaccttgaaaacatgcctagtgacataagccagacatacatgattctggttatactcagtatatagaataggcaaattccgtagagacagaatgtagattagtggttaccaggcatgggatgtggtggtgggtcggggaggggatggggaattattgcttaatgtttacagagttctgtgtggggtgatgaaattgtagaaatagtgatcctaatgtttgtacaacattatgaatgtaactaaagctactgatgtacacttaacaatagttaaaatggcatattttatgttatgtattttttccacaaaataaaattcaaacaactcatgacctaggttctactattatcaccattttacaagtgaggtttagaagaaataagtaactagtatgagttcatgtagttaagtgacaggcatctagacttgaactcaggtttgtctgaccccagatgccatatttttaatcaggtggttctctgctttcagaaataaaagtggaggtgccgtctttcctattccttatttccattcttttgcacagaaattacttctgattctttgcttaattgtttgacatgtatccttttagaccctcttcctatgcatttacttaagtatttatgtgcataaatgtgtacaactttttcattaatttacatacacagcataattaacattgtttatgtgactgcttttttcatttaacagtatgcattagagatgtttctattaattcatgtgggtctacttcattttatttgctgcatatgtttcattgtatggatatatgtgggtatagtgttttgtggcatggatatcccgtaatttatataccatttcggtatttcctaggtagtttccagtttttcattctaacatacagtatttcagggcttatacatatactcagaagtggagtttttgggcaaaaggaatgtacatttaaaatatttagttactgcaatataatatttcttccacttcaaaggataccagtttaaacttctaacattgggatatgagagtccttgtttcctaggaacagagtattatcagtcttttttgcctatcccatggtcagtaattgatacttgttacattagtataaaaatctctgatttacttccagatgttagacattttccatatggttttcttttgttttgaattgccagtgcctatttttctaatattaatatttctcttagtgatttgtaaaatctgtatgttctatgtgttaattttttgttacgtaaatatttgttacaaatatatttttccaatgttgttttatttttcttaaactttgttcgtgttatcttttaccttacaggagacttaagttttcatatagataaatcttctttttattattaatacttctggctataaggctctgtgttgctaaaacagctttttccatttcaatgttatgtgaatatgctccattatattttcctagtattactgtagattttaaaaaattgtttctctaattatggcattattttttgagtcgtgtgagctatggattaaagtttttttccaaatagttcatctttctcccactcatttaaaatacctatatctgagcccatttctgcttcattgttctattggttcaggggtcagcagactgacacacaagacaaatctggcctactgccttgtttttgtaaatacagatttattggaacatagccccacctatttgtttacatactgtttttgttttgattatgcaagggctgagttgaatagttggcacagagactaaagagctcacaaagcctgcaatatttactgtggcctttacagaaaaggtttgccaacccctgaattagtctcttttctaataccagacttttttttaaataaaaatatgaatatgggctgagcccagtggctcgtgcctgtaatcccagcactttgggaggccaaggtagatggattgcttgagcccaggagtttgagaccagcctgggcaacatggcaaaccctgtctctacaaaaaatacaaaaattagccaggcgtagtagcatgtgcctgtagtcgcagctacttgggaggctgaggtggaaggattgtttcagcccaggaggttgaggctgcagtgaaccgggatcacgccagtacactccagcctgaatgacagagcaagaccctgtctttaaaaaaaaaaagtgtgtgtgtgtatatatatatatgtatatatatatatacacacacactatatatatacacatactatatatatacacacactatacacacactatatatacacatatatatatacactatatatatacagaaaaaagtatacatatatacatacacatacgaaatacacatataaatgtgtattaatgggtatgtctgtctcctccccatatcacaatattttattttatttatatacaccctggtgggggcgtgtgtcttttcctaccagatatcaaaactaataaattatagtaaaaatagagtctggtattggaaaagaaaccgactaaaacagaattggcaaactttttctgtaaaggctacaataaatactttggctttgtgagctgcaccgtctgtgtgccagctattcaactcagcccttatgtcgtgaaagcaaacgaaaacaatacataaacaaatggatggggctttattccaataaaaacacctgtgttttaaacatttttcatgattattctgcatttccttttgcacctgaacttcagaaatagttaacttcaatcagataccacattaggctttcttttaaggtgaacttaaaaaaatgttacttttcagagattacattttatctacaattttgccattttaaaatacttgattaagtaatggtactagctccagtgcctgggacatggtaggtgtacaataaatacgtgctactagtatgaattgtccaagattctttctcccatgtcttttgtctggtgatatggtttagctctgtgtccccacccgaatctcatttcgaattgttaccccctcgtattgagggagggacctggtgggaggtgattggatcatggggccaatttcccccatgctgttttgtgatagtgagggagttctcacaagatttgatggttttaaaagtggcattgcccttgcttgctctcctgctgccttgtgaagaaggtgcttgcttcccttttcaccttctgccatgattgtaagttttctgaagcctccccagccatgtggaactgtaagtcaattaaacctcttttgtttataaattacccagtctcaggtagtatctttatagaagtgtgaaaacagactaatacactggaaaatcgcctgtctgatgggtatagatattcattagcaggcatctatcaccttttatctgataacatctatgtgagttacatacatgacatgatatagattgggttgtacattttttaaatgcttaagggccagcaaacattcctaaaacattaaaatatagtaatactattccttcccctctcacaggtcttctagtagatgatcttatctaagctttaagagatcactggattcctaaagtgattttattttgtagaattatgtattctgttatgtattttagtaacagattagaactaaattggatatagtagggatttcattcataaattatcattcaacactaaatgcaaattcttggctgaaggagcttatagaaacaaaatctattttgttataaaaatgcataaattaagcattttcttagtaggaccaaactttttgtgatatgttagagtgactaacttttctttcctaatcttataatatccttctatagacatttaaagctttacttctttatgccttccccaacaactttaaaccaatgttttcctgctctttctgatctcatgctaaagactgtatgctagatgatctggttgtaaatcccagttccttcatttatttgagtatttaagggccaacaaaattgtcagaaattagtttaaaaacgtaaataaatggactatttaggtcacataacttaaattattttgtttcctcatttaaaatgaagctagtaatgccacctatcttcatagtttgttgtgtgtcttggacaagttggtatgcaaaacataaaatgtcaagcttgttaaatggagttaatctgaagattatgagtcctattctgatagcctaattgagaaaaagtgccacaaacgtgtagtttcaatttacagatgcagagataagttatagactagtgataaacttggcaaaatttccaggtaagattatctttcactggatagaattatattttgacttcatgcttaatattttttaagagcatttgcttggccataacaatacaactaacagtgatcgaaaacacaagaacatttattactaaataagtcttgaggcaggcaatcccagggttggctctctggctcagcagtgtcagtgttagattgccaggtctctgaagctcttctgtttttcctttcatggttgcaagacagctcctaaaagagcgtatcatcatagtaatatatagagtagagagagagcattcgagaaagggctttctccttgttcttcttactcattaaggaaagaccctagaagctctcaggccctttctattactggtcaaaataatataaagcaagtttcacacagatgcctgaccaaacaatggaaaaggggaaagggcttataccaattatactggcatataccaattataattcatcatctggggtaaaacaaattgccactacccagaaatagggtctgtcaatgaggaagaagggaagattagttgttaggtaactaggcagtctgccacatacgatattcatttgtttaagtaacttacaaagtaacaaggtttaaaatagtgaacaaagcggacttaatccccggaattcttgagtttttttctttctctgtttaagagatgccatttttgttgggatattattatgcattttcttgtctcctgaatgcttataagtgttagagttcaaacgaggaccttctttggaccatcagtttttaaaaatacattccttagaagaaaaacattcacttaaaaatgttttatttttacaattatgtattagtatttctagtttcagagagaaggagaaactagggtctagaggataatatacccaagttcaaatagatgggaggttctaattgtaatatcagttaataatttttgaatcttttttttttttttttaagacggagtctcgctctgtcgtcaccaggctggagtgcagtggcgtgatcttgggtcactgcaacctccacctcccggattcaagcgatactcctgcctcagcctcctgaggagctaggattacaggtgcatgccacaattccgcccgccttggcctccctcccaaagtgctgggattacaggcatgagctactgtgcccggcctaatttttgaaacttttttttatgccccacgtactgtacttaactacttttcataaattgttttattaaatcctcatgaccctgtgagggttatgaggtataggtacctgtacaggtagggaaacgaagccttttgtgtctaaataacttctcaaggtctcacagctactaagcggcaatcaaactaacacctcagggttgttagatgccaaagtgtgcacttttagcacgctttggtgtttgtgctgctggtcttcctaccactgtggaagtggttgctgggatcttagtacacagcatgttaaagtttctgttaaaatttggtactgttagttttaccttggatacaatgcaatgcaggatttttttcctgctgcgtccatgaagccaaatcctcagtgtgatgacagaaattgcagaaagaatgtaagaaaaaggtagcagcactgcccaaatgaggttatacaagaagaggaagagataatccatgaatataccgagtggggtattgagctggtatctgaggtttcagaagaggaattgaaaaattcttcaggtccagttccagacttacctgaaggaattacagtggcatacacaattccaaaaaagcaagaagattctgtcactgaggtaacagtggaagattctttaaagcttggaagacctcatggccaagattaagaatatgtacataatggactggcatatattttatttctcatgttaaagcctcttaccttgaaattaaaaaatttttaaaactgataaaacttagggcaacattaattaatgtatactcttaactgaattgttatactttttgaaaatcccgtgacttgcttgtttctctccactccaactaaatcattaactctcctaaaatgtatgtttcattctagtaagaaaacctcaaaggatattgtaggatataaatcttatttgaaaatatagccattgaaatgttttggccttttggagttggggaatgacaaatctgatcctggaatctttttcttttcagtaatcccttgtgtctgttgcctgagggacatggacaataaagcagtacatgatcctcagatatagggagaaggacaaggcatatagcttatttattagagccagcaagcatctgctcattatgtttggaattgctttctataagaaaattttgccctctactactaacttgaccaaaaatgaatttaaaattgcctgtgaaataacatcctctcaaatgtttgctgatgaaattcaagttgaaatgtattcattgcaaaagtctataaacctgtggattatatattcaaaataagacaaaggcaaataaaaagcagctgtcttcatgaatagacaaaagttgctttcaggaagtataaattatattctgcactgaccaaagaatagaagttattgcatctgtggaacatatatctggagttactatactaaagagcagggcatgaatttagactaaaatccatccagattacactcattctttaggccactgctggatacttcatttacatttgattcttggctgggcacggtggctcatgcctgtagtcccagcactttgggaggctgaggtgggtggatcacctgaggtcaggagttcgagaccagcctggccaacatggtgaaaccccgtctctactaaaaatacaaaaattagccgggcatggtggtgggctcctgtaatcccagctacttgggagcctgaggcaggagaatcacctgaacctgggaggcggagactgcagtgagccaagatcatgccattacactgcagcctgggtgcagagcgagactccgtttcaaaaaaaaaaaaaaaaaattgattctcaaggtaatgagatatcagcaaggtttgtcttgtatagtgggcataaaagaaaagttctaaattgcctctgcaatgacttgtagtcagtggaagagtatcattttccctgatcatgaccactatgctgtaaattgatataaattaactttctctggagttacacacaaataacgaagatttaagagaaccttggctgaaataaatttccagttgtttggaggctaataaataccatgcaggtgtaaccattaaaaaaattattatataaactactatattatgtaaactacaatattttagcttttctgtgtgagaaagtgatttttgtttgtttgtttgtttgagacagagtcttgctctgtcatccaggctggagtgcagtggcacgatcttggctttctgcagcctcagctcccgggttccagtgattctcttgcctcagcctcccaagtagctgggattacaggcgtgtgccaccatgcctggctaatttttgtatttttagtagagacgagatttcaccatgttagccaggctggtcttgaactactgacctcaagtgatctgcccacctctacctcacagggtgctaggattacaggcatgagccactgcacccagccaaaaatgatttcataaaaattgttaaaaatctgtagctgctttgttaccttcattaactctttgtgattaaagttgcaagtttattgcagtaatgggaatgtgttagattaatgtttaggtgagttatattttaaaatctgtgtagtatatttatagttcacccagtaattagaacaccgtatcttaattccttattgttcctctagtgtgtcacaggaggtagagtttattgatgctgggattgcggatgtgaaaataacattctcagggtatcttatgacttaaaaggccgattttttaaccagtgatattttctgtgattgtctataatttggtttaaatttatacttaataagggccagaatacatggctatattcttagtatcctttagaacattatttcttagtcaaagtttatgaggcatctagcaacattttacatgatatataaaattggaggtaaatgtgctttttcttgggaggaattcatagccttaaactattcttggaggagtgtatgattctctctctgtactttcccctgtttttcaattattgcttcagagtattttttttttaagtttgagtactttaaacagcatagagaagtaaatcagtgacttaaaaattcattattttgtcctatatgcacatgttaatatatcattacatttcagtggcagtatttttcatttcttgttttacttggttaattttcaaatctctgttctttcattatttgtatttcttctatctcttgaatcattgttacccacttcatggtatttttcagattgtattattctcttgagttcttgagggtactaatcttcccatttgttttgattgccaactttccccattttggtgttctatttcctcatttagtttaattttttttcaattgtgagctcacttttgactgaggtagatttttctgggatgagttcctttccctctgaatgcttttgcttctgtgggataggaactagatttaatattaatttcttggcagaggatttctttaccacatgggcagtatggattcagactccaaaccctcatgttttcctcttatgctgccccccaccaacaccctaggttgcttcactggacaagaatcagaatttttattattccctgacatggggaaagggtcctggttttaatgagaggtaatcattccagtcctctacctctgttaagccaccagcaataccttcattcgttgtggatcgaatgatccctaacaggcacctgttagggcctgtgtccaggacttttgcttttgcagcatctattcctgtgtttaaagttttgattatttttctttatttctaattcttggggatttctcttcctttcttctccttcattagtttgattgtatatttaaaatctctcatggttgtttgttttatttagcatttctatgtgtatttgtgtatgtgtgggtctgtgttactgcttactgtgttgtcaagcccatatatgcacaaggactattttgaaactttgtttaataataactcaggctaggtgcagtggctcaggcctgtaaccccagcattttgggaggccaaggcaggcagatcacctgaagtcaggagttcaagaccagcctggccaacatggtgaaaccccgtctctactaaaaatacaaaaattagctgggcatggtggcacgcacctgtaattacagctacttaagaggctgaggcatgagaattgcttgaatcctggaggtggaggttgcagtgaggtgagatcacaccactgcactccagcctgggcaacagagcgagactctgtctcaaaaaaaaaaagaaaaactcaattattctgttaaaaaattttaatatgaaagatactggtgcttaaaggtgaataatactgtttttaaccttcatgtgtctgtgattgtaaaaagtctcaaatataccacctgtaactttgttctttgtatattttaactcattaatgtatagtcacagtattacagaatttagacacagagcctggcataattgataacagttttaacatggctttattaactactcttttctcattcttccctattctcttctagtctcccttttgcatggattattctcttttcctgccctactgttggttccagattcttatgttattccattgcagataagggagaagaagaatggtagaatggaaacccactggagaaattcgagacgaaagagaagaaggaaaataatgggtgaagggaaggttaaaatttcttgctccagcactctctgttctcttccaggccttatcttactccttcagactctgtcttcatttctcatcttttcttctcacttatgcctctgcctttccactctctctttctattggacactttctttctgacaccatggtagctatcactagtggcttgcaatctatgttcctaccacctggccagtagttgtagggaggaaggaggttggatgggagaattgaaggagactctgttgttggatcttgggctacacagagtagggagaaagatgtaccttgcaaggtataccaggcattttgtattggaactttgagttctttttcttctagaattattcatttatctgttcattaaacagtaaacaatgaaccccaggcagtgttttaggaactcacatagaaacatactttgttcttttcagtttcagagatcttttttatttaacttagaattatttcacttttgaatctcttaatttcagcatctgtatttatttcaaaccttttgggaataaagaggcaacttgttatgttggaaaggcttttggagcctgatagatctgagttttaatcctgtctttgccacttactaggtatttaatattcaggaatttatattatctttctaaaccttttttttttttttctgtaaaatgggaagttcttagtatggtacttggcttagatggctcccaataaaataatgcctgctaagattattattatcacacagaaagtttgtccttagtaccaaatcaattttttttgtttttgtttttgttttgccaagaagtacttttaaaactagagtacattaagacaatgattaaaggattctgttttgtttatttttatatcacattcttgaagtttggacaagttgtcataatttacataaaactattacttctaagctattacttctaagttggtgtgtccttctctgagctagcagtgtcatagtagtactgtgacttactagtcaagtttaggccagcaagtcacatactatgtttaggccttataatattaatgagggttaataatatgtaccttatggattttatgataactaaattacgtattagtttggaagctaaccgtgtctagttgtgagctaaaaaccaagtatttagtgtaagattcaggcttgttcgggaaatacaaggcagggatgcaatcaaatgtcaggaatcgccagaacaccgggaataaggaagctatcggaacacttccttttctcattgtctttgcctttctctatgcatctgctttgtttttctctttcattatgtggaactgattttctcttttgcttttagagtcatagtaggatgtttgcctcaacacttgctgagtttgtatgttaattcttgtaatcatctgcagagactgactctgagttaggtttgaaattacttgtcccccagcttaggtcagatttccatattggttcagttaacactggccagttaaatggggcctcacaatatgcacttggcccacccctgagaactgagggggagattttcagaaaaagggagggaggcggcgtggcagatacctcagaaagtgtgtgccacaagtgaagtcatttaagtatacagcactgaagaaagtcaaggtattgttatcattagatgatgtagtactttgtgaaatatcactaatagcatctattcagaatagcatgtcatttatctaccagcgcaacagatctgattagtcccttatatgcccttgatttttgtcaagggcagtagcgtagtgattattatatctttttcaattgttgtaatgaatataaagtcttttgcaatttgtcttttatattcaaaattattttaatcatagagtgtgcttgaaatgaactttttggtggatataatttttaagaacatatctttgtataaaagcacttttgttaaatattgctaatgaacttttttcaggaataagttgaacgtataatcagttttttttcaaatgcagcatagaaataagaaatattcaagtaattgatacatgattcttttggttctatcattgaatttctgaactctcagctataacttgttaaggcatcttttaaattatcccagacattatgaatggagtgctcagtaatgatttgtataacccttagctttgttttgattcattttctttatactacttagggtatcatcatatgacaagataactgtatacccctgtatagacacttaatattgttaatatttattgttctgtgttcatatttaccccaaattagaatagtcaataggtataatttacctgaagtgtcagctctgcagcacactttttctgaatttctgatttagtctgggtgggggagctaggtttcggtttaaggatgcctttgttttgtgattttgtgatatagtagaaatcaaaactgcttttaagtatagaagactggtgttacaattatttggggggatagtcaggctcccattttcagttcttttttttttttttttttcccggacagggtttcaccctgttgcccaggctggagtgtagtggcgccctctaagcttactgcagccttcatctcctgggctaaagtggccctcccacttcagtctcctgagtagctgggactgcaggcacaagccaccgtgtccggccgatttttatatttgtatttttgtagagacagagttttgccacgttgcccacgctggtcttgaactcttagactcaagcaatccgtctgctttggcctcccaaagtattgggatacaggcgtgaaccaccacacccggccccatttttagttctagtaggtctgtatgtatctcccaccttgtcaaacttgtgtgtaattttttttaaaaaaggtttcacccaccttgtcaaacttgcgtgtgatttttaaaaaagttttcttccttttttttttgagacagagtctcactcaatcgcccaggctggagtgcagtggcataatcttggctcactgcaacctccgcctcctgggttctagtgattctcctgcctcagcctcccaagtagctgggattacaggcgcccatttactaccatgcccatttaatttttgtatttttggtagagacagggtttcaccatgttgaccaggctggtcttgaactcctgacctcaggtgattcacccgccttggcttcctacagtgctgggattacaggcgtgagccactgtgcccagcatcttcctattttgttttaaaacagctttactgacatgattgacataccaaacaattcacctgtttgaagtatacaattcagtggttttgatatcttactaatttttaaaaattgtggtaaaatacatataacaaaatttgccattttgaccacttttacatgtacaattcagtggcattaaattatatttataatattatgtaacaaccatcacatagctgtctctaaaactttttcattatccaaagagaaattctgtaaccattaagccataagttttcattctcctgtcccccagcccttagtaacctttaatctactttctgtctctatgaatttgcctattctagatatttcatttaagtgacataatatatttgccttttaatgcctgtcttactttacttagcatgttttcaagatttatccacattatagcacgtatcagaccttaacttcattttaaagctgcttaacatactggtgtgtgtgtgtgtgtgtgtgtgtgtgtgtgtgtgtgtgtgtgtgtctgtatatatatatcacattttgtttatctgttcatctcttgatggacatttggataatttccaccttttggctactgcgaataatgctgtcatgaacatcattgtacagatatctgtttgaatctcttcttttgattctcttgggtacataggaatggaattgctgggatatgttggtaaatctatgtttagctttttgaggaactacccaactgttttacaaagcagcagtaacattttatatcccctctagcaatgaatgagggttccagtttctccacatcctcttcatcacttgttattttctgttttatttaattaatttaaaaaattcagctattgtagaggttgtgaagtggtatctcattgtggttttggttttcatttccttaatgattagtgatgttgagtatcttttcatatgtgttttggccatttttatatatcttcatatatatattattttacttttaagttctgggatacatgtgcagaacatgcaggtttgttacataggtatacatgtgccatggtggtttgctacacctatcaacctgtcatctgtgttttaagccccgcgtgcattagttatttatctaatgctttccctcctcttggcccccaccccaacaggccccagtgtgtgatgtgcccctccctgtgtccatgtatgttctcattgttcatctcccacttatgagtgagaacatgtggtatttggttttctgttcctgtgttagtttgttgagaatgatggtttccagcttcatccatgtccctgcaaaggacatgaactcattcttttttcatttttatttatcttctttggagaaatgtttattaaaacttttgcccatttttgaattggggttttttgttgttgacttgtaggagttctttgtatattctggtattaattccttagtagatacatgatttgcaaatacttttcccattctatgggttgtcttttctctctctccatatgaattttaggatgagtttttctatttctgtaatgccattgtaattttgatagggattgctttgaatctgtggattgctttgggtaacattgtcatattaacaatattaagtcttcttccaatctagaatactggatgtcttttcatttatttaagtcttctttaatttttatcagcaatattctgtagtttccaatgtacaagtctttcacctcattggttaaatttatctttaagtactttattcttttggatgctgttgcaaatggaattttattattattaattcttttattactgctttttaagagacaggatcttgctgttttgcccaggcaggagtgcagtggtatgatcatgactcattgcagcctcgaatacctgggctcaagttatcctcctgccttagcctcctgtgtagctaaggactacaggcatatgccaccacgttcagctaatttttttctctttctcttctctgttgtgtcaattccagatagaaatcctctctctctttcctttccttacaaaacacaaatacagaaatccctagtggacttgactaagcagtgtggaatacccgttttcgaagctagttagagctaaatgcatacttttagatcagagcaaactgtatgacctatctgtttcactcccctaatgctcaaatatgtatctttgatcttatcacactctggctccatggaaggaagagagaagacagctacacccatgtattatgatcctttaatcatatcagttcctttttgtgttagaataaagagggacctgaggagaggaaaggagacacgtttaaacttctcttagaggcagcagaatatgtaattgctctaatcattaaacatttttaatttaccagttaaaattattaataagattacatctaaattcctttaatgaaattcaaagccttttccattttctagctcttatttctttgcatttcttctcacaattctttaccatggattatttaccattaacaccccaaattcacttcctcgcctctcttcatttgcttatattgttccctcttgcctggagtatctacattgtcaaacccaggcaaagcagcaagattctatttagtcctcaaggttcaatttatctgttatctcctttgtgtgaagcattagtatccacataattagtgttctattctcactaattctgaagcacttaaaactttatcacattattaaattttgtgtctgcatatatgcatacttgtctttcttgaatgaagactaagagtttcttgaaggaaagtatcatatcataatcaaatttttaaaaactccagtcattgccaccatgctttgtatactgtagacgtttgctaacgcattttaaaggaacaaatgaatgagtgtgaaagaaacatgagtttacttggtgggaattgctgaagagctctctggtgccatgaaatctgcttttattttttatatctttgtgttaaaatatatatttaatgctgatagaatgttaaaactggtctcatttttacatcccccttgctttgcattcttttatatgttatttggagtttaaacatacatttctcaccaaaaatgtaattcatatgtaagaaataaaagaaaatatattgttttcttagggaaattaaatctgtttattctcatatagtttgggatttttaaatattttgacaattcttaaatcataatttgaatttttgtgtaactaagaaacaccattatttatatctaatttgcatcacattttattaagaaaataattgatattgctctttcatttcagctaaaccatttacttctaaagtgaaacaaatgcgattacatagagaagactttgaaatattaaaggtgattggtcgaggagcttttggggaggtaagagattaagatttgatagaaggtctgctaattattttggaagctgttgaacacaattgtgagagaatttaacaagtattgttacttgttacttgttaaacaatatttgttaagttgtgattgatgtgattgattgtgctgttgtgattgatataatagctcagaaaataaagtgttgaatgaaatatttctactgggaattttttctaagatcatgttttcacatgtacttcatatgtcaatgtaaatatgagtgtgtttctagactcttcttttgttctgttggtctgtcttattctagtttataaggaatcttgatacctggtagtgtatatactctgacttcctttttctccccctctcccatctctcctcccctgatattttcatagtattttattttatgtatgaatgtatgtatgtatgtatgtatgtatgtatgtatttatttatttatttatttttgagatggagtttccctctgttgcccaggctggagcgcagtggcatgatcttggctcactacaacctccacctcccggattcaagtgattctcctgcttcagcctcccgagtagctgggattacaggcgtccgccatcaggcccagctaatttttgtatttttagtcaagttgggtttttgccgtgttggtcaggcgggtctcaaattcctgacctcaagtgatctgcccaccttgtcctcctaaaggatattttcatagtattttagaatcagtttttcaatttcgactcacttgtaaagatacacatgcacgcgcacgcgcgcgcgctctctctcgctctctctctcgctctctctctcgctctctttctctctctccctcctctctctctcctctctcttctcttgtctcctctctctctctcttttttccaggaatttgggtaaagtttgtattgaaactggagatcagtttgcagacaattggcattttaacaatactactgagtcacctaatccatagacgtggtatattcttccatttatttagggctttataaatttatctcaataccgttttgtagtgttcattcagtatagaagacctccacatctttcagcagatttattcctggatatgtgatagtcttgatattattttaattagaattgtttgtgaaatttcatttcttaattgttttcttggtagtagttagaagtagaatttaattttattgatcttataccttgttatacttttcaaataggaattttgttgaattttattaatactttatctgcatcttttgggacactcatggtttttctcctttactttgttaatgtgttgaattatgctgttatattttcagatgttaaacaatctttgcattcctaaaataagcccaactgacttgtgatatgttaccccctttatatgtaccaacggacttcatgtgctttttgtgtgttgataggatctttgcacctgtgttcttcagataaattggcctgtaattttcctacttcgaatgttctgatcagattttggtatcttggttatactggtcttacaagaagagggaaatgttccctcctttagtattctccagaagagttagtgaagattgttattatttttttcctgatatgcttacaagatttaactggtgaagcctctgagaccagaggttgctagtgaaaaagttttaaataatgtttaatttctttaatggaaataaaatgacttgttctgtgtatgtattagttttgctaggtttatccatttcatctaatttttaaaacttgttactgtattgatcatttctctctctttttatttttcttgctaggggtttatcagtttattatcttttccatgaattaacttttggccttttgaattttgttattgtagatttgttttctggttctttaatttctgcactttataatattttctttggttttatcttgctattctttttctgatttttcaaaatagcattgatatctagcccttcttttcaaacctgtgtttaaggctatatgttttcctctaagcatttttagctatagcagacatattttggtatgtcatatatatgtatatattttttgcttaaaatacagtgtgattttaaatttttgtttttgatctatgaatttaaagttcattaaaaaaatttctaaacatttagaaaattttctagtttttcttattgagttataatgtaatttaaaagtggttagaacgcatactctatgtgattagaatcctttgaaatttgctctgcagtgttctgtaaatatcagttaggtcaagttcaaaatcttgtttaaatcttccatatccttactgtcatttcctgagggaggatttctttatttctttcagtttttgctgataacaaatgtacagttattatatcttcctggtgattaactcattcgctattatggagtgttcccatgatctctatagtgttttttgatttaaattttccttaatctgatattagtggaactactccagctcttttttattagtattttcattaaatatcttattctgtccttttcgttttagctttctataagcatatggtatgtttactgttcagtagaatataattagattttgttttttaatcagttaacatgtaatgatgttactacttgttttatatttgttttgctcattttgtgttgtttttctctcatttcttgcttcttcttcgattaatggaatattttaaatcatacctcccaattttatcttcttttatgtgttcccctggcaattaccacatgtatccttggtaaatttatattttgccaattcccatttacccaatttctaccttttttttttcattgctgtcatatattttagttccatttatatttgaaaccccaacatatatattgtacctatttatttaggtttacccacatatttgtattttctctgctcttcacattcatgtacttccctttgggatcattttccttttgacttaaaaattccctttaatagttctttttaatatttagagacaaagttttgctttgtctctctggctgcagtgcagtggtgtgatcatagctcactgcagccttgaattcctgggcccaagtgatcctcctgcctcagcatcctgggtagctaagactatagctgtgcaccagcatgcttggctaatttgtaaattttcttgtagtgacggggttttcctacgttgcctaggctggtctcaaactccaaggctcaaggagtcctcctgcctcagcctcccaaagtgctgggattacagttgtgaggcactgtgcctggccctctttagtatttcttcagtgtaagtctgctgataacaaattatctgattttatttgtctgaaaatatattttttgaggtcctaaaattaaaaaaaattgaagtatattatggtatacacacacacacatacacacacacacacacttcagtttcaaaaaatatacacacatataactcgatggatttttacatttttttagggtacaaaacaagcaatagcattttattgttatgccatttgaccatagcattctttttttattttttatttttttatttgagatggagttttgctcttgtcgcccaggctggagtgcagtggtgcaatcttggctcactgcaaccactgccacccgggttcaagtgattctcctgcctcagcttcccgagtagctgggattacaagtgcccactaccatgcctggctagtttttgtatttttagtagacatgtggtttcaccatgttggtcaggctggtctcgaactcctgacctcaggtgatgcacccatcttggcctcccaaagtgctgggattaacaggtgtgagccaccatgcctggccttattattattattttttttaatttttgtgggtacatagtaggtttatatatttatggagtacgtgagatattttggtacaggcatgcaatgcttaataattgcatcatggaaaattggatatccacgttatttcaaaatggacattcaagcatttatcctttgtgttacagaaaatccagttgtactcttagttatttcaaaatggacaactaaattattattggtcctttgtgttacaaaaaatccagttgtactcttttagttatttcagaatggacaattaaattattattgactacagtccccctcttgtgctaccaaatactaggtcttcgtcattctgttttttttgtactcattaatcatccccatctcctttttctccacatcctcaccagcatttgttattgcctgccttatggataaaagtcattttaactggggtgagatgatacctcattgtagttttgatttgcatttctctgatgatcagtgatgttgaccacttttttcatatgcctgtttgccattcgtatgtcttttttatagaaaggtctattcaaatctttagcccattttaaaaattggattatcagattgtttcctatagagttgtttgaattccttgtatattctggttattaatcccttgtcagatgagtagtttgcaaatatttcctcccattctgtgggttgtctcttcactgtggtttgattctgtctcttcactgtgggttgattgtttcctttgctgtgcagaagctttttaatttcgtgtcatcttatttgtccatttttgctttggttggctgtgttttaagaaatttttgtgcagaccagtgtcctggagattttcaccaatgttttcttttagtagttttatactttgaggtattagacttaaatctttaatccattttgatttgttttttatgtaaggtgagagatagggatcaaatttccttcttctgcatatggttatccagttttcccagcaccatttattgaagagactgtcttttccctaacattcttggcaccgttgttgaaaattagttccctgtatgtatgtggatttccttctgggttctttattctgtttcattaatctatgtgtctgtttttatgtcagtaccatgctagtttggttactgtagctctgtagtatcatttgaagtcaggtaatatgattcttccagttttgttctttttgcttgggatagctttggctattctgggtcttttgtaattccatataaattttaggattattttttctatttctgtgaaagaatgtcattaatactttgacagggattgcattgaatctgtagattgctttgggcaatatggatattttaacagtattgattcttctaacccatgtacattgaatattttttcagttttttgtgtcctcttcagtttctttcatcagtgtttcatcagtatattgatgtatgtgtgtgtgtgtgcatacctcacatgtatgtttatatacttcaattaaaaaatacacatagataacttgatgaatttttacatttttttgtaggggacaaataaataatagcattttattcttatgccatttgaccatagcatttgtgttctgtagttttcactgtagacatcttttacttctttggctgatttaaatcctaggtatttaattttatttgtggctattgtaaatgggattacttttttcatttctttttcagattgttcactgttggcatagagaaatgctactgatttttgtatgttgattttgtatcctgcaactttactgaatttgttcatcagtttgaataagtttttttgtgtgtgtgtgtggagtctttaggttttccaaaatataagaccatatcatcttcaaacaaggataatttgacttttttctttccaatttggacacctttcattctttctcttgtttaattgctctagctaatacttaaagaactatgctgtacttgaatattgatatctttctctagatttggaaagttcttattatccctttaataaactttctactcttattttctctaccttctctttaaggtcaataactcttagatttaccctttgaggttgtttcctagatcttatacatgcttcattctttttcattcttttgtcacctctgtgtattttcaaatagaccattttcaagctcactaattcttgcttctgcttgatcagttctgctattaagagactctgatacattctttaacatgtcagttgagtttttcaactctagaatttctgcttgatttttaaaaattattgtttgtttatttatttattttgagatggagtcacactctgtcacccaggttggagtgcagtggtgtgatctcggctcactgcagcctccatctcacaggttcaagcgattctcctgcttcagcctcctgagtagctgggactacaggtgcataccacgcctggctaatttttgtatttttaatagagatggggttttacagtgttggccaggctggtctcgaactcctgacgtcaggtgatccgcctgccttggcctcccaaagttttgggattacaggcatgagccactacgcctggccccgtttttaattattttaatctgtgaaatttatcttatagaatcccaaattccttctctgtgttatcttgaatttctttgagtttcctcaaagtagctattgttttttaagatggactgtcactctgtcacccaggctagagtgcagtggcacgatctcagctcactgcaacctccgcctcctaagttcaagccattctcttgcctcaaccttctgagtagctgggattacaggcgtgcaccatcatgcctggctaatttttgtatttttttaaaaatagagatagggtttcaccatgttggccaggctggtctttgacctcaagtgatctgcctgcttggcctcccaaagtgctgggattacaggcatgagccactgcacctggccaaaatagcttttttttgagacaaagtctcgctctgttgcccaggctagagtgcagtgacacgatcttggctcactgcaacctctgcctcccaggttctgtgattcttccatgtcagcctgctgagtagctgggattacaggcacccaccaccacccctggctaatttttatatttttagtagagatggggtttcaccatgttggccaggctggtctcgagctcctgacctcaagtaatccacccacctcggcctcccatggtgctgggattacaggtgtgagccaccgtgcttggcccaaaatagctattttgaattctgtgtctgaaatgtcatgtgtctctgtttctctaggattgatccccagtgccttatttagttcatttggtgaggtcaggttttttggatagtgttgatgcttttagatgttttcggtgtctggacattgaagagttaggtatttattgtagactttgcagtctgggcttgtttgtacctgtctttcttgggaaagcattccaggtatttgaagggacttgggctcaaaggccaataacacagtggtttttgcagtcttatataggtattgtcttggtggtcttggataagatccaaaagttttctctggattaccaggcaaaaactcttattcttttactttactttcttccaaacatattgggtctttctttctgtatcgagccacctggaactctggggatttggtgatacaagcacccctgtggccaccatcactgggactgtgctgggtcagacctgaagccagcacagcactgggccttgcccaaggcccttcccttcagggtggtgagtttccccaggccccgggtgtgtccagagatcctgtctggtagcctgcgattctagttaaaaaccttagcaatctacctgatgttttatttttactacgtctgtgctggcattcaaaccacaatacaaaaagttcttcccgctcttctctcccctttccacatgcagaggaggagcgtctctctgtggccagcaccatgactgttccatgagcagttctgccaggccacaccaccagtgttcacttaaagcccaagagctctttcatcagcttgtggtgagtgctgccaggcctcggactcacccttcaggcagtgggcccccctctggcccagagccggtccagaaatggtgtccaagaggctggacttggggatctgaaaagcctgccttttgttctgtcccactgtgatacctgaaagcaagacaaagttccctttacttttccctctgcttttctcagacagaaggagtctttcacagtggccaccacaactgggaatgtgctgggtctcctttgaagcaagtagtctcagacctcaaggcccacagcatactccctgggtattgctggtgcccagtggcaattcattgcccaagaactctttagtcagcaggtgatgagtcctgccaggactggatccttctcttcaaggcagccggttcccttttggcccagagtgtgtctagaaatatcattggggagctagggcctggaatgggggcctcatggctctgcctggtgctctgttctagtgtggctgagctagtatccaagatgcaagacaatgtcggggctgggcatggtggcttacgcctgtaatctcagcactttgggaggccaaggtgggcagatcacctgaggtcaggagtttgagaccagcctggccaacatggtgaaaccctgtctctactacaaatacaaaaattagctgggtgaggtggcacgtgcctgtggtcccagctactcgggaggctgagacaggagaattgcttgaacttgggaggcagaggttggcagtgagctgagatggcaccactgtactccagcccaggcgacagagtgagactccgtctcaaaaaaaaaaaaaaaaaaaaaaaaaaaaaagatgcaagacaaagtcctctttactctttgctcttctgttcttaagcagaaggaaggagtcactctggttcctgtgagctgcactgcctgggttgggggatggacagctgatgcctccctaggtcatgtgcaaccctagttcactggctctaagcactaggagttgcctaggaattggagtccttgttagaacccaacttgactgccattcaggtttacctaggaccccagagcacttcagcctgcagtggtgatgcttgctgggaaactcgttctgacttgctgggatgggtgactcccctttggctagagctagtcaaatgcccctccaagcatggccactggctgagcccagcagggcttcattctctgctgtgacagggcagcactgagtctctgcgctttccctccctgaagtatattctctctctctacctcatggccattgccagggaataggaaataggggtggcatccatgattcaagactgtctctcctgtcttcctcaatgcctttttttcaagatatgaagttaaaaccaggtactgtgattgcttacctgagttttggttcttgtgagagtgcttttctctttgcagatagtttttaaaatttgctgttccagcatggtgtgggagagcttgaatggtgtaggcttctattctgctaccttgcttcacccctggccatagccagatttttacatttgtatgagtttgtataaaccatcaagcagatcaagatatagtacatttctagcattctgataggctcccattgcccccccccgaccatgtgaataatcacttccttgttaactattctgttaactattctgacttctatcaccatagattttttttgcttgtttttgagttttatgttaatggaattatgtaatatatactctggtttctggcttcttgtgctcaacagtacacttttgtaattcattcttgtttttcactggttcacagtatttttcttaatatgaatatgtcacaagttatttattaatggtgttcaagttgtttatggtttttggccattatgaataatgctgttagaaacatttttgtgcatatcttttgggagctatgagcacttatttgtgtttgatacaaatttagaattgctgggtcacagggtaggcttatttagctttagcagataatgccaaataacttcccaagtggttaatgatttatattcccgtcaacagtgtataaaggtcccagttgctttataacctcgctaacatgggtctttttttttttttactttagtttttttttggtgagtgagtggccgtattgtgttatttcatttcgcatttcttgatagcgcctacatttgaacacattttcatatcacctcacatcacttagatatcctttccatgaagtacacattttggggttgggtaagtaggagttgcatgggtgagttgtctgacttatcgtcatattctagattcaaatctgttgttggagatgtgcacacgtgtgtgtgtgtgtgtgtgtgtgtgtgtgtgtgtgacaaatcttttccgtggcttggcatttccctctctccaaagatcttgtattgaacagaaattcttaatgttaatggagtgcaattaattaaacttttacttataattagtgatttttgtgtttttaagaagtttttttctaacctcaaggtcatgaacatattttccaataattaaaataaattctttctttgatctttcaagacaaacctttcacatttagatgtctcatctctctggtattgactgttttgtgttttctgaggtataggtcaaagttcactttattctttgtggtttctgattattgtttgttttaaagactacctttttcacatttaattgcaaagaaacttttaagttgggcccctatgtgtgccacttattaattttttaaaaacagttttcaatgtagaagtcttttttaaaaacagttcttaaggtcaggggtacacttgcaggtttgttaaataagtaaacttgtgtcatgggcatttgttgtacagattatttcatcgccctattaagctcagtacccatcagttattttttcctgatcctctccctcctcccaccctctgtcctctgacaggtcccagtgtgtgttgttcccttctatgtgtccatgtgtactcatcatttagctcccacttacaagtgagaacatggggtatttggttttctgttcctgtgttagtttgcagagcataatggcctccggctccatccatgtccctgcaaaggacatgatctcattttttttctgtggctgcatagcattccatggtgtatatgtaccacatttaaaaatctagtctaccattctgggcatttaggttgattccatgtctttgctattgtgaatagtgctgcagtgaatgtatgtgtgcttgtgtctttataagagaacaatttatattcctttgggtatatacccaagtaatgggattgttgggtcaaatggtgtttctgtttttgttctttgaggaatatttttcaatgtagatgtcttgttcatgtttcactaaatttatgggtctgatttcttttttttatctgccccaatccaattctttttttatttaatttttttgagtctgatgttttttagtaagctagtaaaggatagttttagaattctattttttattttcttgctgtggtagaaatacaattggctttaaaaatattaatcttttatttaaggaacttgattatattaacttcttctagtttgtacataaattttttgaattctctatatgttcatcttgatcttcaattaatgatagcatttgttcttcccaatatttattccttttatttctttttcctctattattgtaatagagaagatctttaataaaatgttgaatttaagtgctgaaagtgggcatcatccttgtctggtttgtgaatcaggagaaagtgttcagcattttactaatgattaagaagtttgctactactacttttcaaaaaaatgattactctttttccttcttgccctagtctgtagatagcttttatcattaataggagctaacttttatcaaaagctccatctatatctgttttgatatgattttactatgatatttggctaatattgatattataattttcaaatataccaatattacagtactatagaaattcagttggccttttaatatatagttgggtttgatttgctgatattttgtttataatttttttgtttatgtgcatgagacagattggcctttgtctattttataatcttacattttggaacgatattatttaagctcataagacaatttggaaagtgttttttctcaattctctgaaagcatttgtgtaagattggtgttatttctttttaataatgccaagaagacctcactggtgaagccatatgaacaaggagcacaaaatctttttgttgtttgtttagatcttctgaggaaaattttacatacattgaaataccctaattgtatatatacagttccttgaggttttggaaaatggatcacccttatcaagatacagaatatttctgtcaccctgggaagttttcttcctcgttggttccccacagcttccatcaaatggcaaccgctgttctgagttttaactgtagattaattttgactgtttgaaaatttcatataaatggaattatacagtatgtactcttttgtgtccacctttcactcaggataatgtccaagactcatccttgcaattgcatgtattaatcgtttttattactgagcagtataccattttatcaatgttccacagtttattcattttcctattgatgaacatttgtttccaattttttggctattagaaataaagctgatatgaatattctcctacaggtttttatggaaatatgtttttgttttctttgggtaaataaccagaagtggaactgctatgctaggcagtagttgcatgtttaaatttaaaagaaactggcatactacttccagttggattgtaccatttttatattcctacttatgccctgttcttgcctaatattttttgttgttaatattaattttagccattctagtgggtatgtagtggtattctgtggtcttaattttcattgttgtttgcatttgtgttatatatcagcataatcatggggtttttgtgtgcctattaaccatttttatatcttcctttgtgaggtatatgttcaaattttttgctcagtctttagaaattgaaatgtttgtatttttattgttgagctgtaggagttctttgtcagatgttttagcaatattttctcagactgtgtggtttgttcattttttaaaaaaattatcgtttagagcagaagttttcaattttgaaggtataattttatcaattgttttctattacagttagtattttctgtgttctaagaaatccttgtttatactcagatcacaaagatatctatgttttttttttctaaaagccttctatttttagctactatatttagattggtgatccatctcaaattcatgtctgatgtgatatcggcattgagattcatttttttttttgtatttgtagccagttgtttcagcaccatttgttgaaaaggctgttttccctttattaagtcattttagcaccttagtaaaaagaccatgtgctctattgctgtcctctctattctctttatctcattgtctgcctttatgctaataccacagtcttgattcctatagctttatactaatgttgagattgtgtaatgaactctgagtgtatggagtcctactttgttctttttcaagattgttttaattattcgaagacttttgcattttagaatcaatatatcaatttttataaaagcctactgatgtaattgagtatgcactgagtacatagataaattttgggaggaccaatatcttaataatgttgagtctgttagtctctgaagcagatatttctctttgttttttgaaggtctaacttaattttctcaaggatgttttatagttttaatgtataggtcttcttcatattttcttaaatttattcctaagtatttaagttttttgactctgataaatgatatctttaaaatttttacattcaaacactatgctgctaatatgtagataatatagttgatttttgtatcagactttatttcctacctactaaagtcatttattttttagtagttttgttgttattttggtccctggtttcttagtttttctgtgtaagcagtcctgctgtctgataataggattttcagaatagggacgcatcctttttactttgtagactttctatttctttgtcttgacttactgcactagctcggacctctaatacagtgttgaaatggtgtcacaagggtggacatccttaccttattcctgaactgagggaggaagaattcagtgttttactgttaagtatggtgaaattattgttaggcaatattttggtaggtgccttttatctgattgaagatattgtcttctgttactataatagtttgctgagcatttaaaaaatatagttattgataggtgctgaattctgtcatatgctttttctgtatctattgaaatcattatatggattttttctttcagttagttgacatagtggattactttgattaattttttaatagactttatattttagagcagttttaagttcacagcaaaattgagcagaaaatacagagttcccgtatacctcctgttcccacacttacccaatcttcatctcccaaaacactgaaccagaatggtacatttgttacaatccaggaaactacattgacatattattattacccaaagtctgtagtttaccttagggttcactcctgatgttgtacgttcagtgggttttgacaaatgtatgttatgtatctgccattgtagtatcaaacagagtagtttccactgccctagaaatcttctgtgctctgtctgtttattcatttctccctcaccactgacccctggccaccactggtctttttttataatctccatacttttgccttttccagaatagttagaatgatacagtatgtagcctgttcagattggcttttctcagtaatatgaatttatggttcctctacatctttaaatggcttgatagctcttttctttttagtgctgaataatattacattgtctagatacactgtgtgcattgaacattccatgtacacttgaaaagaatgttttttctacatttattgtttgttgtgttttatacatcagttagctctagacggttgatgcagtcattaagctcttccataatttcctaatttttgtttagttctgtcagtgctgagaagagggttttaaagttttcaaatataattgtgggtatgtctgtatttccctgtagtttcctctctttttattttatgcattttgaaacttaattggtgcatacatgttccaattgttcatctttcttatacactgagcctttaatcatgcaatgtttcccttcaaggtagtactcgttgtcatagtctgatttgcctcagtatagccagctttcttatgatattaacctttttcatatagttaggttttatattttttacatattctgataatctgtattttaagtggactgtttagactgtgggtcaacaaactttttctgtaaagtgacagtaaatattttagatttcacagttcatatgttctctgtggcaactactcttctctgtcattgtggtgtgaaaactagccatagacagtttgcaaatgaatgatggatgtggttgtattcaaataaaattttatttttaaaaacaggtaatggaccagatttgaccttcaggccatggtttgctgaaacctggtttagatgttgtgtttgtttgtttgtttgtttttttaatgagacagggtctcgccagtgtagtggtgccatcatagctcactgtagcctcaaactcctgggctcaagctatcttcccactcagcctcataagtagctgggactacaggtgtgccccaccatgtccggctaattaaaaattaaaaaaaaaaatagagatgggggtctcactttgttgcccaggctggtcttgaactcctggcctcaagtgatcctcctgcctatgcctcccaaagtgttgggattacagacatgagccactgtattccgctagaaataaaacttttttttttttttttttttttgagacagagttttgctcttctttcccaggctagagtgcaatggcacgatctcagcccactgcaacctccacccctgggttcaagcaattctcctgtcttagcctcccgagtagttgggattacaggcatgcaccaccacgcctggctaattttgtattcttagtagagacagggtttctccatgttggtcaggctggtctctaactccgacctcaggtgatccgcccgcctcggcctcccaaagtgctgggattacaggcatgagcccccgcgcctggccaataaaacttttaataaatggtcctgagacaactggataaccatttggaaaagtccatttgtggaacaagttaaaactcttacatattccaggacaaaccacagatggatcaaaattcaagtgtaaaaaataagagaaacacaacccaaatatctgacattaggcaaccggttgaataaactattggtatagtgatagaatagaataccgtgtagctgcagtcaagaaagttcactgtacctatgtagcgcgatctccagaatgtattgtaaagtgaataaaataagatgcagaatagtatttaagtgtgtcttttttttgttaaacagaggtaggtagaaatattgtttacacttgtatttttatacagtaacaataataggtagtcgagggaatggggtagacaggagtgggagtgacacttataagtgggagcctttttatttttgttctgattttttaaagcatgtaaatgtattactcattttaagataccatgtaataacttttccagattgagctgaattcatgcaagttccatgaatcatttaaggaagaagtaattctaaacttaaacttccccagataatagaagaagaaggaccattgcccaagtcattttatgagactagaatgaccttgattctaaaacctgacatgaagcaacataggaaagaatacatatgagacgctttttttttttttttttttttgtggagaccgggtctcattctgtcatcgaggctggagtgcagtggcacaattacagctcactgtagcctcgacctcttgagctcagttgatcctcatacctcagcttcctgagtagctggtaccacaggcatgtgtcaccacaactggctaacttttgtattttttgtagaggtggagttttgccatgttgcccaggctcgtctcacacctcggtctcccaaagtgctgggattacaggcatgagccactgtgcccggccttatatgatatgcctctgtcacaaaagtcgggatcatggttaactccattggaaaggtgggggttgtgatcgggtagggctgtaggggcagggttctgtgttattgcatgttctatttcttgatctgagttttagttatatgttgttcactttatcattgtataaacataatttgttttgtagttgtatgtaggtatgttatgatcaattataaatggtataagataatttaaaaagaaggtttaataattatggaacatatgtcttacaaattatttttaaaatatatacactaacacttgatagatggaacttgtatgaggaaatacaaaaactttgaatagtttaagattagcacttttaatattcctatatcataaattgataagcagataaaaaatcaagacatagaggtatttgttttttttgaagggagaaacttttaatggcacagttattccaattccacgttaagatgattggtaaagtagtgatttttaaaaacagcaaaagctggtttctcccaaattagtttccccaagggattagaggtcagtctgtcattcagtcaaatctatatccagtagggaccacccagttttgttttaggctagtctgttgggatatatagtctgcaactagcagagttgcagctctaacaagtgttagaaatgttcattcacagaatcacctgaggtacatagaaaataaagcttctattaaaagaattatgctacagaaaagaacagagaggatatatatacaacacaggaagactcagctgattcagggaccccaggagaaagtggctttcttcatttaaaaagctacttaccatctctcctgttaacttattccaaatctagaaagaaagtacttgccaagagatactgattatatatgggaatttaaatcctccttgttaggaattccactagcaattcttttggaaaacctgaaatcaaaccatttaggttctcacagagttttactctctggagatgctctggttgggaggttggttggagtaggagttggagcagcaagcattgcgtagaatgctagtctgtgtgaaagtccttattaggaaattaaaagggttcactttgccttgtcagcggtgaaaatcagccctcaaggaggctgagaggtagccaggtttggagttatttcctttgttagggaactctatctaacccccaaaacataattcaaatagtatacacaaaagtttggtttagtccagttcttcaggccctgaagcacattagccattgtctaaactcttgaattatatctgaactaaaagaataaaaggacttctcacccatccccacttgctatattcaacttgccatatttaaaaaaatactcaactcgccatatttaagaaaaaacttttgcaggactggctggacagactacatactaaaactttttctagtactgatcagtgtgtgagatttctaaccaggatttctgatttgattccatagtggcctttttctgagagaattcgaagaaggcattgtcaccctagtcagttttctaggaattgacagtcagccagtagggggtggcagtcttcattctcccagaccaggctgcagatggtgtaggttctcctcatttctttggtgtcatgttggacccttcagactttgattgctggaaatcagcatacacagagaaggctccatctgctggaccattcttgtaaatatcagccatgatctctacacttgaaggtttactgtaaaacagaggtgtccaatcttttggcttccttgggccacattggaaaaagaattgtcttgggccacagataaaatacactaacactaacgatagctaatgagctaaaaaaaaaaaaaaatcacaaaatctcatactgttttaagaaagtttacgtatttgtgttgggtcatattcaaagccgttctgggccacatgcggcctgtgggccacgggttgggcaagcctgctgtgataaccattgctgtagtgcttgtcatctttgtaggaaggcgtgtagtgaggcttacaaattttgaacttgggaatgtctgtctgcctccgtcacatgggagctgagagccatattgaaatggtgctcacgtggtggatagaatagggtcttcaggctacgtgggagtcatagaggctacctgagagagaacaggcttttcttggttcagaaattctaggtttgagaaggaaagcctacatacagccatctccacactgcacaataggtgagcagttttcagcagacatctccacattggtgttcttattggtctaaatgtacacctggtcagaaatggttttcatggccccagatgttcaggaagagctacatgaactctgttgatgatgtctttttaattgttagacattttggccactgttcctggatcagcattttttgggcagacccattttcttaacaaactgtactctctgaggcagcttggatctatccaggaaagtttccactagttaatttgcatagctcatgttggcattatggagactcaacagtttatctgcagggtgtgaaattggggtattagtcagcaccgtaaggcagaaaaggtggccagacccactctttgggctctggatcctggatttactgaaaccgcagataccctgagcctgaagatgccatgtccagtcctaatacctggttcctggaacccacctccaccactgttgccacttgaacaacataaaaaaatattttaaggacatgttatcttgctgaacctaaaagataaatagagaattctacatgctgcagagactacacattcttttctagcttatttatgtatttatttatttaaaattgagacagagtctcgtcctgtcacccaggcaggagtgcagcagtgtgatctcggctaactgcaacttccacctcctaggttcaagtgattctcctgcctcagcctcctgagtagctgggattacaggggtccgccaccacgcctggctaatttttgtatttttagtagagatggggtttcaccatgttgaccaggctgatctcaaactcctgacctcaggtgatctgcccacctcagcctccaaaattctgtgattacaggcgtgagccaccgtgcctggcccaaaatggctctttgaaagggttaataggatatttaaaaattgttggggtcttgggaggaaaatcacatgattatttcaataaaggaaaaacataactttgataggtatatgttacaagaactcagaaaataggaataaaggggaatatccttaacttgataaaggatttatgccaataacctaaagcaaaccttttatggagaaactttagatgcattctgtctaagatcaggaacaggacaaagatggttaacactattacagctttatatgttactggaggtcctagatagtccattaagaaaagaaatagaggccgggcgtggtggctcatgcctgtaatcctagcactttgggaggccaaggtgggcggatcacaaggtcaggagattgagaccatcctggccaacgtggtgaaattctgtaacatctctactaaaataaaaataaaaataaaaaaatagcggttctaagatggccgaataggaacagctccagtctgcagctcccagcgtgagtgacgcagaagacaggtgatttctgcatttccaactgagcacatggcacaccaggacatcatatcccgcacctggctcggagggtcccacgcccatggagcctcgctcactgctagcacagcagtctgagatcgaactgcaaggtggcagagaggctcggggaggcgtgtccgccattgctgaggcttgagtagctttgaagggagtagtgcttctcccagcaaggagtttgagatctgagaatggacagactgcctcctcaagtgggtccctgacacacgagtagcctaactgggagacacctcccagtaggggccgactgacacctcatacagccgggtttccctctgagacgaagcttccagaagaaggatcagacagcaatattagccgttctgcaatatttgctgttctgcagcctctgctggtgatacctaggcaaatggtctggagtggacctccagcaaactccagcagacctgcggctgagggtcctgactgttagaaggaagactaacaaacagaaaggacatccacaccaaaaccccatctgtacatcaccatcatcaaagaccaaaggtagataaaaccacaaagatggggagaaaccagagcagaagagctgaaaattctaaaaatcagagcacctcttctcctccaaaggaacgcagctccttgccagcaacggaacaaagctggacggacaaagctgcaggcatcacgctgcctgacttcaaactatacacaaggctacagtaaccaaaacagcatggtactggtaccaaaacagagatacagaccaatggaacagaatagagcccctagaaataataccacacatctacaatctgatctttgacaaaccggacaaaaacaagaaatggggaaaggattccctatttaataaatggtgctgggaaaaccggctagccatatgtaaaaagctgaaactggatcccttccttacatcttatacaaaaattaattcaagatggattaaagacttaaattttagacctaaaaccataaaaaccctagaagaaaacctaggcaataccattgaggccatggcatgggcaaggacttcatgactaaaacacgaaaagcaatggcaacaaaagccaaaattgacaaatgggatctaattaaactaaagagcttctgtatggcaaaagaaactatcatcagagtgaaaggcagcctacagaatgggagaaaatttttgcaatctactcatctgacaaagggctaatatccagaatctacaaagaactcaaacaaatttacaagaaaaaatcaaacaaccccatcaaaaagtgggcaaaggatatgaacagacacttctcaaaagaagacatttatgcagccaacagacacatgaaaaaatgctcatcatcactggccatcagagaaatgcaaatcaaaaccacaatgggataccatctcacaccagttagaatggcaatcattaaaaaaatcaggaaacaacaggtgctggagaggatgtggagaaataggaacgcttttacactgttggtgggactgtaaactagttcaatcattgtggaagacagtgtggcaattcctcaggtatctagaactagaaataccatttgacccagccatcccattactgggcatatactcaaaggattataaatcatgctgctataaagacacatgcacacatatgtttattgtggcactattcacaatagcaaagacttggagccaacccaaatgtccatgaatgatagattggattaagaaaatgtggcacgtatacaccatggaatactatgcagccataaaaaggatgagttcatgtcctttgtagggacgtggatgaagctggaaaccattattctgagcaaattattgcaaggacagaaaacaacaccgcatattctcactcataggtgggaattaaacaacgagaacgcttggacacagggtggggaacatcacgcactggggcctgtcgtggggtggggggagggaggagggatagcattaggagatatacctaatgtaaatgacgagttaacgggtgcagcacatcaacatggcacatgtatacatatgtaacaaacctgcacgttgtgcacatgtaccatagaacttaaagcataataaaaaaaattattagctaggcttggtggtgcgtgcctgtagtcccagctactcaggaggctgaggcaggggaatcgcttgaacccgggtgtcggagattgcactgagccgagaccacgccactgcactccagcctggtaacagagcaagactctgtctcaaaaaaaaaaaaaaaaaaaaaaaagaaagaaaaaagaaatagaaatcagttagcaggattggacaggaagagataaatctttaagtattaatagaccacctacacataaaaatgtatagatgcaatagataaaaattttgttaataagataattgagtattttcagttaagggatcatcttacaaaaatcagtagaattcctctgtattcaaaataattaactagaagagatgatgatacaagatgtcattcacaatagcaaaaacaatcatgtctctaggatctgggaattaacctaacaatgcataagatctacatggaataaattttaaactctgttacagtattattcaaaagacttgaaaaccagatacaccctattcaaagatagtacaacatatttgtaacgatgtcagttctccctaatctataaattctatgaaatgtcaatcaaaatccagtggaatattttgagaaatttgaactattaaataatttatgtggagcttctgattctgattatgacacttttgcttatattacaccagctgttctgctgaaaacaactagaaaaccttgactaaagtataaatttatctgtttgaaggtataagaactatgaaatcagagaggtaaactcccaacattcagccctacctttctctttcaggcagttgccattacctaggcagtagcagaacagctagaaatctcataggcaggaaaaacataacttggagttcaggtttcagtaaagagtagggctctgttaagtatcctaggtattcagtttcgatccttgaaggcctatcacctttaggtgtaaagacaaatggaaacgagtctgccattgtaagacagaaactcagttgcaatcaaatcagttcttaagactccatgacaggataaatatgaaaattacacccagacacataatcagtaaaacttctgaaaaccaaagataaaaaatttgttaatacctagcagaaaaaggacatgtaatttccagaaagcagtaagacagttcacttctaacagaaatgatagaagttgaagataatggaaaaatggcattttaaaactactgaaagaagaaaagccatcaatctaagactttatgcccggcagcagtatctttaaatgaaggcaaaaaccagaagatattgctaaaagaagacctaggcaaacgaaaagacattgcatattcatggatttgaagacaatatttttaagatggcagtattacccaaattgatctacagatacagtgcaatccctataaaaattcaaggtacctttctggtagaaatggacaagctgattttaaaatttatgtagaaatttgagagttccagaatagccaaaacaattttgaaaaagagcaaagttggaggactcacacttcctgatttcaaaacttactacaaagcaatggtaatcaaggttgtgtggtactggcataaggctcatatataaatcaagagaaaagaattgaaagtccataaataaatttttacatttatggactattggtttttgacaagggtgctgaaaccaaggaggtaaaacagttttttttccaacagtgtggggcaactggatatccacatgtgaaagaatgaagttggactctacttcataccatattcaaaaattaactcaatatagatcatagacataaatgtaacagataaaactatgaaactcttagaaggaaacataggtgtaaatttttgtgaccttggcttaggcaatggttcttaaatatgacacgaaatctcaggcatcaaagaaaaaacaaaaaataagatttcatcagagtttaaaacttttgtgcctccaaaaaacaccatcagggaaggaaaaagacaactcacagggtgggagaaaatatttgcaaatcatgtatctgatgagggcccagtgtctgtgttaaaagaactctgcaactcagcaataaaagcacaacctaatttaaaaattgatgagtgatttggatagctgtttctccaaagaagttatacatatggacaatatgtacatgaaaagatgctcaacatcactggtcattagagaaatgcaagtcaaaactacagtgagataccccttctcacccactgagatggctgtaataggcaagacagtaacaagtattgatgaagatatagggaaattggaaagcttatgcattgctggtagaatgtaaaatagtgtaaccactttgtaaagaaattagagagttcctcaaaatgttaaacatagaattaccatatgatccagcaattctactccgagatgtgtacaaacgcaagagaattgaaaatgtaggtacacacaaaaacttgtatatgaatgttcatagcacccttaatcataatagctaggaagtggaaacacttcaaatgtccatcaactgatgaatgcataaacaaaatgtggtatatctgtatgatggaatattatctagccacacaggaatgaagaagtactgatacatgctacaatatgaatgagccttgaaaacatggtgagtgaaagaagtcagaacaaaaagccacatattgtatgattccatttatatgaaatatctgtaggtttgttacataggtaaacgtgctatgatggtttgctgctcctgtcaacccatcacctaggtgattaagctccacatgcattagctgtttatcctgctggtctccctctcccggcctgcccccacctccccaccccataggcctcactgtgtgttgttcccctccctgagtccatgtgttctcatttttcagctcccacttataagtgagaacatgcagtgtttggttttctgttcctgttagccaatagaacaagtagacaagaatcagtaatggataaaggatatgaacaacctcaacataattgacatagaacattatactgaatcactgatgactacttgtttcttgttcagtacacatggaacatttatcatttactagtgtatagacatgcagttgatttttttttaatatggaccttgtatcctgtaactattgtaaatccacttagttttaggacacagtttttgtatatggtcccgtgttagatttccgccttctatcttgtctaacctttatttcctgtctttaccagttttatggtcaccagagggaaaattcaaagtctccaaagtttggcagaaagcatcagagtgaaggccagctatgatgcctggatatccattagtattcttagatgcacttaattttgatgccatgccagtttctttcatctcactcagaaatgtatttaaatggttattttaacagtttctcacatttatgatatataggaaagtaattcaatgtatctgatcttccatactgacagaagttgaagtttttgttttttcatatgaatgttagtctcaacttgtctgcttaaaaaagtgttattttcattgggataatttgaaatttacagatgaaaggagaattgaagtccagtcctctttggattacaatgtgatttataagttcagagaagagagatggccagttttataaactttttacttgtttcttacgttccttactagatttttttagtagctgttgacgggatattttctactatgtttttttctttctttttcttgaaaaatatgaaaggcatttgtttatatatcctacttcattaatattaattacctttgttaccagctaccttaacaaattaccttgtttctagtagtttcctttttgtttattttatacttttttagttatgcaattgtgtcttctaccaaaaataataaaattgggagtctgtacattgagtttccctattagctctcttgactccttgagtttttattttcagctttaactctctatgtaattgctcttacctgtgcattaattttattctgcagtgtagtttctccattttcttttctctccatctaaccctattctgttcatccttagatttcaggaattctttaaaatatctaatcagctgatgttactcttactactttccagtttattatggtttatttttgacaacttatattgtctgtcatctcatgagcttgaaggtgggagggaagtaaatgagtctagtttgccatctcaaaacagactctatttagactatttaaatactaacttaaaaagaaaacatcaacaaaagtcaatgttcctggctcttattcttcagtaactgaatatttcttggctggtcttcgcagctgaggttctttatactgcttcagtgtttctgtcctttgagaacttcaaatctttcctgtttctgcctctacatgtcattgatgaaagtcttcattccttgtaccatagatcctggaagttttgatactttccatctatggactatgatataattgcatcctgtttgagctgtaaggaggtgaggttcaaacctcttctctttgcttctgggttttaactagtatactagagctagatcaggttcaaaattaccggattttctgttaaaagagatcaagacttaaacctggagccaaactggagatttccaattattttctcctaaatgcaaagttgggttctgacttggaatctcggagtagactccaagtatatgtatccagctagctgacttaaggtttccagccagccttacaaaccaattggctagtggaacagagaacagtattgaaatatgggcatccaggccaattgcaagaggacagatcttgagtcctaatccatttgagtttattacaaggacagaagtgaagtgaaagggaattactcatttttgtttttcaacatttgcctcttgaatcaagagagagggtggagcctctcttgcttgtgaggcagggtgtttccacatactcgtaacttgaactctaggaagaaaaaggtagcaggataaattttacagaaaagggaagtagagcagcatgctttgcccaagcactcatctcctttgatacagttccttcagatactttgaaatgactaatgcattatatttaaggccactagtactagtcattgtgttttcaaggaaatcagaggtattccctgcttcactaaatgtatttgtcatgaccaattgcttctttaaaattatcccatatttggaaatcagtgaaagaattgcggcagaaagaaatcagtgaaggaactgaatacaagaataaagtcaggagatgaatgagtttatgaaaatgggaataaagatataaaatacttttattgttctaatggggagatgagaacacctatacaacttcttcctagtatttatacttaacaactattaaattccttatttatgtgtttaatgtttgtgttcaccaataaagtgcaatctctgtgaggccagggagtgaatttctcttatttgttttattccctacttagaacagtgcttttaaatgtaattttttaaatcaagggctaaaaacaggaaaatggagttctttttttctttcatgcttccctcttttgactaatgaaaataatacttattaatcatctactgtaatccagtaatttacatgcccttgagtagggttgcagtttaatcagcaagggggagaggtacacaggattgatacccacttctgtaatttgagggatatttttccaattttatttctattcacaggtaacaaaattgatgtagttttataaacattaatataatgaacttttcttaccaaatatttagtctttttcccatgtattgaaatatttctttgtaaatctgatttcttatggccccaaagaagtccattatgtggatatttgataatttacttaaccatttccttattgttaagctagaatgtgtgctccatgaatataaagattttttttccctctgttagctcattgcagtttcccagtacctacaacagtgtctagtgcgtagtaggtgctcaatatgtatttgtagcatgagtgaaagaatgtgctctattttttccatattataaatgccttataaaacgtttttgtacctaatgctttcactacacttcaaatgattttctaagaataaatttctagaagaggacttgccagttgaaaagacatagaaaaggttgtatgatttcaaattagttttctaatccacaataatgttacctgtggctatttttctttacactttaattttataaattttctataacatattaggattaaaagagttattttttttcttttaagtacatttctatttattcagagagcagtgagcagtcttctaactggaatataaggtttttgttggtggtggtgaaagatgaggcttgaaaggtcacagctgggtgtggtggctacacctctaatcccagcactttgggaggccgaagtgggcgcatcacctgaggtcaggagttcgagaccagcctagccaacatgatgaaaccccatctctactaaacatacaaaattagccggggtggtggcacgcacctgtaatcccagctacttgggaggctgaggcaggagaattgcttgaacctgagaggcagaggttgcagtgagctgagactgcaccattgcactccagcctgggcaacaagagcgaaactccatctcaaaaaaaaaaaaaaaggaagaaacatcacttgggcaaagcagtgaatggcttggtatgctatgctaaggacctttattctgcaggaatcggagtcctataaactattaataagcagaatacaaaaacttcataaaataaataaaaacaaaatacaaaaaagcagagtacagatgccacctgtcttttgaaagtgttatgtcccagtgaacccactgtaaattaaaaataagtcttgaagatgcatttaatatacctaaccatcatagcctacctaccgtagcctagcctaccttaaacgtgctcagaacacttatgttattagcctacagtggggcacaatcatctagcaatactgtacagtgtagagtgtcggttgtttgccctcatgactgcgtgagagctgtggctcgctgcaactgaccagcatcctgaaagagtctcatatggctttctattgaattcatgttgtttttgcaccatctgtaaagttgaaaaattgaaaaatcatcagttgaactattgtaagttggacatcatctgtagtggtatagttagaactcttctagaaaggtaatagtgatagtgctgagcaaggattggttagagaaggggacaaagggaggtgctagatttctcactctctagtctccatatctcaccagtcactgactcagtatttcttcagtttcttcctctttttcttagttagatcctctcacctcaattactgctataactttccaatgtctttccctgccttaaaaccattctccacagagctgctagagtgatctagctaaaactatctgcgcagaatggttcccctgttatctgtatttttaaggttctagtttattaacatgacatacagcttctctgctctttggacccttcagcaccatttcttgctactccctgttgtgctctttatgttcctaggaaaggaagctgcttattgtcctgtgtatcttctcccccacacatatttcctgcctgtgtgccttgcttatgctgtccctacttcccaaactatcccatcctcagtcacatccatttcagtccccttttttctggagttcactgtgtaagactcaggtgtcagccacttcatttaaaaaaacttttaacatacgtattcacatactatacactttcctcctctactcccactttggggtcaaggatctctcttctttactttttataggaccctgatttttatctctcctttccctgtagtactgttatggtactgtctgtgtactactgtttaggtgtgttttgcacaatggactgtgagaatttggggggcaagaacttgtgggggccaagggtccagcccttgttattggtccaacctttggctctctgaaagtttgctgaaaaatgaacatgcacaaggcagattaataggagaaaaggcatacaaatttatttaacatgtatccgcgggaacctgtatgatgaccccaaaatacaggggaaattgcccattcttatacttaggttcaataaagtatagacagctgtgtagaaatgtgattggagaagaaaaagggtatgatctaatgctaacagactgagtggggaaacccagcaaggcctgtgtgtctaaagtcttcttggcctctctgtgcagcattccttccttccaggtatggggcaggaccctctctggaacgggggtgtgatgacctacaatgaaacaggtaaaatctgatagtttttttgttgttgttgtttctttgtttgagacagagtctcgctgtgtcccccaggctggagtgcagtggcgtggtcttggctcactgcaacctcagcctcccgggttcaagccttcctcatgcctcagccacacgagtagctgggactacaggtgcctgcctaatttttttgtattttcagtagagatggggtttcatcaagttggccaggctggtctcaaactcctaactttaagtgatccgccttccttggcctcccaaagtgctgggattacaggcatgagccaccgtgcccagctatttttttaaagacaaggtctcattctgtcacccaggctggagtgcagtggtgcaatcacagctcactgcagtcttgaccaagctcccatctcagcctcccaagtagctgggatcacaggcatgcaccactatccctgactaattaatttttttttttttttttttttttggtagagatagggtcttcctatgttgctctggctggtctcaaactcctgggctcaagtaatcttccctctgcagcctcccaaagtgctgagattataagcatgagccaccatggctggcaataatttctttgtagccagtttttataccaaaaggctgagggaaaactagagtaatatttttaggttttatggctggctttgaggaaagggagttctagtttctgtgacctagagaagagggattctagtttctatggcttaccttgggggagaatgggactgagagacagagcaggagaaggtcaaagaaaaacttctgtgtctaatgcagctgagtccttcattttggagtattgttttctgagcccaacaaactttttattcagctttgtgttctcagcagttagcataatgtatagcatatagtaatgtggtaaaaaaaaaaaaaagcctattatttttctgttgaaataatgaggtcttgaactaggccaggggtagtgggaagggagggtgggtgggagaaacattcaaaaaagtcaactttggatgttagtgataagttaaaggtatgtaatttatagatgatgagatttgcaacttgagtgagtttggtatacagattgttgacttcttaaatttaagacatttgctgaacatgctagagagatgaccagtaggcaactacaaataagggtatagatagtaagagatagggatttaggagttgccagcatattggtattaactgaggctttggaatagataatataagtcaggaaggagaggagatgtaaaaaaggtggagtagagatccttggggaacaatatttaacaggttacagcaaaagaaagattagagaagtatattaattgtcagcagtgtccagtaatgaagaaagggtaaagcagatgagaattgagaacaatttatttggaaattagacagttgttattgaactttgagagaatagtttcagtctagtagagtgaaatagaaattagattgaaataggtgtttgaaagaggttgagaagcaaacaaatgatgagcaagtggaaaaagcaagcatgtactactctttcaaaatgtgctttttggagaaatgtgttgtggaaagggatgtggtatcagttccttgaggaaaaggtaaaatgggggtgacgtttttgttcatatttttgttcttggatggggaatggtcttggaaaagtttttcaattgatatccaggagaaagggggattgaaagcaggaaagaaatttttaatggaataaggacaggaagggaaggaaggagagggtttgggatcaagatgtcaaatgatactgttagccctgaaaagtggtagggttcaaaaggaaaagaggtatgaagcgcaaaaacatttagagttggcatgagggaatgtaaaaggtctgttatctataaagtaggcaaccaggtcattaattgctggtgaagagtagataggacaggaatgggatagtttgtggtggcagggtggcacaggacattttggaagattagtaaagttctgttgagagtgatagaattaatgttaggactgcaggtagatttgagggttcagctgagattgagctatggaaataaaaagcaataataaaaagacaaagtatatatatattcttatatatattattccttacataatatatatttccttatatataaccttattttatatatatccatatatgtatgtgtgtgtgtgtgtgtatatatatatatatatatatatatatataacatgactgtttttcattaaagagactgaagaaaaaaagggatttgaaaaattacttgtagttgctcattaggatatgtaaggcagttacttaaggacagccaaatttgctctactgtcctgatacttggaagaattaaagaatggggtacagggctggggagttggtggcagcagagagcagtccagcagtctgtcaagtcctgcctggtcacaggtcagtgccatttaaacctccaggatcaagtcagagcacttgacgcatgactgataatatttgagatttagtgtgaggtgtctttttaaatttttttttggtgtgttcttgctgtatgtgtgtactgattaaaatatgtaaaataaaagcaacaggttgctggtaattaactctgttatttcaagacagaattgattttgtatagaaaggtagtattggctggattatgagctttactgcttattcattcctgatgaaaatatctctgctgggcttacataccctgtagtgtttatgaactagtaggtttagaaatgaagagaccctagagagataatcatttcctccaagatgttttgtcttctaccaggaatttgtcaatagatatataggcccagtgggtaggctcacctctgcatgaatatttgataggagcatttctatcagttatgctctttcttaaaaaaaaacatgaaaataaacgcatcatttatgtaactcttataagaaatgcataaatcatgggaaacagtgagcaagatataattgaaattcagcagctcagtgagttacgaaccttctctctacaactcaattgtgttagttactgcttaacttaacgctgggttttcctttttgctttcaggaaaacctttaacttacgccctgggtgttgcctgtcaacagtttcctggtggctcaccgcttctgcctgctccagccttgctccggcagcttcaaggcttctgggcttcagagcttgttgtgaaagcttgctgctttggcctgctacccctgcttcaggtgtcaaaagcttttaattagcttgcttcctctaattactataactaggcccttacgtgtctgcataatccagttctagggtctgaaaggagaattacctttagccattcattcttctttaaaagatcttttactttggccctcctggcattcatgggagagtaagaagttatattgcatgattcacctttgatggtgctagtcttcagtctgtaggggcaaactggtgtgggcatactgtattttatagccagtgtttgttctgattggttgatgcccatgctgtcccaattttaaagtattttgactattgctactgaatatataaattgtgtctccatagaaaggatatctctagctggtttaataaactctttgtagtgtttttagtttgcttattaaagataatatttagttccctcccccatttttaataaatattttgaacacaaattggtcgctcacagaacagtttttaaaaatgcagatacagtgatttctatacattttgtaatgatcaccattaacatgtgtcaataggtatcagaattataggtatgatgaatgggaagagctcttcagtacttatataactattcagtacttggagtagtaaagtatactagctctcttatcagacagccaggtacatattctggttctattacttattaattttgtcactatgagtaagttaattaacttctccaaacattagtttactaatttaagtggggattgtagaatctgctttagattggtgtaaggattaaatgaggggtaatatatgtaatatgcttagcccagtgctaggcacagaataagctttttataaataagagttattttttgttattattattaatcataaatgatagtagatgatatgtaggggaaattaaaaggtgaaaaagacaataagtaatgagcagcaacagacaaaagatattaaaggccaagttgtgaagtcaggacttcgatgcctgttaagagagcagagtgagctggaataagctgatagatgtttaataaatcacaaaatacacagttgtatctcttttgtcataataccttatttttgagttttagtgtcagtttagttgtatacttgcagctgagaatattgatgctgtaaactttgcatgtatatacacccacatagattaccagtcattcatcattaattcagctaatgtttattgagtagctatagattcccaattactaggtgtgtactagtgaacaaataggaaaaaaaaatccctgccctcattagcttatattgtagtgaatgaagcaaacaatgaacaaagtacttaaatagaatacatagttcatgaaataatgacaactaatggtaaagggaagcaaagaaatgggttagaaaatgtcaaggggtaaagtgacatttcattgaaggagtgagagagaaagctgtgtggcttcgtggggctagaatattccaggcctagggatcaacaagtgcaaaggctagttgggattgagtgtggtgatcattatagctggagcatagtgaaccagggggagagtagcacgaggtcagggaaggtggagggccagaacatgttggccctataaggcatagtaaggattttgacttttacttggagacagatgggaaaccattggaagctgtggagcagaggagtgtcatgatctgagctacatttcaagaggatcaactgtggctttagtgttgaggatgggttgtgggatgagagggcagaagtagtagagaggtcaggtaggacaatccaggcaagattttcttttttttcttttctttttttttttgagacagagtctcactcacttcagcccgggctgaagtgcagtggcgtgatcttggctcaccacacctccgtctcccgggttcaagcaattctcctgcctcagcctccctagtagctgggactacaggcgcccgccaccacacccagccaatttttgtatttttagtagagatggggtttcattatgttggccagactggtctcgaactcctgacctcaggtgatccacccacctcggcttcccaaagtgctgggattacagacatgagccatgtaatagtagcttggaatgggttggaacagaggagatatggtgaatgaattcttgctatatgcaggaagactagatttatagaactccgtggcaactgtttctgtattctacaacctgtaacttaagaatgaacataaaatactgttacagcattctgtaactgcaatttgtaactagtttgtagcttcaaatgataatacattctgtttaagaactgatttgtatacatttgtcacttagtgattatttctgtagtgtttacagaaataattcttgaattattattacaggttttctaacatcatgtttttactatgaagtctttcaagacattgctagtgtttttaaaaatatatctttgttctgtgttaaactatagtcttgcagttcatactaaatgtacttttaattttgatcaagctaaacaagaaaggacattttaacagactacactgttatatgttaagctgtggttcttgaaaatacaggacctgaggttagagttctaagagaatcctcctcttcttgatttttagctactgaaatgaatatcagtatgacttgcagtgttaaaggagaaaagcaagatcagtttgattttattgtattactttgtaagcctcatattcttttctcatcatttatcaattcaataattcaagaatatttcctgaactgacttttatttcaccacactcaccttattccagaaaagattttaggtagctcatgaaactgttataagataggtttgaaaatgttttccaccctgcagcagcatggcattcctgatgaccacatacatgtgtagtctcattttagccatgatcagttataccacaataccttgataattagcggtcatttcatgaccaggatgtgatgtgtggtatcccatactacctcattccatgcttcaaattttatgtgttttatatagaaaatgtagaaaagcaagtagaaattgcatcacttagaaatacctgatcttttgataaagcatagaaatttatggtacatattttatgtgtgtgtgtgtgtatatatatatagagagagagaaaaagtcacagtgtacatactttaataaacttatttgtttaacattatactgtgaacttttccgtattaacattcctcagtaattttttcaagtgtatgagtaatttttctgtcatactgtgagataccatatttagtcaaattgatcatgtagaatatttaagttcatttcaatattttactattgaaaaccacactgtggtgaatattgtggcttagtttttatttgtatatagatatattcagttgagggtatttctatccatatttctatgtgaatatatttgtcataagtctaaaattacagaaagttagaaattaaagagatgaaagatttttttattttggtagttaaactgaagaaattgagagttatattctttgccacatattagaggtagagttggcagtgaagagattgggctgtaatgtgcttagtgcaatgcctggcacataatcatagctaacacttattgaatgcttactaagcaccagacccttgaaaaacttgaaattatttttcaccctgtggcagcatggcatccctgatgactgcacaggtgtgtagaatgggtctcattttgtccctgatcagttacaccacaatacctcaatagttagtggtcaataatcccgagtacaaggattatacttgtattaatttttcaaatctttaccacaatgatataagtattgttattaaccccatgatcatgacaaggacactgaagtattgaaaggttaagttttttgcctagagttatacagctaaggacaaaactaggattcaaacccaagtagtctggcttcagaatccatgctatcaccactccccaaaacagccacaataaatgctagctaagtgtttgagtgttcaataaatattaggcgtcatcattattcctactgcacagttgcatattccagaggtggttttggttccaatatttttggattttagaaagataatttagtttgtatactctattttatgtaacattctcattggggtttggagcagcccttcataaacagatacattcacgtttcttcagtgaagcatgtttgtttttatactaagtgagattaaaaagcaagaatattaatagcctgatgtcactacatgctgccaaatgagttatgaaaaccttttgtctttgagaattgcagataagggattatggacctgtataaaactcttaccatagtgcctggcatttagaacacactcctccttctactagatttatactagtagtattgggtgggtactactactaccactactgttattagtagttatcatcagatacatacttacagaaaactggtgaattgtagagtactgttagtacttttctcacactgtgaccttgggaggggaagacacctaagttttggagtctttcagctattcttccctcaatcagaataagagggtgtaagtcatagtgttctgtgtgttttccccgccctaaattgagtcttgtatagcttagtgctcctgtccctctatccatgtgcactcccagttgtgcagggtttgattttagagtctaagcaatagctcacgtggtgtgttcaccgcttcagagatcttagtgaaacaacgtttcaagtttttccttattctttattacagccctctttatgagcccattgggagaacaatttaattgtttgaaattgagatacttccttttctttctcatccattttaattgatgaaaaactataatcacagtcattataaccacttataacaattaccaagaaatatttagtatgtatacacttactgtaattatatggtcatgttacttttattgcagtctgatccaaatataaatttattaaataaccttgatatgcacatttgctattaagtttttttttttttgcacttgaatttaaacctatactctgtttttggaaatatcccgttgaacagcagtttgaagatctgctgtatatagtttgacctccctttctgcatattagagtagcatttctcttcctccccaccaacccccagtaactggaaacatcctcgttcaacctttgttttcctcttttgtgggctagcctgtggcctatggcaagtccaatggcctggaagtggtcttacagattatctggttaaactgaagcagagagatagaaatgtaatcaatttgttgagagtgagaaagggattttatttattgtatttgatagtttttgatctgcaattgaaatataaaatacagaaatagatgttcctttttttttctttttggaagggatgtgtaaagtttagtttttgtaaaatcgttagttttaattgctctctatgcgtatgtatatgtgtgtatgtggtttgggggttatggtatagtatgtaagagtatgtgatatgaaaaataggatcgttttcagtttatttcatattgtttgatcctcacaaaacatatttgttttggcagctggccttccttgtaacatggtgttggaattttgcatgcattatttaataagctctagttctttcctgttgagcattttgttcagtttcttagtgatagtagatattgagtttgaattccagggcagaaaagttggaaagggggattaatgtgtaattaccaactaatacagtgttcaaaaataccaactaatacagcgttcacaaataccaactaatacagcattcacaaatacagttattagtttggcacatgttcactaactttttcatctggcaacattatttaatttaggatcttagaatttgaactgcattgacataattgataattggtatttatttttttacagaggaaaaaggaaaatggatggctgaaaaaattatttgtcattattttaaaatttatatttcctttatcttttctaggttgctgtagtaaaactaaaaaatgcagataaagtgtttgccatgaaaatattgaataaatgggaaatgctgaaaagagctgaggtaagagtcttatgtattggaatatataaatattttagaaatatttttcaggaattataaaattccatagagctcattaaatattacttggtaacattttgtctgtggtgacattttttaaggcataaaagacaaattcaacacaattgtgatataattatgataaaggttacaatccataatgctaatttaatattagtggaatactaaatggaaacccttgtaatggggggcttatgcagctatatttaaaggatgtctgcaaactactttcttctatagaaagacattttgtaatggttgatttctaacgtgacgtaacttgtttatattattctgtatttatctgtattctgctacaagcaaaagtcagcaggagttttgaacatgccctctagaaattctgtttaatgctgattatgcagtcactttgtattttgttttatattacaggagttgtagtaaaatctagtaatggaagaactgcctgcatttttatgtagatttgagggaaggattgactggagtgatgttaaaaatatccagcagctggtatggcctggtcagaactgctgtcacacatagatttggagtggacttctggggggtccgctgctgtgctgtatcatggtgaagggttgaatgggataggcattctggggcttcagggaagcagaacttaactggtgtagaattctgtcagtattttaactggtaaaaggtgatactgacgagcaggctgagtaatagccctggaagttagtaagattagtaagaaaataatgacaaattcgcctttagaaatctttatctgtcagtacttgtagcaacccaaagatgaacaaacatacagggaaaagcaaaaagtgtttccatgatgacagatatggtggcagtgaaagtaaatacttcattatttatttatttggagaaatttgaaaacacaaagaagaaactaataatcacttttgttctgttacccagaactaataacattttggccaagtttttgcttttagtttttttttttaaactaaaaaaaacaaacaaacaaaaaccctcagaagtgaagcacaaatatattctctgtaaaacaaataatagcatattacagataaagccaaagtgttctttcaccacccaagtccatcctttctctaactcccagaggtcattattacttttaaaatactatttcttcattaaacattttttttgtttttatatatttaggagatacaggtgcagattgctgacatgcatatattgcattgtggtgaaatacccatctagtgaacccatcacccaaatagtgaacattggacccaataggcaacttgtcagccctcacccccttcccaccctcccaccttttgtagtctgcaatgtattattccactctgtatgtccatgtgtactcatggtttagcttccacttataaatgagaacatgtggtatttggctttctgagtttttttcacttgggataatgatctccagttccattcatcttgctgcagaagataagatttcatatatttatttatgtgtatatgtacacacatatatacatacatacatacatatatgtatacacacatcacattttcttcagtcctgcgttaatggacagcaagattgagtctgtgactttgctattgggaatagtgctgtgataaacatataagtacaggtatctttttgatataatgattttttttcctttgggtagatacctagtagtgggattgctggatggaatggtagttctgagaaatcgacgtactgttttccataaagattatactaatttacattcctactaatagtgtataattgttcctttttatctgcatccccccagaggtaattattgtatagtaatatcctgtttgacaaagtatctgggagctttaatgtcacatgtatcactgggatatgtgccagtgtccattatattcaacaccttagtttttacttttaatttccctaagactgaattgccttactgtcactggttggatttctcaaggagtaaataccgagattgagtttattgtgcagatatttgtttgggatttcttttgagaaatccctacctgtagaagggaagggagggagataggatttgtaaagttgtcccatcagcctcagttgattccactgggagctctgtagcagaaatggctcttgagaagtgtcctacattggatcaaaatgtctagacctttatacacctgcctctactagtcagtggacatgggctacccctgaaggacatggccttggactggtaggctttcttcagctgatgcaaaccctgaaggtgtgacagcatcagcagctagagcaacaaatcctgttattacaaaagagtaaataattatgttctgtttagcagaaataacaactgtagtcagtttactgtttcagagttagaacttttaaaaaattgaattaaagtttatacttttttcaatgaagctggatatgttggggaagaattgtgaaaaggatgaataactgggttagctttcattacagctggaagtggcaaaattcaaaataaaacagtataatttaaaatcaaatattttattcaattaacaagtatttatatattctgtttctgagacaagatggaatagactcacttctccctgttctcctgtgaagtatagctgaagattctggacatttatatataaagcaaacattagaagaacccaaaaggtagagagaataaggtagaccagctagggacctcaggaccttacggtataagacccattttggaggtcagtattatgctgatagaaaaacctgacaaggaccatacaaagaaagtttaagataaatagtcctcgaaataccatttgacccagtcatcccattactgggtatatacccaaaggattataaatcatgctgctataaagacacatgcacacgtatgtttattgcggcactattcacaatagcaaagacttggaaccaacccaaatgtccaacaacgatagactggattaagaaaatgtgtgacactctcccaagactaaaccaggaagaagttgaatctctgaatagaccaataacaggagctgaaattgtggcaataatcaatagtttaccaaccaaaaagagtccaggaccagatggattcacagccgaattctaccagaggtacaaggaggaactggtaccattccttctgaaactattccaatcaatagaaaaagagggaatcctccctaactcattttatgaggccagcatcattctgataccaaagccggccagagacacaaccaaaaaagagaattttagaccaatatccttgatgaacattgatgcaaaaatcctcaataaaatactggcaaaacaaatccagcagcacatcaaaaagcttatccaccatgatcaagtgggcttcatccctgggatgcaaggctggttcaatatacgcaaatcaatcaatgtaatccagcatgtaaacagagccaaagacaaaaaccacatgattatctcaatagatgcagaaaaagcctttgacaaaattcaacaacccttcatgctaaaaactctcaataaattgggtattgatgggacgtatttcaaaataataagagctatctatgacaaacccacagccaatatcatactgaatgggcaaaagctggaagcattccctttgaaaactggcacaagacagggatgccctctctcaccactcctattcaacatagtgttggaagttctggccagggcaattaggcaggagaagaaaataaagggtattcaattaggaaaagaggaagtcaaattgtccctgtttgcagacgacatgattatatatctagaaaaccccattgtctcagctcaaaatctccttaagctgataagcaacttcagcaaagtctcaggatacaaaatcaatgtacaaaaatcacaagcattcttatacaccaacaacagacaaacagagagccaaatcatgagtgaactcccattcacaattgcttcaaagagaataaaatacttaggaatccaacttacaagggaagtgaaggacctcttcaaggagaactacaaaccactgctcaaggaaataaaagagtatacaaacaaatggaagaacattccatgctcatgggtaggaagaatcaatatcgtgaaaatggccatactgcccatggtaatttacagattcaatgccatccccataaagctaccaatgactttcttcacagaattggaaaaaactactttaaagttcatatggaaccgaaaaagagcccgcatcgccgaggcaatcctaagccaaaagaacaaagctggaggcatcacactgcctgacttcaaactatactacaaggctacagtaaccaaaacagcatggtactggtaccaaaacagagatatagatcagtggaacagaacagagccctcagaaataatgccacatatctacaactatctgatctttgacaaacctgagaaaaacaagcaatggggaaaggattccctatttaataaatggtgctgggaaaactggctagccatatgtagaaagttgaaactggatcccttccttacaccttatacaaaaatcaattcaagatggattaaagacttaaacgttagacctaaaaccataaaaaccctagaagaaaacctaggcattaccattcaggacataggcatgggcaaggacttcatgtccaaaacaccaaaagcaatggcaacaaaagacaaaattgacaaatgggatctaattaaactaaagagcttctgcacagcaaaagaaactaccatcagagtgaacaggcaacctacaaaatgggagaacatttttgcaacctactcaaagggctaatatccagaatctacaatgaactcaaacaaatttacaagaaaaaaacaaccccatcaaaaagtgggtgaaggacatgaacagacacttctcaaaagaagacatttatgcagccaaaaaacacatgaaaaaatgctcgtcatcactggccatcagagaaacgcaaatcaaaaccacaatgagataccatctcacaccagttagaatggcaatcattaaaaagtcaggaaacaacaggtgctggagaggatgtggagaaataggaacacttttacactgttggtgggactgtaaactagttcaaccattgtggaagtcagtgtggcgattcctcagggatctagaactagaaataccatttgacccagccatcccattactgggtatatacccaaaggactataaatcatgctgctataaagacacatgcacacgtatgtttattgcggcattattcacaatagcaaagacttggaaccaacccagatgtccaacaatgatagactggattaagaaaatgtggcacatatacaccatggaatactatgcagccataaaaaatgatgagttcatgtcctttgtagggacatagatgaaattggaaatcatcattcttagtaaactatcgcaagaacaaaaaaccaaacaccgcatattcttactcattggtgggacttgaacagtgagatcacagggacacaggaaggggaatatcacattctggggactgtggtggggtggggggaggggggagggatagcattggagatatacctaatgctagatgacgatttagtgggtgcagcgcaccagcatggcacatgtatacatatgtaactaacctgcacaatgtgcacatgtaccctaaaacttaaagtataataaaaaaaaaagaaaaagaaaaaaaaagatgaaaaaaagatatgccagtaaaaaaagaaaaaaaaaatgtgtgacatatacaccatggaattactatgcagccataaaaagtgaagagttcatgtcctttgtagggacatggatgaaactggaaaccatcattctcagcaaactatcgcaaggacaaaaaaccaaacaccgcatgttctcactcataggtgggaattgaacaatgagaacacatggacacaggaaggggaacatcacactccgggggactgttgtggggtggggggaggggggagggatagcattaggagatatacctaatgctaaatgacgagttaatgggtgcagcacaccaacatggcacatgtatacatgtgtaacaaacctgcacattgtgcacatgtaccctaaaacttaaagtataataataataaaataaaataataaaaaagataagtagtccccatgcatatagatgcaaatatccttaacaaaatattagcaaatacaatttagcaatatgttaaaagaattatataccatgcccaagtgtgatttattgttgggatgcaagactgattccatatttgaaaggtactctcctcaaactatttgagaaaacttttatcttttagacagaaattctgcattgaaacttttaaggaagaattcactttaaaatagaaattgtatttctattcttgaataagcagatctttttaaaaacaaaaacatttctaaaaatccttctgaccatgacttatccaactttggaaaacaagacctaccagtgatgcttaataaattacaaatgagcagcaaaattccttattttttatttgtgtattgttgatgctttaaaacaattttttcagaggtttatgtacaacttatttaccttattctgtttcaagccatatgtcaaatatataattaatgatatacgggttactcaaaggtgctaggaagcctcctctcttttctatacctgggcagaaaggtataataatataataattgtccaataatttcacagatttatcatttacaaactttgctagtttatcaccaataactctacaagttcatcatttacagactttgctagtttatcaacagcctcctaaattaacaaatgaactactgctctcaatctgttctgtaaattttacttctgtcttcacatttgttcagtgagtactgacttcgctcattaacagatgtccttactcatttcatgaggagatgcggtatagtttacagaaacactcactgtcttgagagttaaacctttttataaaaccttttaaaaacattttatttattgaggcagagtcttgctctgttgtccaggctggagtgcagtgttaggatcttggctcactgcaacctctgcctcctgggctcaagggatcctcccaccttagcctgggagtagctgggactacaggcacatgccaccacactcagctaatttttgtattttctttagagatggggtctcactatattgctcaggctagtctcaaactcctaggctcaagtgatctgcctgcctcagcctcccatagtgctgggattacaggtgtgagccactgcactgggctgagagttaaacctttctaaggctcaattttctcatgtataaactgaagataataaggtctcataagtagggtaacagtatatcttagtttatctgtgacagtgctgatttatgcctattgttttattagtttttctattgttgttatccttgtttggctatttttataattgttagtaaagtactacacaaagtaaaaaatgaacaaatgcaagttcccttttctccctcaaatacccctagtccttctgtcttaagtagatcattagtaagggtttcttatttatcctcaacagaaaattttattataatgaaatcatattacaaggctgatttttcatattgctttttcactaaaatatgtattgttttttcacttctatgtatttaatacatttataatatctcattctttgttaatagcttcattatactgaatggctctctgtgtgtgtgtgtgtgtatatatatatatatattttttttttttttttgagacagagtctcactctgttgcctaggctggagtgcagtggctagatcttggctcactgcagcctcctccttcagggttcaagtgattctcctgcctcaccctcccaagtagctggctgagtacctcccaagtacctcccaccaccacacccggctaatttttgtatttttagtagagacagagttttgccatgttggccaggctggtcttgaactcctaacctcaggtgatccatctgcctcagccccccacagtgctgggattacaggcgcgagccactgtgcccggcctgaatggctatattttttattaggttgattttttttttaagtttatgggaatttagattgtttccttatttttactattttgaaccatgttgtaaaaatctttatatatactggataaacttttgtgagtttatcggtagtgtgaattcttacaaagatgaagtgtcaaactgtcaatacatttcattattttaccatttatctatctgcatgtgtttggccaagagaaatttcaaaacaggagagaaactagtgcttcaaaactagtcatgctacccaaaaagtatatgccatgtaaaatttaaaaaaattttactatgaaatgctgttagagatattggtgtgggctaccctgaggcacttcagcttgtgtatactgaagttgaaatgatttcaaggataatttttacatgaagggattatcattttatgcttgacctagaacttatccactccctaaattaaactccttgctagtcaaactctgggattttagttggggaggggagacattaaaactttgatagctattgttccaataaaattatcttctataaaagttggactattatctttgcactcatagtatgtaaacatgtctgttttctcacacacttggcagttgtggatgttaccaaattatttctatatttaaaattacatttaaaactttttactgaaaaataagttaaaaatgatacatctcatgtttgatttggccatgaaatatatattaaaacgaagacttaaaagtgaattgatgccatggtaatacttggaattcttacattttgcaatttaaactttcactgatttagattttaaaaatgaaaaatgttgttctttccctcaaatattagagaaagattcagttattatttgaaagagtttctctgtgatagtattctatggaagagtgaagtctttattcctactaaatgataagccaaagtagatataatcatcagtggattttgtttctttttaactcatgctttccaagtatctgtgcagttttgatgagcagtataagcttgttagtctgatgagtgcatcttatcaatacgatgaagagaatcctgtgtcaactttccagaattaccatttatgtatgtagtccttactaacttgtgtttcaacataggcattcctgttcctgtttatcccaatagtccattaaggataaaatttaaataatatagactgaatattttttaaaaaggaaaactgtataacagttgaaaacatcactgtggcaagccgaatgtttggatgaacatttggttgagaaagtgagtgaatccgaggaagtgatatgatctacagtagcccttcctacatgtttgttattaatacattcttagatatcaaatattctgtttttaaaggggggagatattgtttagttttttttaaaaaaggtaatgaagtagatgttactaaagcctttaagcatgttgaatccacagagcacctttatgaatatatatatacccagggtatatgtgcatcatgatttggttaatattgatctagaacagggcttctcaacagggacttcattgacaactggggccagataatgctttgttggaggggttgtcgtgtgcattgtagaatatttagcagtatccctggcctctacccactaaatgccagtagaacccatcctttcccaattgtgacaagcaactgagagcagtgtgatatgttgaagcaaaaacgtagcctgtgctttatatctagactcaggattcaaggcattgataggaatcatttaatagctagatatttatggataaaatcatatgctgtctggggtttgcttaaaaatatgtatatgctcctgtgtgtgtggtgctaggggatggtagtaagtaggtgagggtctaaatgaaataagattgaccaagaaaataattgttgaagttgagttcattagtaggagggggtttttatactagtttctctacccttgtatacgtttgaaattttgtatcataaagagtttatattgtttattttcttattttaattaatgtttttgaagagacagtcttgctctgtggcccaggttagagtgcgatggtgtgatcagagctcacttcagcctcaaactcctaggctcatgtgatctttctgcctcagccttccaagtagctaggactacagaggtgcatgccaccagcctggctgattttttttttttttttttttgcctgtagagacagggtcttgctatgtcaggcaggctggtttcaaacttcaggcctcaagcgatcctctcacctcacttcccaaagcatgggactgcagatatgagccactactcctggcctattatttttatttttaaaaaataatttttttagttaaacagtttagcatagattagaactcaaagtttaatatgttctgttgactaaagtctttggagctttttttagttttgagttattagtcaaattaaataacgaatgtttattaaattctaactagaatgtttgtaaaatttcggattgctattatagtgagatgaagccaggagaaagtagtcttctaggctctgacagtctttacaaactctctaatatgttagcagtaaaccataacctcaggttggttaaatcataaaacaggacttggtagtaagtatgtaaagtgctttctgttttccccttaaaaagaaccagagcggccgggtgtggtggctcacgcctgtaatcccagctctttggaaggccgaggtgggcggatcacgaggtcaggagatcgagaccatcctggctaacacagtgaaaccccatctctactaaaaatacaaaaaattagctgggcctggtggcactcacctgtagtcccagctactcaggaggctaaggcaggagaattgctcacacccggaggcagaggttgcagtgagctgagatcacgccactgcactccagcctgggcgacagagcgagaaaaaagaaccaaagcatagtgttgtatagattttagtaatttaattgttttgtggatcatggctgttgaattttccttgtgggaaataataatcacttattatatatagcatactgtgtgataatgtttttagtgtatctggaatatggtgtagccattaagaaaaaattaggtagatttctgtgtactgatacggaaggatgttcaaattcattgttacttgaaaaaaaaaaaaacttacagagcactataatctcatttgtataaagaccaaaaaatataggggaggtgggataccatgtgaatatataatctgtgtgtgtgtgtgtgtgtgtgcacgtgcacatgcatgcttatatagacacagattttctgagaggattttgaccaaagagttaataatggtaacctctgcacacaacaattgggcaatgaaactatgggaacatgactcagggagacttttactttcttagagttatctttgaagtttctgtcatagtgttatttttgtttctttttagttaatgtattacaaaattatgatacataaaattgatttttaggctgggtgtggtagctcatgcctgtagtcctagcactttgagggggctggaagattgcttgagcccagtagtatgagaccaacctgagcaagatagtgagacctcgtctctagaaagagataaaaaaaattagctgggcatagtggtgtgcacctgtagtcccagctactcgagaggctaaggtgggaggatcacttgagcccaggagcctgggctgcagtgagctgtgattgtaccactgtactccagcttgggcaatagagtgagatcctgtctcaaaaaaaaaggttaaacaattatttttttatttttgttcatatatttctaaggttcttcgggatatacttttatagtagtggaagtgtattctcagaataaactacaactttaaaaaatcaatttgaaaaaacaatccattagagaaaatggacaagggaagtgaacaattaaaagacaaagttcaggtgtccagtaagtgtatataaagctatttatctgcacctgtaattaggaatatgttaattaaaggaataatgatattatttcacacccgtcatgttggcaaccacttaaaagtctaataatgtcacatatagttgaggatgtggggaaaagcagagctttcatattctgggtggagatagtaattggtatagcactcaggaaagacaattggctaaatggtggagttgaaattttcatttagcatttctacttctaagtgtatacagtggagaaactcttgcacatttgtacaaatatgtttattccaacattgtataataataatacaaaactggaaataacctgtgtttccaccgttgggggttgcataaataaattatagtgcattcaaacaatggtatgccatagcagttaaaagaagaacagtcattcttgagaaaaaaaaaaaatcaaattgcaaaaggataaaattataccattcacatagattttttttgtttttgttttctttctttctttcgtttttttttttgttgttgtttttttttttaaggcaaggtctcactctgtcacccaggttggagtgcagtggtgcaatcttggcccactgcaacctgtgcctcctgggttcaagcaattctcccgtcttagcctcctgagtggctgggattacaggtgtatgccaccacaccctgttaatttttgtatttttgttagagacagggtttcaccatgttgcccaggctggtcttgaactcctgtgttcaagcaatctgcccacctcggcatcacagagtactgggattataggcgtgagccactgcgcccggcccatgaaaattttcaaaacacaaaacaaatctgttgtttgtagacatatacatgtgtgatcaagtcatataattggactatccactgacttcagattattacagcaggggagaaagggaagagaatgggttcacagtttggagtaggaaggatacaatttacatcttatttctttaaaaagatcagaagtaaatatggcaaaatgtagacatttatattatctggatggtgggttggtccatggttcttaaattattctgtgcatttttctcattgttttgaactataaaagtattatttaatataaaatatatttttaaaaggtactattccagtagcccagtttctgagggtggtttgaaacttgtgttattgaaatgtcttataatatcaggtgtattccttttccagaacagaaataatataataaacatagtaattctttgtatatttcctagttttacataaagtatgctctaaaaattttctttttgcgtttcagacagcatgttttcgtgaagaaagggatgtattagtgaatggagacaataaatggattacaaccttgcactatgctttccaggatgacaataacttagtaagaatcatttttgtatttctatatactgtttttttgttggaagaattagcattatttttatttaagcattgtttaaatgtaggtttcatatagaaaattaagtgcatttcacacagggtaagttgttcacttgatatgtggtgtttttttttctccctggaaatttgataccttttcattggaattctttatgctttttaataagcaagtcaaaactaaatccaaacatttaatttccattggttaagttctatagacggtcttcaggagtttttcatgatacctttaacaccagtaccattcctcttcccttgttcctgttgaagtccagctagtactttaaaattgtgctcaaattcatcttaaaataatcatttccagaattttccactgaaattaatctttctcatctcattctaactttacatttttaaattatttttattacataactaacacatgtatctctgattttttttgatacaactttaagaaacatttaaataggccggatgcggtggctcacgcctgtaatcccagcactttgggaggccaaggcgggaggatcatgaggttaggagattgagaccatcctagctaacacggtgaaaccccgtctctactaaaaatacaaaaaaattagccgggcatggtggtgggcacctgtagtcccagctactcgggaggctgaggcaggagaatagcctgaacctgggaggcagagcttgcagtgataatgccactgcactccagcctgggtgacagagcaagactcggtctcaaaaaaaaaaaaacacatttaaatatgttacatggactgtaccatctttacctaaaataaaattaaaggtttttccaaccccactggaaacaaggaaaaaaaataaaggtttatgcaactctataatgctcatggggtttatctgatttcaggccagatctaggtgttcaaacagtgtcattactagtcttttatatcccttacatgcttatagagtataataatgtttttttaaactacagattgtgacccattagtgggtaataaaatcaagttagttacaagcaacattttttttctttctttctttctttcttttcttttttttttttttgtttgctggatcttgctctgttgcccaattagagctaactgctgtatctgattcctgggctcaagcagccctcccacctcagcctcttgagtaactgggactacaggtgtgtgccactgtgccccactaacttaatttttttgttgttgttgttaagatggggtctcagtttgttgctcaggctcgtcttgaactcttggcttcaagtgatcctctcagcttgaccccctaaggtgtgtggattacaggcatgagccaccatgcccagccataaggaacatttttaaagattaaatagaatagaaaatgacagagttcatgacaagtaagtagtaagagtgaatatggtctcatgacacatttcttttataatatactgaattatcatataagccatatatattattgtgggttatagtgagaattgtgtaaaaaccatagcatagagttttttgtatgtgctagatgcatagttgttaaattgaatagaatgatgttttaactggatttattatacttaatatacaaaattttagggatgaatgtatcatgcaaagataatctgcatgggtttgttaacaacatttgtaagagcaaattaaaacacatcccacatctttgcctagaaaaattatagcaatgtatcatgataattttcaaaatgttttcaaataatgaataaaatccactatgacggtgtattctgtaatagataaatttaacactgcagatttgtgcattgcttaaaaatctacattttggtataaatatagaaataattatataattttgtaggtatattaatagaaataaagtacccaaataccaaattttatatagcaaaaaaagcctgaactgtgatttatctctctcctcttgtttagttttatttgcattcttcgtatttgacattaggattaacatttctttttcaaagacatttttcagggaaaaatagtctgtatatacccctcaataattactaaaatagaattaaagacgtaactggaggaggaatgaaacactctagtagtcaaatctcctgtaacttgccagtgtttctttaggggagccttcatcatatattcattcatctcaagagtcagaggtcttggttatcatcaaaggttgccttttgaacatttatacaatattagaaaaaatcattaaaaaatgtcgacaaagctaattttagataccaaaaaaggaaattttattatatataattgtttttcaaagtaacttttaaccctgatagcttttaaattgccaagtaaagaacataatttataatgctatacggttctagaaagctgtggcaaataactttcatttctaacctacttactacaatagtttatgaaaaaatttgcaattatagcaagtagactttttgtgtgtttcagaaaaaggcacataatttaatgaatggtagatattattatgtgattagatctttttttctttaagaaaagctcattaaatacaaagctgcttagttatatcaacattgacacataaaatttttatcacttacataacactagaatataatgaaccagtgaaatttgttcgttctgtactttcttattaattgtttcctgattagttactttatggttgaggaaaaaaaggaaaatatcttgcactgacatatgtaacttgtttaaaaggtgaaagatacttgaattttaaattaattgtagtaactcatatgaactctaaattctttataggaatccaatttgtgaagacagaaattattagaaaagtaatttttttaagaaaagtattgtttagggccgggcacggtggctcatgcctgtaatcccagcactttgggaggccaaggcgggcggatcatgaggtcaggagatcgagaccatcctggctaacacggtgaaacccggtctctactaaaaatacaaaaaactagccgggtgtggtggtgggcgcctgtagtcccaggtactggggaggctgaggcaggaggatggcctgaaccctggaggcggagcttgcagtgagctgaggtcacgccactgcactccagcctgggcaacagagggagactctgtctcaaaaaaaaaaaaaaaaaaaaaaaagtattgtttaatagtaaaaagaaagcaaggaggattaactataaggtgaggctctaaagattgactgttaatctctttgttatggcagcatgagaattaaaagagtttcctgatattcctacgaagacttctagaattatatctgttccttgaaatgctcattttgggacacataactcatttattgcatgttttcagatgttttagcattgtttccttggactggagctctttaaaaaaaaaaatccccaaagctgccatggtctttttagtaatttaaccagtgattcctcaaatcaatgattgttgctttggctttcatttccctagtgactattgaggttcagcatgtattaattggctgattgtgttcccgcttatgtgaattatctgttaatattcttagcctagttttgttggattgtttgtccctctcttaacaatcggaaggggctctttattttaggattatatccttgacctattatgaatattacctctcagtctatgttttttaaaaacttgtataaatataccttagctacattgggtaatattttaaaagtagtttgtgattaggagtaccaaatattcaattttactataaaattaaccaatcagaacaatatgatattataataatataggaataggtaaccatagcagaatagattaggaacagatataagtatatttgggatcttaaaatatgacgaagggacatttcatttgaattgaggagaaagaatggattatttaactgttactttagacttttagtcttatagactctgtctataagaaaatagtcttcacactttaatcaaaagtaaattgtcaggatagggtcagttcaacgttagggtaccccgacgtggtggctcacgcctgtaatcccagcactttgggacgcccaggtcaggagatcaagaccatcctgggcaacgtggtgaaaccccgtctctactaaaaatacaaaaattagctgggtgtggtggtgcgtgcctgtaatcccagctacttgggaggctgaggcatgagaatcacttgaacccaggaggcaggggttgcagtgaaccaagattgcgccactgcactccagcctggcgacagagcaagactctgtctcaaaacaaacaaacaaacaaaaacgttgggcctagtaaatacagcaagtaggccaaagacacaggattttaaatatattctgcttccctaattttcaaggctttaaatatgcgaaattagtttctttggtttaacacacatttcctatgtttgtaattttaacatatagtaataaaaggaatttgaattttttcaagactctttaaatcttaatgtcaactcagtaatatccctgaaaacaatatcttcattctgtaataaaaccttaatttcaaggcatttatgtgtcttgtactcactagccttcatgctaagtgctacattatcattattgtagttaggtagatgagaaaatgaaatgtcacagagattaatttttcaacgtccacttacaacatggagtcagtatttgaatacaggtttctctgactacaaaacccatttttccactatgtctagttttctccctcacttaaaaataatatataatgtacacacaaatacacatgtatatgtaatacatgcttataaagcaatgtagaacataaagttataacatcagctgtaatctaacaacctacagaatctcagtgaacagtttgatgtactcttctcatcgtttctttacaccacatacatacccataaggatccatatctaaaattagttggcttctgtttattatatacacctgagaaaagcccattctgatatttttacagtattagaaatgggaagagacacatatggtgataattactatattctacgtagtagtggtttctaaatgctgatctataggctgatgctggttctaataaagttcttactgatccaaggtgaaataaggaaaacataatgataatatgaatttctttcaatgtagggactatcctgtattccaagttttttctttcattgtttttttgggggagggggtggattcaaatgttcattcttctaaaatcttggaaagctgtgttccattgttgtaggtgaagatgataataatgatgatattagctctttgcaaaatacaacaatggtaaccctgtgttgttctctgcctcagtctttaaaaattttgttttactgctgtatgaaatttagaagttttgtaatgactgctgatcatcatcttacacagtttcttgttaattagcagcatatgtaattaactgcataaatatttgacccagtcctgttgaccttgggatacagtatattgatttttcacccttattagtttactgagtacattttatttttttcattcacagtacctggttatggattattatgttggtggggatttgcttactctactcagcaaatttgaagatagattgcctgaagatatggctagattttacttggctgagatggtgatagcaattgactcagttcatcagctacattatgtacacaggtaaaacagtccttcattgtacctggctgtgagttaccatgtaatatctagcaacagaggcctatttcttgccagatttattaattatggtcttggatacatttatcgtcaacaaaaaaaaattattttgagatgattttggttgttttgctttgttgttattattatttgtcagcattacagaaccgatgtgcctgcttatacatacatgtacatgtatagtatctaaaagttgtcactcctagaataatagacattggagactaggaaaggtgggaggaggtgagggatgagaaattacctatgggtacagggtacactattcaagtgatgggtacactatattgcataactcacaaccacacaatatgtccatgtaacaaagctgcacttgtaccccctaaatctatgaaaaaaaaagttctcggccggtcacggttgctcacgcttgtaatcccagcactttgggaggtcaaggcgggcagattacgaggtcaggagatcgagaccacggtgaaactccgtctctactaaaaatacaaaaaaaattagccgggcgtggtggcgggcacctgtagtcccagctactcggagaggctgaggcaggataatggcgtgaacgtgggaggcggagcttgcagtgagccgagatcccgccactgcactccagcctgggtgacagagcgagactctgtctacaaaaaaaaaaaaaaaaaaaaaagttctcacttacagtcagcatttctagaaatcacttcttaaaaaaccactcttgtttaaatgtacatatgaattccatctcaaaaggagtatgcattggtcagtttccttatagttgagtcttacatatttaaattagctggaattctagaccataagtcataggttcatttgctaaataagtcctgtattatatattttatgtttctcaagaaaatactttcaaggattgtatgtgccaaaagagatttttttttgtatgaaagtgataattttaatgttcatggagtaattgtaaactattaatactttatatttaaaaattttaaaaataccagcatgaatttggaaggacacacaaaaaataagaaataaattaaggatatattggatgatttaaaaggtaccctcacattttaagaggaaagtaggaaacagcagaacgttgttggtgaattaatcattcaaatgagagtatctttggatggtgcaaaatgtgagaaagagaaagatgctgaggagaagtacgtaaagggatactgccaagttagaaataaactaaaccattttctaagagtgattttcaaactttggcatgcatctgaatcctatggaggaagggtggttgaaacccagattgttgagtcagctgcagaagtgctgattcagtgggttgggtcgggctggggaattgccatctctaacaagttgccaggtggtgatgctgctgctggtgtagtgaccacactctgagaactatgctctaagacagcagtgactgtttcttttaagttttgggtggtgagagtgatctctgagaagaacatgaatgtggcaaggagtagaatgtttctgtttcagtgtttgctttttaagatgtgggtgtcttaattgctgtttcatttaattagtattgaagttctaagacattattaaaggaagactactttaattaaacaatttgtattttatcagatctctgtttggcttttgcctattcgtgtgcttttctctgtgggtatctgattgtcacagtttttattgaaaataaagatatggaaatgctttgaaaattgtacaatgcagtttttttttttttaatttaaatttttgtagagatgggggtctcactgtgttgctcaggctggtcttgaactcctggactcaagtgatcctgccacctcagcctcccaaagtgttgggattacaggcatagctaccacttcttgccacagtgcagccttaaagatggtattgtggtctcaagagtcagttatagaatgagaaacgaaagtgctattttgtggcttaaacagtaatccaaaatcctagctgaggactatcatttaaaaatacttacagaaaatactatttcgttgaatatttgaagaatgttactaaacatagagtcttaggcttgtggaattgagagatttcacccagtttatatgtaattcttgagaaggaataactgcttctgaaaattatatgcctagattggtgaaagtaaatatgaggtatttgtagtatctttcattctgccaggcttgttagagatactgttatgtggactgaaaaccatttcaaccttgagctctgcttaagtacatctaattttacaaggtacttttaaatgaatcgtatttattccattgttgggcagctctaatattataaaattttattttatattaagctgaatttcttctcttctcttttttttgagacagagttttgctcttgttgcccaagctggagtgccgtggtgcgatcttggctcactgcagcctccgcctcctgggttcaagcgattcttctgcctcagcctcctgagtagctgggattacagtcatgcgccatgaagcctggctaattttttgtgcttttagtagaggtgggttttctccatgttggtcgggttggtctcgaactccctacctcaggtgatcttcccgcctcagcctctgaaagtgctgggattacaggcatgagccaccgtgcccggccttgaatttctttttattttagtcattggttctagttttgcttttagagtcacatataatttatttctcttctagttatcatgctttctgaagtcttctttaggttgaacatctaaacatctctagtttctctgttataacaatttgtatacacttcctaccttccttgtcttaaccccccaccacacactctgcccattcctacacctcttctccctccctccttttctctacttttacctttctgtggatgctccattgtgttaatgtgctccttaaaatgttattctcattttctggatagactattttcagcaaatgctttactgaggaaatattttttgagtcctcttttataagtaacattgtgctaaactatgtggaattgtaagtaagtgggtatagcatatagttgtggcttttgaaaaggtttcagttgtttggaggcacaaaacctaagatagaaataaaacttaaaataccacaagttaaatcatttaaggccaaagtataagagatgatggagtgagtccagactgtggattgctaaatgagttatacaagcaaattacagtgagctcaagaaatgagtaataagtttatttctaatgatcagttctaatccttatttaactctttgtactattcactgtattctaacacaaattctgtgatttagatgtcatctaaaatcgacgttgccccattttatagatgaggaaacccagcttcagagacattagttagtaagtagcatagcctagattaaaatctaagtcaggcttgtctccagatttatgttctttgcacaacatcatgttttccatagaagactttaatggcggggctttgaaggatgagaagactctgtaggcagataggaagacattcaagataggtgaatggtaggagaaaagcacataagaaggtaaatagtagttttggaaaatacttaatagactagtttggctagagaaaagagttcatgttgagtttagtaggagattaatttgtaaagagattttggttcttatcatggaactcttgaatgtcacagactttgtatcttggtagctaataaactacaatgaatttcaggcaatgaaatttgaaggtaaacttcatgtcttagagttttatgaaatttatcaaatttccaaatgtgttataattcatgaacttaaaatatagggatgtggagaatacaagaagggactagtgagactgttgtgtttaaggaaggagatactctcctttttcttgtgttctttttaaggaaatatatttctagacttgtaaaagtatgttgtttgtcttctatcagcacaaaaactcgcatatgaaaaaatttgcacaaaggtttaacaagagcaatggaaaatagcaaatggtacgaataattaaagtaaaatattttctaataacattgagtagccaaaaagcactggactaggaatcagaagatttaattccatcacctatcagattccccatgtgttaaaggaggataataaataatgtttactctgcttgttttgccagactattgggaaggtgagatgaaatgtgtatatgaaagtgttttgacagtatagacaatatgccattgcaattagggaaacatctttttgcattataaatttgaggcattaaaattattataaataagatagtttcatcctttggttctataattctaaatacatttttctgtctaggtctattgagaatatttcttactcttacttagttttcatttctgattttcccttcctttctttgggcagtatttttcataagcttttgtgtgtgctaaattttatttaagaaatgcaggggtataagcatatatttctaaatttgcttgcatttcctaaggaaagttttacaactgggatacaccggcagttgtaaaaatggttgcctaaagagagtagggtggagagaaagtgggatggacagggataggaatgaaagctcaatttctaagtgaatacccttttcatatatactttttaaatcagtaaacttccttatttagagcagttttaggatcttagaaaaattgagtggaaagtatagagttccccttatatctactcccgcccaccacacacacacacgcacagcctcccccactatcaacatgccttaccagagtgtggcacatttgttaacatcaatgacccttcattatcacctaagtccatagtttacatgtttacattcgtgttctctgttggtgttgtacgttctgtaggctttgacaaatgcagaacaacgtgtatccgccataatagtattatgcggaataatttcactgccctaaaaatgctctgtgttccacctctttattcctcccttcccgctaacccctggacatgactgatctttttactgtctcataggtttgccttttccagaatatcatatagttggaatcgtacagtttatagacttttcagattggcttctctcgcttagcaatatttaaggtttgatagctcatttctttttagtgagtaacattctattgtcggcatgtaccatagttttccatttacctacttaaggacatcttggttgcttctaagttttgggaattgtgaatgaagctgcattttatatgattgcttttttttttccctctctctctttttagcatttcaattatacttctattttttaacttttcttaatggttaccctagagtttgcaatataaatttacaactaatctcagcccactttgaaatgacatggtactgcttcataggtagtgaagtaccttgtaataacagaataatcctaattccttgttcttgtcccttgtatcattgctgtcatttatttcatgcataagtaataattcatatttgagtacattgttggcattcttttgatcaaactgttatcttttagactaattaaaaataaaaggtaaaagtttttattttaccttcacttattcattctctgatgctgttcctctctttacatagttctgtttctgatttgtatcatttttcttctttttgaagaacttttcaatttttcctgaaatatagttttcctggcaacaaatcccctcagtttttgtttgtctgagaatgtatttctctttcacttttgaaggataattctgcagtatatgaaatcataggttggtgaggctttttttttcttttaaatatttcacttcactctcttcttacttgcatgatttttaaggaatcagatataattcttatctttgttcctctatgcataaggtgtttttttcctctggcttctttcagggtttttttctttatttttttatattctgtagtttgaaaatggtgtgcctaggtatagttttttttttttggggggggcgggtgggggggtgccatttatccttcttgatattttttgaatttcctggctctgtggtttgatgtaggaaaattttggggaattatcagacattattgcttcattccatttccttctggtattcccattacatgtaacttataccttttgtagtaatcccacagttcttggatattctgttctgttttttttttttccagtcttctctttgcttttttcattttgaagtttctattatattctcaagctcagagattcttttctaagtggtgtctagtctactaatgagctcatcaaaaatattatttatttctgttagtgtttttgatctctagcatttctgattctttcttagactttccatcttatttatctgttcttgagtgttgcctactttaaccataaaatctttaacatattaataatagtttttaaaattcctggtctgataattgcaacattgttgccatatctgactctcattctgatgtctgtccagtctctgtcccatttttagtatgccttgtaattttttgttgaaagacagatatgatgttgttaaccttaaataacaagattagaaaataagtatagactttgagcccaaagcttgaggatggccaccagagagcatagattctatttgccttgaatatacattctgattagcagtgttacaagtgggtttttaaggaaaaaagaaaaggcagttcctaagttgttaccaagaatttacattaaaatatcagaagctcttggttagctatacattcgttctttgtatcacaaactccaggaacgagaagacgatgggtgagacaggtagccagtaaaatgccttcaaacaattgcccctgggtttgaggggccagtgactgaagtcccatactcatgtctctctgggcctactaaattttgcatgcctcacatagctcagactgctttgagctctttttcttttctcaatgtattgggcaaaaggaattgcagtaagtgggccttgagtgatgtagtggcaaggggcgtgtgtgtgcatgtatgtgttggtaaggggccattctgtggtcctatgattaggtcccaatctttagtgaggctttgagactgcacagtgaacaccagggcttctcagcttctcagtacccctgccacctctgtacctcccttcctgccttcctgcttagttgggacaggatggctagaaggtgccagacttgggtattgcccctccttctggtaggttagtctctgataataccccagcaggttaggctctgaaaaattccaaacagattaggctctgttaaaatagtttctcctgagggcaggccttcttaagaagagaatactctggcatatttcagaatgcagcagatattctcttttctcctccctctgttggaagcatgagggtatgtttctctggtattcactatgaggacctggttgagctcttagtggtaaaactcaaaaataaatgtgcggcctccctatgactgggctgcctttggagtttttaatctttcaggcttgtccacactgaacctccagcaattcttcagtcacagtttaggttttcctaaccaggcactggttctctgcaaggtttctgctgtgggtttttaaactgccaagttgtgattctccatagccacctgtctgtccaatttggggacaacagtttgccctgtgaccttacttctcttacatatctaagaagagttgttgatttttcagtttgttcaggtttttactttttgttaggatgctgtggtgacttctggactccttacatactggaagtggaagtctccccataatcttttaaaacttagatttcaaaacaacatgttttatcattgtgatacggctctgatgagtggaggaacaccagattcttcctcttaagttgaattagaaaaaaaacgacatggacacacgtggagtagttttaaggagcagggagtttaataggcaagagagaagggggaggaaagaaagaaaaagctcgcctctacagagacagagggaggggggctccaaagccaagagacagaaccccactttcaggtagtatcaaccagctatactggaaggctggaggaggcagtgtctgatttgcatagagctcagggcattggtttgaccaggcatgtcattcacgtagccggcgaaaaaactggccctcccaccctctctttttaatatgcaaatgcggggcaccatgatgttccaaacacgtggggatatgtaggggcagccatactgccaggcacatgtgggggcaagggcaagaggacaacggtgggaatcgccatgttaggtggacccagtttctaatggctaacatttgcatatcaaaggttgccagcccgggtctaagacccaaggctttcattctagataagagctgcgaaaaatcttccaaggacccctttttttcctatgtgcctaaaataatttcttaataactcctacctcaattgagggtacagcaaccaagaactttgtttttttaaaaaaaaaaatttcttgtaaagatgaaaagatttccaaagatgctaattaaagtgttaaatgactagctggaaaaataacagcttgttattttattgagaaacctaagaacggtatgatgttataaagataaacactcaaaggggctattactattattttctactgagaattaccccagtagccttctcccttttgaaggaggcagggaaatgatgcagtggtattgaggagtgaagaaaaatggtattagtagagcagctaataatactggtgcatattgtttggaggaagtagaaataatgagtggctagtagatttgaaatgctttaagcattaaagatgtcagttcttcccaaattgatctacagacttagtctttattctttatttttttttcatgttctttcatgaggtggcaaatctataggtttattgcagtctctcaaaatcctagcaagattcctttagatagagacaagcttattttaaattttatatggaaaggcaatagaactggagtagttaaacagattttgaaaaaggagaataaagtggaaagaatcactctaccagatattaagccatgctatattgccaccttaagcaggaccatgagataattgcgatgtaataaacacacagatcattggacgacattagcaagcccagatgtaggctaacagaggtattttcaactgattttgagaaaggtgcaaaagcaaaaataatcttgaacaaatggtgctcaaataagagaccaaaaaaaaaaccccaaataacttccccaaagctctcataaatacaaaaattaattcaaaatgggtcatggatttaaatgtacaacataaaactataaaactgttggaagaaaatattggagaacattttctggatctagggcttggtaaggagtttttagatatgacaaaaaaaatcaataaaaatgaataaatgggacttcattaaaattaaaattatgtgctcaatgaaagaccctggtaagactataaaaagacaagctacaaagtgggagaaaatagttgtcaacataccttacaaaggttttatatctagactatataaagaactctcaaaatacaattaaaaagcccaagcaatctaattacaaaatagggaaaagacaatagacatttcacccaagaggagatagagatggcaagtaagcacatgaaaagatgttcaatatactgatattgaattttacgaaaatgccagttaaaaccataatgagatattattacccacctgtttgaacagttaaaataaaaagtagctaatactaaatgctagcaaggatatggagaaagtggatctcttacatgttgctggtagaaatagaaaatggcacagctactttggaagacagttggacattttttaaaagaagttaagcatgtaacatgatctgacctgtcagttacattcttgagcatttatcccagacaagtcaagatttatattcacagaaaaatctgtacttgtgtgttcatagaggctttatttgtaatagcagaataccggaaacaacacaggtatccttcagtgggtgaatagttaacaaaactggtatatccatgcagtggagtactactgaacaataaaaaggaatgaacttctgatatatacaataacccggaggggcctcaagggtgttatgctttgtgagaagaattaaatctcaaaatgttacatactgtatgtttccatttatataacattcttgaatgacaaaattacagagatagagaacagatgaataatttccaggggacagggcctggggaaaatgaatgggggagtaagtagaaatacaaagaggcagtcagtgtgagggagttcttttgtgataatggaacagttatatattgtgattgtggtggtagttacatgaatctatacacaagataaaattgcacagaactatacatgtacacacatgtacatttttaaaatggcatgtactaggcttctccagagagacagaaccagtaggatatatgtatatataaaagggagtcagttagggagaattaactgacacgattacaaggtgaagtcctatgataggatatctgcaagctggggaaagagagaagccagtaatggctcagtccgagtttgaaagccggaaaaccagggaagccgacatggcagccttcagtctgtggctgaagtcctgagattccccgggaagctgctggtgcaagtccaaaggctggtgaacctggagtctgatgtcccagggcagggaaagcggaagcaagcttcatgcactgaaagaagaaagagccagaagactcagcaagcagaattatcccaccttctaacacctgcttgcccacattgagggtgggtcttcctctttcagtccgttgactcaaatgtcagtttcctctggtaacactgtcacaaacacacccagatacaatactttaccagccaattaggcatccttcaatccagtcacactgacacctaatattaattatcagagggtgaaaactgaaataaggtctagtctaacactaatgtaccattgtcaatttttttgttttatattgtgctacagttatcatatgttaccattgggggaagctggtggaagagtacaagggattcattgaactatttttcaatttcctgtgagcctattatttcaaaataaaaatttttaggaaaaggaagggcctctatgacttgcttttctaaagactggaaaccaagcccaagaaattctgtaccttatatttcacctgcagtgcatataaatttggtgagttcctcaagattgccaaatttggttaggctctttggcctacaaggaagtgatttttcttaccagctataaggctgggatctgtaattcaaacttttgggtgttattaacatgtagatgctatgcaaatcatgggataagatgaattcactttgcgaaacagagtaggtaataaagagagtgcaggaccgatgagccatgaggactccaggtgttagagactggatagattaggagtagccaataaagaaaactaagaaggagcagtcagaggttaggaggaaaatcatgatagctgagagcatacctacaatgtcttacggaaaataaaagtaatcattttccccttatatttaattacattaatgtcttaatggatacatttcaggcaaacatttattatgtttttcctgtggatgctagtgtcctgggtgttggaggttgaagtagggaaggctgtagggtattgcctgcttttatgtaagtaatatactttttaccttcaatttactgtctttggggaaatgataaaagcttaagaaaatacttctacttcatagttgtgcccaagttggacagatgttcatctcaattttaattttgtaaattcattcatttatcatgaacgtattaggctgtacagtttttcctgatattctagctattttcttgggtaagaggtgccatagaactggaggaaatactctttagaggagctacttgaagattaactgttataatagagagtaagcgacaaccaaaacaggtttaaaacaaaaagctgcaaaaactatttttacgtctccatataaaataaaagaaaatagtagatgaacaagtgaatcaggaagatctcttggaaaaagccatgtttatcataggaagaagatacttcaaaaaattgaaatagcttaaagaaggaggaccaaagtaaataatatgtacaaaaaatgaccattgaggttctgaaataatattttatcattattttaatgttaaagatgtcacttgaatacatagaaaaatgcaatatgaagatcttaatattatgaaaatatgtccatctttatgctttttaacaaaatggtgggtttttttccctgtgttaattgaaatactattttggcagtcatatctttttttcaattatctgtaaactcagatttgtgcatctatgcactatgcatctgtaaagtgaatagatgcactttaatttcagctgaaagtttgaatcctagcattactgtttttgatttgctgcataggcagtttgcattttaagggtgtaccctttcttttcaattatttttttgttcaaatacaaaatttgggagtagaggttatttctaatgacttaacagcatcatgtggtatgtctgattaaggttgattaaagttgatgccaaataggttgtggtaatgtaaggcctaatttcaggttgaaaaaaaaaaggccccaagttatttctaaccatgtttaaactcagcactagacttgctatttagattgtgaactataacttcaaaaatgctaatgatttaagacacagaatcctactagaacaagaaagaaaaactaagtttcatctgattagaatgttatgttcgattatcatgcgcttcagttttagatgacaaaaatgggaagatgaggaacttttgtggtgtttttcagcactacgtagataatttacatagatagtcagaatgttgattctggcttctggcaccaaaatgttaccaactgatattgtgtgtccttcagtgacttagatcttaccttgattctgtttgtggtatagtttcctacaatatcaccatttaaacaatgttgcacactttgccttcaaactggaaggaccctattagaagccattttgattgagaagggtcattgctggttggcgtattggtctgttaaagagaatctttattgttgtaaaatatataatgctttaagcttgtgtattagagaggataacagtttcttttcttagatcacctgtcatttcataaaatgaaattttatattaatttttttttgaaaagttcttagtggaaatagtagaatatttaacagctttttggaaaaaattgagaattccttctttattttaaacccttcaaaggcagattgattgattgattgattgatttatgacggaatctcactctgtcacccaggctggagtgcagtggcatgatcttggttcactgcaacatctgcctcctgcgttcaagcgagtctcgtgcctcagcctcctgagtagcttggattacaggtttgtgccaccatgcccggctaagttttgtatttttagtagagacaggtttcaccatgttggccaggctggtcttgaactcctaaccccagtgatccgccctccatggcctcccaaagtactgggattacaggcgtgaacctccgcatcctgtccaaaggcagttttaaaaaatgatctaaaaatggtgaacttttctttattagctttaatcataataacactagatattatccttgatagttaatattaatagtagtcatttatatattactacttcttaagacttttttttttttcaaacttttttgccctttggaggaaagatctctacctttttgtataacttgccagtggtattcagtggatcctcattaaaaattattgtgagattaaattgtaaatagtcttttaaaatagctttttttttttttttttttttttttgagatggaggctcgctctgttgcccaggctggagtgcagtggcacaatctcggctcactgcaagctccacctcccgggttgacgccattctcctgcctcagcctcctgagtagctgggactacaggtgcccaccaccacgcccggctaattttttgtatttttagtagagacggagtttcaccatgttagccaggatggtctcaatctcctgaccttgtgatccgcccgcctcggcctcccaaagtgctgggattacaggcgtgagccaccatgcccggccttaaaatagctttttgagataaattcatataccatacagtccactcattaatgtgtacagtggtttttggtatattcacagatatttgcaaccattactatgatctattttagaatattttcatcatttcagaaagacactctactctataccttaagtctctacaccctgtttccctatttccccagccttaagcaaccattagtctaccttctatctctgtagatttgcctattccagacattgcctataaatgaaatcatataacacgcggttttctgagtggcttttttcacttagcataatgttttcaagattcagtcatgttgtaggatatgtcaggacatatgatctcaaccaggtagactgcctggtaagactaagacaccaacattttccagagttttgagaaagaaccaaaagtgtacatgctcatagcatacacagtgcagtccaaaattactcagtctatcaggaaattatgatttagtctcaagagaaaaaacaactaatggcaactgtatttagtccattttcatgctgctaacaaagacatatctggccaggcctggtggctcatgtctgtaatctcagcattttgggaggccaaggtgggcggatcacctgaggttaggagttcaagaccagcctgaccaacatggagaaaccccgtctctactaaaaatacaacattagctgggcgtggtggcacatgcctgtagtcgtagctacccaggaggctgagtcaggagaatcacttgaatccgggaggtagaggttgtggtgagccgagatcatgccattgcactccagcctgggcaacaagagtgaaactgcatctcaaaaaacaacaacaaaaaagacaagacataccccaagactgggcaatttataaaagaaagaggtttaattggacttacagttccacgtggctggagaagcctcacaatcatggtggaagtctaggagtagcaagtcccatcttacatggatggcagcaggcaaagagagaatgagaaaccctgataaaagtggaaatccccgataaaaccatcagatctcatgagatgtattcactatcatgaaaacagtatggggaaaactgcccccatgattcagttatctcccactgggtccctcccacaacatgtgggaatcctgggagtacagttcaagatgagattttggtggggacacagagctaaaccagcaactcaagatgatccacgtatgagaattattggacaaagactttaaagcatttagtttaaatatgttccatgaactcaagaaaaacacatttgttttcttccaggtgaaagacatgatagaagtatctcagcaataaaataaaaactataaaagaaacaaatggaaattttaggacggaaaaatacagtattggaaaaaaaaattaaatgtctttgagaggacaaaggagattacataggaataaagtatattcaaattcaatttggatatagatcaataaaaattatcccatgaaaaacagacaaaaagattgaaaaaatgagcagattctcaggaatgaaaaggtgtaacatgcaagtaattggagttccaggtagtgagaatagagacaataggacagaaaagaataatagctccaaacttcccatgtttggagaaatacatgaatttgtagaataaaaaattgcatagtgttacagctcttttagaattgcaccagaaccctcccctgccgtccaccccacggaagaaaattgcagcaaacccaaaacaacataaattctgggaaaaccacacctagctcatcaaaatcaaagtacagaatacaatggatagagaaaaagtcttgaaagcaccagaggaaaacagtatatcacatacagggaaaaaaaaatggaatgatctcagatttctcatcaagaccacgggggctaaaaggtaattaacatcttcagagtattgagaggaagaaaactatcaatgcagtattctgtatctggggatattttcttcagtaatgaagtgggggtgagggggagacattttcagataaaggtaacccaaaagaattcattgcttcaggccttccttacaagaaatgctgaaaaaagcacttcagaatgtagggcaaagatagcagaaggcaacctggatttttaggaaagaagaaagagaataataattagtaaatatctcagtgtatatgaaatactgtatatatatatattttttttttcttacatctttaaaatatatggctttaaagtaaaagttgttttttgggatattgaatgaatacataatgtatatgacagctatgagataatcgagagaggtagggcataaacaaattctagaataattatttacctttaaagttaaaaaaaaaagtagggggcataaagagccagtggattagaggggagagaaaactactttatcattagacttcccagtgatgaagctttatgataaagatttataataccctagaaaagaaaatgtgaacaaaacagactttcaggtataaaaggcatgtacaaatgttatcaaatacaagaattttaggaatgttgtttccatgaattcctcttgaagaagatactaatctagttagtatagtgtaataatatatactagttagtatagtatacattttctagtataacctactagaaaatgagcttcagatgactaacatggctatagagatatcagtataaggactggtattgctcattaaatagtcagttgtagaactaaagcagaaaaaagttaaaaggagtgagcacaggattaatggctatatgctgtggcatgagcatgtagtataactgtaaaagtaggtagtgataattgggagaatttatataaaatttttttcaaactgtttgcagtaatcaaattagtggtgacagtatagttactgttattttgacattgttgtgtttataacgtgaaatgaaacagaatgattatggagtattctaattttatcccttatgttaagaatcagaattcttggtgtagaagaaaggagatgctgatgtaatctggaagagatcattaaaaaccctaatgtcgtgaatttgaattgaagatatcattgtgggctgggcatggtggctcacacctgtaatattagcactttgagaggccaaggcgggcagatcacttaagatcaggaattcgagaccagcctggctaacatggtgaaaccctgtctctactacagatacaaaaattagcccggtatggtatcacacgcctgtaatcccagctactcaaggagctgaggcccaagaatcgcttgaacctgggaggcggaggttgcagtgagccaagatcgcaccactgcactttatgtcagtatgaacttctgcagtatttcatacacacacccagtacaccatacatacagctctgtgtggtgaaaataactagaaacagtgaccagcacgtagcagtgagcatccctattgacagattatcgtcctcacatacgatttcctactgcagggaaaacaggctacttggagaaattgctgactcaagatctagataggaagtgtacaagatgagccctgtatgtcttgtcatgccaaatagcaaataagctttcaaagacttttagggtcatgtttgaaggatacaggagtcaaattggaaaggttactagtggcgtaagatgggaaaatttcaaatttaatagggataataatagaaagaaatagaaactcatcagatgtgcctgtattaatgagttttaatgagtgcataatgtacctgtaccagcctcagtgccttggagatgaactgaaaatatgaacagtacatgtaatgaatgattacttggtagtttatcatatgctgtgttaaagtataggatactacccattatccagttatttatttatatatatatatatttaaattagacttcaagtttgcagaggcatagactatgactttttttctattgtactcagtggatatattttatgatgtctctctggaggttttcttcatctagacctgcagccttgtggtttcaagggcttctttctgggtgttggttaataggttgctgcaacacagcactacagttgtcaagtcatattttaggaacagttgctgttatatctggaatttagaccatgaattctatataagttccaaaaggggttatatcagtcatgattgcatggaatgtacacgaaaatatatttcctgataatcaaattatttatctagttatttattcctttcatcttcactgtgttccataatgaatttagataattagaaataagaatgcatgcaataaatcaatggcagaatataaaaaggaataaagaatcacaaaatggaaattatattttaaaatgaagaccccaaaccaaggagaaataatgaacagaagtcgagtcatatgcttgaggcacaccagctttctgttgcgtagaatctctccctgagggcaagcaggttacatgaaacttagtgaaacttagtggaatatacgtgctttcctatctttgttttaaatcttaaatactggattgaatagattcttaggatggaatgtatttttaaagtaatttttctttaccctagtccaaaaaactaaaatgaaaaaccagttaacttatttatggttttcttcataactctttttatctcatgtagtttcatagtcaagatttttgattacagcttttattaacctcatctctttaatgttgataatattttatataatgtggttgagtaaagtgtaccgatataggaaatagagcaatcctattttaatagaatttgttgggggggaaataccacataattcatatctagagagtttttcatctgttatcccataagaatttcatgaaggattggtatgtgctttgcctataccagagctttgcgctgatttcttctctttcagcttagatttgtgggtatgatttggcagttatcctcttggaatgtgtaaaatcattacttttacaatgagatagaaatattttgaggcctaaggcttactcaaaattctctcagtctcttacaggcctgagtcacatttcaaaattggaatttttcatattgctaatggaggaactatttttatagtattgtgaaactattaaagaataggtaatgttatatggatgctcctcgacttacagtgggctatgttccagtaagcccatcataaagttgaagaatcataatttgaaccattgtaagttagggaccatctatagttaaaaatatagttaaaaactgttacactagagtataggtgaactcaggatgtgcacaattaggaatttttaaatataaatatagttactatattacagttcagttggtatttcagtaactacaaggaatttggggcaactacagagttttagaggaaaccttccagttagaccactccaagttctgacaccaattggaagttcgaggggttcccaaaaccacctcttagttttgataatttgcaagactcacagaactcactgaatgctgttatacttaacagttgtattttattacatggaataccgattttattacatggataccgatttaaatcagccagaggaagaagtattaatatatagggtagagttaaaaaaaaaataccacatgtggagcttacatcattctttcttggtggaattaggacagcccagcatacatatatgatagtgcttaccgagtattgccagccagggaagcccacctaagcctttggtttttatagtttttttagggtggcctcaattacttactgcctgtgtgactgtctttagtctccagcccttcccaaatgttgggctgatagctttagtctccagtcctttccagaggcaaaacatagtatgactcaaataaattacatgtatcataaattacattggtagattgtctagtagtcaaagacactcctatcagttgtgctattccaggagcctagcgaccatcttccagtagctgaggataaaagccagacctctctttgggcaagattaggccttcactatacagataattccgtggtctttaggcaagctttcctatgatatttggacatcttcatttagtgtaatgttcatatggcagatacagcaatagtatcggtttgaatgtatctgtcatttggaggagccaatgcagggtaattgaacgaaacttctggtctatcctataaagccattatatacgttttcatatttttgtactaatgtaattacttgtttcctccttttatctgtcactatttgtacttgtacagaactatttagcatagaagttacttgataggtagctaaatgtagttattcctcaggctggccatttgattagatcctgaggagcctttaactgaatctcccagtgagtattcctgagatcagatgctattggctatactgtagttttagaaggtttttcttttagtgatatattatttatctacatacaagtatttgttaaagagagttagtgtaaagacatgttaatatataacaggataatgagaagcataaaataattctataaatttgctcaagatgatttttttttagcttagaaaaccttattttgaaataagtttggaatcacaggaaattgcaaaggtagtacagaaaggtcccatgtacctttcgcccagtttcccccaactatactatctatcctactcatggtaggacttcttccttccgtctcagtgggcagtggtttaatctgcattttattgccccattactccccgcttttctcttctttttctaatttgctgaatttgaatgtttattttcataattacacataatttgaatttcatagtagtctttctttgaattacattttaaatgtggtatgtgggttgttttacttgttctccttgttgattgtatctggatggtttttggaaagatgcagagaaagattaggatttgtataaccaccattattttgcagtacctggaattatagaatcattagttttttattcccataattcttaaataatagcttcactacccagtggaaaagattagtttgagaaaagtttactgacagaattttttgaataactgttttctcaaatgtaggatgaaccccaaattaatgttccataaagaattctaatttctgactgacagaaaggctgttggtgatgtaattttaccagtgtggactttcttttattactgatgtaatatttctttgacaaaattaagagacaatttcaaaaagcagtttcattttaatataatgcagtatgctgtttttatgagtataaaaaaggaggagtaggaaaattagaaaataaaagtattgttttagaagagtccactaccctaacctatataaatgataaaaaggaaccgaatgggagatattaatgtgatgttgctgggaagagaaatgcctgtcttaaattagtggttgtcagggaccaatcccctgtgcaatattttgattagtggtgtctgggttggggccaaggtacatgcaattaaaataaccctctatagttgaattcagtgtccagccagaattgaaactatatagattaactaatgtcgtatccgcattttggtcttatgggggtaatatttgtaataatcatgcttttggtactgatgttctagtgaagttaaaaaaaaaatcacccatagaatgttgccctttcaatgtaatcgttgttttagaatgctttttttttgagacaggatcttgctctgtcacccaggctagagtgtagtggcatcgtcacggctcactacagctttgacctcctgggcacaggtgatcctctgacctcagcctcttgggtagctgggaccactagcgcaggcgtggaccactattcttggctaatttttgtagtttttgtagcgatgaggttgctcacgctggtcataaactcctggactcaagtgttccactggccttgcctcccaaagttctgggattataggcgtgagccattgtgcccagcatagaatgcttttaaagtttttaaaactattattttaaaactaaaatcataaaacttttaacccaaatcttatttacttttgtttagggacaaggaaaattattcattttagaaactttaattagtgtacatgttttgactaatattcaaattatggaaaacttgaataatgtttttacagtaatagatattttatctcttccaaacatttctcagttttcatgaattatcaaagttgaaaataatgttttacctgaactttcctttcactccttttattgacaattcttagtatctggttgtctggctgccacgttatttgcaagacgtcactgactcagagtggttttgaatgccagatgttattttgctgcttcttccagaaaatggaatttgttgggctactgttccacttaacaattgctgtgtaacacattatgttctccagaagcaggcactgagacagaatgtgtcatgcagggtatttattaggggttaacacctgtagaagggaattggagaaagcaggtttgggtggaggagaagtcagattacactgaggccaaagcttcagtcagccctactaggagttctgaagcgtctgtggtcacctgaattgttgctagtttagtagaaatggcatggtctttcttatttccttatcagtcattcaatgcatatgaagttctccaagaaacacaggcagttgtttaaagaggctctctgtaactttgaaggaacctggcagtggggagctatctgttgacatctggggcaaagctcatcattgagggagaagataatacatctctgtccaccatacaccctgaaacactatagtgactaatataaaacaacaacaattttttttttcttttttttttttgctcatgagtctgaaatttgggcaggactaggcagcacctaattgtatcttctctatgcagcatgagttgcagtagctcacctgtggttggaagaatcactgtcaagatggctcacacacatagctagccaattggtgctgcccatcagttgggagttcagccagagctgtgggcatggggtttttgttcttcccatgtgggctgcttgactttcttaacaacatgataagtatgttctctagagcaagtgtctctagagaacaaagcagaattgcatgacattttaatgacctagccttggacgtcatagccagcctaagttctatcatactgtattgattgagacagtcacaagagtccatccaggttcaagctgaagggacaatcttcttgatgggtgagtggcaaggttctagatgaaaatgtggagtgagagatattattgaggccatgtttgaaaaatatggttttctgtattcataaaacttaaaagttcagggtgccttaattagaaataggattggctacatcttttagaaaattcaaattaataatggtccaaatgtatgaggagggctagtgactgaatgaggtccttctgggctacggccctcacagaagcaactaaaaatctgagcatagcacaaagaccattatcctaaaggcactaaagagtgaacagaagcggggagaacctgattgggaatacatcctcaaagaagatggaggtatatgaaatgacatctcatgttttgcagctctcagcctggggctgggtgaaaatggcatggtgtacattgtggcatgggagaagataacaagaagataagtagaagaactctggtctctatggcctgtgaaagctgaatttggggaaactatgtttgttagagaggggatccaaaatctgtctactgacagctcacatgacccctgagtgatagcttcactcagagctgctcagctaaagacaaaagataagaaacttcttcccaagaaacagtttgcagtttaaatgtgtttaatcagctgattaaaaaaagaaagaaaaaaatgctaactggagaaaaattaaagaatccagagtttcagaaatttaacatttattatgttcagggaacattcagaattacccaatgtataaagaaaaaggagtatgtaactcctttacaagagaaaagagtatccctgaaatgacccagatgtagaaattattaatagcagactaggatttttaagtggctgttaacaactattctcagtaaaggtaaataaaatgtgttcacaatatatgaaaaaataggaaatctcagcacagaactagaaacaaatcaaatggaaattctagaaatgaaaaatacaaaatctgaaatgaaaattttcactgcaaaaaattaacagcctagtggagatgacaaagtaaagacttgaagttaaataagccagaagttattgaaggtaacagagaggaaaagattaaaataatagtagtccaaggacctgttagaaaatatcacaaggttttatgtacatgtactttgagatcaagaaggagaggaaagagaatgggccagaaaaaatacctgaagaaataatggcctcaaatttgatgaaagatataattttatgtatttgagatgctcaagaaacaccagggggccgagtgcagtggctcacgcctgtaatcctagcactttgggaggccgaggcggatggatcacgaggtcaggagatcgagaccatcctggctaacacggtgaaaccccttctctactaaaaatacaaaaaaaattagccggacgtggtggcaggcgcctgtagtcccagctactcgggaggctgaggcaggagaatggcgtgaacccaggaggcggagcttgcagcgagcggagatcgcaccattgcactccagcctgggtgacagagcgagactctgcctcaaaaaaaaaaaaaaaacaccagggaagataaatatgaaaaaggccatgtcaaggttcattatagtgaaactattgaaatccaaagacagagagcaaattttggaagcagcaaggaaaaaatgacaagttacatttgggggaactacaattccattaagagatgaattctcattagaaaataaagaagctaaaagagagacttaagaattctatatatccagtgaaaatatccttcaaaaataaatgtgatatgatatttttaagtaaaagaagaacaaaaagaacaagaagccaggggtgatggctcatgcctataatccctgcactttgggacactgaggcaggcggatcacttgaggttaggagtttgagaccagcctgactaacatggtgaaaccctgcctctactaaaaatacaaaattagccaggcttagtggcgggcacctgtaatcccagctactcaggaggctgagggcaggagccttgcttgaacccaggaggtggaggtttcagtgagccaagatcatgccactgcacttgagcctgggtgacagagtgagactccgtctaaaaaaaaaaaagaacagaagaatttattgccagaagacctgcactgtaagacatgctaaaggaagttctttaggctgcagggaaatgatactacatgaaagcttggatctgcagaaaggaatccaaggcacttaaaattgtaaatgtctagatatacatactagactataaattttttattcttactaatttcttcacaaaccatatgactgcttaaagcaaaaattataacattgtcttgaactcatcagagatttgaggtcatagggcaaccaactaacctgaaatctaagaaatgacaggtacttccaaggaaaagatcattgaggtacaaaggagagcagagagctgcatattaagaaagggcagcattaggggaatggaaattatgcttctgggaaaagaggaaccaggaaaggaaactgagaagcattaattttagatgggagaaaaaatagagaagaaagtggtatagttgggagaataaccagcagtgtcagatgtcatagagaagtcattacaggattcaaactaaaaagatgctactggatttggcagtgaaaaggtcatttgtcatcttaaagagtattttctgcataatgaatagggaaagggaaagccaaaaaaaaacttttggacaacttcttttcagcactcaaattccttttaagaattgatcatacatttcagtggtaatgtaaggacgaggacttgcattttcttggagcaggtataatcataggtaactttatacttatgaccttcattatttaaaaagattttaccaaatatggaatgcaaaacattttctaggaggcctctagtttttattcttatgtagtattgtaagatgccaaaatcttagctcagtgatcaaattatttgtacagagaataggtagatgtttatctacctacttttctttgatcctacttcttcaagatgttggaaccttttgggtttgtgggtactgtcaattaagtgtaaccttctttctgttcaggtgacactagagagctcttgctattcctcctctcttgagcccccctcccccaagtttgaatgaagtatctatgcttgttttactgcctattgagaacatccttatgaagtaattaagagccttctttactcagttgatagatgttggtcagcagaggcattgaggcacccccactcccgcccctagtgagcactaatgggaattggaatgtgtggcttctcctcactttgtttacactttagagtacagaaagaaccagggtggtccttgtataagttcagcttagttcacatcatactttgccccaaacatttttcttatgactactataaatttagatttaaatgaaaataataggtaagtctgaaggaaaaaggaaacctcagaaggccaagctatagtcttggtcaaatgttttcttttgctcgaggtgataaagtatgtgaatttatgtaagttttacgccttactttcacctggtaatttagtgaaatattcaggccagctagatggaaaagccattttctcttttaccctatgaatccttcaaattctgcctcataccagcattatctattatattgcctcacccccaaaacaaaaataaaacttaaatattttcaaacacaacagatgactagggagaagaaatagcccaccctcctgaggcctcgttatttcgaaggttaggagagtttccgtctgcccaaatagtgaccattttaagaacagagacttcagccatggaataaaatgagtaaggatgtggagtgagtcagtaggctacatactttattaaatgtataattgtactcattgttggataaacagagaccaattaatattgtcattttaacatcatggtgcttatctgtgatattgataaccattaactgtttggactaaaatatattgcggactgcagatgatcttagtggcttagtgaaaacattttcatattctttgattaaatgattcagacatttacacatgtatttttggtttttaattaattttaatcaaacaatacttaacacagtaaagaaaaaactaatacaaaaaaaggttatattgaaaagtagtctctccagcatacctcttatgcctgctccctagtggcagctatattcaattctattttttaaaagttatttagccccatatctctaatgtcctgatatgttgatgtttctagatttgcgaattttggacagtttgtaaagacttcttttgcctgccttctatgctagtggaggatttagctgtttgtattcctctttctacttttcctccctgcattctctcactatacttttatcactatttttgatcttcttggtagataatggttgcatttttgagactttgtaaatattgtttattgctgagccaagaaatatactataattttttcctttcttatctaacaatttttcctataattaataataggctcaaatttgttttcagttgtttgtcagtactcttaaaatttccttttatgtgtactaaatgcctaacagtacattttcaagtattcgagtacatcaaatcatgtatcaagtcaatttagttcctttctcccaaccttgtaggcagttataattcagcttccttggctctgcattatttatcatatgtatgttctccatctttgaaaattgtgttgacacccatcatcttccattgtcttttcttacattcacttgtgggtttctaccttttagttcttgtattgtcattttactgtagttctggagggagcagagataaacatgtttactcccataaatatttttggggtgatgagggaataagaagaaggatttctgttttctacttttatgtactcctatagtgcatagtattgcatttaattcagtgctgttgcatgctgtaaatgtttaattaatgcttattgaatgttttactttgtcagttgttacattcttatcccaaccctcactctacccccaacatgcttatgggatcctgaaattttatttcctaaaacctgtctttctctcagctgctaatatataacataagattatttatacaagaagactatgtattgatttggaagaaaatgtgaagtgataagagtaatgttgagccctttgcattttagtgatttattgagagtttactgaatgttttgaaactaggatctggtttccagagtctgaatttttaaaaatttagttaggggtgtgtgtgtgtgcgtttgtgtgtgcgtgcacgtgtgtgtgcgctcatgcgcacgcacttgaaaagagataggagtcttgctctcacctaggctgaagtgcagtggcatgatcatggctcacaacagctttgaactcctgggctcaagtgatccttccacctcagcctcctgagtagcctcctgagtagctgggaccacaggcatgcaccacaatgcctaagatttaatatttttttttatagagacagagtctcactatgttgcctatagatgagacatgtaagttctctgtggaaaataattcataactgaagaatataaaagaagacttataggtttagtgtatatacagatgggaagtgtcacattgcaaagatgtcagttctcttcagattaatctataaaatcatgattcccataaaaatttcaatatgtattatagaacatattaagctaattccagcattgatctagaagagtaatggcaattaaaaaatatgctctgccgataggaagctcattattttataattaagacactgtggtattggtgcaggtagaggcatatggatagggtggccattcatcttggcttgtgtgggagagttccaatttatgcctgttatcctggcaattattaataggctccctttcactgtaaagattgtcaactgaaaaaaaatagagtaatacagtaatatttttgctcttcttgagagtcctgaaacacacccactaaatttatgacctatgtggtattagataatagtggtgaaaggatatcattcagtaaatggtgctggggtagaaagaaatgtaactgccctttatctcacactacacacaaaaattaatccccggtagatttaaaaactggaaaagcataactttaaaacttacacaagagaaaattaggacaccatccttatgatgtctgggaagacagaaatcatagtataaattataagactacattagtattttatatatatatatattttttttttttacaaataagaaggacattaaaaacaatggaaaaacaagcccacatgcagggagaagggatttgtaacacatcatgaagaactaagttcgagtaaatagaatccctgaagattaaacaaaaaacagtgcacacacatttttacaacggcaagggatattaacaggcattttatagaagaggaatgtaaaatgaccaatgataccagtgataatatgaaaaagattctcaactgtattcgtaatcatgctagcaaagttgaagatgtccatatctttcatctcagaaatttcacttctacatctatagtctggagaagcattcatatatgagtactagaagacatttattaaaatattcatccagcattctgtgttttaacaaaataaaaatgggcaacaatcaaaattccgttatcaggagaattgataaattttgatatgtatataattaaaattaattaaaattaatgaattaaaacaatatgtcccaaaatagatgttttagaaacatatatacacacacatatatatgtttggaaacatacacacatatgtttggaaacatatacacacatatatgtttggaaacacacacacatatgtttggaaacatacatatatgtttggaaacacacatgtttggaaacatacacacatatttatgtttggaaacatacacatgtttggaaacacacacacatgtttggaaacatacacgtttggaaacaaatgtttggaaacatacacacgtttggaaacatatatgtttggaaacacacacatatgtttggaaacacacaaatatacacacacatatatgtttggaaacacacacacacacacacacacacacacacacacatatatatttattttattttatttttttcttttggagacagtttctcattccatcacccaggctggagtgtagtgacacagtcacggctcactgcagccttgacctcccaggctcaggtgatcttctcacttcagcctcccaaatagctgggatcgcaagcatgtgccatcctgcccagctaatttttcatttttattttttgtagagaaggggtctcactgtgttgcacaggctgttcttaaactcctgggctcaagtaatcctccctcctcggcctcctttggcctcccaaagtgctgggattataggcatgaactatcacacccagctgatgaagcataatatttgaataaaccaagttgcaaaaggcaatgcacagtttggtgtcatttataaagacattttaaaaagtacatagttttatgatgttgtatataggcataatagtataaaatatgcatggatacagcaaacaccaaatttaggatagtagctgtgtctgggtagtggagggagaggatgagatcaggaatggtaatagaatacttatgtttttcagtactatctgtaatgtcttgttaatttttagtattttctattttatttatatagtattatgaataaaatatattcacataatatgctttatgtatatgtatatagtttataattactatctataaaataagcaaagatattattgatgcatataaatcctgtctttgcagttcctttaatctcctttacaatctaagaagtactataccagtagaagtaattcgtagtgactgtatagcttaatttttaagataaagaataaaatattttttcctcatctaaaaatggtcttagtaattgctaagcttccttccagaactaaactgtacttctttgatgtttcccctacagcagatctttttcaacaggttagaatacagtcaaatgttgaggattataaagctaataccctatacagattgttgttttcaatcaccctgtctctctttagcaaccatctatcatctacaataggggtttgaaaactttttctataaacagcctgagagtaaatatgttaggctttgtaggccatgtggtctgtgtcacagctactcactttgcctttgtagtgtgaaagcagacataggcaatacctaaatgaatgggcatggctgtgtttcaataaaactttatttacaaaaacaggcagcaaaccagatttggccgtgggccataatttactaactcgtgatatacaacacactcagagaaattgcttcagtaatcaaagtaatactgttataatattttacccttattatgggtggtaaaactgaagcatagagatcttaaatggcattattcaggttaaagtagccctcccatattcctgtaatttacatggcaatgtaatttttaaaaaaatatataggttctccagagattctttttctccctctaaaagtagagtgagttgggtgggtataatctcttagttattcacaattacaacctttactatgattagaaacttaatattgttaagtccctgagaatcttctttacagaaccattttatatatgattatttttctaaggtttgcattcacatgcaaaaacaaatacaaaagctggtatatgatatagtttagatttgggtatcttacacaccattgacttaacatccaaagacaatgaaagctttaaataatatagacaatctattaggcagttgttttagttcttctgtaaaatcaactttatgaagcaatttctttgtgttgtcatgacctgcagtaattgagactttatctatctgcagttatcttaaggtctagaaagtatatttttccctgtccagtttcatgggctagaggatagtattttttgtggttatcaggtaatgttgactcttctgtatgcagaaagtaggtctaccatagtcacattggaatctgtgtaaatcaaaaggaaagatagtaagattaatttaaccagaatgaaaattaccacactactttttatctctagaagaatactgaaattacatgtgtttgagaaagttttaatatgaatcagaaccaggatttgaattaataaagtaattaaatagattttcttagaatattacttttaaaataagttgagatttttttctgacaaaaagaatatcttaccaaacaatgaattgtttatttttccactaaaaattgaatttatgtattttttcagagacattaaacctgacaatatactgatggatatgaatggacatattcggttagcagattttggttcttgtctgaagctgatggaagatggaacggtaaataaagataaatcctaaattattttgttcacagacatttcccttgctatttgtctgatatatctgttttagaaaccatctatttatactatctacccatccatccatttgtctatctatgcgccacctccttccacaaaggcatttgggcatacttacagtggggttgtctaaaaagtcacaagccatttcagctgcttatatcagtgatcttaagattgataaaatataccctttatgagacccttgtttgctccttctttagttggtgacttctgttgtactttagatatttcaaagttcaccgggggtaaaagaactaagtctcacatagaaaaagaaaacgcagctttgcccgcacttttacttttttgtctttctttcctaaattgcttagttattctaagggatagtgttcaacaaatgaatccctttttcagaaagttcacaagttgatttagtttcatgttcccatttgcttaacttttgctaaccctaaacccgtcagttggttttttttgtttacttaatagctgggcatggtggtgcgtgcctgtagtcccagctactgaggagtctgaggtgggaggatctcctgagcatgggaggcagaggttgcagtgagccaagattgcaccactgcactccagcctgggcaacagtgaagtcctgtctcccaccccctcactcccccagagccacccagaaaaaaaaggctgggcatggtggctcacgcttgtaatcccaacactttgggaggctgaggcaagagcatcacttgagtccaggagtttgagaccagcttaagcaacatagtgatactctgtctctgttttaaagaaaaaaaaaagtttatatgccacagttttattctgtttttttttcttttaacttcatttttttctggccatataggagaggtccaaaaatatatcagtttaattgacagaaactttttggaggacagtttagtataatacattccaaaagccatgaaaatgtcacattactctagaccagtaattttaactttggaaaaattttattattttttttttagacaaagtctctgttgcctaggctggagtgcagaggcgccatcttagctcactgcaacctccgcctcctgagttcaagtgattcttctgcctcagcctcccaagtagttgggatacaggcaagcaccaccacgcctggctaatttttgtattttttgtagagatggggtttcaccatgttggtcaggctggtctcaaactcctgacctcaggttatctgcccacctcggcctcccaaagtgctgggattacaggcgtgagccaccgtgcccggccaactttggaaaatttgtcctacagtgataactttgttaaaaagaattatgtgacctctgtctctggccatgatagagggattaaattggtaccaaattagcactcccactgtaaggaactgtaaaactggatacaatagttgaagctactgttttccagcagtggacaacaggcagcataatactattatccctgacataaatgaactcgtgaggtattcctcacgattgcgcaaagactggggcagttcctgactctgatgcagaaagctagagtgaaagcagagctggactacctgcagcagctggaatatgcagggcttggcattggagaggagggaattgtgcagagagagggcataaagtgcctcatgaaggttgggctgtgcattgtaccacaagaccatatgaggcttgacaatagcaaatgatagcatttgaaaacagaccagaatactaatgatcaagcagatctcccaggcaggaaacagtgctaggggaaattgaattttcatccctggagagccagctaacacctttgtatcacctaagacctactctctaacaacctaagtccaagccctgattagatccatagagtttggaagtcttccttccaagtagaggggcttatgacaaatcttagatttttgtatgaacctacaatcacagagtgtatctgtaagctaactcatggtcaaggagatcagtcagtaatttttttatgtgcttacataagaattgtttttcttcataagaagataatagaatcaatactctctataacatcttattcacaatgacaaggattcaaaaaaaaaaatcactgaaccaaacaaaaatggggatgtggatagtgatacagagtcagataaaagcagtcagtagaggcctggctggcacacatgttggacttagcagacagagaattaaagaagtgatcataggtcggtcacagtggctcacgcctgtaatcccggcactttgggaggccaaagagggtggatcacctgaggtcaggagtttgagactgcctggccaacacagtgaaagcttgtctatactaaaaatacaaaattagccaggcatggtggcacatgcctgtaatcccagctactcactcaggaggctgaggcaggagaatcgcttgaacccaggaggtggaggttgcagtgagccaagatggtgtcattgcactccagcctgggcaacagaatgagactgcgtctcaaaaaagaaaaaaaaaaaaaaagttataaaaatatttaagaaaacaaaggaaaatgtagccttaaagattacacaaataaggaatttcagcagaaaaatgagtcaaatggacattttacgattagaaagtacagtaactgaaatttaaaattcactggatgaggttaaaagtggactagagataggagaagaaacagccagtgaacttgaagacagatgagtagagatgagccaatgtgaaaaacagagaaaaatattgagactaaataaatagaggcttagggatccatatagaagaagagagtgagatgtggggaaaaaaaaatgtgtacgtaatggctcaaccttcccaatgtgatgaaaaactcagatctaagaattttaccatatctcaaacctataaagagaaccacatctggccatgtcagatgtggttaaagtgccgaaaactaaagattaaaacatttaaaagcagccagaaggaaagacatattacgtacagaggagaaatggtaaggactactgacctgtcatgagaaatcatggagtgatgtctttaaagtgctgaaggaaaaaaatgtgtaaactgaggtttctgtattaagaaaaatatccctcaaaaatgagagtgaattagacatcttcagataaatggaagatgagaaaaattaatctctagcaggcctgaatgctaaagaatgtcctccatgtcaaaggaaatgatactgtttggacatttggatttatagcataaaaatatcatatgcaataaataagtgggtaaataaaatgtctatactgtctttcttatttaatttacataaaaggtacctaactgttaaaagcaaaaataatgtaatgtgtaattatttgtaatgtgggtttatgacatgtagaagtaaaggatatgacaatagtcacataaacaatggaagatatgtatatggaattatactgctctaggataactgtatataggaaatataaagtgagaagtgggtagtgccaggtatgacattggaagagggaaatagcatggtggtgttgctgagttgagaaaacagagattgaaatcagggaggctttagcatctggaatttggggccaccattccagagaggatagagctgcagggaaaaataactccacggagagagctagtgattctccagtcctgcatattacaagcccgatggactcaaagttggagatagagtccctgtagtctgatgtttgcctctctaaattgcttttgaatgcattcgaaatgcaaattccagaatgcatgagtaatatttctgtctctgagtttcctccaattatatttgttcttgaaagtctggtgtgtcctcccagtcattcattctgatttttctggacaataggataatattttatgttttgtttctagcatacttattgatgaactcaaaatggttttcttggacataatatcaagttggacttccattttcaaaaccataggctttgagaggcctatggcaggcctctcaaagtctttgagactttgggaggccaaggcaggagaattgcttgagcccaggagtttgagaccagcctgagcaacatagcaagacctcatctctacaaaaaaatataaaaaattagttaggcatggtggcatgcacctatagtcccagttactcaggaggatgagatgggaggatcgcttgagcttgggacctggtcaaggctgcagtgaccgtgatggtgccattgcattccagcctgggtggcagcaagatcctgactccagaaaaataaataaataaaaataaataaaaataaaaaaaaactataatgaagaaactttattttatttcagatttttaaattaatttttagattttattgtcttattttttgaggcagggttttgctctatcacccacgcaagttcagtggtgctttgagcatttcatttacttacatatttcccataaccatatttgtgctgctgtctcttgattatttatttttagatactaactcatactatcctaccatggtagatgaggattcaggtcttttgctctgctcttgcccatccagtacacatcctctctatcctcccaatgtaattatcacacaatacctcgttaaatcctgtttggtattttattatgacaatacaaatactcatcgtcgtgtcatagtttttttaaattgcatttcttttcttcagaacctttttattcttcctggtgtaagtgaccactttgctctgcttaatttcccgtgtaattgtcaccaatttatcctcaaactctccaccacgttgaattacttaataaaaggaatttagcagttcctttttttcccatggagatattcatactggagtcttgcattctcctaatctggactgttcgttctagagacctactgcataggtactatcctaggactacctttcatcgtcttcatggaatttatgttgttctctagtattggactctgtttcctggatcctgtgtggtgatagctgcctgaggaaggttccattggaagtaaatatttcataatcttccatgtctgaaaaatgccttcatattgtattgatagtttgtctggacatagtattctagatttgaaactattttcctcttagaatttagaaggcattatgttgtcttttaacttacagtgttgctgttaagacatctgacaccattctgagtctcagtccttttattacagcctttctctctatatctggaagcttttagaatgttttatttattcccaatgtcctgaaatgttatgattgttgactttgtgtgggtcattatttattatctggacagctgcctcaaatctacgagcttttcccaagtttgtgactttcaaattaagtttcaacttactttttctgttatatctcggtcaaaaccataccagctgaatcaatttaccttaagaagtcaatttctgatggtgaggtgtttgctataattctttttcttgctctaaaattatcttggctattcttggcccttcatttttctatgtgattgttagaattagcatattgatttatatccatacatgtacatacatgtgaacaatccttcataaacactggcttatgtatttaagacctgctttgaaattagcttaattttgggaacatttatgtctataacatggaatcttctcatctatgaatgttgtgtttctttttattgtttcaggttttatgtctgttaatacagttccatgatgtttactattaatccattacctagatgagttagatttgctcctaaacgacttatggattttttaatgttacctgtattataaatgatacttgtttaaatttgcagtttaatagtgtgttgctcttctttaggattagaattgattttgtatattcatcctgaatctagcaatcttgctaattttttatcggtttaaaatagtttatagattcttctagatttttcatataataaatcatgtaatccgtaaattatgagaaactttgggtttgtttcttcacaatctttatatgtgctatttttttctcttaccatggagtctaatgaccttcagtacaatgttaaataggtgcagtaatgtttctgtagcttcaaattaatttaatgtttgcagttagcttttggtatatatatcttatcaaattaagaaatcccttctcatttctgattaccagagtttttttgttatgattctggttgtaactaaattacttttctgcatccatcaagatgatgttaggttgtttttcctttaatcagttaatctgataaatgacagcttttccattgttaaatcattcattctttgagtaaatatgactttatcaagatgttatattgcagtattttaacacattcctagttgactctactactattttatatagtattgttacctggcattcatacatgacattagtcatatctcagactggagtataattttttttccttaaattgttcttgtctgatttttaggtttaggtcattttagccttataaaataaacagaggacccatattttccccttacccattttggctctagaacagtttatagtacagttatctgttaacttatgtcattggtagaatttacttttaaatcaccttagcctgggtgaagttttgttttcttctattttattttgttttgcagatagattttgtactattgattcaaatttttaatgattataggtctgttggactttctctttcttcctgagttttaatacgttatagtcttttcaatgaaaattgtccattttctctgagttttcaattgtattgccataaagtagttcagaccaccttcttcctagttttaacatctttgttacagtgtcattttgccccccttttcctagtattgtttatttgtgcttctcttttccttcgtaagttttgatacagagcattctactgttcaattctaaatattttccaacttaagttagaattcttctatgggccactatttagtgatatgcctttttaatattcatagtattatatttatttgtttagttagttattcctggtttcagtttactgtagattgggtctatttggtatcagatttttttggcattttttgaaatttgctttgtgattttgtaacaggccagtttttatatgttctatgtttatttgaaaagaatacttttttttttctaacttttgggtacagatgtatatgcccataagattaaaattgctaattttgtcattcagtttttttgtgttcttaaccattttatttctctgatctatttattcgttttttaaaaatccttatgatggtaaatgtgttaactttcctttgtaatcatcaatgttttgcacagctcttttgtagctttgctgttaggtatatattattcagaattgacatttcttccagggtattttcttttatattatattaatgaattttaaaaaaatcttacaatgattttggcatgaggatatatttaattgatattactgtagctaccttaagtttcttttctttatcatatgcctgatatatccctttccatccatttacttttaactttcctgcgtccttatgttttaagtgcatcccttttaaacagattattgctgatgtttttcccatttgttcattttggttttgtgtgttgtcctgtgtaacaggtttgtttaacaggtgaatttaatccatgtataaattttgtgattattaatatacttatttctagtatcttacgtgcagtttattattggttttgcttgtttctttgcatgtatgcttgagtttgttttcttctttcttgccttctgttgacttgatcaagtttagcttttaaatttctctttgtttggaagttacatttctaagtattttagtgtttatctttacatttttaaacatttgtagtttacctgctgtctagagtttatcggtttttctgccacattccctatcaaaacaagaaacttagattacttcacactgtaattaactcaacgtgtaacactcacaacaaaaattaactcaaaatcggtcaaagacctaaatacaaaaactaaaactgtgaaactcttaggaaaaaaatagaagggatacttttatgaccttggatttgtcagtgatttcttggggatgacacaaaattacaggcaacgaaagaaaaaaatagataaattcggcaatatctccattaaaacttctgtacatcaaagggcacgatcagagtgaaaaggcaacatggaatgagagaaaatatttgcaaatcatatgtctaataacaggttagtctctaaaatatgtaaaaactcctacaagtcagcaacaaagaacaaaggaccctgttaaaaaatggacaaggtatttatttatttgtattttattttattttagagacagagtcttgctctgtcgcctaggctggtgtcagtggcgtgatctcggctcacttatcctgcctcagccacccgagtagttgggatttcagacatgcaccaccacacccagctaattttttttttttttttttttttgtagagactgggttatgacctcttggccaggttggtctcaaactcctgacctcaagtgattgacccacctcagcctcccaaagtgctgggacaaggaacttaaatagacatttctccaaagaagatatttaaatggccaattagcacttgaaaaggtgctcaacatcactaataattagagaaacacaaaaccacagggatacaccatctcacactcagcaggattgctactatcaaaaaacagaaaataacaagtgttgtcaaggatgtgaacaaattaggatccttgggcactgttggtgggaatgtaaaatatggaaaatggtataggtagctcttcaaaaaattgaaaatagaattaccacgtgatccagagttcctcctctgggtatatacccaaaagaagtgaaagcaaggagttgaacatacatttgtacactcatttttagagcagcattgttcacagtagccaaagggtgacagatgaatggataaacaaaatgtgctgtatacatacaatggaatatcaatcaattttaaaaagaaaggaaattctgacacgtgctacaacatggatgaaaccttaggacattatgttaatgaaataagccagttagaaaagtacaaatattttatgattccacttatgtgagataactatagtaatcaagttcaaagaaacaaagtacaatggtggcttccagggtctaggggaagggggaatgggggaattactgatgggtacagagttaaaattttccaagatgaaaaaattttagagaaggatggtggggactggttgcacaacagtgtgaaggtacttatgccattgaactgcacacttctaaaaatcattaagatagtaaattttatgtctatttttaccaccattaaaaataataattaaaaatttaaaaaacctaagaaatttagaatacttgaatcctgtttacaccctcagtaatatgtttttgtttttccagtgtttaatttccaacttctttttttttttctttttttttttttcagggaggtaaagtgggtggtttttaaaaatttttatttttctatttgtaaactgacaaattatagttgaacacatttatggggtaaacttatttttcagccctaaggtggttactcctgcctctttttctagtccttgtcctccttcttcttcattatgttatccaattaaccaccatttaaatgaattagtaaagtttactaatatctttgttcataattgcttctagtatcacattcttccatcttggttaaatttcttgttttccgaagtataccctttagtgcttgtttagccgaaaggaaatgttagcgatagtttactcagtcttgtgtgaaaatgtatcatttctgtatcataatttaggtaggtaggtaatactatattgacaattattttttcttatcactttgaaggtacaacttcattttcttctgactgcaattcttagttattaataattcagctgttggtctaattgtcatttcttagtagataatctctattctttaatgtttttaaaatctctttggctttggtattctgtagtttaattccaatgtgtatagacatgagtctggttttatttatcttctgagtactttttgttttttttgtttttttttttttgagacagggtttcactcttgtcgcccaggctggagtgcagtgatgccatctctgctcactgcaacctcaacttcccaggctcaagcaatcctcccacctcagcctcccgagttgctggtagtacagacatgtgccaccatgtccagctaatttttgtattttttgtagagatgtggtttcacgatgttgcccaggctggtctccaacttctgagctcaagtgaaacacccgcctcagcttcccaaagtggtgggattactggtgtgagccactgcgctcaacctcttctaagtactttatgcccttcctgcatccaggaattcgagtcttatatcattcttgaaaaaatttaagctttgtctctttggatattgctttccactattctctcttttatctcattctcttatactatgttgaacttgtccattctagggtctgtacgtcttagcctttcttccaaattttccgttttttaagtcttcatattagatgctagtaatttcttcagctcttcttcaggtttattattctcgtcaactgtatctaatccactgtttacctatttattacgtttattttatttcagtgactattttcatttctaggacttccgtttgatttttaaaaacttttgtgtttgttgatttttttaggtctttaataattttaaacatgcttatttcatagagtggatctgattgttgttagggatctattgttctgtttgttgggatttctgagttactcataatagacagtttactcatttggtttatattattggattgtgagttcatattcagcaagattttatgtttaagaattatatctgccaaatttgagcatgtgtcctccagagtagatttgaatttgttttgcttcctagaggcaactaaccttgggtcagtgttaatgccggtttcttggcttgggaacttttcaaccacagaagcggtgtgcatttgaatcccaaatgttgagactatgtgatgtaatgttacgaatttctacaggaacaatatttttcattcatcacacattagtcaagatagatgaaatgtcttgtccgtctctgctttgtggcagatttttataatgtaccgtttttcttacagtatacgttttaaggcaaaaggtatgtatatagggcagcctttgtcttcaaactcgccccctcatgtaagctaaggataaggtcttttcttttgttcttacctcatataagcataaggtactcgcttttgtgtatgttaaaatttgagtacctaggaaatgaaaaccaacaattccccaaccctactcatctctaatgcttattatcctgattctcaagcatttcttgattactgacctctaagaatctctctttgtaaaaaagttcttttatatgtttatacaaataattgttatattttatctagctttttttttttttttttttttttttttgcttttgagaccgagtcttgctctgtcgcccaggctggagtgcagtggcgcaatcttggcttactgcaagctccgcctcccgggttcacaccattctcctgcctcagcctcctgagtagctgggactgcaggcatccaccaccatgcctggctaatttttttgtgtttttagtagagatggggtttcactgtgttagacagggtgtttgtctagcattttaagagtttgtaatggaagggttttcaagtcatttaatctcagtattgctaaaatgtaagtttatcctaattattcagaattggaaatgtagtctagaaaatccaaaatattaactggtattctactttaggaatgttcatgttttggataagttcttctaaaacatgtaaagggctaatattcttttacttcaaagtaccttacaacatgcatacccaggatttttctagttacctgttttattttttcttccacttttttacagagatagggtcttgctgtgttgcccaggcttgtctcaaactcctgggcccaagtgattctcccacattggcttcccaaagtgctgggattacagatgtcagctcccatgcctggttttaattacctgttttctaattaaacctaacctgctataacaagtactcaactttttttctctattggaactttagattttgaaggatatacctcttcttttatagttttttaaattagtaagtagtttgttgtacattattgacgtgtattttcttaggtttctattaaggagtttgaacataatctccaggcttctcatagtgttggcctaaatatcaggagctttttccccccatctcattgtacaattttgacggtttcatttttttttttttacacgtataggtttgacaggtaattatcaataatgagtgttctaaaccaaatattcagtttgttcttaattggtatgatggtttaaattaaattgttcctctcaatgtcatcagttaagtaacactataataaaagttctatagtgttatgaaatcctgaaaactattaatgtggggataatatacaatttaacatatatacaaggattattattatttttcagataatgacaaagatgtgtttatgagataataatgtctattgtgtatcttaagtaactatttaaaatattttatttctaatataggagattaataaaaatgtaaatttcttcaggttcagtcctcagtggctgtaggaactccagattatatctctcctgaaatccttcaagccatggaagatggaaaagggagatatggacctgaatgtgactggtggtctttgggggtctgtatgtatgaaatgctttacggagaaacaccattttatgcagaatcgctggtggagacatacggaaaaatcatgaaccacaaagttagtatgttttacttttgttcaaattaatgtggtaaataacacatataataaaaccatttttaaagagaaaatatttttctaaaaacagtgaacaatcacatttctatcctagtatgggacaggacaacataaatagtcaaaatccataaattattcataagttattagttctaattttcttgtattttattagagatgccgctaagtaggaaattaagtggtctccccagtagaataacaaaggtcgtaagtagagaatttagatcattgcttactatagtatatgtataatcatgactagaatatgtgtaaaaaaattttcaactttagtggtaataagtgcaatacaaatttaaaataaggatgagatacttttttttcttgtcaaattagcaaagatcaaaagaaaaatagtactctgtaccataaataatgcagtgagatgggcattctaagacattaataggaatataaattggtgtaacctttctgttcaggaagtttacaggtgtgcacctagagccttaaaaaatggttagaggttttgattgagcaacttcagtctaggaatctaccctatgacattctcagtagaacaaagatatatgtacaaggatacctattctaacgttatttatgatcacagaacactagaagcaacacagacacacaacaagaacaaattaatgaaacgttaatatatttagtactaagtgaaatagactgttcataataataagaagaaaggttttattagcagtcttagaagattgtgagcagttgcaaatacttagaatatgaatatccgatagaagggaatgataatctattctttttaatttgttctagtgaagcaattatgattagttgctttacatgtataaaaatcagttttatagggtttatattcttcttcctgtttgcaaagataatgtttcatatagttataaagagttgaaagactggaaatagcagcagggttgtaaatagcatgtacatcatttgaattagaaaaaaatattgtagtatatatttaacagcacagattacagcttcagttcaaattctggttctgccacttcctagatgtgtaatgttgagaaagttacttgaattctgtgtgtctttatttcttctataatattagcatagtaggagtacccacctaaaatgactgttttgagggttaaatgagtaacgtactttaaatagtgtcagatacatattatgtcctttaaaaatatttattattagttaatcattagtatgtaacttatatgaccattatcaatcatctatcttgatttaaatagagtcaaattccactactttgaagtaatttctcttcattctgatagcagtttaagatatctaaggtacaatgtattcttctttctatagcgcatgcatgattgagtcatatatgaaattaaaagtcctaggaaaatgatcttctgtcattcatccctgtattcctttagtattagtactttgcaccaacatgcagttattagatatatttaaaaataaaatgtatttttaagaaaaataaataagtcaaccaactacttagtatgttaaacactgatttatattttgtttctgtttttctctgtttaggagaggtttcagtttccagcccaagtgactgatgtgtctgaaaatgctaaggatcttattcgaaggctcatttgtagcagagaacatcgacttggtcaaaatggaatagaagactttaagaaacacccatttttcagtggaattgattgggataatattcggaactgtgaagcaccttatattccagaagttagtagcccaacagatacatcgaattttgatgtagatgatgattgtttaaaaaattctgtaagtatgacactgtgaagaagttctgtccctctgtttctttctactatggttatatagtaattttttggtttataagtattttgctaggatgattttacaatgatcagttattagtcagtgatcatggcttccggtgcttattctgtcacaaatcaaattaagaagaaagttaactgaggtgaacatagttatttgtgactaagaacaaaattaaaattcaaagttttacctcttagataagttatgccattaagcttcgtaagagaacaaattagatatttaaaaaagcagattttatgattgtaaagtgcagaggttggcaaatttctccctaaaaccataaaactggacaaaattatcaaaatcaaccatgtcagggcctgatacaacaattgaagagaaataaattgagaagcatttatttgtgaaaaactgcttaactttagtttaaagtcacatataccacacacgtccatccccactactgacttggtctgggacagtagtactagtagcatggaactggatagaaaaatcagcagctttgctgatacagagggcagattccaggactaaatacccaagcccagtgtgttgctgctaacaatagcaaacttggtaggaaatcaatagggaaaatagtccacagacgtggtatagctctgaggtatagccctgcctgaggtgaatacagacctcaagccaattattcggccagtccaatagccaaataattcaaccattcttaacttgtgagccatgccaaaacaggccaacatagtatattgacctctgctataaccctttaggttatatgagattaaaatttaggaaaataagaacttaaaatttttgagttttttgcctttttagccttcctcttttttttttttttttttttttttttttttgaaaaaaaattttgctttttctcccaaggtggaggggtggggcacaatctcgggcttattgcaagctcccccttccgggttcacgccgttctcctgcctcaacctcctgagtagctggaactgtaggcgtgcgccaccacacctgggtaacttttatgtttttagtagagacggggtttcaccgtgttagccagaatggtcttgatctcctgaccttgtgatccgccctcctcagcctcccaaagtgctgggattataggcatgagccactgctcccggcccctttttagccttcttaatttgagcttttatttttattcagttattttatttactatggctttaatcctgtactaaaaatttttatgttaatgttatatgacttaacaaaatctttatttttaatttttgtgggtacataataggtacacgttatataacttttcactgaaacagtgaatactatttcaatacaataagctgtgatcactgtttgataactgataattgtaaaaccatcctagtactgtattcaaggcagataaaaatttctcatactttcctgtctaaacttcacaaagaataattaaactgacttctcttaccaattttttataaatagaattatatttactgttaacaaattatgaatcattccaaattaaatacatagtacagttgatcctaattattcatggtggttatgttctatatagttgccttgaacactgttagctaatattgaaccaatgctctgagggaaatacagggttaggtttctgtgagcctctgatcaactgatcaatatatagtcttgttttatgtgtgcttctctttaaagacactttatataacatatgttgttgattcattaacactgaactcaccactgaactcaccaccagcagcactgtaactcgtgcctgaatgaagcttttctagcacattattttctccataaaggcacatcactgtcctctggcgctaggaacactagacagcgcttcagcactatgcttgggagcctttgcaagcagtgaatcacatacagagggcccaaaaatgtgaaatgcatgacaccaaagagaccctgtagaggacacttgtttataatatgagagcttaaatgagaaggcagagcctctcctgttcaacctcagctgggaatgtgagcataggacatctcaaatttttctctgctctgcacgtgtctacaaatgatcataaaattaccacaagtattgatttggggtttacaaataaatattagcttctacacaaatttgcaaatacagaatacatgaataataatgactgtattctcaatatcagactttgaatcttcattttactttaattagaattttagagtagcagtgttcatggtctaaattttatatcactaaacaactataccaaacttaccagtgttttaaaattgaacagtcacaatttttttttaaatatttctttttggcattttatatttatagccaagcctacagatcatatagctatctatttcttaccagatgttttttcctcctttaggaaacgatgcccccaccaacacatactgcattttctggccaccatctgccatttgttggttttacatatactagtagctggtaagtttgaaaagttaacatagtttgggccaagtgcagtggctcacgcctgtaatcccaacactttgggaggccaagacaggtgtatcatgaggtcaggagatcgagaccagcctggccaatatggtgaaaccccatctctactaaaaatacaaaacttaggtgggcgtggtggcaggtgcctgtaatcccagctactcgggaggccgaggcaggagaatcacttgaacctgggaggtggaggttgcagtgagccgagactgcgccattgcagtgcagcctgggtgacagagcgagactctgtcttaaaaaaaaaaaaagttaacatagtttggacaatttaaagcaaattaaattgaattgaggttcgtaaaaggtgtatgagaaaactttgtgaattaaaactttttaaaaaatagaatgagcctactgctggaaatagaaagttaagcattacaaaatgaatagaacgcatgaaaatcttaactcgttttggaggggaaagaatggaacagagagctcagaatctaatccttaccaatgaagcagtaattagaagagtttcacgctgttgaatattctctagatgtcaagcaggagaactttcaacccattaattctaccatcctttcactgaccctcacttcacttatattccctatttcccccttaccctgtttaaattccattgagaatgattataatcactccttgtatgccatttcagttcctttgactgtctctcattctgttatatttgttttgctaagtgaacattaactattacaattttattctctacctgtacagttccaaatgactagaaaaaatagctgactattcttactggtctactttaaattcatgattaccaacctcaagtcccccttgtctgacaaatgtactgtattaccctagtatattcactcttccactcacctagtaactattttttactttctttttttttaagagatggggtcttgttctgggcagcagaagggctggaatgcagtggtgcagtcctagctcactatagcctcagactcctgggcacaaatgatccccctgcctcagcctcctgagtagctgggacaataggcacaagccaccatgcccagctaatttttaaaacgatttagaaatggagtcttgctgtgctgtctaggctggagtgcagtggcacagtccatagataactacagcctcctactcctcaagccgtcctctggccttaacctcccctgttgctgggattacagatgtgagccactgcatctggcctatttctttctacttgagtttgaaacctctgtcatcctcactttcagcttgcatctcctttcactgaaaggattgaggcaaaccgcagacaacttccacaagctccagttaccatatttaccgcctacttattgttggacccaaattctctgttcttttcatggatgaactgtgcaagcctctagtaaggccagtatcttccatcccttcctggctagtctagtgcatgttttgctttcatgtcatgaatttttttcttcttgtgtcatctgcatcatcagatcatttctatcagcttacaaatgtcagttctccatcttaatgcaaaatataaaatctggattcctcttcctttacctcttatcaaccatgtctctgctttacattatagaaaatttacttcaactgttgactatattttgtttatgtttctcatactgttgtttcttgaatccattccagtcaggtgtgtgcccccatcacttcatgcaaactacttttatcgatgtcactgttgaccaacatgttgctaaatctaatgatcacttgtcagtcctagtcttacttatttgcttagtctcatcttatttgccacagctccatacttttaaaataccaccttctcctgtcttctaaggcacttgactgagtttctctcaccttgctggtcattccttctcagtctccatacctgatccctcctgatttccctggcttgttaatatcagagtgtcctatggctcagtctttggacttctcttttccatctaaactctgtagtgttctcccaaagatttgctgatgactcctaaacgtgcatctccaactgagacctctcctctcgtctactgtaaaaatctgtcttaaccacccacttactatgtaaataactttcttttagttctctgatcctgttcaggtgatcctcttaaaagataatcagaacatgtcactcccctgttcagaaccagtccacccatccccaactccagtggcttcatattttaactccaaatcaaacttcttacaaggccctacatgactgccttacttttatctttctgagtttatttttccttgtttactcaactccatccacacaggcctcctttctgggctgtgaacatgccagacatactcttgtctcggggctttagtacttctgtttcatctgcttggaacattctttcttttgatattcccttaactggtcccctcacccttacttagctttttccaaagttactttagcagtgacagctttcctaacttctttatctaaatttttatctcctcctcttcaatactttatttctccctttcctgctttatgttttctccttagcatttaacattttctaatgtaatgaatatctccattattttctttatttctgtctcagtcactggaatataagctctatatgggcaaggatttttgtctgttttgttcaccaccaaatgtccagcagtttcagcatttttatttaaattgtattgaattaaagacagaaacttgcttttgaatttcaaattctagtaaagagggccgtggttggtgctttgactactactgtttcacctannnnnnnnnnnnnnnnnnnntactgtttccctaggtggtcaaattgttttaaactaccagtaacccttcagcaaacttcattttataatttgtggggtttttaaaatcataataatttgggggattttaggttttcattttttaaaatgcttctaaaaacagtttatggtgattttgcaaacaaatttttataattattccagctggctaactgcaagatattcagatctgtgttttaaatgagcaatttaaaaatgtttttgccataaatgtggagtcagttaaaaacaatttttaaaaaatgttaacgtttttcttcaagctttttatcacagtatgaacctacatttttttttcttttcttcagtgtactttctgatcggagctgtttaagagttacggctggtcccacctcactggatcttgatgttaatgttcagaggactctagacaacaacttagcaactgaagcttatgaaagaagaattaagcgccttgagcaagaaaaacttgaactcagtagaaaacttcaaggtaaatatattttaaatatattttcactttttgaattgtactcacagtgttatgagctgtttattataagtggttgttttatctttgaaaaaaattattttaagtggtgaaaaatcaagttgtagagatgtatgcagaacacagcataataccatttatgtaaaatcacacagttaatagtaaacaacaacaaaatagctttggtctcctgtgggtacatatttatgtttataaaaatgtgtgggaatacctttggggatggggaaatgggaccctcctcacttcgttatagcactttaatgacttttcctatattatttgctcaatctttaagagctctagctcctttcacactgtcattctctgcagcattgcttctgccatatttggtgattttaaaatgcacatacacataaactttttaagattctgagtgagtttcttgagtcttcaccttcagtgattgtttactccacctggtttcatccagtccatcctatggtaataagatatgtcttaagattacaatacctaaaactcttctttaatcgcagtttccagcaaatcttttataatctcctgctcataatttttagtgctctctctctgaccccttaactccagtatttcttcactgccaccgttacctacaagtctatcaccgtttaactctgctatttataccctcatttttcgttctctggtcagccatagtcataccctggcatataccttcaaatctattgttcctctttctctttgtcaaatacctttggcagaaccccaactgtgtttgaatacaactttgtatcttcaactatccagctgaagacagcttgcttaaaacacccacaacgtgctgactaatattaattcagattcacagacactcacctcaagtcagctcttcatgctatttcccctagtctatttccctatcctgtgttgtaaaatccagtgatgatttctgtcttcaccttttaacctatcagcaatatttgaccctatggaccttttattccttctgataacgctctctttttttttttttggcttctggggcactttatctcctggttttctttttcctcataaacctgtccttcccagtctcctttgctaagttttccttaactcttcgacctttgaatgttagaatattctctatttgaagcaagtctctaattgaatgaggccttcagtgaaggttttcctgaccatcctatttataatttaatcatatatatatccccagtactactgtctcctttttctgcttttttctctaaaataaaggatgtaacatattttacttacatattttgtgaatgaaggaatgtatttatggacaaataatcaggaacttttgtaactaatcagagttttacaatcacaaaactttactgtcatacttttagaggcccagtagagaaaaaggctgtgtagacatcaatagaatggactattttctgtttttccgtaagttgttaagatctctgcatatgaattacttcccttttgtgactgaaatttttctgtttcttttttttcctgtggtgatacacatatagcaaagtgtgcaagcattctacttttaagtataagcctaataaatatttgtatatgtatacactgatgtaaccttcacccatattcatgtgtagaatttgtccagcatagggtaaagtcctcacatgtcccttcccagtttgtaacctaccactatgaaataaacatcatcctaagttctttcaccattaatgaatttttaccggtttttgatcttcatataaatggtatcatacagtatattctcttttgtgtatggcatctttcacttgttttttgaaattcacccatgttttgtatatatcagcagtttgctcttttttttttttgtactgctgtttagtatttcattgtatatcagcaataaaaaattacccagtctgttgttggtaaatatttgggttatttccagatgttgactactgtgagtaaagttcgtgcaaatatacttatctatttattttggtggtcctatgtactatttctctcagatatatacctgggagtacaatttctgggccatatagtagaggattttcagctttagaagatattttcaaagagttttctaaagtgactataccaatatatagttctactaataatgtatgagttctactgtcttcaactcctcaccagaacttatcgtccgcgtttttcacttgtagccattctggtagaagtatagtgatgtcctgccgtgattttaatttgcatttccctgatgaataatgaggttgaatatcttttcatgtatttactatccatttggatatctagtcttgtgaagttttggttcgaggttcttgcccatttttctattaggtctttatatatctttttcttaataatttgtatgttactacttcacaaacaacgtagaaacctttagacaactccttgtatgactgttgttacatagtttactttaatctgtgttataaatcttttaatacacttttactccattgttgctttatgttttctttactcattaattttaagtatctttttatttacccatatatttacccttttggtgctttttatttctccctagtgatccatgtttctatctggaatcatttttctttagcctaaaaaacttcctttattgttttcttgtaatgtgaatgtgttgtgacaaattcttgataggattttgttgatctgaaaatgtctttatttttaccttcatttttgaagaatacttgtgctgggttagaattctagcttggcagctttttctttcagagctctactgtattatttcattgttttctataataacttcccataatatttcttgagaagccagctgttaatcttaagctcttgaatgtaatgtgtatgttttcccttctggctgctttcagtattatgtccttaacttcaatttcaaatagttggactgtgattcacgtacgtgtcgctttctttatgtgaacagaaaatcctggttgagattcattgagcttttgcaattttctttgatgtcttttatcagttttgcaaaattctaagccataatctctgtagatattattttgcctcatgcactttctcctctccttctgggactgtaattgtgtatatgttagatggtatctcacatttcctttatgctctgttctgttccttccgttctttctgttttttgtgtgcttcattaagatattttctattgatctgccttcaggttcactagttctggcttctgctttatccattttgctgttaagtccatctaattagtcttaatttcagatattgtcttttttagttaaggattattcatttgattctctcagtttcagttttctgttgaaatttccccatatttttatgtatttttttctctgttttctttaacattttaatattttgaggtccttatctgttactttcaacaactgaatcttctgcgagcctgcctttctgtttttttctttcatttgtcatgcttttctgcttttccaaatgtctcatgtgaaagatgtcatgaataaaaataacacagagaaggaagtttatattatttttgccccagagaggtttctcccttttcattccatcaagaattgagcttggacaagactaaattgctttttttttttttttttggagacagagtctcgctctgtcgcccaggctggagtgcagtggtgtgatctcggttcactgcaacctccgcctcccaggttcaagcaattctcctgcctcagcctcctgagtaactgggactacaggcacgtgccaccatgcctggctaattttttgtatttttagtagagacaaggtttcaccatgtaagccaggatagtctcgatctcctgacctcgtgatccgcccgtctcagcctccctaagtgctgggattacaggcaagagccactgtgcctgcccctaaattgcatttttaattagactcaaccctacctttgatttcaaatgcccagaatcgggagctggatagagcctagttgggcctctttctccttcacttcctaaactatgggagatttattctgttcttcagccttctgccctataccatttccaaatttggtgtcttgatgaagagactggccatgtatttgaagcagattctcttcctcaagcagctctttgtctctgaaataattctgtactatcggagatttctgtctgacttttcaaagctccctaacttcccatgcagcccccaagtacatcaattaaaatataccatgggaataaaagcaccatatgcttagggactttgagatttccaaattgttgcaccatccccacacatcctccagaagcttcagtggtttttctttccagcatgttatctctcaatttaaaggccaggcttgacctaagtacactcacagaaatggataaggcttccaggtaagaagcagttgtaatagcctcttcacctcagaatgcctcacttccctggctcactttcccaactccaactgaaaaagcaaagagaactaagtaaaataaaccaaagatgtttttaaaaataagattttctcaaattatgtattgaatttttactttctacggcaggagaaacaaagcagtgacctgttgcttcctagtccatccccttatgactaagaatttcctatgttttgggttttttttaaatgttttgaattctttaatttgttcttggtatttttacatgtgtagtatcgtttgcacagagcaaagtgaacaaaaatgtaagatcattgccttttggtatgaatacttgtttcatagcagtgacatagattaacatttaaactgattcattcatttattagctgtttattgagtatcaaacagcccaggagctgtactgcccatgaccaggaatgtaataaaaagcagataaaggcaaaaatacctccccttgttatgtttgcattctgttttgaggagacagacaataaatcacatacaaagtagggtcatccactgagagttaggaaggtagaggagatattagatatttgacaagaaaaagagaaaatgtgaaatggtcagtatggagacgaaaagagtaaatgagctagggaaatagtttaggatcgccagatatttccaagttatttaagttcagtaatcatgagttttaaatgactgtgtatttttttcagccattttcagctacttgggtaaaatcatacagtaggaggagagtagaaattatccaagatagagggccttctagaataagtacaatggaggagagaagggtaaagaaattgaaggtatttgcaaaacagtgatggaccaaagaatctaaccagggtaagtaagaaagtaaagaggaaaaaaatggaaaatgttaggaacacattgattggcatttctggttggaatgaaaaattgttgaagtggtccctagagtagtaagctgtacagatgagagagatgattttggaggccatgcagttattgataatgacaaagtgtatagttgaatttcttttttatttatttatttatttatttattcatttatttttttattgatcattcttgggtgtttctcgcagagggggattgggcagggtcataggacaatagtggagggaatgtcagcagataaacaagtgaacaaaggtctctggttttcctaggcagaggaccctgcggccttccgcagtgtttgtgtccctgggtacttaagattagggagtggtgatgactctcaacgagcatgctgccttcaagcatctgtttaacaaagcacatcttgcaccgcccttaatccatttaaccctgagtggacacagcacatgtttcagagagcacagggttgggggtaaggtcacagatcaataggatcccaaggcagaagaatttttcttagtacagaacaaaatgaaaagtctcccatgtctacttctctctacacagacacggcaaccatccgatttctcaatcttttccccacccttcccccctttctattccacaaaaccgccattgtcatcatggcccgttctcaatgagctgttgggtacacctcccagatggggtcgtggccgggcagaggggctcctcacttcccagtaggggcggccgggcagaagcgcccctcacctcccggatggggcagctggccaggccgggggctgtccgcccccacctcctcccggactgggcggctggccgggcagaggggctcctcacttcccagtaggggcggccgggcagaggcgcccctcacctcccagacggggcagctggccgggcgggggggctgacccccccacctccctcccagacggggcagccggccaggcagaggggttcctcagttcccagtaggggtggccgggcagaggtgcccctcgcctcccggacagggcggctggccgggcggggggctgacccccccacctccctcccggacggggcggctggcctggtggggggctgacccccacctccctcccggacggggtggctgccgggcggagatgctcctcacttcccagacggggtggctgccggacggcggggctcctcacttctcagacagggcggctgccgggcggaggggctcctcacctctcagtcggggcggttgccaggcagagggtctccttacttctcagacggggcggccgggcagagacgctcctcacctcccagacggggttgcggccgggcagaggcgctcctcacatcccagacggggcggcggggcagaggcgctctccacatcccagacgatgggcgggcgggcagagacgctcctcacttcctagatgggatggtggtggggaagaggcgctcctcacttcctagatgggatggcggccgggcagagacgatcctcactttccagactgggcagccaggcagaggggctcctcacatcccagacgatgggcggccaggcagagacgctcctcacttcccagacggggtggcggccaggcagaggctgcaatctcggctctttgggaggccaaggcaggcggctgggaggtagatgttgtagcgagccgagatcacgccactgcactccagcctgggcaccattgagcactgagtgaacgagactccgtctgcaatcccggcacctcgggaggccgaggctggcggatcactcgcggttaggagctggagaccagcccggccaacacagcgaaaccccgtctccaccaaaaaaatacgaaaaccagtcaggtgtggcggcgcgcctgaggcactcggcaggctgaggcaggagaatcaggcagggaggttgcagtgagccgagatggcagcagtacagtccagctttggctcggcatcagagggagaccgtggaaagagagggagaggaagaccgtggggagagggagagggagagggagagggttgaatttcttaattggtgaaccacaagcatttggggtgaggtaattttttcacatgagagagagtttcacactctgagatattgaacattctgagtttccagatgttaaatgcttgtagcagtctcctcatgtgctgtgtcaacaactgactcccgttctcctgtccccttacataaggtgatggtaccacctgcagtagagaaccactagtttaggatgtggccagttaatctgatggttatgctggagagaagtctatcagctagattgttttgggctgcagatgtgagaattcttaaataatatggatttattttctcacttaagtcagaagtaaatggtttcagtgccaattttagggtagcttccctattaatttcttggctttccttacatgattccacaatattgtaagcctcaactttcagatgtaaactttggaaggtaagacctcccttcatacaccagattcagttcttcatatatcatctgacagacataccaggcatttcatttatcacacggtgatgttccctgctaggacaaaaaggagctaagtagattcattggcagagtacaccaagaattccatcttcatatctgcacatatccagagacccagaaagtggaaagattcatttagtttcatacatctttcatttaatgggagattaaaacctctccctgaagccccctagtggacttcttctcaaggtaccatttaagttctaactaggtcacatacctaattcttgaccaacacttgcaatgtagaatggaattattatgattaacttaaaccagccagttttgactgggcccaccttacatgtgcacactgtagcctgataattgaatttggttcacaagacagaagagatgaatagatgtcgggtaaaaacaagcagtatctgccgtaggggatttatgtgcctcagaagtcagtgttagatggattatctattttgatgttaaagttactaagaataataagaagagtagtaatggaaagaacagcagagaacaacatgtaaaatccttatttaaaaaagagtaagatagggaagacaagtggataaccacataagcaggaaatggtcatttcagtagacctaggaattttgaggaaagaggtggcaataatgagcgaggatgcctgccccaccttcatacctgtggtaggtggggtacaggaggaaaagtagctcctattaatgtccatttggtattctttggtcttttgtttgtttgttttttttgagacagagtctcgatctatcacccaggctgggtgtagtggcatgatctcagctcactgcaacctctgcctcccaggttcaagcgtttctcctgcctcagcctcccgagtagctggtattacaggcatgcaccaccatgcgtggctaatttttgtatttttagtagagacggggtttcaccatgttggccaggctgatctcaaactcccaacctcaggtgattcatctgccttggcctcccaaagcgctgggattacaggcatgagccaccgcacctacacctcccctgcattgggtatttttatgtctccccattttctacccccaatttagatgtcttaaaaaataaaaacaatggatgttcttttcaaaataataaatacggaaaggaccagtttttagtatgttttgaacccaaagagaaagaaatgtataagaaatacttataggatgcagaaaatcaacatcagatgcacaatctagtatatcttatacctttaattcaaattaacaaatatttattgactgccaaacatatagggattatggtttacacacacacacacgtacatatgcacacacaaagtaatcagtaatgatttgcactaactcataaagaattatggtaagggagagacttattatgtgagaatccctccataaatagcagaatacctattttagaacaaagggcaatttcatttctgatgtaaaatggtaaaaaatgttttctttgttccatgtcttttgatgatatctgggaataagcctcaatagagttattgtttttttaaaaccttgtattttctaattagaagtgcaagataaattaatattttctattttaactaaatattttagctaaattgttgagccatggttacctgtagctgggcctcaactttcagacataaaatttggaaggccagcatttctgcatatgtatattaacatgcctaactctccatttgtaaaccagattcagttcttcgtatagcatctgactgacataccaggcttttgagttatcacatggtgatgttcctactaggagaaaaaggagctatgtagttgattcattggcagagtacgccaatagtatttaaaaccaaaaattattagaaggtggattcttttgtatatatatatatgcagtggattttttttttttttttttttttttttatgagatacagtctcactttgtctcccaggatggagtacagtgccatgatcaaggctcactaccacctcaacctccggggctcaagcaattctcccatctgagcctcctgagtagcgggacttcaggcatgtgccaccatgccctgccaatttttatatcttttgtagagaaagggtgtcattctgttgcccagtctggtctcatactcctgtactcaagtgatcctcccactttggcctcccaaagtgctgggattaccatgcctggtctgattttttatgacttaatttttgagtattcacatgctttttgtcagtgagtagatgttttgttctataaaattgtataagaatttctctccaaaaaaataatgttattagttttaagaatttagagtatggtgatatttttatttcttttatccttttttttaaagagtcaacacagactgtccaagctctgcagtattcaactgttgatggtccactaacagcaagcaaagatttagaaataaaaaacttaaaagaagaaattgaaaaactaagaaaacaagtaacaggtaaatatctgtgattcactgttaattcaatttaggaaatgtttaaaaaatggtttattcacacaaaaaacatctattgagaacctgtctgtgttatgtgatgtgagttgcatgctagtcagggaagggcaaataagaatgggttctgctctcaaggagttcacattttggtctgggaaacagataaacagttattgcaatacatatgaaaaggctatcaggaaaaataaatggaggtacgtggagagtacataggaggatcacctgacacagacttacgtgaacaaggatggcttttttggaaggcctattcattttggaatttgtctggcaaagcatatttgtgaggagaggaaaaggtgagttaggagtgaggaagaaggacactagagacaagcagagtgcatgtatacaggcatcaagaaaatttattttctttgttaatgcaattattttttcagtgtattactgagattaaaactaaatttgacaagtgttcgtatacctattattcatttattgcattttttcaataaatatattgagcacctactatgtagcaggtgttgttttaggctcttgggattcaacattaagcaaaacagactaaacaaaaattctttgctttggcctgggtaaaggacagaaaacagaaaccagtaaatatataactttgtaaatgatacaatatgttggaaggtaataagtgctttaaaaataagaatagaataaggagaattagtcagatgtgcaaaatgaggagttttatcattttaagtatagtgctccaggggtaggcctgactgagaaggtgacatgttagcaaaaatttaaagaaggcaagggggttagccatacagttatctgcaaagtatgtgttccaggcagaaggactagccggtgcaaaaactctaaaatcaaatgtgccccaggtgttcaagtaaaacagagagtactggatggctagagcagaatgagcaggcagggagaatggaaggaaatgagaactaaaggggtgaaatgggcttgttgcctgatcagtagagccttactgattgttgtaaggtctttggtttttactcagaatgaaatggcagtttttcgcagggtttagaggcattacatgatctgaataagcagagggatagggagagagagcagaaggtactattttaacgagaatggtgaggcctctccagaaaggtagttttaggcaaaaataagtgaaagagaagaaataatctaatgtagcatcttttataaaaatgactatttaaaatgccatattaatactttgtttatattaaatatatggagatcttagttttgagcataatttatgaatgctattttgtgtttttaagaggctctcctcctaagaaatatctatatatctaatctctgttactttatctttccaaaagtaagttattgtagattgggaacatgataagtgaatcatggagtttgctttggtaccaacaataacattaagagtgccactgaatgtctgtgttagagttgtatgacactaaaaaagcagtagaacagaagaccagctagaatggtcagtgattcgtcttcagagaagcgatatcgatttggaaaacttattcataagcatatctctcctctttttaatagtagaatgtggctttgaatggcagtagcttttgaaagcttgctaattaattttaaaactgccttgaaatagatggtattgatgtaaaatattcaaatcaacaatctagttgttatgtcattcactaaaatagttgtgagcttctttgttagctctatgtaagagctgaatttttaaatattaaaacgttttgctgtcattcacaataatgttctggcactatccaaaggaaacctttgttgatgtctaacccagtatatatagaataataataagtaatttcattttctgagcaattccaaatggtatcgtatttttaattttattgttcatgtgtttattgtaatatatagaaatatagttgatttttatatgtttattttgtattctatagtgttgctgaactcacttattagttctaggagggtttttaaaaaatagattctttgggatttttaattctgacaatcatgtcacctgcaggtgggaacagttttatttctccctttctgatctatatgctttttgtttccttttcttgccttattgcaagaacttgcagcactgtgttaaaagaagaatgatgagaagaacattctagcttcttaatcttggggaaaagcattcattttcctctgtttctgtacttctaagggtttttaaaatcattaatgggtgctgaattttgtcagattctttttctgtatcaattgatatgaacatacgattttcaaaaaataacctgttaatatggtgaattacattggttgattttaaagtattgaaccagccatacatccctgttgtaaaccatggtcatggtgtacaattatttttatatattgctgaattccattcctaatatatttttaaggatttttgcatctatatacatgaaggatattagtctttagttttccttttttttcttctttttcctttcttaccttccttccttccttccttccttccttccttccttcccttccttccttccttcctttcttgtactgcctgttttctctgtcaacgtagctggtggctcctgtcaccagcagccaccagaggcctgaacaaaagagtaccagcaggggatccacctgcatgcatcctttctgaagcaaccttgcagtcaggagtcagattgctggtacagtgtcaatgacaacctcgatgtgaacatttaagagtttttagtacttaccttgtggcacgtggcacccctggaggccacacacctggaggtcagggagtgcaggcagacagagaactttgagcaggagagcatggcaactagcagtatatattagggaatagggtgtaggtcactttaagtttgtaagccagtgcttgaatggtccccttagaggaggcagctggaaggcaggatcccagtctgctaggtgggagagatgtctcctgcttgagccccttgggtgtggtgtagaactggaaactgcatcaagagtgactaaaccctgcctctgaaacaagaaagctaaacctgcattcaaagtggatgctgaggcaacataaaattattataaaattatacatttattataaatttatgatttattttaaagtaaaattaaatatgctgtaaatttgtaaatacagttgatccttgagcaacatggatttgaactgtgtggatccacttatatgcagatttttttcccaaccaaatagagattgaaaatacagtatgctttggatccgaaacccaacccaggttggaggtagaagtccaggttccccgtgtggtttctactgacaatgtgtgaggagaggcttgttgtcagccagtgagggttaatgtcctgtctttttctgacatcactctttttttttttttttttttaatttggagacagagtctcactctgttccccaggctggagtgcagtgacgcgatctcagctcactgcaactaccatctcccaggttcaagcaattcttgtgcctcatcctcctgagtagctgggaccacacccagctaattttttgtatttttagtagagacggggtctcgctgtgttgcctgggctggtctcaaactcctgtcctcaagtgattcgccctcctcagctttccaaagtgctgggatttttgggatgagtaaccacacccagcatttttctgacaccactcttgcagatgtgtttgagcatctcattatagcctcatgaggttggaaatctagctccccctgcacttgtccattgttgacattggtgacatggggccagttttttttttttctgtgatgtttatctcaagtagaattgttactttctgtgagttgtctgtctttctggtctacccctttcctaatcctttggctagaaagagcaggcttttgttggggctttttctctttacagctgttgtcatttatgggttaccatcttctttagttctaattctgggatatatgaaccaagaagaaaccccagaaaactcaccgttatgttgtttatcaggtcccagtgtcctgaggaagtttttttcttcacatttcggagtcgtcttatgtttgttttatgtataatgtcttagggttttaagttatacttaaaatgggaagaatgggaaatatatctattccttcttcccagaacttgaagtctccagagtaagcttttaaagtataagtctgttgaggtcacagcttcaacatacctatcttcagtggatcctcattgcctccaggataaagtagaaatttctcaattctaatacaaggcccttcattattatgcatgtatttgcctctcgagctttgtctcttgttactacttccttgcccagtgattccagccatgctgaatcacccagtgatgctccacttccttcacatactgtttttttcctaccctttctttctttccttccctttttttaaaaaaatacaaattgaagtctccccttaggacctgcctgatgagtagccccaactttaatttggctctgaagaaaaaaatatatataccttcaaagaaaaaatatatacctccaaagaaaaatacatacctctgaataatatatacctctgaagaaaaaaaatatacctccaaagaaaaaataccttcaatctgtcattcctccagatacttcttgagcaaagtctttgtgtatacaaatacccaccattaatgactgtttctcttgtgtttcccagcagcagtgttctccaatatttttaagtttctggaaaaaaatgcgacagttttataattattgacaacttctttttaatatttgcttctttaccatactctgagcttctaaaggttaggggtaggttatagtaggttttcccactatattcctagtgccaagcttattgccagtattgtataactgacactatatatatatttgttgagtatctgcagacaaaaataaaatttttttaaataaaaaattcatacctcatttaatatttttgtatgagaaaatagaatttttttgtctcctaaatgaaaaatttattataatctataatgtacttctgagactctcgttcttatgttgccttgaggttggggatatgtgcctctgtgtactgtttctgtttctccaaccccaaactaacacacacacactctctctctctctcccctccctacctcctgtgaataggggcattgataataaaggagcctatttttactgattataattagaaataaagtttcttcgcatctcagaagagttacataatgggaaaaatgaaactatccatcttgtggatcagggagatgttttggttattctgccaacctcccactgggtctagtcttaactcctttccagtttatagtgttccactttgaatactgctttaatcatgcccattttttgacctcaattcctatagcacatacagcctgccacacaatttgcatttgattttctactttattatttttttaaagagtgttgctgttgtctaacttacctccttcaagacagattccatgttttatatttctcttccatgccatacagtgccaagaaaataaccccatatgtatgatgccttcattaagtcctcattactaacaagttgaattttatagtcctaaatagcacccgtgagctaagaaccatttagttggtatcagctaacccagtttcgtagtactgttttttcttttgagacagagtctcattctgtcacccaggctggagtgcagcgatgcaatcttggctcactgcagcctccgcctcctaggttcaagtgattctcccgcctcagcccttctcgagtagctgagtttataggtgtgcgccacacctggctaattgttgtaatactagtagagatggggtttcaccatgttagccgggctggtcttgaacccctggcctaaggtgatctgccagcctcggcctcccaaagtgctaggattacaggcatgaaccatggtgcccggcctagtacagctttttctatttgttcactataattagtttttaacaactttatctaaatgtcatggctaattgtgttgggtatttataatagtctctggatttctttcagaattatagccatatttgctatttgttgtagtgaaagtaaaggctctgctgccttactcattttatagtatttgaccaaagttcagagcattctggatttttcgtgatatggggttttcatcttttgggaatgcaacattctgtgttttccaaattacaagtgctgtgtttctctccataatggaggattttattaaatattaggggaggggttatcttttccaacacttcttaatctagtacattattttttacttctcataattcacatactgttgtttaaatgaatgttttgagcttttttcacttttgaaaagtgactgaagtataaaacagggaaatttgacatatattctctaacaactccattgcaaagcagccagaatgaaatggctgtggtgagaactgaaattaacactatattgaaacaaatcagtaaaaatatccttaaagcttttagaagctatcccctgtgatatcatctgttacaaactttatatggtagagaaattttatgaaatactgaattttttgtttctcagagctgttgaaataagtcagtaacaataatttctgaaaaaacaacatgttagagaagaacacttcagaagtagaagaaagaaagtgtgctcagaatggattactaaatgtactatattgctactctaaattgattttccttaaccataagattagattaaaatgaaccaggaagtgacagaagtcagctgccttacaggctacatttttactcttaattatgagaaataacaaaagtaaattaatatttactttagaaattgttgaaatccgggcaaggggcagtggctcatgcccgtaatcccagcactttgggaggctgaggcggggggatcacctgaggtcaggagtttgagaccagcctggtcaacatggtgaaaccccatctctactaaaaatacaagaattagccagacgtggtggcgagtgtctgtaatcccagctgctcgggaggctgaggcaggagaattgcttgaacctggaaggcggaggttgcagtgagccgatatcacaccataactctccagcctgggtgatagagcaagactccgtctccaaaaaaaaaaaaaagaaaaaaaagaaattgttggtatcctaaaaatgttaaaaacttaaactagaaaatatttaaatgtgtttgaaagaaattgatttaaatagaaatattcccatatgaaatgccaaaatttgataaagtgataaaatacagttttctgaatataagactaaacaccatgattaagattaaacaccctatgatttttagttttcttcacataagcattcattaattgatactgttcagatactatgaaaaccagttaatacaactttacatcttcaaaagttaactatgctattaataactatactttcaaagtaggttttaaatctatcaatttattgcactgatatcagttatttatggaatgtagaactatacaatgcatgatacactaatacatttaagtaatttttagactccaggattcatctacacaagtgaattgctcttcccaataggactaacatattaacactgcttcatttattgaaagcatcagtctcaacaatatacaacactttgtgcactggttcccacacattaatgtacaaatgaatgacttgaggatactgtatagattctgcttcattaggtcttgattggggcttgagattctccatatctaagttcctaggggatgctcatgtcacttgtctggggatggtacttagagcagagatccccaattctcgggcgacaaactggtaccagcagactgggccgcacagcaggaggtgaatggcaggtgagtgagcattagcgcctgagctccacctcctgtcagatcagcggtggcattagattctcatgggaccgtgaaccctattgtgaactgctcatgcgagggatctaggttgcatgcctcttatgagaatctgactaatgcctgatgatctgaggtagaacagtttcatcccgaaaccatctcccggactcctgggctgtggaaaaattgtcttccacaaaactggtccctggtgccaaaaaggttgggtattgctgacttagagtaataatgtgtaaagaacagaaaatatccagagcagaaccgataatgagtgttgtttagatttctctgtatcactgacttcaattctttactagtttacttttgataccaggaacatttcctatcaatttattacattttgaggcataggttaaaatcaataatgttgtttcttaaaagcccctcttatctgagaatgttaaacattatttaataacattatcttgtaaggtggaaaagtcagtgcaagatttcctgggctctagtcttagctctgatacacattttgcaatgtgactgtaggcaaattattcaactccctgagtttcttcatctgaaaaataggagaattgatttagatcaggagtttttcagatatttgctctttagaaatcagagtttattcttcagataaatagtggtgaaactagactgagtgatttggaaccaccctgttagaatcaaaaccgtcttacacggtaatttgactaacatcagcactaggatgcttaccaacactgcagtaaaacttgccctttttttggttggttttattgttgcttgttagttttaaatttttaatttttccgaaggtgctgtttttggaagcatgctttttttttttttcctaatgagtatgtaaggcattgttgcacatcttcacactggcatacaccattcaccgtctttacagtgaactgataatgcatttaatttgtcaagagtcatactggtatacactgtcagatctaacactaaattgaaaataccaattttaaaaaaaattttagttgttttaccttaggttcaatcatttgcttttatacaaatagtaaaagaaacattgtcttttgtttttagaatcaagtcatttggaacagcaacttgaagaagctaatgctgtgaggcaagaactagatgatgcttttagacaaatcaaggcttatgaaaaacaaatcaaaacgttacaacaagaaagagaagatctaaataaggtaaaatatgtgactagatcattaaagctttttctctattttgaatcttgtataataagaatactgaaagcattcataatgaattacaataatacatagtatattttatcttcttaaaagttggtctaaaaatttaggattattttggtgtatgttatttggttagtagctagaaattaagaaactttgttgaagcttgaaagttcagaatcttttgttcttttagaaagttaatagtcaaaggactagattaaaagaatgttattgaaaaagaaacatgaagaaataagaaaatagtagaaactgtttagcatctatagaaaaaatttaaaatacaggcatattttcattataacttactgatatgaacattcttaaaatattataatttaaaatttattggtgatttgttttaaatgtcagtttttatctttcaaagcagcaggggtaaataagctaaagttattttcataagaactttgtgaaacatttaaacattattttaaacatcaaaaaaatttattgatagctaagcctctgtaacacagcccaatagggtaaataatattcaagcactagagaaaacacttttcttgaaccacatgctgctaagcaccgtgactttcattaacaaatagaaaaatgttgggactaatactagccattgcaaacaagaaatcagtagtttgtggcatagcataagttgtattcaatgatgtaatatattaatataatgcactgataatatagaatgaatgaagtcctcctggggaagatttgaaagtcttggtgccaggaaggagatggagggaattaaaaataaaacaaataacctttagctgatgaaactaatcaaaaggactaaaagagaacgctattaaagaatagattgaaatagaccagtacatttttactttctgcttaatgcggtttcaagcaatttttttgtataggtccatttaaattcattgccaatttaataatatttgtcatttaaaactgttagtgttctattcgctttaataatattatagtaatattaatggtttaccatttttattggtaggattactcagccaaatttagttaacataatcggcgttatgcttcctgctttctactcttctgagcgatttccttggaaatctaaagagaaggatcttggattctttctctaaccctttggaaagaggatgaaaatcagatagctagatttatagtgacaggtggtcaccaatttttaatgttttagtattaaaaatcctttttatatattttgagaagatgatagtgctgacgaaaagaaacagattataccaataaaggactttgagtgatttgcttaatatcctataggcagtagaaaatgatttttctccacattccttgccaacacttgttatcctttgtcttttaaaaaaaatagccattccaatagatgtgaagtgacagctcgttatggttttgatgtttacttctcttatgactagtgatgttgaatactttttcatatacctattggccatttgtatgtcgtcttagagaaatgtccactgaggtcctttgcccattttttgattgagttagcaaatattctgattttttataatgccaaactgttttctagcataccaacttccatttccacgagtagtgtagagtttctgtgttcatatttccagatttattacttttggccaatctagtctaaattgagattttaggttttagaaaatgtttggttgtggtcttaatttgcatttctctaattagtaatgaggtcaagtatcttcacatgggtctattgtcgtttacatttcttttgtgaaatgcttcttaatatcttttgtatatttttctgttgggtttttttcttactggttataagaattcttggaaatatttgggcattctaatcttttgtgagttatatgtgttacaaatatctctcagtggcttatctttttgcttttaggaatctttttattaaaaagtgttcttaggtttgttgtggccagatttatcaatatttttatggttagtgtatttgctgtcttgtataaaaaatctgagattttaaaaaattctcctaaatttttatcttaaagttttaaatttttaggtctgtatctcaactattttttgaaaactgtgaagatggaattttgattaaaactgcatttaatcagctgggcatggtgacttatgcttgtaatcctggcacttttgagaggctgaggcaggaggatcacttgagtccaggagtttgagaccagcctaggcaacaacagaatgagacccagtctctacaaaaaataaaaaagttagctgggtggtgcacacctgtcatcccagctgctcaggatgctgaggcaggaggttcacttgagcctggtagattgaggctgcagtgagccatgatcatgtcactgcactccagcctgatcaacagagcaagaccctatctcagggggaaaaaaatgcactgaatttataggtcaatttagggagaattaacatcattacaatattgtctttaagttcttactatctatatttattactatttttctgaatatacacacagatttccagaatagaacggattgttattacttacatacagcaaagaataactacatggctcccttctgtgccaagtcttaccccaagagaaataccctgagagctggcaggataaaaccctacacataaggagtctgtgttgtaaatgaggagccttgaaaacatttatgcaggtgcttatatagaggaaagaagcaactgcttcctgctttctcctgttctgagagagttccttggaaatctaaagagaaggatcttgggttctttctctaaccctttggaatatagataaatattccctctggaaatataagcccagagtctcctgctccatcaaaggaagcccccttggtccaggctcctactctctcccaccccaccttgaaatgtaaacacataacctccagaaggtaaatctcttagttttctcactactaaatcagttataagttactttttaagactttcatttagcccaggtttcggtaaaatttgttcaaaagggaccagctgcaaaaacataaaagtttctctgcattcctaaagtctttttatttgtaaacatgatatctctccatatatttaaagttcttttaaaatgttcttccataaaatttcatacagaaaagactttcatttcttttcttgttttactgtattagctagaacctcagtacagtgttgactagaaatgataatagcaggcatccccatcttgttccccgattttaaagaaattgcctctgatatttcatcgctaataatgtttgctgtaagtttttaataaataatttttatcaggttaaagaagttaccttctattcctattttgttaataccttttatttagaatggatactgactttattaaatgtgcccttctgtgctctattgatatgattatatgattcttgtcctttaataagttggtgtggtgaactacgtttgtagactttttctaatattaagccactgttatatttctgggttaagcccaaatttcttataatactgtgatagtatagataacatttatgtagcaaatatctgttctaatagcactgttgtgaatgctttacatatattaaattatttaatctccatcacagccccatgagttaggtgttattctcattttatgaatggtgagaccaagcacagagaagttagttcacttgctcaaggtcatagaaatagaaagtggtggagctagattttcagcacaaggagtttagctccagacttcatgcatatctactttatatatcactggaactgatttactaattttcaaaaagggttttcactgttttttataagtgagattgacgtgtttttttttttcctcttttcttttttttgatattcagattatactggtccatagaaagagttgaattgcattcctgctttttctcacggaccaatgagttatttataagtatcttttaaaattttcaatatatggagttttatctttttttgttgacttctaatttagttgcagtgtcattacagaacttgctctacgtggtatctgttctttgaaatctactgagacttgcttcatggcgtagcctgttccatttttaaaacattcaatgtgtgttcttgaaaatagttcgtgttccaatttagagagcaagcttctgtgtatgaagcttttcagatcgtctttatattcactatttttttttttttgcctacctggtttatctattaatgactaagaaattaaaatttttcattatagtgatgtttaccaatttttctctgaagttctaacagtttctttttaatgattttgagactctcttatagaaggcacaaaagtttaggataatatttcttcttagtgtattgaattttatcattatgtagtgatattccttatccttagtaatactttttctactgaagtcaagatttcatttcttaatccagtataatagatggcctttaactggttactaatgtatatggatgtgtttctactatctttttttctgtttgtcatgatttttgcatgctgctttttatttcctttcttcccttctcaaaagtttttaactgtggtaaaatacgtataacataaaattaccatctaaacaattttcaagtgcacagttaagtaatgttaagtgtatttacattcttgtgcagcagtctccagaactctttttgtttgcaaaatcatttcttcttcactgaaatttgttgttgttgttgttgacattttagtttactctctagtcttttggtggttactcttggcttctaccatgtgtattaaccaagtttaagtttaagttaaatatcctaaaactatttcccacaaacaatttaaagacgtttaattttacagattgagtatcccttatctgaaatgcttgggaccagcatttggattttggaatatttgcattataccaattgagtatttctaacctgaaaatccaaaatctgaaatgctccagtgagcatttcctttgaacatcatgttggcactcaaaatgttttggattttgaaacatttcagatttcaaattttttggattattcaagctgtattacctctttacagctttatgtgttgttattggccagtattttagctgttatcttttgtttactgtacaaagttaacatttttatagtataatcttatatacacatttgtttacattcacattcatgttaaccattttattagctcttcatttctcttgcattagatctctaaaatctttttttttttttttcctgaaataaagctttactggttcctttagtgcagatctgattatggtaagctctgtcagttttgtttatatgaagcctcagacttgaaaagtaatttttggggtatataattttaattatcttctcatatctttgaaaatattttaccctgtttctgttgctgttgttacttcttcagtgtctgttcttagtctaattttcctcctccccgccttatttgaggatggggaataggtgatttgtctttctttccctctgatttctttacggtcttttatttctctttggtattacccagcgtcattttttgaacacttctggtttgggttttctaaattttttcttgattcataaatgtagagcttcctgtggtgatggatgcatgtctttaattttaacatttatattttttcacttcttttcttttctttttttttgagttggagtctcactgtattgcccaagctagagtgcaatggagtgatctcagctccctgcagcctcagcctcccaggctcaagtcatcttcccaccttagtagctacgactataggtgcataccaccatacctggcctttttttttttttttacattgttattttttgtagagataaggtctcacaatgtcgcctaggctgatcttgaactccttggctcaagcaatcctcctgcctcagcctcccaaagtgctgggattatagatgagagcaactgtgcctggccttatgttttctatttctaaatgttccatacagttcttctataaatctgcttggtcatttttaatagccttttgtcatttttttgtttttattatttcatcctttattccttgaaatatttcatatgtagcaattttgtgttgatatctaatgattttaataccaagatacctttgatgtttatttttattgtttttgttgtttgattgtggtgctttgttctttgagtattttggaatctgtggttgtgagctcattcttgtttgttcttaatatgtgaaaaattcagatttcattgcacttctttcccccaaagaaaatttgcatttccttttgctgggaggcaggtagtggtactgccctgagtccactttagctctcctctaagtttctcattttaattagggagtctccggtttagtttctatctccttgcttctggcttgaggcgtagcctccatgtcaagggcaacacttgttttttatgaaaacaacctcacacatatttattaaatttcacaaatatttattttgaccatcatatcgtttattcctaaactttttcctcatttgtggaaactaaggtactataaggttaaatcagcagctcctgtgcatttactgtattagcataaggaattgagactatatcaggacaccatgccgttaatatctgttcagaagtagtttatttcttttaatttgtcctaaggtgaataattttggctcttcttgacttaatgatagctattctgatggccaaatttggtctctaggttgcagaattggtaacttatattctttttacaaaactgtaggaactagtccaggctagtgagcgattaaaaaaccaatccaaagagctgaaagacgcacactgtcagaggaaactggccatgcaggaattcatggagatcaatgagcggctaacagaattgcacacccaaaaacagaaacttgctcgccatgtccgagataaggaagaagaggtggacctggtgatgcaaaaagttgaaagcttaaggcaagaactgcgcagaacagaaagagccaaaaaagaggttagtagtcaggcaaattttgtagtgcccatgggatagtgattaaagctgctttttgagactagtaatataatatataatttttaaaatatttttaaagttatttttatagttcattaatattatttaatatggatttaaacatttgaattatcttcctgttacaaggtaaattctcattgggataaatgttttgtttggaaaaaaattataaccgtgaaatctgcatttttcattctgttttaaagctggaagttcatacagaagctctagctgctgaagcatctaaagacaggaagctacgtgaacagagtgagcactattctaagcaactggaaaatgaattggagggactgaaggtatcttttgacttcacatttatgaagcttaattgattttcttctctttaggcttgttaggagtgttagcttgtatactgtattcggtgtgcagtgaaaactagcctttcttcaaacctactattcataattctcgttcttaccagtaggaagctaatacagatttcttaattatgctgaagaaaatggaggactatgtttagtgggaaatatgaatctatcaggagggagttagaatgtttatgcagacctacacaagatgtactggtaatctgcatgggagtcatggttagatatccagaccctctatatatctcataaatgactcaggagaagctcaggggtagctttgcagacacttgtgtctgttaggataagatgataatgggcatagtctaaaacatctcagcagaaggtgctttgcaagggaaattcttgagccgttctggagttagactacctattcttaacttcttgctttcaccagttacttgctgtgtaacatgtaacattggacagaacattatggggccgggcacagtggctcacgcctgtaatcccaacactttgggaggccgggatgggcagatcacttgaggtcaggagttcgagaccagcctgaccaacatggcgaaaccccatctctactaaaaatacaaaattagccaggcatggtgcgcacgcctgtaatcccagctactgaggaggctgaggcaggagaattgcttgaacccgggaggtggaggttgcagtgagcctagatcgcactattgcactccagcctgggcaagagcgaaactccgtcttaaacaaaacaaaacaacatttaacctcattatactttaattttctcctctataaaactatttcatgtcacgagtacatacatacatttgtaagctgacacattataagtgttcagtaactgttagtataatagtattatgtatattacagtgagtgaattaagatcaaattgacgtgatatttttaccatactaagtaatagtctgaatgtggtatctgccttttatcttttttgactggaaactttttccatcctgttttctctgctatatctgtcagcccccatttcacctacttcttttaggaccatgcttagtctagtgtctaattatccataacatgtaagaatcatactccttaaaatctacatactcctgtactgcttcttggaattctgttaatacctgtagcttagctcttattgtaattatttatttgcaaatctgtatatttccaactaaactgtgacctagcttattgaaaacacagacagttatattgttaatttggccctggcaaaggaccagatattgaatacttgtttaataaacctgttaagttaatgaatgaatcatgtggtagaattggtcagagcttactttgtctgacaattgaatttcagtggttgcaagtcaaattcctttgctgtaagtagaaatataaactcttgattttattcttgtttttgttttgggagttttagttcatcatgaattcatagtcctggttagctttacgaagtagagatgtgtcttctatatacctctagagcaggattagtatgtagaagtaattacatcaccatcccagatacttttcagtcagggcctgctttgtttccagcactattctgggtgctttggatgataagaataataaagtacaaactggaagaaatcttaggcattatttttatttgagaaaattgagggtcagaagagttaaatattttagagtcataagtgatagagtacaatgtaaacccaggttttctaactttcattcacatgatttatctaatggtttgctttttatttttttctacaataaacattacttcattggagactctgtatcatgtttacctacttgccaaacacatctctgtgtcatttagaaatttgagataatataacacctacctagtaaataagttaatgcatttaacatgtatagaccagaacctggcatgtagtaaagggttaaaacatattttggcccttttaaagattgtaggaatcttcacttcattgagaatacttttgacagacttaaagagagaaatagccctacaaaaagagttaaggacttcaatatctcacttctaataatggatagaacatatgcacacaagatcaataaggatattgaggttgtgaacaccactataaccaactagacctaacagatacagagtcagcccttcatatccatgagttctgcattcgtggattcaaccaaccatggatcaaaaatatttggaggaagaaaattagatgggtgcatttgtaccgaacatgtacagatgatttttcttgtcattgttccaaatgtaaaacctacttatatagcatttatatcacattaggtattgtaagtagagatcgtgacgttttgatcagccacagactgcatatacaacaatggtcccttaagagtataacggagttggaaaaattcctatcatctagtaacgtcttgatgatcttgaccctgtgtaaaccctatattaatgtgtgtgtttgtgtcttaatttttaacaaaaatgtttcaaaagtaaaacaaataaaaattttacaaatagaaaaaagcttatagaaaatggatataaagaaagatagccaggtgcggtggctcacacctgtaatcccagcactttgggaggctgaggtaggtggatcactggaggccaggagtttgagaccagcctggccaacatagtgaaaccccgtctctactaaaaatacaaaaattagctgaacgtggtggcgcatgcctgtaatcccagctactctggtggctgaggcacgagaatcgcctgaacctgggaggcagagattgcagtaagctgagattgagtcactgcactccagcctgggtgacagagtgagactctgtctcaaaaaaaaaagatacatttttgtacatctgtacaatgtgtttgtattttaagtgttactacagaagagttcagaaagttttaaaatagtaaatagcttataaaggtatagtaagccaagttttttattggataagtaaaattttaaaaatagtgtagcctaagtataaagtgtttatctatcaggtctccagtagtgtacagtaatgtcctaggccttcatgttcactcaccagtcactaactcatccaggccaatttccagtcctgttaagttccattcataatgctctatatgacataccattttttattttttataccttacttttataccatatttatatgcgacataccttttttatcttttatatgatgtttttactgtatcttttctatatttagagacacaagtgtttactgttgtgttacagttccctgcagtattcaggacagtaccatactgtacaggtttgtaacctgggagcaataggctataccctatagcctaggtgtgtagtaggctgtaccatctaggtttgtggaagtacaacttatgttcacacaaggacaaaatcacctaatgagatgcatttctcagaatatatccctgtcgtttagcaatatgtgactgtaatctagaggtgatttaaagtatatgggaggatgtgtgtaggttgtatgcaaacactgtaccattgtatatatgagacttgagcatccgtggattttaggtattcaagtagggtcctggaatcagtgccctatggatactgagagacaaccatatatagaacttttcacccaacaacagcaaaatatacattcttttaaagtacacatggcagattctccaggatagaccacatgttaggccaaaaaacattttagtaaatggaagaagattgaagtcgtatgaggtatcatctccaaccacaatggaatggagctagaaatcaattacagggaaaaaaaaaacctggaatgctcaaaaatatgtggaaattaatgaacacactcttaagcaaccataggtcaaagaataaatcaccagggaaattagaaaatacgtgaatgaaaatgaaaatacaatacaccagaacttacagatgccataaaggcagtgctcaagagagaaatttgtaactataaatgcctgtattaaaaggaaggactaaaagggaagaaagtaaaaatcactaacctaactttatatcttaaggtactagaaaaagaagagcaaactgaatccaaagatagcagaaggaatgaaataataaagagtagagcacagctaaataaaataaagactagaaaataatagaatcaataaaacaaaaagttagttggttagaagtcaacaaaattgacaacctttaattagactgaccccttcccaataaaaagagctaaagagagagagatgttgcaaataactaaaatcagaagtataagggaaaattttattactgaccaacagaaaacaaaagaattataagaggaacaactgtatgcccacagagtagataacctagatgaaatggacaaattcttagaaggatacaaataacccagtatgactcatggcaaaatagaaaatctctgcagacctataacaagtaaagacattgaatcagtaatcaaaaaccttctgacagaccctcctgcactccacaaccccactccagcggcttgttaaaaaaagacaggccaccttctaacaaagaaaagtccaagaccacaccacttcactgctgaattgaaccaaacacttaaagaaaaattaacagcaatccttcccaaactcttttttgagaaggcacctgtcgtcccagctactctgggggctgaggcaggaggatcacctgagacttcggaggtcgagacttcagtgagccatgattgggccactgcattccagcctgggtgacagagtgagaccctgtctcaaaaggaaaggtacttgagacttgagcttaaaataaattcacccaagttccaggctgcaagatcaacacatagcaatcagttgtgtttctataaggcagcacgaacagtctgcaaagaatgttaagaaagcaattccacttaacaattgcatctaaaggaataaaatcccttgggacacatttatccaactaaaaatgacaaaacattgctgaaaggaattaaagaaaatctaaataaatggaaagacatctcatgtttatgaatagtgggatttaatattgttaagatgcagtactactacctaaagttatgtacagcttcaattcagtccctatcaaaattcaaagtcttttttgcagaaataaaaagctgatcctcaaattcatatggaattgcaaaggacctggacagccaaaacaatcataaaaaaaggacaaagttggaggagtcatacttcagaacttactacaaagttacagtaatcaaaacaatgtggtactgtcataaggatagacatacagaccaataaaatataattaagaatacagaaataaatgcatgtatctatggccaattaatttttgacaaatgtactaagtccattcaatggggaaagaatagtttctacagatgttttgggacaactgaatattaacctacaaaagaatgaaattggtcacctgcatcacaccagttatacaaattaactcaaattggatcagcaactgaaatcttacagctacaactataaaactcaagagaccttggatttgataatggattcttagaaataatactgaaagaagaattaacaccaatccttctcaaactctttttaaaatagaagaggaggaaacacttcctaactgattctgtgaagctggcattaccctgaataccagagccagacaaaaaaatcacaaaattacagaccaacgtttcttattaatatagatgcataaataacaaaaaaagtataaattgggctttatcaaaattaaaacttaatgtgtatggtatcaaaggacattattaagaaagtaaaaagacagcttaagggatgcaagaaaatatttgcaaatcatatatgtgataacaatttaatatccagattatatgaagaactacagctcaacaacagaaagacaactcaatcaaaaaatgggcaaaagacttgaatagacatttctccagagaagatatatataaatggtcaataagcaaattaaaaaatgctcaacattactaatcactaggagaattcaaattaaaaccaaaatgagataccacttcatatgtactgggatagctaaaattaaaaagaaagaaaaagaaagtaggtcttaacaagaaggtagagaattggaacccttatatgttgctaaagaaagtataaaataatgtagccacttttgaaacaatgtggtggttcctcaaaaagtcaaacagaactaccacatgatccagccattccactcctaggtatatacccaaaatatttgaacataggaactctaatagatgctttacacaagtgtttattgaagtattattgcagtagccaaaaggtggaaacaatccaagtgttcatcaacagatggataaagaaaatgtggtatataaatacaatggagtatcatttatctatgaaaaggaatgaagtcatgatacatgctacaacatggctgaatctcgaaaacgtactaagtaaaagaagccagacacaacaggacaaatactatatgattccacttatatgaaataatttagaataggcaaattcaggcccgatgcagtggctcatgactgtaatcccagcactttgggaggccgaagcaggaggattacttgagcccaggagttcaaaaccagcctgggcaacagagtgagaccccatctctacagaaaattaagaaattagctgggcatggtggtagatgcctgtagtcccagctactcaggaggctgaggtgggaagattgcttgaggtcgaggctgcagtgagtcatgattgtgccagtgtactccagcctggacaacagagtgagaccctgtctcaaaaaaaaaaaaaaaaaaaaaagagaataggcaaattcacagatacaaagtagaatagaggttaccagggtttgggtggagtggggaaacagggagttaattcttaatggttacagaattttttgttggaatgatgaaaaaattttggaaatagatactggtgatatttatatagcattgcaaatgtaattaatgccattgaattgtgcatgtagaatggttaaaatggaaaattttgttgtatgttgttttaccacttaaaaactattaatgcagtataacaagataattgaattttacacttttaaatgagtgatttgtatgatatgtgaatcacatttcaataaaaaaaatttttaaagaaaacttttaagttgttatatatgatgctgtgatgttactagaggtatttttttttccctattgtgttagtttgttggggatgctgttgtgaagtaccacacactgggtggcttaaaacacagaactgtctcagttctggaggctagaagttcaaaatcaagatattggtatggttggcttctggtggcatgctgacagtctttggtattgcttggcttgtaagatacatcaccctgatctctgctttcatgttcacatggagttctccctgtgtgtctgtgtccagatttctccttttataaggacgcagtcttaatgaattaggggtgtgccttaccgttttgattcgatttacctcttaaaaccatattttaaaataaggttacattctgaagtactggtggttagggcttcaacataggaatttgaaggtaatacagttcaacccatacacttacatttttcacttaatagtgtaataagcatttttactttcattaccaaattgtcacaaacataattttaatgattttaagtagttcaagaagaattttcacatacctagttatttctatttgttattacaaaattatcacaaacataattttagtgattataaataattcaagaagactatccgcataaccaattttctattgttacctttaaaaattctttgcagtattataattgccaaaaacacctttatgcactgctttttctgtatcattttcttagaacaggcacccagaaacgaagttagcgattattttaaggctaatttatgtgataaatcccacaaatctttgcaaatacgagaggccaaaaaagttgtacctctttatttcttggtttattagtggattgttttattaataatattttctgttcataaattctctctttatttgctaatttaatttagtagtcttaatgtttttcttaccaattcataaaactctctgcacagtaaaaattttactttttgaagtgttttcagcaactatttaaccaatttatcatgtatcccttaattttgatcttgtttctttttaaaattatagaagatttgttggtttttctcagtgtttcttctgtcgcttcaaagcttggcttgtccttctctcagagttttgataagtatttaattctgctttcttttggatgtttgttgggtctaaaaataattaactgtttaatctattaggagtttgttttgctaaatggtatgacatgagattgatttttgttattttattgctagctggtgattccaatttcatttattaatcattgccttacccaatgactgaagatgtctcatatattgaattaaattatttcggggagcagatctgtcatagtgatactttagtaaagagttatagtatgttttaatatttgaaaagtatattttcccatgcaccccttggtattttctttgacatttttaccagtttattcttatccacggagtagaaacatgtctccatgcccttctatctccacatttataaagaacttttaattagaattatgtggaagataacatgaaaaatattgaatatgtacatttaatcttcccttttaaatactttgtttcaagacttgtaggtttctcatataaatcagaaatttctcactattatccttcaattaacaatgaaatttttgttatgtttattgcttattttaatatctggaagtattgatttgtgtttatcttatatgctattgctttactcaactatcaattctaacacatttcactagatttatttgtattttctgggtgaacaattaaatagaaatgataaatatgttatttttctttacaatattcatttttttaactattttacatgttggtcagagagacaattttatatgttggtctgaaatgaatgtcgcataagagtggtaatggaagacatcctttttgttgatatttctgaatttactcgatgtatctctgatgtttcacgtaaatagtattttgatagctcaaaatggatacttttaaattaaaagaattactggttttttttatgtaatggttttattgtgggacttagagtagtatttggaattattactggattttataaaatgctattgggtaggggtacatactgagattatcatttggatttttacatactgagattatcatttggattttaaaatttaatctattaaaataattcattttatgacacatttattaataagtttgctgacattaaattatcttcggtgtctgcaataaaacctattatttcttggtaggtttttcttttttacagatggtttaataatatcatttgtgttctatttggcttttcacattcttatcaataagtgatatgcatctgtaaccataattgttggtatctttgttaggttttgatttcaggggaatttttttttcttgaaacttactgtacatgctgctgccatcccaacagtcatcacaataaacagaacatcaaactaaaagtgttcttttctttaagtagttagaaaaatgcaagaatagtacgggtcagtggtaagtgccacagatgtcaatttagttatcattttcatagcgatgcttcagagcaagaatctgccttgctctggttcttctcaagttaagcagacttttgcatgttaggctgcctcataaaccattggacgatctggatctggcacatgaaaagtattttttcgataaatggttaatattgcagtggacacgccagggctatttctctggcccacatcacttcctttgctctttttaaattatatacttcaaacatgaaggagtcagggtatttttccttctactttaagtcaactacgcttaccagtttttattgcccttatgactgacttcaaaaaagaatacagtgacctccagatcttgattctttgttgtccctaaacttaagtgcatagattcttttagttttaaaagttttattgcaactggggagacagataagatataacaaattaatgacctttggaaaattacaaagcttaggaaatcttacagtcataatctgtagtctgtatgtttctgtttaccctttaggcagtacctggaagaatctttggactttacattttcttttctttcttattactgagaggatatttcagccttatttctaaaaaggtagatttcaacccaatttcttctccttcttaatcatacctaaaagtgaagagacctaccgttttttttctagtgtagatatgaaggagatgttagtttagacctttgccatttctcacaggactattgcaaaaggtttctgtctttatttttgtacattctactctaccattcctttgcccattttaatttttttaagacacagatatccttaaaactttttatcagttcttcatcagatttaggatgcagttagatttttctctcactccatacaccaacaataattgtaaataaattagaaatttaaatgtaaagcaagaaatcatgtaagtcccagccaaaaatttgaataaatatgtaatctttgtgtgaagaaaactttttaaaaacagcaacaaagacagactattaaggaatgtaaactgaggaaaatatttgcaatatatggcaggcaaaaagttagtagacttaacatagaattttatttttgttaggatgtacaaaaaatgcagttatatgaatactcatttatattacagaaagaagttatttaaatattttattattcttgtagatgcactaatcttttgttattatttctatagcaaaaacaaattagttactcaccaggagtatgcagcatagaacatcagcaagagataaccaaactaaagactgatttggaaaagaaaagtatcttttatgaagaagaattatctaaaagagaaggaatacatgcaaatgaaataaaaaatcttaagaaagaactgcatgattcagaaggtcagcaacttgctctcaacaaagaaattatgattttaaaagacaaattggaaaaaaccagaagagaaaggtaagaatcacattatttactatacagtaaatacttattgagtacctgttatgtgtcaagtattggagatacatagacaataaacatatcctagacctaaaggagaccaaagtcaggattggcagatagaaaattataatacagcatgatagaaaagcaaatgggatgataattctgaacaaaattacttctagctgggtatgatggcacacgcacacacacacacacacacacacacacacacacacactagctgggtatgatggcagacacacacacacacctataatctgagctacttaagaggctgaggaaggaggcttacttgagcccaggagttcaagcccagcccaggcaacatagtaaaaccctatctctaccagggaaaaaaaaaagaaagaaacgataaacttctaagctgagatataattagtaagaaagaagtagctagggaggagtgtccagggaggatgtgctaagatctaaaggaaaagggggacatgccacattcagaaaatgatagctgtttactatgactgaaacataaatagatgagagtggtgagaaatgaagctggaaagtaaaatagaggcaaagtcatgacagaatacaaggcatttgcacattatcctcaggccattgaagaaaggtgacatgattatatttacatttttgaatcaccactcttgctactgtgtggagagtgaagtgggaggagcaggctggaggtaggaatccagctgcgagagatgaggtgtgggcctcaggtggtgatagtgaagatggagagaagtggatggattccatctactaagaagatagaacctggagaacgtaataagcagctgtgagaaatgagggagcaggagaaattatcagttatactcagctttctagatttcaggcaactgaatatatgttattgtcattctcagcttatattggaagcccaaatttattcaatttggtgcatttattcattcagtaatattttttaacctaccatatctgacgcactgttgtagggactggtacaaaaagacaataacaatgaaaaaaattcctgtcccatagaactttctataagtaaaacatagaatacattagataatgataaatgctaagaacaaaaaagtaagaggaaatccgttctatgaagcattcaagggagggttaaattatataaatacaggatggccagggaaaactatactgagaaggtgattgatttcttagttaaagattaaaacaagaagagatgtttatacagatgtgtgaaaagagcattctacatggagaagaataagttttttacaaaatagtgaggaggccattgtggctcaagtagaatgatgggaaaagaatagtagagacgagagaggaggcaacagggggccacatcatgtaaagccttgtaagtcatatttagtggatgcatagtttcttattctgagtaaaactattgcagggaattacagtcatgcattgcttaacaccagggatattttctgagaaaggtattgttatatgattatatacaagattttttttaaaaagggtacacctatataaggcacttaccatgaatgaagcttacaggactggaagttgctctcggtgagtcacaactagaaggtcttcagggacagttacatacatgaaattgtcatttcctatgacaacagtgccttcttcgggaatacttcctgaaggacctgcctgaggttgttttacagttaacttttaaaaaaatattaagtagaagccgtatactctaagataacgatacgaagtatagtaaatatataaaccagtaacacagtcatttattattaagtattaggtactgtacataattgtatgtgctatacttttatactgccgacagcacagtaggtttgtttacactgacatcaccacatacacgtgagtaatgctttacattagtacttaaaacagttacaatatcacaggtaataggaatttttcaacaccacatagtgttcccataagattataataaagttgaaaaatttacgcagttatggactaatgcattatggactaatggcagtccatagttgatcaaaatgtcattatgcattacatgactatattgtagaattctgagcagaaaagtgacaactgatttgtgttttaataggatcactctgaatactaggttaagagtagtctgaattaggtaagagcaaaagtgatgagaccacttagaagacgattgaagtaatccaagcagaagatgacagtggctttggcctagagtagtggcaaaagggataagaagggtttgaattttaatgatctttaaaggtaaagatagcaatgtattgatggactaggtatgggatgagggagaaaaaggaagactcaaggatgatactaaggtttttggtccaagcagttgggagaatatattactagaaaggacgactacaggagaggcaagtttgggattgggagtgttatcaagggcacattttttcccctgacaagactctttcataaacagaaagagctcattttaaacagttacagttgagatgcttattagacattcagttggagatgtcaaataggctgatgtatagaccagcctggatttgggggcaggaggtaaaatcctgagaatatttatcacttccatggcatttaaacctctgagactgtatgaaaacacttagtgaatggatatagatagaaaaaaatttcttagaactgagccttggggtactccaatttttggaagtcaagagatgatgaggaaccagcagagtggatagagaaagggcagtgagaatgacagcataataacaggcaaatttagtgtccttgtgttagcagcgccgaatccatatgtgtttgcagcagcctcaattcttgcctcctcagaataaagaattagaccaaggggcataagacagggagagactgaggcaagttttagaacaggagtgaaagtttgttaaaaagctttagaacaggaataaaaggaagcaaagtacacttggaattgggctaagtgggtgacttgagagattcaagtgtatgatttaacctttgacttggtgtttaacctttgacttggcatgcttctgtggggttgcatcccttctctgattcttcccttggggtgggctgtccacatgcacagtgacctaccagcacttgggaggggctgcatgcacagtgtgtttactgaagttgtacacgtgctcatttgaggcattcttccctgactggttgagtgctcctagaagaaggtcatatgccaccagttaaactccaccattttgcctcttagtgtgcatgcttgagcccacttgcctaactcctgagtcttattgggaagctgctgatcaccagtttcaggcttttctatctagtggtagactgccttttcctggcaccaggtgtgaccaattattattttagagagacagtttaacaaccacctgatcatcacctgatgtggccattcctggttgggggtgtcctcctgtcctgcttatgtctgcctgactgcctactgtaacactggaaaccaagtgaagaaagcatataaagtctgtcagcatgtcaaataaaatgagttctgaatcaaatgagataaggactaaataatgacattgagtttagcaatatgaaggtcattggtgcctttgataagaacaattttgtggaaaagtagaggcagagaccccacagtaggatcaaaagagactggaaggagagcagttagaggcagaacatagaggtaaaactattgtactttgttgtgtgggtgtgtgtgtgtgttttaagatggaaaaaataacagcatgcttgtaatctgatagaaattatagatgattggaggagagaggagaggaggaaagtactggagcaaagtacttgaggatgttcataatgaaggggttggccttactcgatcctgttttgttttttttaacctatcatgaaaagagaaagtagagtatatggacatagatggggtggtgggaacttgtctgtgtgttgagctctcttgttgagttgggcatattgaacttgtggtgcttggaagacatctttgtggagatgtcaggtcagtttgaaggccacaaaagcttcaagtgtagattcagagatcatcagctgaatacataaataaaaccagctggtgtcattgatgagctaactcaggcagcatgtgtagagagggaagagacagcacctaagacagaatactgaggagcattgatatttacaggaaggatggaagagtcacaaagacagctgaaaagaagccaggaatgtagactaaaactaggagagcatgggatccctgaagtcaaagccagacagcatgtcaagagggcaagagtggacaacactgtcaaaagtggccagcaggttggatgtaatcacaggcagtgttgagttttgagagagctgttttcttgagatggtggaggcagaagccagattttggtgtggtggagagcaactaagcgatgaaaaagtagttacagaatctgtttcaggatgcttaccagtgagtgggaagacaaggatttaggaaatgaattgtgacacagggtatggggagagtttctttatttttaatatggaagagacttaagcttgtttaatgctaacaggaaaaagaatggttttacatgaaaattttactcaatttatttgtgtacatatttttctgaaagtctttgcagtttttttattgaagttgcttctgtgttaggaaagcttcctaaacataactaaatttaacatctgtctatagatagtagtgaagtgacaaatttgcttggagatggaggttggcttattttccttgaatttgtcatttaaattataatttatcttcaataatgttatgtaggagatttttaagattacacttttaagatcaaccattacttacattgtttgttcaccagaaagcaatcatataaaattacccagaactgatccacatggaatttcatttattgctaacatcctatagcatccatcaaattgctgtagatatatgcctgtttgtcacataacaatacagtcttcaaaaacttattccttgctgggcacggtggcccatgcctgtaatctgagcactttgggaggctgaggcgggcggatcacctgaggtcaggagttcgagaccagcctggccaatattgtaaaaccccatctctactgaaaatacaaaaattagccagacacagtggcatgtgcctgtaatcccagctactccagaggctgaggctagagaattgcttgaacctgaaaggcggagattgcagtgagctgagatcgcaccactgcactccagcctgggatacagagtgagactcttgtctcaaaaaaaaaaaaaaaaaaccaaagaacttattccccttttttccttagaagattttttaagtcttattaaaactgtgtttacttctaagggtaaataagtcatcatgatccacagaactatcattttaaataagctttgtaatattataataataaatgtgtatatatgtagatgtaagaaggaatggctgaaagtattgatagaactgttggaaatttaattgaacttttaatacaatctagggttgagaacaaggaatcattttgtggttcatatatcctctttccctaacctctcctttcaccatgctttctagtatatacataatcaaataaggtgagcataggaagtctacacatcacagatgttttcctcagatttccaaatgaaacacaaatagatgttattaagtatacgttcattgaactcattaaaagtaaatgagtgtgacttttaattttaagggaaaggatgaggatttggaagcatcaggctataagtaaagcagatggtgcaatagccaatctggaatactgttattttcaataaaatcaggcaatgcagttccattctggcattttctgctgtcatttgcagactgtcatctgaagacacctaccatagtgtttctccaggatgctaatgtgccctctcactggtgtatttattaatgccttggtgaaaggagtgctttgtgggtggctgaccaggttgcacataataggtccactctgacattctcatctctttgagccagtagaccctttatgctcagtaagtcaaatatctcgagctaattgactgtaggccactattgggtccaaagtccaggcttcaattactcagcctagcagtgaaaaactacacctgaatattgagtcaacacattaaaccacaaatcctagatgagtatgtgcatttgataaatttggctgtatttaattttttatttacccaccttggggtaacacccttctgacctgaaggaagaagctgaggcacaggatatgtttaaagagtttgtttgagccaaagtgaggacagctgccaggaacacacttccaggttaccttgaggagtgctccattaggtctttgttacaagtagggttttaaaagcaaaaggaggcaagggctgggctgatacaaagttgtttgacaggcattttcactggtttacaaaataacattgattagtgactggctattcattgttgaactatagcgtgtgagtcatggtctccagtgtatggcattttatggctacttggcatcagtttagagcccacatagcaagtggcctcaagaggtgattatttagctcaaggtgggagtgagatgtgactgctctcacattttaaatacctctctgggcctgataatttaaatggtctcacattcctcagataaaaaaagtttattttctttctcagctcttagaattcatttctgtatgctgcccatactccttccaatataaattgccaaaatgctttaacaccttcattgcttttatatctagtctctgctgtgatcttaactgtcatcataggcctgaagacaatgacaagtgattgggtaggtgttaaagatagttggcctccctttttgatgtaactatttcaggtgaagttaataaatgatttatgcaaaaacaggttggttgcctcaggttatagctgctttgaggaaggggatgagattgcttctacctgcagaggtttgaagcaatacgagggttcagagttctcagcactgtcttttgctgcctgaaggttccaatatgttgtccaaaagacagaaactgagacaaaattaatatagagtttatttgggccaaggttgaggactgcagcccaggaaacacagagaagtgctccagagaacaaaaaagaggctcaggtttttaaagaaaaatcaggagagggaacatacaaaagtctttggtcaggagttctcactggtttgcagaaataacattgattaatgattggctgtacattgttgaactatagggtatgagttatggtgtccagaatatggcattgttgggttaatttatagctattggtgacactcagtctggagtccacatagctggtagattacttagctcaactgggagaataagatgtgactgcagtcacatttcagtgcctgtctggccctgctaatttaaaggggctcgcatttctcataaaaagtttcttttctttctcacatctcatgtcttggggactgcactcattctgaatccacacccttagcctaagaggtttggtcagtgttgtgcagatcaacattttgcaccttctgatgggatgcactgagaaacattgttcttgtggtattccagccaaaaatgaattctttgagactagtcataaacatcagatggccccaggttgagtgtcactgtacataattagagacctatacattataaaactgttgtgagagatagggaagactgaggaactgttctagaccaaggaaaattgaagagacatgactactaaatgcaacacatgtttctcagttggatcctgggcttcataagaaaaggagacatggtagggataacttgaattgtgtctgtgaattcagatggtaagtgttctagtaatgttgatttttttaaattgagaggattgcttcgtgattatggtagagtgtccttgactttgaaagatacacactggagtatttaggggtaatagggcataatatctgcagctagctgcctctcagtggttctgaagaacactaataaaatcggtatatacacaataatagagaaaatgcagtaaaatatttacaattggggaatctgggttaagagtatacaaaaattctttgtactattcttgtaactttgctaataataatgaaattatttcgaagtaaattttttaaaatttctgaactcataaattctaattatgaacaaaatgtttggaatactattgttaatcaaattgataacactaggaagtggctctggcctaatactaaataagggcccagcaactttgtattttgtaagaatattgtggtggaaatacacaaagaaaagtatctaatttggtctcaaactaatttgcctttggatagaacgtaatgcatgtgaagcaaatggcaattattattttcattttggtccattatcaaccttgatgtacgttctctgactatataatctaaagtagccaccctctcccaaatactttttgcattgccatatatatacaatttagaacttattatctgaaattagttcattttactcactcttcattggcagtctgcctctaccagaatataagctctgtgagagcagtagctgtcaagacctgcaacaattccttggcacctagtaagcactccataaatagttggtaaatgaatgaacaaatcagtattattgtaatataaattcctggaacatgctttcaccaggcctgtcattatatataaacaagctgtattctatggggacaacatttctgcagttcaatctgatattttgtgttttatgtaactattggaaaaataatttcaagttttcaaatgacctttaattcccctttttccttttgtgttttgaattgtgttagtcaaagtgaaagggaggaatttgaaagtgagttcaaacaacaatatgaacgagaaaaagtgttgttaactgaagaaaataaaaagctgacgagtgaacttgataaggtgagtgatttaatctcatattaattgagtagtacatgctagatgttcaacactgataggaattgtgggtataactttatatttgacatacatttttgtttaatagtctttctcctgagttatgttgcttctatgatatatgcaattctttgttataataatctgataggagtcttctgaagagagagttttgcttatctccttttattctttgtcccatatatttttttgagcctcttatttatttcactctccatccatattagtccattttggggttactataaaggaatacctgagactgggtaatttacaaataaaatagatttattttggcttatggttctgcaggttgtacaggaagtgtggtgccagcatctacttctggtgagagtctcaggaagcttccaatcatggcagaaggtgcaagggcaagggagggagcaagagagagactggggaggtgcacacacttttgaacaaccggatctcagtgagaactcacttatcaccaaggggatgatgctaaaccattcatgaggggcctgccctcctgatccagtcacctccccccaggccccacctccaacactgggaatcacatttcaacctgagattcgaaggagacagacattgaagtcatatcaccatctgttctctttcttactcttttttttcatatattcatcgtttaccccgtcacggatggctaataagttcaggccaagttaaggctgtcccttggcaagaacagggacaattaggccaaacctggagaaattgtaacagctgattcatattcacaaagaagaactagtaaagctagaaatgataccaggggtaggtgatatctaaagaaaccatctcagcctgatttctagcttgcatacagaatcatgtattgaactgagaagtagccatattcatgaacaaaatttcaaatacttaaaggaatttttttcccacttagtatctcaaatttcccaacttcccaccattctgcatttcttgtgctaattcctagcaagcagttgtaaaaatcctaatattaaccagaagtttattgtttgtttgttttgatacagtctcgctctgtcgcccaggctggagtgcggtggtgcaatctcgtctcactgcaacttccgcctcctgggttcaaacaattctcctgcctcagcctcctgagttgctgggactacaggcgcacaccaccacacccagctaatgtttgtacttttagtagagttggaatttcaccgtgttggccaggctggtcttgaactcctgacctcaagcaatccgcccacctcagcctcccaaaatgctgggattacaggcgtgagccaccatgcctggccacatgagccactgcgcccagcccagaagttttaataattctacttaaatttaaataataagggctggttaattgtattttataatgatatctagtattttcctccaataaaaatattatgttcaattattagttattccaataacagactgaaagaccttgacttgtaaaatagagactcttccttttgttttctgtatagctctgtatttctcactattttccccatgattattcagcatttaaaatgctaaataagtatttaaaacattttttaaatgactgcttgttttctaagcctttaaaaaatgactagttgaaaactacttgagaaagaatactgttcacatcataaagtcttgtaaagctaacatacacaaacattgtaggaagtggctgaacagtaagtaatataaattgattttttaagccatgaatagtgcatgaactttaacagttatttccattgaaataagactaacaatatttgaactttatccaagcaggtcaataattgttatttcatgtgttgactaatttgattacaaaatttaatgtcattttgtcctataagacagaattttaagattctgttgagttacctactaaaagatgttattacattttaagaaaaattgtattggaaaataacttgaatgtagatcaaatttttgcattcaattattggtttaatttcattgttagccatacatttctgctgagtttatccaaaatacatatttagagagaatttttcaaaacttctcatttgattaaaattgtgttgcagtctaatacatccatgagaaatggacatacttaggcctagtatatgtaatgaagcctccttgatgaggcctgaaggatagtttgttttaaaaaaaaaaaaaacaaaaaaaaacggaattacccattcttgctaaattgaaagataggtaggatatgagtagcaccttaagagcaaattcatatggtaggtaaatatttaatgtccagtggttacagggcagtatttattcctgaagaaatgggagagatctggaatctctatttccatttgaacctctgctctgtttctcaaacttttgttcaatttttgcatctctctcttcctaattctcagtctacctttccctttttaattctgtctgtctcagtagtgataagctgaacttcactgtacatcttcactttccatttattcattttccaatttggttagaagtgggtagtttccccaaggatgaccacagaagcatttgccattgctacaccaactgttccattgggaaccagcttgaagagaactggatctcttcttgacagaattacagatctagaagtcagaaagcaattattaggttaattttgaaaggtacatattacagattttcacgtttttaaattccaaaaataaaaacgtttaaataaatttttaaaaattagatgacatttttgtaaatcttataaaccccaccttctattaaaagataattcattgccagctgggcacagtggctcacacctgtaatcccagcactttgggagtccgaggcgggcagatcgcttgacgtcaggagtttgagaccagcctggccaacgtggagaaaccccatctctactaaaaatacaaaaattaaccgggtgcggtggcacgtgcctgtagtctcatctactagggaggctgaggcaggagaatcacttgaacccaggaggtggaggtagcagtgagccgagatcgcaccactgcactccagactgagcgacagagcgacagtctgtctcaaaaaatatatatatatatcattgcaaatataattagccatctggtttccttcaatctcataacaaatttttaaaaatcagccatttatgccagtgagtaaaattaattgtttacagtaagaaaagttgtaatttgtttttaaaacttgttttgtttttaaaatattttcaaaaaatgtggaaaatatttaacatgaaagttacccataatgtgatcaccacaaaacaggtattttaattttgtatgttcctttccaggtatatccttattattacataattgcaatcatgtatacaatccacttactaatctgccttttcacttaaaatgttaagagcattttttcgtattcctatgtagtcttcttataagtcaaatgtgtcaatgtacaaatagtaaaacctcattttcaaacatgaaaaatgaactaatacttaaaatttactatgtgcaggtcatcttcggaattacttcatataactcatttaattttcacaataactctctaaggtgtacccttttgttcttgccattttatagatcaggaaactaagtcaaagaagggttaaactactttcccaatcacaagaataagaagtaggatttgagtacaggtagtcgggctccggagtccatgcttttatttggtaataatgcaaagattaacttttttctaaccgattccacatacattaagcaatctattgaaaactgcttgtcactgttgtaatatgacagtgcagagtttgcattttcctccttctcttgcaaatctccaactgaaaccccaaccacaaatgtggaagggctataaaaaataatggtaatccaaaatggatattagtgcaggaattaatgtaagagaagatgctgcacatacaactctcaaaaacagctgatagacttctggctatgctaatatgaagaaattgattaatcttctaaaaaaatactaaaaagcaaatgcaattgtcaaaaacaatcatttcagaagtcttggagttaaccagaggaagatgacagctacagaatactgctaagataaattaaggactatctgaacaaatggagagataaactatgttaatggattggaagactcaatattgttgggatagcaattctccccaaattgatctataggttttgggacatccctatcaaaattctggtaggctttttgttggaattcacaagcttacccttaaacttatagaggaatagaaaagaccaagaatagttcagatattttgagtaaagttagaagtcggaggattaacactatgtaattttaatacttgctataaaaatgcagtaatcaaaaacatgtagtactggcataatgaaacgaagagtccaaaaataagttcttacatttatggtcagttgattttctgcataagtgccaaggcaattcaatggaaaaaggataatcttttcatatagtactggaacaattggatatccatatgtgagaaaaaatgaacttggatccttacctcataccttatacaaaaattaactcaaagtggatcacaatctgaaatataaaaggtaagcataggagaaagtctttgtgattcagggttaaaaatgtcaagataatgacattaaaagcacagtgcatgagagaagagtaatgataaattgagcatcaaaatttaaaaagaacattaagaaaatgaaaagacgtgcacagatggggaggaaatatttccaaatcatttatctgaaaaaagacttgtattcataatatgtaaagaactcttaaggtggacgtggtggttcatacctataatcccagcactttgggagtctaaggcatgaggatcacttaagcccaggagttcaagaccaatctgggaaatatggtgagaccttatctctacacaaaatcttttttaaatattagctgggtgaggtggtgcatgcctgtagtcccagttacctggaaggctaaaacgggaggatttatgtgagtccaggaagtcaaggctgcagtgagtggagatgacaccactacccagagtttgaccctgtctcaaaaaaaaaaaaaggaaaaaaaaacctcttaaaatataaacaactcagtgttttaatgggtaacaaagccgaatagacatttcatgaaagatgatacatgaataacaaataagcacatgaaaagatgatcgtcattaggagaatgccacttaaaaccacagcaagatactgctgtataccctcgagaatggctgtaatcaattggcgtgaccgtatcaactattgatgatgatatggaggaactagaacttacacactgctcataggagtataaaatggcatagcacttgtagaaaatagttccaaaaattctcaaaaagttaaacatatacttaccatacaacctggcaattctcctatttactgaaaagaattgaaagcatagtccccttaagacttacatatgaattttcatagcataatattctcataatagtaaacaactggaaacagtacaaatgtttatcaatggtgcatgataaacaaagcatatcatacccatatactagaataccactcagcagtaaaaaggagtgaaattctgatgcatgcaacagtattgaggttcaacttcaataatctaagtgaaagaagtccaatacaaaagactgcatattctgttatttcattgatagaagatttcagaaaatgtttctacagaaacagaaagcaggtcagtgatgcctagagaaatcagagtgcggaccaagatcaattataaataggcatggggatatttcttgagtattagaagtgttccaaaactggattgtagcaatcactgtacaactgttgaagtttgctaaaaatcattcaagtatacacttataatgggtgaattttatgttatataaattatacctcagtaaaagtgtgaaaaaaaactggtaaactatattttcagaggcaaatggcacaccctaaggtacaaacttaaatctcttgtttgcaataggcaaatcagtgtgtgccagcagtgagtgatagcttccctggatctcatttccttttgggaagacaaggaggttggaggtaggattaaatggggtattacagacaaactctatatgcataaaacaatttgttgttgttgttttgccagaggggaagaaatgaaactttgctttgtgatttgtgccaaaattgcctatctctgcacagaaatacaggttcaaaatgcacaccaataaaatcctgtgtcatcagtagaattcctaatagcttagaattgagagttggcaaactatggcctctggccaaatctagctcccagattgtttttgtctgacccacagaataaaaatgatctttacatttttaaatggctatttttaaaaagaagatgacgatgatgtgagattttatgtggcccacagaggtgaaaatacttgcatctggttctttactggaaaagtttgcagacccttgacttagaacatggttgtggcctgtattagttagggttccacagagaaacagaaccaatagagtgagtgtatgtggggggtgggaggtggtatatgtatgtgtgttgggagcaggggagattataaggaattggctcaagtgattatataggctgacaagtcccgagatctgcattgggcaagctggagacccaggagagtatattttgtagttccagtcccaaagcttggcaagcttaacattcaggaaaagcttctgcctcgtttcaggtctgaaggcaggagaagctgatgtcacagctgatgttcccccctactattggtgcattcagcctttttgttctgttcaggcctttagttgatcacatggggctcacatacattagggagggggttcttctctcactctcccaattcaagtgttacgcagagacaccctcacagacctacccagaataatgtttgacccaatgtctaggcaccttgtggaccagtcaagttgacaatcacatggcccttttatccctaaaaacctgtaaatagatcctctaggagcaactcaccttcatcaaagttgagtaatcaagaagaaactagcacggtatctctacaaacatacaaaaaaggaagaaaggagtggtaataaatagcaggtgagaccatttatcagaaaaaaatgtagctctgaaataaagtagtatgcaaatattttttcatgaaaaaaaaattttttttttttttttttgagatgaagtctcactctgctgcccaggctggagtgcagtggcactatctctgctcgttgcaacctccgcctcctgagttcaagcgattttcctgcctcagcctcccaagtagctgggattaaaggcatgtgccaccacacccatctaatttttttatttttagtagagatggggttttaccatgttggccaggctggtctcaaactcctaacctcaggtgatcctcccaccttggcctcccaaagtgttgggattgcaggcgtgagccaccacacccagcctttttcatgaattttctaaaaacttaaagcaagaactttttgcttctgtgacagattagcttacatcctaccacctcaccaagaagagctataaagctggattttaaaaataatactatatgaataccacttacagtattcatctgtaagaagtcattggagtgacaacaaggaaaccaggactttactgaccaagatcctaaagagaagaatagtacagagcagtgagcccaacatttagtgccacttttcctctttaggcatttgcagatatgaaagcagaaaaacctcagaatctaggaagttgaacatggtattccacagttttacaaggctggagagagaaaaatggaagtgcattgcctatcaagttgcaggatctttagtaaataattcaaactttcagttgaacccattgaaagcctacattttaggagtaagggtgagtaagaaacatgagtcctcagtgactgaaacccagtctccaagtatttcaatctttgatctatctttactctgattgcctaccaaaagtaaaagtaaatactctgtggaggaagatattatccagagtcttaaaattatctaaagtttgtatacacgatatgtggcatgcagtaaaaaattaccaggcatatcagcaaacacaaccaaatcattgaaaaccaagagaaaaattcaacagcaaaaacaaaatcaaaaaagggaaaataaaaaccaggcagtcagtagaaacagacccgcaggagttcgcagtatgaaatttttatgaatggactttaatcgtgtgatttgttcaagaaagtagacatatgaaggatttcagcaaacaactggaatctatacaaaactgtcaaatagaatttccaggagcaaaaaattaaataactaaaagactcaatagaacattttaaaaacagattggagagaaacaaagaaaaggactgcatatgttaaaaaaaaattttttagactaaagtgtagtcagggattaaaaagaatagaaaatatagaaaggaggatctaagacatagaggatgtgatgaaaagttctaacatacaaataattggtttcccaaaaggcaaggagagagatggtggcagtgcaatatttgaagagtgtttatggcccagaatttttccaaacctatgagaaacatttagccacaaattcaagaaataccacaaaccctaaaagtgaaataattaatagaaatcagaccccacttagacaaatcctagaaaagcggccagaaaccaaccaacataaaattctaaaagcaaccagagaagaaagacacattacgttcaaaggcatgaaacttcaactgaaaactgacctcccaacagaaaaattaaaaacggaattcagaagaaaaagaatgtcctaaaagtgctatagtaaaataactgccaacttacagtgttatttacagcaaaaaatgccttccagaaatgaaggcaaaaattcagaatgtggaaaactgcaggacaacctacattctttttttttttttttttttttttttttgagacagagtctcattctgtcacccaggctgcagtgcagtgacgcaatctcagctcactgcaaactccacctaccgggttcaagcaattatcctgcctcagcctcctgagtagctgggactacaggcacatgccaccacgtctggctaatttttttttatttttagtagagacggggtttcactgtgttacctaggatggcctcgatctcctgacctcgtgatccacccgtctcagcctcccaaagtgctgggattacaggtgtgagccaccgtgcccagccaggacaacctgcattcttcaagtcaaaagaaaaataaaggtgttgcaggggaacctataaataaaatagacttaaaagatgtatcgggccaggcatgctggtgcatgcctgtaatcacagtaatttgggaggccacagcgggcagatggcttgagcccaggaatctgagaccagcttgggcaacatgccaagaccccatctgtacaaaaaaatttaaaaaataaaaaaataaaaaattagctggatactaattttttttagctgtaatcccagctactagggaggctgaagtgggaggattgcttgagcctaggaggtggaggttgtaataagctgagatcacaccactgcactccagcctgggtgacagagtgaaaccctgtctcaaaaaaaacatatatccagccattaagctgggttttcttattggtatcatgattcagtttaataaacaagttttgtgatatgttgaagtaactggaaacttgaactattactatttatgtgatattaggaaatttgtgaaatttgtagtatgatgctatcgtggttatattttaaaagagagatccttattctttagagatacatacagtgatatgtatggataacattgtatgatgtctagtatttgttcccgaatagtacaggaggaggggataagagaacaggcgtataggccaggcgtggtggctcacacctgtaatcctagcactttgggaggccgaggtgggcttatcatctgaggtcaggagtttgagaccagcctgaccaacatggtgaaaccccatctctactaaaaagacaaaaaattagccgggcgtgatggcgcacatctgtaatcccagctactggggaggctgagacaggagaatcgcttgaacccgggaggcagaagttgcagtgagttgagaatgtgccattgtactctagcctgggcaacaagtgcaaaactctgtctgaaaaaaaaaaaaagagagagagaacaggcgtacaagtgaaacaagattgatgttgaagtaatcattgttgaaattaggtagtggggctcatgggaattcatttttattttccttttctgtatatgcttgaaattacatgctattttaaagaaaaaatgacataaagacatttttggagaaaaaaaaaagctattaccaacagaaatgggcaattcttaagccaagtagagaatgttcacagaaccaagtcactgtaggttgtaagggaaaagcaaaggtagcaggagaattcagagtacttgatatccaaattcagtgagcaaaagggattattataataagacaggcatttttgggtagatgatgttgagtaatcaaatttgacaatacctgtctgaacacctcgaattcttggtcctaaaagtcatgcatgtcttaaaactatctttcagcttactactttgtatgagaacttaagtatacacaaccagcagttagaagaagaggttaaagatctagcagacaagaaagaatcagttgcacattgggaagcccaaatcacagaaataattcagtggtgagtgctcttttaaacgtttttttgtggatgattgtgagtatgtggccaagttggtatgaattccaggtggctactaataatggtttttatctcatttgtcagatgtgataaataagttactctgaaaagcacagttttattactgatctgttgtgttctactgtgttttctgttacattcctctgttccctaactctaacaaatgtcccataccagcatttaacacagcttcattgaaatctgattattattactttctaagaaaatgttaccaccttcagagtcaaagttggaattcatctgctacctatggatgtttctttctaagaccaaaagtcctgtatttgtgagtattttaagaaatggaaggcctgcaaaaaaaacttagaagctaaagatactgtactgttatcttgtatcatgattataccagattaattcacacatggcatttttaaaaccttaaatggttacattttagttttatcagctctttcttaagctcttgggatttagtttggaatattcatcttagtaaccttcatattcatttattaaacgattattctggtgctggtaaaaaaatttcatattttaataatacatggggaaagtagaatctaatgaagactgtatttccaagtaaaaataggtaacttaactttttatatttatttaacttttgaaatccattttggcaaatggtaggtgtttaatttatatcaaaacaaatacaggttgagcattcctaatctgaaaatctgaaatctgaaatgcttcaaaacgtgaaactttatgagtgctgacgtgacaccacaagtagaaaattttagacatgactttgtgatgggtcacagtcaaaatgttgtttcatacacaagaaattattataaaacattgtaaaaagttaaccttcaggctatgtttataagatgtatataaaacacaaattttgtgttttgtcttgggtcccatccccaagataatctcattatgtatattcaggtattccaaaatttaaaaaaaaaacctgaaatccaaaatacttttggtctcaagcatttcatgtaaaggatacccagcctatagtttcatcatatatttatagatcagtcatttgtagggaattttaattcctgtatgcatattttggttcaaatgtttttccattaagagctcttaatgcttataatgtttctattcagtgcatataaagggaatatgcagattgtggaaatttatttgggttctagacagtacagttattggttcattaatttatatctatatcatcaagtctgcatttctgaagcaaaatccattttaatgtttaaccaagtttataatatcaaaaattatgacttgatatttgtgattccatttttatgttatcaaatatatatgtaaggaggagctgaaaatgacttcagaagacagagtcaaaacctgtgatactagactactgattttttcaagtgtctggcatgatgagttcagctggaatgggaagtggtgctagtaatggaaggaagctttcaatgtgggctttgtagaaagggttaatagaaagggtgaagatgaaagttttatatgatgccacaaaacactcactatacacttttcacattcaaacgaaatatgcagaaatcagaaatacgaggaagtcactttgcatgaaagctgttattaaccctactttttatttagaaatggagtaatagatagcagattggcaaatacccaaagggaaaaagtaaaatatatattccctgttcaactttcttaccacttcaaagttaactctgtgagatacaatatccaagtttataaaggtcagaagactgagttattttcattttttgagtttagtttacagattgagtattccttattcaaaatgcttgggaccggaagtattttggatttcttttggattttgggaagtttgcgttatacttaacagttcagcatccctaatccaaaaatttaagcatcttgtcagtactcaaaaagttccagatttggaggcatttcagattttggattttcagattaggaatactcaaccttcttttgaaaaatgaggtaatgctcaaacaagattataggacctcatgacatatagcactttaaatgtttcctgggacaaatgaacagttttaatacaaaacaaacactgcagtatataaaaccttttacgaagtcacttctaatggctcttgaactccacctcctctctcttttgctatgaaatatccctttcatcagaaactcttttagagcaaggacatcttctcaccgccgttagagtaggaaccttccttctcgccattggctcatcagaggacttcacgttttctctttgaaataatattacaaagagaggttagattaataggctagtccataagatttattttttaaatgtgttttcctcacataaggcattcttctggtacaataagagatacagagatctataaatgtagtacttttctttaagtaacttgtaactaaacatttaaagtttaaaaactattttgaccagagctggagataacttatgagacttaaatataaatgccttacatattgaatagatactgactttttaaaaataagaatgtagtgttgagaaaattatctcagactagttgctcaagaagggtgggaataatcagttaaactagactgtgtgttctgaaaaccctgagtttagtaaaatatcctacacctagtttgaccaaattggagaatttttgtaaaagaatagtaggaaataagagtaataaagttactgcaacacagttgtcatgaatactaggtaagaattttaccttcacttatttgtaagatggagaaggtttttcaaaggtaatggtgttaattaaaatgatgattaggcaaatagctcttaacagtaatgttcagaatgaagtagaaggtaatgttggaaataaagtgaacaaatgcaggagacattgtgaaagaaaactctataggacttaatactatttacaaatgaagggcaagagatgaggagaaattagtattcctgaattgtgagttaaggaggttaagaaaatgatggcatcattgaagtaggaaagttaagaggggtaaccacccttggggaagatgatacaatcaattttagctgttattttattttgtttgttggtaagactgaggtgatcctgagacatcttagtagagatttctcatgcttttgaaaaactaacataaagattaggattagaaatgtaaatttgggaattaaattccttgtgaataaggccagtgaagtggttgagatctttttaaggagacaagaaagaaggaggatggtaagggctatattataaaagctagatgaaacagctctgtagaggagaaccaggagaagggtttaataattacaaagcgtttcagtaatgaaacctttgtctctctttatttaaatccactcacatatttgaaacattcctcattcagtaatgctcatttgtgtatagtaatagcttctattgattaaaacataagagtgttctggtcattaggctggaaattcacatgtattcttatttagttattacaccggtcctgtgagatagatatttttactatccccattttataagtgggataacaaagagaagtaatttgcccaaagttacacagctggtaagcaccagagctaggatttgaaaccaggtctatatgacttaagaatcaaagttcctaattactatatatgccccaacttttcaggaactgtcccttatttgtcttctcaatatgcccaatgcaatagtatttaataagtattaactaattgatcatttgagagaacttggaagggaaggattaaatgtgccaagagaacacatagatctaagttggccttctgtagttgagttttaacagattagcttcagcaaaatagttgaggcaggaattagacttcgggaagtatagagtagtgaatggagggtttaatactagagcaattacacgtatggagtgtaacaatgaaagagggaaaatgggatgtttggggatttgtcagtgaggaggacagggaacttttggatttggtagagtttgtacatgtttaaaggaaccaatggaaataatgaaattgaaaatatgaggtgtagtagggagggtgattatgtatagtaataataactgttattaagaaatatttgaatgatttagccaaataaaagataagacacctacctctaaaactggttttaaaagttaagagaatgaatatgaataaacttaaccaaatggcctacaagcaaggagcaagagatagtgttggggccaggtgcggtggctcacgcctgtaatcccagcactttgggaggctgaggcgggtggatcacctgaggtcaggagttcaagaccagcctggccaacgtggctaagcctcatctctactaaaaatacaaaaattagccaggcgtggtggcatgtgccagtaatcccagctactagggaggctgaggtaggagaattgcttgaacccaggagacggaagttgcagtgagctgagattgtgccactgtactttagcatgggcaacggagcaagactctctcaaaaaaataaataaataagagagaaagatagtggtggggttggagaaagtaagatttttttttaatgaggtggaaaaatagacaaatagcaatgattatgagaaggagttgtgtgagacttagagtacagtggaacttaaatggcatgagttgccctccaaaccagtaattctctatagccaagaaaaagagaaataggtttaatcaaaagttaggaattagtggaagactgcagttgtaaggtgttcaacatgtaaataaatgtgttcaacatgtaatgtgttcaacatgtaaataaaaagcaaagttagaaaaaagagattgtcatataaaattccaaataacttaaagtcagtttacaagaggttgatttccaaagttaattaaacacattggacttcacagtttcttggaaatgtgcataaactaagatctctgagccatggtagatactaaaccagcactaggtgagacctgttagcctgggcacttaaaggataatacagttttctttattgttactatataagtatcttttttgtattctaaaaaagataatcttccttcatcccaatttattctctacactatagatcaggtaattaaatgaaaagattacagtcacttgtagtcaaagaattttagaaggacacaacccttgctacacaaacattcttaaaataatttccaaaaccatttttctttaacaggcaataaaaatgtgtacatgtagatagagcctaggaatgtgtaactgtcccttaccatggttttattgcatctccatattttcttttgcttatactccaaagacaatctcccatctaagaggagagagcaacaagcctcttttaaagggcctctttaagatatatataaatttgactcctcaggagtttacattctaagagatactaaagcagagctcaggagttaaacatgtctgctgtatgcatcggatacatgctgatttacctgaaatatactttcctcatagcttcagtgacctggattttatctgaagacaaccagcctgcacaaagacaaattgtcttccccaaagtgtgtttagaatacacttgtgagcattgcaaatttcaactccattttttttgtttgtgcagtatggctttctgtataccagtggttttcattcttcctgagaataggaccttcttttttagattcttgtagatatgctggactcctcccctcaccttctccccacttgagggaagtgtaagaaatgcagacgttttcttgagaaattctaaaatgtggaatttgtcttactaaatataatttgaaaactttctgtctaaaattataattgtatcgttagctagatagccttaagacagtaagtagaaattaggttttggtagactttaatcttccctctttgttattttattgatcatttcagaaaacagaagagcttaattacaatataaccctgatttttgcagaatatcctacattaaactttctaaatttaatatacacttcagttttaagaatggtatgttgtttttatgatataaaatggcagaatccaattgagaaatcttattatttagaaaacctcacacatttgtgcatctgtgttaattgttagatttaaagagtgttaattctgtgtttcaagggtgaagttttaataatgtatcagctatgagtaacttggacttttgtaaacatcacctgctctaccatgtacataaaagaacagttccataaacctcttcccagttgttgacaggtagtatactatggtagaccttttcaaagaaattatttaagactatctttgaatttcctaccacttatttctttaggaagctttaaaataacatatattgcactgaagcttttttgttttacttttgtactttgtctttatatattctagggtcagcgatgaaaaggatgcacgagggtatcttcaggccttagcttctaaaatgactgaagaattggaggcattaagaaattccagcttgggtacacgagcaacagtaagcttctatgattttgcatggaggcttatcatattagaaaaaagagatttaaaaaacactataataaatccaattacttatatcagtttgtttagactttgagcctactaaagagttttttatgagaaaactattagtttgggtcttttccagtttagaaaaacataacacacagctggttaactataataatttaaatgttaaaaataggatggaactaaaaacacagtcattatttcataggatatgccctggaaaatgcgtcgttttgcgaaactggatatgtcagctagactggagttgcagtcggctctggatgcagaaataagagccaaacaggccatccaagaagagttgaataaagttaaagcatctaatatcataacagaatggtaagattgaatcatatagtcccactagaataatttctaagtttgtcataattatttggagagaagttacatgtttagtttccattgcttgcttttagaaaaatgatggccaaatatgttacaaagatttgaaaataactagatgcttttaagccttgtagatagtgatgaggttttaatatagccttgttttcctctggcttgtccataaagtagaattaatagtattaaataatattccattatttaattttcctcataaaatttagttgactctcaaattttattaaacctaaaattctctcatgtttaggaatattatatttgtataataatacaacatatggtgagaacaaaatataatcatttgaaaattgtggcctctaccataggtcttttataaatgaaaattctattgatggcaaatttcataatgatataatatcttttaagtataattattgtttttcatgtttctttatgtatttagaaaaagtgtatcccacagtctctgaagcagttagctaatgaacacatttcgtcaattctggtcacaacactagagtgaatcatactctgaatacataaatgattcacagttagactgattataaacatggttcaaaataagccagtttaagtgacatagaggaacaatagaaacttcacatcacaagtgaaactacaaatacgaaaatccattgactgctgagaataccaaaagttccaaaagcctgaaaagaaatgcaggcccatgaattcagtttttaatggtgtgtagtcatagtccttaaactgcaattgtttatctttttaatcaaatcaaaggccagatacatgatacttctgttggattataaacttttggtttataagctttttgctataaataatgcagacattgtatgttttacttaaatgtgcttatgtggcattgaggaggggaaaactttttctggagaaagtgaattgatcccagcataaagagctaatgaatatcattctctgtgttgtaatttgaaataagtaatatcttttctccttacgcctaggacagatgagtttacatattgttcataagtcaggaagatgagccagtttgaaaaattataaacttactcctgtatgtgctccactttcatggttgcctgtaatggtataatatctaacaaagtctaacagaattctaaaattaacctttgattttgtaacatttttaaatttaataatttagttcagggtccacaaatgttttctgtaaaggttcatttagtaaatattttaggctttgattagcttccatcacagcttctcatctctgctattgtagtgtaaaagcagccatagtcaatacttaaatgaatgagattggctgtgttctagtaaaactgttaatctacatgaaagtaggtagcaagctgtatttgttcttcccgcagtaatttatagtcccctaatttaccatgcctgacatttaaaccaagtaagtttggctacatccaagatggctcactaaccagcaataaaaattaatgacatttttccttttttctccttatttaagtaaactaaaagattcagagaagaagaacttggaactactctcagaaatcgaacagctgataaaggacactgaagagcttagatctgaaaagggtatgggagtgtgtgtttgcctcactcaggacttactgttatgttataaatatattttattacagaagaatcaattcattgcataggtgggagaattgttgacataagcaattctggcactcatttatgaatatacgtgtgtatatatgatgcatgtgcatacacacacatttggaagactgacctctgagtcattttgttttacttaatatgcattgtttaatgatttcatagtaacagaaaaagacactttgccataaatttgacgttttttttcagtgcagtggactacttcatgaataacatttttgtgattggaaaaaaattttattttataatgtactttataatattctcaaatgctatgatatgaggttatcactctgacagtggcaatgtgtgttaacatccagaacttggtaagggtacttgaacaagtatttcctagcactttttatgtgcctagaatgtataaaaattggggggggggtgggattcactttcagggtagttgaggaaacaatgtatgaaatagctgaagattaatttagtattaatggagcaattcagtaaggaaaattttggggggcattttaatgtcttcttccagtttgacacttaaattgcttgtcctgtaggatatcccggatttattaccattttcttgaaaactttacttgaacatttttccattcatgtgaaagcttagtcagagtgcttcatagctgtattacatgaacttcttaaactttccttaatgtggatgttaagaaagtgaagatttaataaaaagaaattcagatatttttaaaataaagggatatcttatatacatttatttgaaattgattttcctgacaaaggccatcactgaatactgaactcgttaatgggctttttattaggtctaagtgctttcctatcatttctcagccatgcaacacagtgagataaaactggtggagtctctgcatctgcatgtaaaatgatagagaacagagagtaatcacataaagaagctaacaagaaaaagcctttacaaatagtggatgctcaataaattcttgtggattgaatttcttaattaacaacaaaaggaaggagcccagaaaaagtgaatagtaattcagattaggagcacaagccatcatgccaaaatgaaaatcaagctaggttttaattaatggaaatttgaaagaagatacattatttagtgaggaaaagaaaaagaattcgtaagacatggacaactcgtggccctaatggcaaataggagagagactagtgcagaatatgacttgcacgccagatgacattgattgcatcatttttagaatagacctaaacttgaaagagacttaagtttatcactgaaggcatgtttaccacagctttttaatttggattttgaatattgcagaacaaaattttattgtataacataatgtatactactacttcagtaatataatgtgatatttacagtatgagggcaaggatgtttttaatgcttcatttactattatttccccaagtctctaggacagtgtctggcacactctcaatatttgaatgaatttctgaacatactgccttacctgataactgtgttatattattagatagtaatatgaaaaacacaaatcagctgatgtcaattcctaaggatatggaagtgaaaaatgtgggattggctgttttctggtttttttctacctagtatttttatttgtatgttgtaagaattacttgggaaattgtgcatttaaagttaatgccatagtcaagagatatgtatgaatttttaaaaattcatctgagaaaaactggggtttggagtcttcatcctgtcaataaaaaaatacctgaaatcagattggcagtttttttgtaattaagtgtatttgtttcatcactgcattaatttcttataaaggattattcttattagtgcatactctgtgctttgataagagtatttcaaaacataagcctgtttggtgttgaattagcttagaaaaatcagatataaacagactcctatttttaaattctctcaagcctaccaaaagatttaatgtggttttttattatttactatacacaaaaagaaagaaactcagtaacataaataagcatttcatggaaactgatgtaattgaactattaagtgatattctaggaaacactagtgtattttagttttgtttaaatgtagagttaattatagtgatcaactttgaaatacagtagtttgtgaactgcttttagtagattcagacagggaaggttatccattaaaatcaattttaattgttgtagccttaaaaaaagttgctgctgtcttctaggtatagagcaccaagactcacagcattctttcttggcatttttgaatacgcctaccgatgctctggatcaatttgaagtaagtattaaacatgtcacatatcctctggggtcaatttaaaattcactttgtaatttaaagttcacttataataaaaccattttattgcttcccacatcagaggacctagaatcttaaataaacaaaaacatattagcagaggaatatgttctgcattcttatggagactataatcccacctctgtttaacaaaacatctaagaaagatttaatcatgagagtcattataagatttaaaagcaatttcatctcccttaagaaacattaagagcagaacaaatcccaacttttgaaataatttgacaatttttttattctccaaagcatagtgccacaataaaaagtgagaaaatgtcaaagctgacaataaggtattgctggttcttatttcaaatcaattcatcttttaaaaactatacagtaagaagtgcttttgttaaaaggtgttacatagactctgttaggcatatcacacagtaatgaaattgtgattatgtaactcttgatcattcgttggtatttaaattctttgctttaaaaaaatcaacttactgttatcaagtatatttttgtctcagtaaatttaagtgtgattatatctttattgaacaaataataatcatgtagctttgtgcaaggcactgttgtaagtgctggagatatagtagtgaccaaaatagacaaagtccttcccactagaacttacattccagtgggaagaattagaagcctcaggaattccattgcttacttttagttgttacttcaaaagtacttacatttaatttgattattaattatttgtcatgagcttcattttattacattttgggcacagtatgtgaattgtgtttcgttcctttaggaaaaggaaaaataatcactctttaaaaagtaattaatgaatctcttaagtttctcaagaacaatgatagtgactgcttttctcatttatttatttgaattaatgtcatttgcctttcaattgttttacaaagtttttgatttattattcttattaaattgcttcatccatattttctatttttttattgctgtattcgttaaataggattccttttcttcttcctcatcttcactgattgattttttggatgacgtaagtctttgattttcaaaccaatgcatccttcagtaaaaaagatcagtgaattgatttgaagaatttgcctagtaatgcagtcagttttagaaataccacatttgaaaatttatgtgagccaaaatatgcttctttcttatcttaaatataatgaaggatttatcagtattaaacataatgaagtaaaaatttactttcaaaaatgctttgactgttgtagaatcccagtattttcagtctttgtaagaattctaaaactagaatgcttatttaattattttttatatatatataaataattgcttatttttatttttcctggcgatttctattttacttttcccttttcttttcagcactttactgtttaatttctatgttggttaaaaataaacctctggtggtataatcaaaaaaaattacttttaattaatttctccagtaggttttattgtatgacaataatagagtactgattttaaaataaatcaatgtttaattgatttatttctaatgctgacagaatttctcttctaatgtgcaaagtgattttagttaacatattcactatatgcactataaattgactgcattatcactttctctcctgaattctatcaatttaataatttagaagttcctgttaaatgtgtatcattcattctctttactacctggcataacattctctctattccacattaaatgcacagtaattcttgtcctaaagcatttattatcctgttgaatagtctttacagaaatggggaaataaaatggattcaaatgcaaatattgtgcatagaattgttttttaaatcgggagattttttaatgttttattctttctgttttgttaggatacttatagtcagaagagtagttagcataatgttctgctaaaggaacttcttgatctagatgtttaaaaattattgaacaaaaaagtgattttgaatatgcttggaatatttatactgaagtttgcacattttcattctaaacaaagtgttttgctattctcaaaacaaataattactctggttaactctatagtgtgataaaaagagaactctcctcgtgtacagtggtattgcatgttataatctgctgctcaggatgttaaaatattcaatatgatatttccatacagatattattcaccttaaaaaagaaagggaaggaagtttgagtaaattattttggttgttctttccactagaaaacctttttatttctaacaaacttctctccatcccttttcctgtcacaaaaaaaaaatgtacatatatttgattgtaaaacctaagagcagtatgtattttggcaaatacatgaggtcttctcagcagacctttcaggaatgcacaccccagccaggtcctcagcttgtttctgctctcacaggcactctgccactgctgccacccacctgtaatgcctgagcaccccgcagccatttcccagtgttacttttcctccaggaatatcctttctcatcttctaccctctattaatgcaacatgagctcagtataaatataaaagtaaattttattgcactgcaagggaactcctagtatgaagtaaaaaggagctgagttcacaagagctgccaaatcttttatttatattccacctcctaaaaggcatgatttcccactcatgtgctttctaagctccattctcccaatttattggttaagtgcataagcaggcacaaatcctgcctgtacaactttctaggtgtatggtttggaagaaattaacctttctacaccctaaatttgctcatcaggcaaatagtaataataatataactaatctcataatgttgtttgaagatttaaggaaacagtgtatgtaatgccgtcagtaataatgcccttattacttttaataagctggttccctccctttctgtattctctttagcttcttttctcatccatcagttcagtttcacttaaatgctttcttagctcttcatcactcagtcatctatccaccaccaatattttatggcacaaactttgtgtttctctttagagtttgtacctatgatctcccttcccagagttatcattcattcatttgatcctcaaagtcatagcagaatgatgttttgaacagcagtattcaatctttgacatacttgagttcagcatccaagatatttcgcataacgctcaagttcttctgtagctctttttctgctaggaacttggattctttttttgtttttgtttttggttttttttttgagataaagtctcgctctttcacccaggccagactgcagtggtgctatcttggctcactgcaagctccacctcccacgttgacgccattctcctgcctcagcctcccgagtagctgggactacaggcgcctgtcaccgtgcccagctaattttttgtatttttagtagagacggggtttcaccgtgttggccaggatggtcttgatctcctgacctcatgatccacctgcctaggcctcccaaagtgctgggattacaggcgtgagccaccacgcccggctgcacttggattcttaaggagcaaatcaggtctctgttgttctctgtgccaaatctagttatattgtgcttgatgcttatatgcctgccctttgcggtaaggctctaatgagtagtacacatccttctggacattcttttttgttttctgtctcgttgctatagaggagattatgttgaagacctaaagtttgaattgcagctggaacttgtacgtatagaagggaataagactagtattgactagaattgcaggaatcaagtcctttggtatcaacatattatcattcttctccctctgtctctgggaacccagtgttctcatgctgccctctgatttgttggagctttaccaccagtgcctctcctcctccaaagttaccttctttactcctacttttacccttccacacaggcctctaagtgtagttactttatttaaaacagtacggttttaagttaaaatgttcccatccctattttttcctccacatgttttgcctgttaactcaaaattcaaatgaatgctggaatttttggcacaatgggaagatgttcggagcaacatgctttcctttttaagtcacattgtttggccaatacttgtatgtgtaagagtagctttgaagagaagaaatatatgtaataccagtcattcccttccctctgggaaaatgaaaggaggcaaccaggcagagcagagcatattctcaacactctctgcttctctaacttgggggaatgtaggcatttgggcttttctacatgtagaatcatagagttttgaaggagaaaaccttagaaagtatttaatccagttgttttaaccattacagcactcttctgttattaagagtggataggttactctcccatttttaagagtgacttttttaaagttctcaatctttggggattaggacagtgtggtgatacattgttatgtaaaatattctttaggacagataaagaagggtttgagttaatttatttatttatttatttatttatttattttgagatggagtctcgctctctcacacaggctggagtgcagtggcgcgatctcagctcactgcaagctccgcctcccgggttcacgccattctcctgcctcagcctcccgagtagctgggaccacaggtgcccaccaccacgcccggctaattttttgtatttttagtagagatggggtttcaccgtgttagccaggatggtcttgatctcctgacttcgtgatctgcccacctcggcctcccaaagtgctgggattacaggcatgagccaccatgcctgactgggtttgagttaattttaaaagaaattgaaactactttgataagtggtctcttgagaaaggcattcccattcaacctattaagaaagcagtagaacagagataccaagatcccttctgctgtagaaattattcccacttctaccctcatcttttccttcaaccgtgcatgcccagtcttggactgtctagtccatgttagccccagcctctttctctaggtaataaacagggtgaggagcaaatggagctcagctgcttagattttcccatttctttcccccatctattcccttcatcccaatagtaacagatccagggatcatcacaccaagcttgtagcaacttcctatactctcttcagttcattttcttttcagccgaacctgaaaaagcttggcttgagagtattatagataatactaattacaatcatataaattagctgcttgctctttatatttgagaaacgtattgtcatatattaaatatgtgctaagtaagttttgaatttgattaaattacaattctacactttgttcatttagtcccagttcacaaaaatagaaattgcccatttgcctggaaaatgagaatgtactcaacttggtttttcagagttttcttactctgatatactgatgaaagagtaaatcaaggattaagctcgggaatataaaggtcagacacagagaaacagagatagtatctatagcccaacagtctggtccccctaatattgttttccattcttatattctggcatataagaaaagcaagtagtctgacaacattggcaggctgagttctggacttgattttaaccttgtataacatcttactaagatgcttcaaaatatttaatttcagtcatggcttctaacttctttaatttatctcttgatttcacatggttttatctcccagcttcttttacattctgttacaatcagttacagaaatccgaggcttttacagaaatctgaggcttggcagcatagtttcagcgatacttgggggaatggtcaattgtatgtgagaattggtaggaaatagagttttaaaacatacattttaaagatttttagtcctatgaccaaaatcagacctagcttctttggcattcctgaaatttgctcattcaattcttgtttttacaatttctatccacatattcaccacaaccctacctgaagattgcatatttcagaaagttacatgcattttttcctcaacttaggcaatatattatgttgaatataattgtatatattacgtattttatcgcatatataatataataatatatttagacaatctaaagagaaagaaccatgattacatagtccatttcttctttggtattttattaaataagacctctaggattaattgaaccttaaaaactattagccaaattatgtactcctgagattatttcttttggtttctgcttgtgtgcataccttcatattcagtcttaaagttttgttggcatgtcatggacctatatatgaaatacatgttttaaagtggtatttggttgcacattaagacaaggtgctggtttatagtgtttgccatgaattcaatttcagtgtttataaaaaccacagtgtgtgtaggtatatatgagagagaaagtgtgtgggtgtgttttcctctaactcaccttttctgttgaaaacataaaaatagttgaaaacgtaacaatattgaaaacataaaaattagacaatttctaagtaaacactttggtaagaaatcattgctgtggtgatagtatcattattagctttaaatggtattggcttagaaaatgtattgtccttcattttcaaagatagtttcgggagacattttaacttgtttttagtatagtgataatatatttattttgagatattcatgctcttttccatggttttagcatactccagactctagtctagaagtagagatagccaaaaaagttttttttttttttatgttaaacattttagcactttttacatgggtggtaatatactgggcataatggatacaaagacaaagcagacgtggaaatggacctcaaagtacttattttataattagaaaaataattatatagttacacactatgcaatgtggaaagcaataaatgccatgagaaatacctaggctaagtactatggacattcagaaggattcattcaatatttattgagaatatttaagtttgaagtcttatgctaagcaaaatacaatacttcctgcccttaagcgtaatatttcctgcccttaagcataatacaatatttcctgccctgacagtctaattggaaagacaaatatataaccagataactataacaccatgtgggaaatatagtaataaaagataaagatcgtatatcatgggaatacaaagaagagagatttctgattgaaccaggaggatccagaaaagcttattaaaggaaataatgcctgagctgggtcttcacagatgagtatgttcagcaaaatcataaaacaactacataagaggagtatattctagacaggcagagcgaacttgcggggggagggagcagggagttacgggattgtggacagagggagatttcaatttagttagaattgtcccttgcttggcaaacaaaaagggtgaggaagggattgagaaatgagtatgaagacatgggcaaaggccagaataagggtctatgtttctcaactttatacttcatattacagagcttaggtttaatcagtcccatatgtaataattattaaaagatttaacagaagagaaaggtgacagcttcattttggacaagttaagtttgagattcctaagggacatccagttggagatgtccagcagacagtcagatatttaatatatgcgtcaggagtttaaggaagaggagtggactggcaacagagatttggaaataatcagagttgaaagtggatgagattccccaaggggcagattgtagcagtgagccaaggacagaaacctggaatctccattgaagaaagggtgaaggaataggactccacaaaagctaagaaagcaatcgttgagaagtatgtggttacttaggaaagtacctgtcagaaaaactagaagagcagataattttaaggaggggaatggtcagtgtgatggtatggcagaggtggttggtcagataggacaaaccagaaatactgacctgagtatcagatcaaagcagaatctgaattgcactgtattagaagttaattgggagtttagaaaatacagacggcatttgtataccatttttcagaaatctggctgaggaagttttgaatcctggctctaccacttaatagttttataaccttaggcaagttgatccatcttctacatctttaaaatgaggatcataattcctaccttatagggtttctgtgagaattaaaataattcatatatgtaaagcactttggaaaacgatctgcatatactctgcacataataaatgttattatgattattaaggaagaagggagatagatgatagctagaggaagagttggagttgatttgtttattgtttggttgtttggttttgggggcaagaaggaatgagagaggttcttcccattaataactgaggaaaagtaactaggtttgaacctgagactttagcctccataatggaaaaacttctttagaactctccgggccttctaatcctggtttactattaactctgcaggacacagtgttcaagttatcttccattccatttcctatggaaagaaagaatatttctggccaggcgcggtggttcacacctgtaatcccagcactttgggaggccgaggcaggcggatcacttaggtcaggagttcgagaccagcctggccaacatggtgaaaccccatctctactaaaaatacaaaaattagccgggcatggtggcgcgcacttgtaatcacagctactcgggagtctgagggaggagaatcacttgaacctgggaggtagaggttgcagtgagccgagatcgcaccactgcatccacctgggtgacagagcaagactctgtctcaaaaaaaaaaaaaaaaaaaaaaaagtacctatttacttacctgccagagttattgtaacactaatactgaatttttggtagtgtttctcattcttgaacttttccttttttcttaaatcttctataactcctgtagacctagtctataagttccctcctctaggtccccaagaaaatcttctctgtccctcctctaggttcccatacaaaccttgtacatatctcagtgatagcatttgtcacattgttgctggcatgatgtcttctctaccagtctgtgaacttccatgtcttgtttatctttcaatcccttgcaaagtcggcttagtatccaacgtattgtgagctcacaacaaatgttaggttgcactgaatgttagaaattgccgaagtagatatggttcatattagtgaagctctactgctggagcatcacaaaaatattttgattgggaggccaaggtgggtggatcacgaggtcaggagatcgagaccatcctggctaacatggtgaaaccccgtctctattaaaaatacaaaaaattagccgggcgtggtggtgggcacctgtaatcccagctactcaggaggctgaggtaggagaatggcatgaacccaggaggtggagcttgcagtgagccaagattgcgccactgcactccagcctgggcgacagagcgagactcccgtctcaaaaaaaaaaaaaaaaaaacaaaaaaaaactttttgagataattgttccttttttcctaattacttaattctttaatgttagaaaggtgaaagtccctcttttgactatggctgaagttgagtcaggatggagtattgttcaaataacttttaattttaatttattatcattaattctgaaatcctaacttagcccttgtcatttaaaagaatcatttttgaaaagagtaaaaatttactaggtaaatgtctaacgccaggctagttcattattttattgaatgtttgccagcactaaatcagaaatatctagatcctttttgttctcatttgttccagtctgctactgccaacctttccagaaaagaaattgtctatcacttgatgaaggcttattttgtgggtcactgtacatgggccagtgaaaagtttgtcaaccttagacgtagtccaaacaactgtttctttaccatcctatcctccagtttttcctaaaaatacatgtacattcaacctatccaaaactgtggctttatatgtatgtttatatactttgtctgttaatacaatagtagtccttgagagtcttatgtgtgtaaatatttgtaacatgtcctattctcctttctctgttgaagcgctctccatcctgtactccagctagcaaaggcagacgtgtaagagacttttgcactttatgtacccatcctgaggccttttacccctctctcatccttttgcctgtgattgcggaacctgttaatgtttttgtggtatattattacatctattgcatgctttgtgctgctgctcctgctgttgctgtgctgcttttatgtttttgtttatgttttttttttggctttggccgcaaagctttaggaatgttcatataaatcactataaccaaactactaaaatgatatcttttttagtttgatcaaggggaaaagtgatcctaaagtatgccttctaagtagtcaaagtacatgagctcctaagaatcttttgtaagcaagtatgtagattgtttcttagagattttaattatgtagaaagaaaacttgatcattcatgttgtacaacattcaaaaatgatttggaacaaattatacatcttattgactataatttatgtaaatcagttctagagaaatcttgtttttttttttttaatgtagttcagaggaatctcccaaactaattatctagatctgaggaaataatgttaataataaagcctttctattctaagataattccaagatttaaaaaatatcagtaacattatttatatatgtattattaacaaactgtaaatatcttcctatactaatcctagctctgtgtttctcaacttgaaatgagtcagccactacctctgtgctacaagattctgacatgcccagagatggaatttttctaaaccagccacatcccaacactagtcataaacacacatacatactccctatttgaaaagcgacagggtagctgacagtgttgggattttcatatactatgtaaaatagtaacattcaaaaggaaagtcagtgatggatacaaattcgtaatgttcaccagtcatctccgtggagactgatcaacactttacattccgaattcttagactcggaaagttctagatgagggaattctgtggtttgtggtaggcatgggaagcagtaggggtggcctgctgcttcctgtggtcctgtctttctggtatgttggagggttggttgtaaaataaacatgaataactctaatatagcatatataatattttcggattacagcttaataatgaataagcatttcagatacctgtactatggccagtagaactgcattcaagtgatatctcattaattattgtgtttatgaaacattactagttcaactgattgcttgattattttgaaacttgtagagacttaaaaccattagatatttggatttatcaaatgaatctgaatatatgttgattacattacataacctctagaagggaaaaccaatatgttgtgtgcccagttatattagggtcaaatcaccagcccgtgaattgactaaatttttaactacttgctgttttttcaataaggttataatttaggatttgtttctctcattttattatggattaattatgtctcacttgccttgcagactttctggggccttggacacctagaacattttttgtgaatcttgaaataagaaaaaaattttggagggaaatagctgttataaggtctctccttaattctatgtctttattctatgagatatgaataaactataattaaccataatctattgacttattattacatatgattattttaaacgtagatataataggcttggttatctgctttcctggcctccttcaatttttctttggttaagtatctcacaaaaaaagtcagccactgaaaagtaatagtgcctgtgaagatgcttgaagaattacaaattactcagctgttaaatagaagcatctcagtggtatagtgtagaacctcagtgttccctgggacatcagtgtatttcagtaggacacatgcagttaccattcttaacaatggcctctgttgtaacagtagcataagattttatgttatataccatgtttttagacaagaaaatatgcagtattttaataaatttaaaattaaataactatgaaatgtcatatgaatgaggtagtattacaaaataatgtttcttgcattcttgtttctagtatgctattaaacctcatttacagtttgaatgattttctttataaatagtattaagtatagtagaagaaaaagtttttattgatagtattgttaatgtccaaatacaaatgatgagagagatttccacagtgctttcattaatagtgagaaacactgaatgtattatttcagattgttcatttagagtttggagcagagattttatagcccacgtatggcttaactttctcttatgttttcttagaatatgtgtaaattctccagactagatattccaatcagtatattgaaaataacttgagaataaattttcttaaagggaaacaaggtgcattatgtataactagtaagcttacccttactggtcacaacctttagtcatttgttaggatttgttctagcgcaataaaatgtatttactgttttccaaatgatatagtacttctgtaatactgtttttgtatcataagttcaattaacagaaataaatatatcactaaaagatttgagtttatacttgttctcaatacaaaatattaaattaccttctactttgttttcaaaatacttctagctagaggataaagaaaattgatagaaactggattttggccaggcatggtagctcacgcctgtaatcccagcactttgggaggccaaggcgggagaatcgcttgagcccagggtttgagaccatcctgggcaacatagggagaccctgcctctacaaaaaaattttttaaaaattagccaggcatagtggcacatgcctgtgttcacagctactctagaggctgaggtaggaggatcagttcggctcaagaattcaaggctgtagtgacccgtgattgtgccactgccctacatcccagggcaagagagaaagaccttgtctcaaaaaaaaaaaagaaaagaaaagaaaagaaactgggactttttaacttacagtttatgtttaccaataacaaacaacttgttagatgcaagctagtttttagtaggttgaccatatataatccatatcacatcttggtgttttttactttgaagcaaggtttcttatgagaggggttgttacttgtgcaaatctgtaattatttaggcagtagaaaaggcctgttgatacatattcaagatgagtggttttagttatttttattatttataggaaaaccatgctgtccataatttgaggaattcttgataagtgggtcatctaaaaaccagttctgtgatcctgggcggcggggggacctttttaaatgtactactgtaaaaatacatttgaggatcttttagtagaaaaatcctaacagcttcagggaccattatgaacattttgcattaaaatgttttgcagtatggacagtaccactaatattcagcttttcctctttttttttttaacagagcattaagtaaaatgatactgaaatatagtagatgtttctaatgactgttcgtcagagtaacaccttaaagtatgtttacttaggaaaagaaaattagctaacaaaaaaaaattgcatttacttaaagatatttcttatattgaatttataatatctctgctacttatgtatgtttattagcttttgctgcattattatacttaatgctatagtattttagcacataaaaaatgacaaaaagaatattttaacttccatggctaaaatattaataatgattatgtttgtttttaaggttttaaaatactttgtaataaaataactaatggtattaatcagttgctttgctgctatactatatcagagtcattgccaattcataattaaactgaatcataggctgtctttaaatgccagcatgtattcattgaatattctgaaagtttaatagaattaattgctttttagtggatagaataagccattaattataaatgcattggtaggcatttaattgtggttaaaattagtaacttcctgaaaattccagtctggttggcgataattaaaacttgaataaagaaaataagtgatttgtggtgtaacacttttgagatattgtttacactggcggtaaccgagaaatgaaatacttaaatctattaatgttattagcattagtttcatttaaaagcctggacatttttttttatatttgaaatgtttttgccctgagtttattcacctttaccaagctttgtcctttgtctttaaaagaaacacactttgaaagtaaagtggtaatttttaaattttaattattatattcataggtttttaaaattaaagatatgactatcactataaatggcatagttcaactttttaatatgcctaataccatctcaaaatatttcagtattttcacttatttttaatattttttataggaaaaattacttgattttgtttaaactctgaaattgatttttatttaagagtatatagttctaaaaagctagtatggtaaatttcattattctactttattacaattaaatattttcatttaaattaaatattttctatttctgaataaccaccttttacctcattcagcaaagaaaatcacttttaaatttgtccatatccatattctctttcatttttattgttggagaaaaactagttaggagaataagacttgacagaggagccaagaggtgtgtgttaaaagcctcaagaaaagagttactaatttgtgtagttgcaaattaaattgatagtgtatttttcctttctccaatatcccactgtcataataccacattttcataggatcagcaaacatgctggtaaatgttttaacaaccagctctgggactagagaaagccctgctttttaggttcactgatttcatgatataaccttccattagtccatatactgacaaaatggcttaaggcagtattttctaattttaatgcaagaaagtagaagacagtatgcccaacttctgtcagcatgtgtttaaacagctcaaagagcagacctgctgtaaaatttcaaccagtgctattgcaaacagcaaatacttggcctttcccatttttaaagagtttttgaaataatcaaacccagataatacaaagccgttatatcagctattcacatatacacagcattacctttcacacaggaacttcgcattttgtgtctttctgtcaagctgtggggctaaataaagccttttgcaacaggtcagaaactttacaattttcttaagatggctcagtttcttacacatagaaagtcccctacttagataccttaaaacttcttgagatgacatttcagtcagttttaagttcttactggtataaaatgtatcaattctgctttgcttctcagttccattttaactaaagcccctctccatggacacctaattcttgcaatgtgtataatcttatgaaaactaggaggaggagatttttagaaactaaaagacatttctgccatattcccaactcagagtcatatttcatatttataatctaaactgattactttctccatggctgtccctgaataaaattgtaaactgggccaaaatgtgtcttgagtcccattttcttctactttttcttttcctggtcaaaatttactatcaggaaaggaaataatataacttagcctccttgattattaaattactttgagtaaattactctgctgtggagatgcctctggtaggacaaaaatagtgctgaatttggacttgagatgggatacaggcctggctctccaacttcacaaggcatactagtttcatgaaatcagccacatcacatgtacagagctgatattcagctggtaagctttagttcagtggcccagttttttaaatattgagtatcctagcagttggtaagtggttgctgctccaagacccctaatgccttcacccttcttctccctactcctcccaaatccagtagctgaaagtttatggcctggtggtggctttgtggttaccagaatacctttccatgcccaccacccaggaaggccgtaggtgggcaccaatggagggcacgatagtgtttcccagcctgctagcctattcttcccatacacttgccagatgttttaaattctccacattgaacagaagttgactgtttttaacatatgcaacatatcatacattgctgatagtattctacattaattatcaataggaaaatatccccaacttgagaaaacaatttgtgcccttagagattgaagccacatgcccagagttctgttgagctaaaaacaaatctatgaagttaatttctaggggaaccttcttttccaagtgtttctactctcaattatttatcattcattcaaagtgaaattttgcttttcacttagttttgagtacaagggcattgtattttaattcattgttttaatagttctactttgccagcagtctcaactgcacctgactctttcagcagtggcatttctaagggatacgttatcatcatgtcttccctctaaaatatagccatagtaagaacaaaggaaaataattataagttcatgtttaaattcttaaactgatggtgtttttatgatttgtgatttctgatctaaacattgttttgctgtacaacatattcaaagtaagttcctgaaccacttttccaaattaccttcattgtcctttcattttaaaaactcaatcctagtaactctgaaacttgccatacagactagtatggttcaatcagtatggttcataagatcttttatgctaaattttaacccaaagcatttttaatttctaaaaatacttggtgtaaaaggatgcgactgaatataagtgctggtttttctactgatgcaaaataaagggaagacttactttagtgtcctgcacaaaactgataggttaggttttttttactttcagttaatgagtagtaaaagaccaaacttcattcttatcaaggacaaagttatctttaatctgccgtatgtatctctattaagtacccggcatttatttcacatgtatgattccttttgtgaaataaaaagttccatgtggaataccaacatagaatgtgcagtaggaccagggctcaaagtaacagatcaacagtgaagatcagggaggagcctttggttaactattctgtcaatccttgtttacattaaaggtagtggttatgcttttgtataatgatgtatccctaacgtgaatattcaaactaaagtttgttatcctcctttctcaagaaatgttgtcataatcatgccttacagaaatttgctaagcctgcacattgttgaaaagtgattttttgggtttttatttttcccgacttattttcctagactgatcccgttgagaacacatatgtatggaacccgagcgtcaagtttcacatccagtcacggtccacatctccttccacatctagtgaagctgagccagttaaggtgccttattggagcatccctgctccccaacttttcacttgtgtcagaaaagagggaattgaataaaacaagtctgtctctctcctcttaaccaaatatgtaataagttacagtctattggagatcattacatttaactgataatgtaggtagaagtaaaaaactaaagagcacattgtcctcaaatagtcaatctgaaatagacaaaactaactagaaagatagtctttattttaattagagaaccatgtagaacagaagattaattatgtaaatttttgaattattagaaactctaggcaaatggcagtgcaggtgccattctcatattttaattcttctaataaatgtaataggtcatgatataactaacctgttacatcataaatagagctgaaaaataaaatagccacttctggcaactgaatagttcattctaactgctaaattggtaacagtcaactaaatcctgacatttaatagtgcaaaaaaaactgtgttgtgtttacacttcatgtcaatcaatggttttaaatagccaataggccaattttcttaacagagatcatttcttccacaagtcaaaagaatgctgtacctcagtccataccatcctaccttaagatcagctctggttcttcattcagcatcaggattcagacaaagctcctttgctttccttttcttaaatctccagagctgtgtctagcattccaatagaagcagatagtccaatctgaagagttgtctgaagtgaagtgtgaggttgtagtcaggttctgacttgatttttccaactctgtgacagactcccccaaattgtttaagttaatgagaccctcttgtgtttttagttataattgttttaatattttgagattatacatttttgttttaatgataaaatatttcaagcattcagagaatataaacacccatgtaaatatcacccacagattaaacagatacgaaaattttgcttgtttttcaggtttttattcaaaaatatttcagaaacagttgaaacatcctcgtgtataccgttccccactcttctttcccagacatatctaaatatcctgaagctgatgtgcatcttccctttcttggttttagatgttcatttcatttgtatccacaaatgatacatagtagtgcttattatgtgtttataatttacataaatattacctgtatccttctgtaatttttttcattcagaagtctgcttttcaggtttattcctacagatatgtgttcatttagttcattcattataactgtggcatataccacaactacttatccattctcctgcagttggatatttaggttgtttccaatttttcattattatcagtgctgaaattaatatcctaatatctgtttttgcaaatgtacagagtttgtttggcatgtatacttagtggaattactgagttgtatgttatatgcattttcaagtttaactggatcttgccaaattgttttccacagtggttggaccaattttcactcccccagcagtgtgtgtgagttccccatttccccacatcctcacctcacagtcacacattaacttttgccagcttgatgggtattaagtgtcatttttgtattacatctgttttctagtgttattcagcatctttttgtatgtttattgaccattgatgatttgcctgtttgtgtcctttgttgttctattgaatcatttttcttatagctttttgtgttcattatatactctagatactaattctttgctagctatatgcattgcaaatattttctcccagtctgtggcttattttaaaattttatttgtggtgtctttttgtcttgcaaaagttttgattttaaaatagtcagatttatcagtctttttctttatgacttctgctttttatgtctttttaaaacagttctctatgcagagattataaagattttcttctatattttcttctaaaagtttttaaattttgcttttacttgtttaggaccttaatcaaactgatatatattgttatgtataatgggaaatgggcttctaatgtctttttcccatgtgaacaagtgaccctgctatatttccccccatcgatttgtaatggctatttctgctttatatgcatggatctgtttggatactctcaattcttttccattgttcttttggtttttcctgcattggcagtaaattgtcttaattacaattactttcactctaaaataattcctgatacctggtatggtgagccatcccacataccttcttccttttcttcttgaccttttagtttttcatattacttttaaggtcagcttgtcaaatttcacatggggcttttgattggaattgcattaatgtatagatctatttgaggagaattgacatctttcatgatactgggtattccatctatgaacatggtatacatattagggattctcttatatccttcaataaagtttcattattttcagtgtatttcatattgcttgttagatttgttcctaggtaccttaaattttttgttgctgtggtgactgggattctttttaaatacattttcttactgtgcattgccttgtctttataaactctagatcttgtatccagcacccttgcttaattttttttaattctaataacctttggactttctatgtaaatttgacgactttgtttcctattgttttttattaacttttgtatagattttcagtgttcctctcttttatctttcatatgtatcttttcaaatgttaaattataagtttggtttaaaactttttcaagctatttaaaagtaaataaaatctaaattagagctaaactcacaaacagaaaactctgagaaactcaactgaattttgttttaaggctgtagtgcttataaaatattctgtttttggaggttgtatggtaacaaagatagattgttgtatagtaacaacaaagatgaggttgtataataacagcaaagaaaggaccatccatttttcttataatattttaaaatattctgttctttatacaaagactagcgttttccttttatttttttgcttggcatcctgaaaggaagttactttccatgtcttttttttttttttttttcctcactactttgctgattaagcagtggctcttttttccccataataacatccagtggaatgattggttctttgcattcaaagaaataatattacaataaaaccccataaaagaagaccatattttacatagctacctaagaaagatatggtatgtgactaaatttgtattaaagtcagaattccaataatattaaataattacaataaaattaaataattcccttctttggaaattaatttctgcacaaaaatcccagtgtcagtctctgtctatgtattcacatccagagctgtgttctatatatgtaatatatgttctatgtatgaaagttgagtagttaaaagaatcttctaaatattttattataaatcattttgagatactttttagaatatggcaaattcagatgaatgtaaaaattaatatcctcatatttatgtaggccacatgctatggctttttttatttgaatcatttgatccaaatgatttatttttatatttccagactaaagtaacttactaattgaacaaatatttcattcgtagatattgcaggcagagatttaaattatcaacactatgagaattttagctttattgaggttttctttattattaattagtagaactttttgccttattgatgttttgttttatttattttggcttctttctagttatatttgaaattaatggagtgtttcatgacatttatttccttttttgctcctgggttcagactgtagactccactccactttcagttcacacaccaaccttaaggaaaaaaggatgtcctggttcaactggctttccacctaaggtactcaatctagttataagcatagtgtgttccaaaaaaaatgaattttatctgctctatgcatttgctgtgatttttttgatattatttcttcaaatttacaaacagaaaaacttataaaataaattatgtgtgaatgcaaataatcaccaataggaaaggtagggagagcgtacagcactttttcatcttcttaaattcaatgttttgaagtctgttataataagaagtagcacttcctctaagcagtatatgcctcagtaaggcattaagttgaggccacttgtggaagaatctgagactgaaatcaataaaccagtgagaaagaaaaacgtttagtgaaaaattcaggtggacttttttttcccattctatttgacataaatatgactgaagtattacatatggtagaatgagaagatttagttgcttgtctcaagctcccacaaagaaattccagaaattggaaaactgaatgatctgcctctccagaacataaaccttggataaaccacaaaatgagagagagaggtcaatagacctgaaaaaatatacaaaaaatataactgatatagaagaaagacaaactgaaataacctgtgcccagataaagcaaaattttaaggccagatagaacatgaatttaaaataaacataaaaaatatccttaaagagataagaagtatgttggtaacatgaggcaggataaggtagttaggaagaaaaataaattgggaatacttgacatgaaaacataactgctgaaataactaaatagatgactttaatagtagaaggggtaaaatggatatcacagaaaaacaagttagtaggagggaagataagatgaaaaacaggaaagttaagatatatgacaacccaaccaaaagaaaacactctagtaaactaaaggtttataaataaaaggaaaaatattgggcattcaggcaaaaaatatgtcacttaaaggggaaagaaaatgagattatcatcagattttttcatagtaacagtatgctaaaaacaacagcagtgaaataacacatttaagatacccaaggaaagtacaagtgaaagatttttaaggatttaatattttaaggatagtaaagcatactccaaaaatgaaggccacagaaaactattaagaacatgcaagagctcaggaaatactgttcccataccccttctcaagcaatgtgctagagaacaagcttcagacaaccaaaatgataagacaccaagtactttgagaggcaagagtgaagcaggagtctagagtaaattagagaattgattcaaagtccacataaagtgttataccagttctgaaaaccagacaactaccccaactcagcaatacgttaattttgtgaagtaattaaaccaaagagatcacaaatttaagggcaccaaccaggctcactggttggtgagagaaagaagtgcaaaaccacaaatagttgagatttggcaaaagtccatgtactgaatggcgagacttcaaatcctttcctctgcttagctcccagaatactgactcttgggactgtagcctcctcctccccactctgcttggagaggatatcagtggatcctcgggtccactgaatggcccagaaaagacctatcgataatgtcatttggggctccctaatgagatggctaggtcctttcttaacaagcacatcatatgtttgctctcatgtggacacatagatagacacaaaatctctttagcttttttactgactcttaaaaatgttaagattcccaacagtcagtagacatttcaaaattatctaacaaaggaaaaagatcagggatacgtggtggctcacacctgtaatgccagcattttgggaggcttaggcgggctgattgcttgagcccaggagttcgagacctacctgggcagcatggcaaaaccctgtctatatgaaaaatacaaaaattagccaggcgtggtgatgtgcacctatagtgccagctacttgggggttgaggagggaggatcacttgagcctgggaggtcaaggctgcagtgagctgagatcgcaccactaaactccagcctcagtgacatggtgagaccctgtctcaaagaaaaagataaatatttcctgtttttgttgaaggaaaaagggacaatgcaaaaagcagaaagaaacagagcaatatgagaacaagaagtctcaagttaaagaaaatatgataaatattttttaaattcagtagatgagtcacaagataaatttgagaattctcttagaaaatattgcaaaaaatcaggtagaaattaggaaaagaaaatatgagaaatccatatccaacaagtagaagttcctgaaagggaagacagaaaaaagaaaaggaaggaaattatcaagtaagtaaaagactttttcctgtaactaaaagacatgtttccagattgcacagttccatcagtgcctactagcactagtggatgacaaatacctgtccccagtcacatcattgtgtgagttttcaagataccaaagatagaactttttaaacctttaaggagatttaaaaaagatcacatccacatattcaggaattgaaatgacatacttacccaaggcaaaatgaaaagaatagttagaaggtaaaatgatttcaagctagaatttttgattccctgtcaaatgtcccatcgagttgcaggggggaaaaaattgcattttgagaaattccagatctcaaagaaatttttatcacccatgtgctactttctaaggaaacttaattagttctaccagaacaaggaaggggacaaggaaagagagacatgaggtctatagaagaaggaattgtacacagaccggttaagagaaagactggaataggaaaccagagggcttcagcagggttgtcttcaggtcaatagaaaggggtaagggggctgaaaggcttccagtgtgtttgaccatgtggtaaatagattccaggggattttacagttccttagaagagtctggaagatttagtgatagagatacagaaaggtaaaagtcgagggtgggaggggaactattaattttagaaaaatcatgaggttatacaagtaagatgataaccatagtgtacttcttattagtgaataatgattctctagtcataataacttagacattacatataaattttaagctaaaactactataacaatattgggaaaaatgaaaaaagggagtagatagtgtaaaagggccatatccttctctaccatagcaggaagtgaacagataacgtctcaaatcaaaaaactgtagtataggctttccaccccagctctgccccgcccatatagatacctattacagaaggagggaaatagcttttaaaaaaggagagttaaatgaaattgcctgtaggaagtaggaggaatacttctgattttgtcataagcattgtaataccattttactttttcaaccatgtgtatgtattaatttgattttaaaaatttaaaacaaaaatgtagaaaaattttttgtgactgggaataaatataagatctcaaatcacaagcataaggtgaaaatattcgtgggctttacatcaaaattaaaaatgcttttacagtgaaaggattgttaacagatgatgacagactagtggaagatatctgcaatgtttaccactaataagaggcttatttctaaaatacataagaaactcctgcaactccatatgaaaaagactagaaatcccattgaaagattggcaagtgatataaataattcccagaagacgaaatcctgatggtcaacaagcatgtgaagaaatgtcccaacttcattaatcattggagaaatttttaaaatttaattttagaattactatcactttctattcaaaatcaagataaaattaagtaagatgacgtcatggatgtggagaaacaggcagcttcacacgtggatactggaagtataaacgggtacagccattctggagagaacctagacagtgtagagtcatacgtaagctgtatgactcagcaatctcatttcatcagaaaaatctctttcctttacaaaaacctccatacagattcattaggcaacatgatgaagatgtttattactgtattttttatggcagtagggagttggaaataggggaaagattcctaaaatgtaattgatatataacagaaacacatttatataaattaaaaacacatactcagactatctaatttataaagatatatacatgtccaaggtattggatcatcaaggatgaaaaggcaaaataagacaaaataaaactagagtgaggcttctatcaagctgatattattttatgaattaaatattaacttagtttgttgcacctaatatttctccccaagaaataaaatttatttcatgttttttaaaagatagagaagtgaatctataattatctcaaaatagaaattttaaaagggaagaaaaaatatttttaaaaagaaaattaaattttttttttttttttttgagacgaagtctcgttttgtcgccaggccggagtgcagtggcgtgatctcagctcgctgcagcctctgcctaccaggtttaagcagttctcctgcctcagcctcccgagtagctgggactataggcgcatgccaccatgcccagctaatttttgtatttttaatagagacggggtttcaccatgttggctgggatggtctcgatctctggacctcgtgatccgccctccttggcctcccaaagtgcttgggattacgggcataaggtaccgtgcctggccagaaaatttaatttctttaaaacattttaaaattttttgtaaaaaagaaagggttgtgtgagatactctttgacttgtcatttattagtggtaaaagtttaattatctacagtaaatttttaagctttcaccaagagttgtacttggttctacttggctcttacatcttctattaaaaacattttactttctccaatgtgtagcttaaaatctacaacacagaaaaaatcagagtagaaaagaactctttctaatcaccactttgaaatttgaggttatggttaattctttctactccaaatgatcacataatctaaaattctaattttagtccatactttaatacctttcttactagatttttttcttttcatttttttctcttcttccagctgtccacaaagggaagtattagatatcaaatattgaccaggaggcaggaaacagggatattagaagccaaggaatctagactctaagatcgatcattcctgtgtctttctttatacattttcacaccaaaacaaaaacaaaatcctattgtgtattacctaaagattgatggcctcagaataacgatctttaattatgttatgttatactatccttagtggagattagtgaaatataaacccgttaatctttagtagtggattacacaacctctcctataatttaaccagcacctaccagttggatgatttattacctaaaattaatgctcagccaaaacactgggttgttatatttttccctgctgtcatactcagaattgtgacatcctgaatagatctcttgctggagaggaagtgtatgatatatatatatatcatatatgtatatgtgcaattcgggaggtattatggagaattactaataaatttgtcaaccctagagttgtttcattgctactgtttcaggtatgtcttgttactgtttagaagaagaaaaacatgaacctacttagagcagttgattaatttttccttctttgtattcaaagcctctaggactaaccagtaaaataacttcacgtcttttgcattaatgcattttgaacaattgaaaaagttagcactagaaaatttactcataatagtttctcaaagagccaaaaattaaaaagtagtatacaggagaaagagcaaaattttatgattttaatagattccttcaaataaggaaataatatctatctttccaaggtggttatttgcatgaaataagatgaattatgtaaagtactggtacagtgtctgtaccctgactgggactcagaagctataggagcagttgcagagttagtaataagatagactgtgttctacttagagtcaaatcttagtgctgctttgaaactgaccaaaagatcgtgaggaacttctagaacacaatgctttgaggttggaccagagctcatgaccactctaaatttgagactctcaccccattctctgactaatgctggtagtcaggtttttctatgtaaaaagataatctacttctatttttttaatttcttttgtagtgtacaaatcatatatcccagttgaaaagaacaagatttatttttaacttgaattttaaagcttcaaatattccctttagttttttgttcatatgttagtgtctactgaaataaatgctacttgataaccttttaaaatcagtattcataatttattagaatatggagtagagttcttagtagtttaaatcaatcacacatttacaagagaaaaaaatagcaaaattcaaataacattgggtacccatggacataaagatggcaacaaaagactctggggactactagagaaggggaaggagggaggggtgccagggctaaaaaattgggtactgtgctcagtacctgggtaatgtggatcggtcatgccctaaacttcagcatcatataatatactcaggtaacaaacctgcacgtgtacccacaaatctaaaataagagttgaaattatttttttttgagtacttagtatgtaccaggtactgtttgaaaggctttgtttatattaatactcatttaaccactccgttaggtagatactattatttatcctgttttgtagataaggaaagtgagacagagaggaatttagtaattttttcctaagttcacacaacagaatgaaaggttatttattatacatcatttattgttaaaacatgctaagagaattaatttggaaaacaaaggaatattccaaagagataaaatctacatttgcttaccaccatgtaaatcaggtaagactaagatacctttaactatggtgtattcaataagaaatttcaatctgccatctgtctctggaataatatttgtgtattcttatctgctcaagtgttatactgcctctttccataggaggaaactgattcgtgaagcatattatttgtccttggtcccagaaagagaaatgcatgatttacagtagaaacaataattttaaataactctttttcctaagaaggtataaagcatattaaaatcctatatttgtatcagtataaatatgcaatattttacttaactatttgctgaacaataatttggataatttattaaaattctgaagctaagatttatgagccccccaaaaaagctattgtttattttatatacttttaaaatggcattgaagagcaattatcatttaacttcttaattattttagcttagtccagttcaaattacctaataaaaatgtttaaagaccataaactataagaagatgtaatgagaaatggaaataattcattctattcctatttagaaaacattctagaaagtggtatggggcataaggactcacattatggaaatcttttaaacaatctgtgtgtttttttatcaatttgttttgcagcgcaagactcaccagttttttgtaaaatcttttactactcctaccaagtgtcatcagtgtacctccttgatggtgggtttaataagacagggctgttcatgtgaaggtaagaacacaaaggaaagaaaatgtgacttcactatctaaattgtaatcaaataagttgattctctttcaaaggtgtataaatgtttgttttacttaatcatttgcttttttgcataacaggaatggaaataatctgtgttgtctgttgaatgttcgagaaactagacatatttttcaggggcaatatatttaaatatggttcatttgtctttgttttgttattaaatttgtagtccctgccaaaaaacattaacctatctgacagaagaaaattcattgaattttaaccttgtttaatttattagagaaattgttattaaaaagtagacagtgtgggaaccttttcagaaatattaaatgtctgccactatgtctgaaagatattttattcctgtattaatagcagaggtttttttttttcccctaacatccatgatttattcatcacatgtttatggaacacccactgtacttctaggcactggggacataatagtggataggggaaaggcatagatcctaccctcatagagcgcacattctgagtgatgaaggagagagggtcaggggtcatgtgaacagaaaagaaagtatatttgtttgggtattcctttatttttcctaggacctctgaagtgaaaatggtttaaattataatggaacatatatttttttaaatgtttcagaagaatacttttttaaaaaatcaaatgaaatttgtcattacaacactaccttatatgaggggacttcaaaaagcttgtggaaaagtagaatttaaaaataaaaataaacaatataaactttttttttagcataagctccattaagttcaagacaactttgtaagcaattataccacccatttagtccatccctgaagaacagagggtcctgagaatgtaaccatgtcagtctagtctcctttacattattaactgaagaaagatgggtgcccttaaagactttttgaaattcagaaacaaaaaagtcagaaggaaccaaatcaggactgtaaggtggatgcctaatgatttcccatggaaacactcacaaaattgctgttgtctgatgagaggactgagcaggaggagcattgtcatggtgaagaaggactctggtgaagcttttcccaggcgtttttctgctaaagctttggctatatttctcaaaacactctcattataaacaggtgctatcatcgtttggctctctagaaagccaacaagcaaaatgcctcaagcatcccaaaaaactattgccatgacctttgctcttgactggtccacttttgctgtgactggaccacttccacctcttggtagccattgctttgattgtgctttgtcttcaggatcatactggtaaagccatgttgcatctcctgctgctgttcttcaaagaaatccttcaggatcttgatcccacttgtttaaaatttccgttgaacattctgtagctgatctgggtgcagtggttttggcacccaacaagtaggaagtttgctcagcattcatttttggtcagaattgtgtaagctgaaccaattgagatgtctgtggtattggctgttgtttgtgctgttattcatcagtcctcttcagacagagcatgaacaagattaattttttccctggcaaattaatgtggaaggtttgctactgcaggtttcatctttgacatcatcttatcccttcttaaaatgagttatccatttgtaaactgctgatttctttaaggcattgtatttgtctcttctcacattgctaataaagatatacccaagactgcataatttataaaggaaagaggtttaattgactcagttccacatgactggggaggcctcacaatcatggtggaaggtgaaagaggagcaaagtcacgtcttacatgtcggcaggcaagagagcatgtgcaggggaactcccctttataaaaccatcagatttcacgagacttaatcactatcaccagagcagcacagagagacctgcccccatgattcaattacctcccactgtgtccctcccatgacacatgggaattatgggagctacaattcaagatgagatttgggtaggaacacagccaaaccatatcaggcattgtccccataaactttttgtaaagcatcaatgatttcacccaagcttcattgtaaattttatatttgttcttacttaattttagcagaattcatgttgttctgataggggctcttttcaaattgatgtctcattcttcttagtgcctcaaactagatccttttcagacatattataacaagttagtatgcatttattttggtgcaaaagaaaattttaaatccatgcatagtttttccataatatgcattttccatgaacattctgaagatctctcatattgatgtatcttatgaaaaagacaagaaaatagttgctaactcaaattctaatactagttgccatttcctgtctcttctgctgttagaatgggaagggaccttaaaggaaatgtagcccatccccatcagatgcatgtgtttcctttataatagacttgtagttagcctgtatctgagtactttgagtaagagggaaattaataacatcttaaggcagtgcattctgtttggttagttctcacagctaaagtgaaagatccagattgcagtattttaactaatccattagatatgtgtgtttaaagccaaataaaatgttcagggttaagaaagcagagatttaagagctaggagtgtattaagttataaacatgaatgacatccaggaagtacatgtagattgagaagagagcaagtacatgagtctcttacatagttaagggaaggcaacaggaaaagatagccaagttaacctgagaaggaatcgcctgtattgttgactaggaaggtgtgactgaaggcaaagaaggagaaaatttgtagttttcaagctttgtttcattttggaacttagccagtaatggtatgtcattttaaaatttgtttatcagaaagaatcctggccagctacctggctttaagtcccatgtcattttttatgcaccacaattacttgcaattcatagtaggaatttttgaggtcttctcagactctcttcccttctagagtcttttgctgtgaaattgtgcctatcttttcttcagaccctttgaagcctactttatgaagctatggggactcatgagactgttcctagcctttgtctgccaggcttttgtaaatgttgtaataatgtggttccagcttctaaagattcctgtcatgttcctttccttgtggttggttggaattagatccagtataggatacttattttgttgcttccactgccctttgcttgtaagtaaaacaaatcataacttgctagatactccacttaaaactgaattgaaggccgggcgcggtggctcaacgcctgtaatctcagcactttgggaggcccaggcgggcggatcacgaggtcaggagatcgagaccatcctggctaacacggtgaaaccccgtctctactaaaaatacagaaaattagccgggcaaggtggcgggcgcctgtagtcccagctgcttgggaggctgaggcaggagaatggcgtgaaccccggggggcggagcctgcagtgagccgagattgcaccaccgcactccagcctgggcgacagagcgagactccgtctcaaaaaaaaaaaaaaaaaaaaaaaagtgaattgaagttgtgtgtgatgactcatgcctatagtcccaactactcaggaggctaagacaggaggatcattttgaggccagcctgggcaacatagcaagaccttctctttaataaataaataaagtgcattgagatgtttggatgatttttgtcatcttgcctctctgctggtttcacagtatccatcagtgatatgtagtccatttatagagatggtaactgtacctctcttagattgttatagaaaaataaaaagcactccataaagttaactattgttattttagtagtagtttaagtatatgtaataataactcctaaaatttacctttaacttgtacttttttagtcattgaaaaactccattttcaaggattggtagggtttgccaaaccgaagatgtcaggtagcttaatgcccactagtgacccttgcaacagaaatttcaaggcagataccaagttgcagtgggatgaagagcaaaaaactctagaaaccactcttttagaaaatctaaatcaatcattttattcacaaggttagatgcttcgtctgttgcttctttttttgatcactaatgtcaatacagaacagcagtaatacaagtaattttttaaatatttactatgtctcattaaaattgtcattggtaagcaaatgaatcagctacccaaatgataaaacagttatttaaaataaaattggagataagcattcatctaacatagcatcttgtatgcagtgagtgttacgaattttctttttgttaaaatgaatttttatcaaacgtttcttttttccctctaaaagtgtgtggattctcatgccatataacttgtgtaaacaaagctccaaccacttgtccagttcctcctgaacagacaaaaggtcccctgggtatagatcctcagaaaggaataggaacagcatatgaaggtcatgtcagggtaagtatgattaagttgggtttagattaatcctttaagatttattaatgaattaaaatcaaatccagttagtttaagaggatagttcttgatggaaaattaaatgaataaaattcatctaataattttatttcatctagaataatgatttgcaaacaaaaacctagcttttacatggaaataagttataaaatgtctcaagtttttccatattgttcctatcttgtgatgaaatcttcataaaggagtttgtggcccctgctcttgcaagagaggtggcatatggttatgaattatgagctattaaggcatagtcttgttatcttgggtagtatttacaatgtaagctgaacttcatcttacaccaagtttgacttttatgctatttagaaagaaacccatgagtgttattttaatgaaatatgagtgaaagttaaaatggtaccattaaaatgtttctcagaataaaaagtcattctgtatttgaagaatgtgttctcacattttctcatagcttaatgatatgtgtctcatctgctactttaccaaccagaatggcagacttataaaacgtgtacattatctgtaggtcttcaccatgcaattacatatgtaatttaagggatattttcatttgagttgttttggctgtttgtttgtctgtctaaaacaaaactaagattcctaagccagctggagtgaagaaagggtggcagagagcactggctatagtgtgtgacttcaaactctttctgtacgatattgctgaaggaaaagcatctcagcccagtgttgtcattagtcaagtgattgacatgaggtaagagtttgaaggtaaaattatttgtatcattgcaacataggaattttaaagtttctgattttcaaaaattgtgttgccatggtttatttaaatgaactaatttcgtattttctaatttgctaatattgctatagaaacagaattgaaaataggaagtaaggtgtatttataggacaggttggtatgtgtgttgagtgtgaggggctaggagttaaggaaaaagaagttggtgtcccaaagacctgtgtaaatggggtaatggaggcctaccatgtgaagtttatattatcacatcacctaaaggcttagagtataatttcaatgtgaggaaagaaaataaaaatcagaaaggcttctggaaaacataatcagtaaacatttaagtaagtacagcccttgaacacttctggtctcatttttcagattcttcctgtgttccttagttctctctcctttaccttttctcctaactccaggcactatccttgagtgatcatatttactcacagcttcagccatctcctctgcattgatgactcccagacttgtatctccagttcagatatatccatccattgctcaaaaccggaatttctagctgtgactcaaattggcacttcacattggcatctcaaacgcattatcttcccctacaaatatggaacatgacaggatagatgcaagaaatattggcaagaaaaatctgatagaacctactaagggactgaatatagatagggaaaagaatcacaaataggactaaggtgtcaagcctgtaagactaagggagtcatggtaccaacaacaaaattaagggaatcacaagaaattattgtggaggaaatacgttgtagatgttgaaggaacaataaaatataacatgagctgggttgagggtacttaagatttattatgggaggcaagagggtccactggaggctccagagacctaccagggttcaaatcccagttctacgtgtatacttaactggccttgtgactgtgggtaagccagcaatgatttctagctcttgtataatgtgccactaggaagttattcttgataaattatttggggcactattaattacaatgggggaaaattaatttttttaaaaaattaaatttgggtgtttggtgcagttaagctttgggtatgtgttatttggatacacaccccaaaccacacacacatagccacatatagtaacttttaatgctttgcttttcgttcagggatgaagaattttctgtgagttcagtcttggcttctgatgttatccatgcaagtcggaaagatataccctgtatatttagggtaagtgtgtatgctgtaatttgatcactgaattatgtgtttttaatgagtatattttgcctccctatataaatcataaattcactataagtaaaaccaaatcttatattcttttatatattccatatcatccagtacttgaaggtatagaaaattggtgctaagtatttaattagtgaaagacctggagaaagcatcaatatatggcttataaaatatgcttcctggtagataatattttttagttccaattcaatttttaaataaatactaccacatggaaaattagataagtgtgaggtttctcaagtttttaaaaaaataaaaatgtctgtcttgaaaaattaatgcgtttgttacttactgctttgtaacagccattccaaaacttagtagcttaaaccaacagtgatttactatttttcactattctgtggaccagctgtacagttcttgtggtgtccgctagtgtcactgatgtgactgcattcagctcatttgggaccagaacatctaatgaccttgctcttacatctgggtgctggctgttagctgggtgatctcagttctcctctttgtgaactccctccatgtggtctctggtcgtcaggtaggctggctgagctcccagtggctggcttctaagagagcaaaataagctgccaggccctttgaggactaggcccaaaacaggtataataacccttctgcattctgttggtcaaagcaagtcatgaggtcagcccagatttaaggggaggggaaatagactccacctcttgatgagaagaatggaattccttaggaggacttgttggtaggcgtctttggaggcccttgccacgatgcaatcttgagttggtaaattaagacaatagaggttaaaaaaaaaaactactgcccaattgttatgcctatctccctatatttgtctatgaatcttgtatctaaggaaggtcaggcaaagattatacttagtctcttagttcccagactgacaactatttattttcctcccagccaccaagggagcctacagaggcccacttatctataactgattttcatagtgtgagtttattgtcactgctacttaataaggaattctttacttatgataaggaattcatataggtaaaatttaaaactgctctgatagtgttgtttgtataatgataaaccttcatttaaatattaatttaaatggtcaaatcttaatgataatgcgagttattgttgtgaagtaacagagaatatatttctgagcacttctgaccttaaaacaagtcacaagtcagtatggtgtggcgcttaagagcatgggcacaagatcccagacttgtagttgtgtgtccttggagaaatgatttaatttctttgcatcaccatttttttcatatataaaataaggatataatgacagtacatcctaaatcatgacgatgttaacagagttaaatgagttaatacttgtaaagtgcttagatctacctggtgctcagtatttactcaataaatgtcagttgttgttgttgttgttatatatttaatgcctctttagtgatagtgttctggcacttttacagaaggaaaatgaaaatgatactaacatttcaaatggtgtctgccacctttgctttgtcatgtattctagtgctaaaggtagttattctgattaggaatagctgaactccagtaatggtatcatttaatgaatgcttattttattggtgagtgctaagcaaactaacataatcatgaaactagcataaagggtgtctgttttctaatatcatgaatattgattaaattgttctaggtcacagcttcccagctctcagcatctaataacaaatgttcaatcctgatgctagcagacactgagaatgagaagaataagtgggtgggagtgctgagtgaattgcacaagattttgaagaaaaacaaattcagagaccgctcagtctatgttcccaaagaggcttatgacagcactctacccctcattaaaacaacccaggcagccgcaatcataggtatgaatttataacatttatcatattattatcattgtttactaatctaccttgagaacagggattggctttcatctaggaatccccagtgtccaacatactgtgcatcatatcataaatgctcagtacatgtttactgaatgaatgagacctatatcagatctttgtttttcagtaaacaggagtataaatacactttcaaagtgccttttttttcctgaaaaaacaacagtgctaagcattagcctactaatctattttctgcttgtgtgatttagaacaagttagtttcttagagtttaatatataaaatacagatggtaattaccttgctaaggttattgggaagtttaaataagatggaatacataacacagagtgccctgcataagaaagaaatccccttaacaaatggcagtttctgtgtctggaggtttaaatttttgctgtattcctgtgtatgcatctaactcttttgttactgtccataaatactgaaaagttcaatgagtatggttcaaatgccattcacaatttttacctcccaaaacaataagtgtagaacaacttaggtttttcattttgtttcatcatttattaaacatttacttctttttgtaagtattgatatggattattctttaataattgttctattttagaagacatgagagatttaccagtataatctgaaaggaaatagtgtaaactgttattgtaattcattttatttccattttttgcaccaaatgttcttatcatctggtttattagcattacatggtttttttcctaataaaaatctttcacattcatgtcttacatagatcatgaaagaattgctttgggaaacgaagaagggttatttgttgtacatgtcaccaaagatggtaagagaaaactttttttttttggagtaattttaaatcatgtgttaaaaaaatttaaactgtagattctaaattctacaaagagcaggatttgctcttatttatcctgcgactaaaattttccagacctaatttgacgtttataaattaaaagattatttttaaatttctataatccccctgaggtttaaaacttcaaatatgtattgtctttcttgtatcattttatttaacagttctgtacgaactcagattgcatttctttttttttttttggagacagagtttcactcttgctgcccaggctggagtgcagtggcacgaactcggctcattgcagcctccgcctcctgggttcaagagattctcctgcctcagcctcccagtagctgggattacaggcatgcaccaccacgcccggctaattttgtatttttagtagagacgaggtttctccatgttggccaggttggtctcgaactcccaacctcaggtgatccacccgccttggcctcccaaagtgctgagattataagcatgagccactgcgcccagcccagattgcatttctaaagatggaacatgaaaaaaaatatatacaacttgattttcacattgttctattagtaccagaagaggcaacagctattaaaatgtgtttgtttggaggtttttcttctagggaaaaactaaatgctgggtgaaataaagagaaaccaatataacttggtattcataaatacagtattctaagttttatctttatgtcaacttagatcatgggaaaacaatgcattgttagccatataaaaacttctgaaaaataagttctctgtttttaaaaatgggaatcctatggtgacgttccctagaatttagtgccagtagaaaagtttccttgaagatttggcatacttttacacaagaatcctttggggtttgccttggctgccagaaatctcaggactcaaatttaatacgtactataggtgagttaaattatcttatgatgggttggataatctttttttcctgtgacttctgatgtcttattcttccgtatgtgcatctgtaagttgacttacatccttttgagtataattgggatatgattaatcaataaaactatattttgatttattattctttctgtgtttagaaattattagagttggtgacaataagaagattcatcagattgaactcattccaaatgatcagcttgttgctgtgatctcaggacgaaatcgtcatgtacgactttttcctatgtcagcattggatgggcgagagaccgatttttacaagctgtcagaaactaaagggtgtcaaaccgtaacttctggaaaggtgcgccatggagctctcacatgcctgtgtgtggctatgaaaaggcaggtcctctgttatgaactatttcagagcaagacccgtcacagaaaatttaaagaaattcaagtcccatataatgtccagtggatggcaatcttcagtgaacaactctgtgtgggattccagtcaggatttctaagataccccttgaatggagaaggaaatccatacagtatgctccattcaaatgaccatacactatcatttattgcacatcaaccaatggatgctatctgcgcagttgagatctccagtaaagaatatctgctgtgttttaacagcattgggatatacactgactgccagggccgaagatctagacaacaggaattgatgtggccagcaaatccttcctcttgttgtaagattctctttcttacggctgtcttatctttatatctcttcttccttttcccttctacttcgtctcccaaatgttgtcattgttgataacagttttttaaaaaagtcattcttaatgcttttcctcagtattgattatgttgttgatgtaaaaaccatatgtagttaggcctgggcaaacagggccagcattagcatgcacgtaatttaaacaaaacatctttctgtggaatatttcactaagatggattttttaccccacatcatattttgaaataacccagaattgttatctcatggtcccccaaaatatggtgcttgtaagcatcagtgtttcacagaacttattctggagaagtagttactaagtgagttatttaatcaggtatgtaaaggtataatgtaacatctataagaggtgttttttcagttttccacttttatctagatgggtagatatttataattgcttattttaccccttcaagaaatattaagacataataaattattgaatactttttttttttttttgagagatggagtcttgctctgtcacccaggatagagtacagtggtatcgtcatagcccactgcagcctccaactcttacactgaagcagtcctcctacttcagcctcccgagtagctgggacagcaggtgcacaccaccacacccagcttatgtttttattttttgtttttgtagagatggtctcattagttgttcaggctggtctcaaattcttgtcctcaagcaatcctcccaacttggcttcccaaagcactgagattataggcttgactcaccacacccagactcattgaatacttttaattatagaatactttggttattgaaaataatccaattattattgaatatgtgccattgaatatgagttaacagcaatgagtaaaatagatacagtcttaaccccacagagcatggggtgcagtggaaaagattcgtgataaagcaagtagttgcagtagagtgtaagtgctatgatagcagaagtagagagttctgtagaagcacatagaagtatttcatccaaactgttttgtttttaatgaaaaatacttgagcatgttaaaatgctgaagggaaaaaaaaccctagggaactacgtggaaaagttgtctatatgggaaatagagtaagaccgccaagaaggccagagaggacaaaattcagaagacaggtgcaagaattacttgggggcaggaggagtaaaacctcttcatttgtaacaaggtaggaggaggagtgatgggtatgggtctagtcaagtttaaaggttcagtgcgagaaagtttgggagggcaacccatgctcattacagaaaaatcagaaagtctgcgtgaacaggacaaaagaaaaatacctctagttctacccatccagacttttttttcccccaccctccatcctgggatgaacgacttaactattttttgttgttattgttgttgttttttgtctttccagcttttcaactgcagatcttgggaattaactatttttaatattttgtttcttctgaaaaatatggtcttaggtgggcacagtggcttacacctgtaatcccagcacttcaggaggctgaggcaggaggatcgcttgaggccagaagttcgaggccaacttgggcaacatagcgagaacctgtctctataaaaaattttctttaattagccaggtgtagtggttgcatgcctgttgtcccaactacccaagaggctgaggtaggaggatcacttgagctatgatcatgccattgcactccagcctaggcaatacagtgagaccttatctctaaaaacacaaacccacacacactctcatactcatcagtggaataaatgctgtcttgtaactgaatcagataataattatgattattctaatgtagagtcagagttggaatgtctgacttttttgtgtagctctttcaataattccctttgtaaccttagtttaagtaaaccattgaatctttctttgcttgtgtcaccatttgttgatattcattctttcaacaagtgtatattgagcatctaccctattcatgtaactgtgctacagtgctttattatacattcttaacagggactagaacacttacctgaagatttctctgctttctaagtttgattcactgtcaataacttaactacttttaggaatcaaatagttcaaatataatgaaattcctttatagaactcatgttgggcaaagaaatagtatacccctggtgtttacagaatccgtctatattataacggctatataatctgaatcatggtataccagttcctcatttacgatatatgttctgtttgcttcaaaaatgttttgatttggctactatatgatagccccataggatttgggtgggattccactctacagttatttagtgtagacctttcaaaatatttggggtttaatagttaaaagtaatccgcaagctgcccccccttcccttctcccaggttacaatgcaccatatctctcggtgtacagtgaaaatgcagttgatatctttgatgtgaactccatggaatggattcagactcttcctctcaaaaaggtaatttaacattaaaatgtgggctaagccaactgagtataagtgaaaacactaaattactacatagtaattttttttttattaattcaacattttctggaatgtgtaattggcagtttgggacttagctaagagtgagaaaatagaaacagaaataaagttggttaatttccttatgcctcactaaaaccatactaattttactatatttggtttatgcaaagtatgattgttattattatgaacaactagaatatatcctacaacttaaaatgtcagtgttatcttctacttttggattaaggagctctgagagagtacatgattattatgaattataactgtcattcacccagtgtttaatcctgtgaggacagatttaaaaggctagaaaagtaattaaacttagaaccaaaactgtttaagtgtccataattgaaaaataaatatttcatatattcctaataaaacataatatttttcttaatatataatcctgtttccataacaaagagaatttttacaattatagaactgctagtttgcaaaaatgaagagcaatagtttcatatagatagatgtgatatatatatagcatgtatgattagttgggggacagggtttagagaatatgtgaaagtagcttagatcatatttcaaaattagcacccatttaaaatcataatagcagtttaaaaagctcttgtaaatactgccagattatctaaagcaactaaaatatactctagtttgtaaatttggattggtcattattacttttctgtggatttttttgttttgtttttgtttttgagacagggtctcgctctgtcatccaacctggagtgcagtgacatgatatcagctcactgcagcctccatctcctgggttcaagcaattctcccacctctgcctccagagtagctgggactacaggtgtacaccaccatgccctgctaatttttgtgtttttagtagagacagggtttcaccatgttggccaggctggcctcgaacttcttacctcaagtgatctgcccgcctcggcctcccacggtgctggaattacaggcatgatccactgcgctcagccctatttgatttttatcatgtaatttttgcaatgtatgtgaaagaatagatatatggcctttgtgaaagataataataaactgatcactcatgaacctacctcctaacctgagaatcagaacatcactcatataactgcaaactgcgtcatgttgtgcttatcattcctttgcttttcttaaaatacagaatttttgcatatttaagtatccctaaataatatatctttcagttttgcttggttttgagctttatgaaaatgttataatatatagtcattttctatcttgctttctttactcatccactgagtctcattcgttcatgttgttatatataccatatagtttattgctttcagtactataaaatattctgttgggtgaacttagcactactgtctgttccctgactgggggacattgaattatttactagctttttgcaattgtgatgttgctgcatctgttttttggaatagttttcaggagtttgagtcatatacctaggagcagaattgatgggacatagggagttcagtcagctttatgagatagtgtcatattattttccaaagtaattgtaccaaccaatactcccatcaacagcataaaggaattcttatcaacatcctagtactttccattgtcaggattcataggttttgtaaatggttacaaagtagtatttcattgaagtttcagtttgcatgttgctaattaactgtgtagttgaaagtcttttgtgtgtttattcatcattcatatttcctcttccataaaatgcctggtcatgctttttgctcattttgctattggattggccatatttttcttatttattgaaattctttatacattgtggatactggctaataatggatatgtaaatctttttcttctttgtgacctattcattcatttgtatttggctgaacagaagttactaattttaatgtagttaaatgtatctttcttttcctttatgtttgtcttttttctcttgtttaagaaactctttcctaccttaaggtcataagatacattctattctcctccaaacattttaccttttttaaaaaatcttttttaaatgtatatacccagcctccaaacattgtaaagcttagcctattacgtttatgtttttaatccaattggaattcatgcttggttccgtataaccagttgctcaatcactatttattgacagtccctcctttccccagtgatctacccttccatctccatcatatgtatgcatttagtttttgatagtaagaattcataaatcagctgtgttgatatctgcttagtataactgttgggtcattaatggctttaaaaagtctaaggtttaaatgtatttctacttatttgttcatttgcttaggatagccttcctttaaaatattatcattgcaagtttgtttgttcaaattagatagtattatacctttccagtcagaattattgttgatttttctgagagggctgcattttgaagtagataaatgagaagtcttaataaggtgtgctttataagcaggacaaaagttttataatttaatttttaaaggttttgttgaaatactaattttgtcttttattttaggttcgacccttaaacaatgaaggatcattaaatcttttagggttggagaccattagattaatatatttcaaaaataagatggcaggtaagaaaatacatatattcatagggatagcttcattggaaatattatttctaagctaactgcccaaggtcacattaataataagtgccagagtccagatttcaacacaggtgtatcttactccaagtctatgatgttggccactatcacattcttaatatattagaataaattactgggcaaaaagtcgttctgtttaagctcacttaagtatttgattctgatgtaccttgttatttttctgtggagtacctgagtaagtagagaaagtgtagctgattatgtcttctgtattttcaatattgtagaacctaaccaaaatttgtaacattaaaattataaacactcatctagagttgaggaatatgtcttcaggaacatatcttctgccagtgtacagggcctagcacttcgaaagtggtgatgtcagcataggaagaagcaggaaagagaggagagtcatgggagtgcctggaatcaggagagggagtagagacccagagtgttacagacaggaggggaggatatttggtcagtgaccaaaatattgaggtcacacatgtcattacataccacacataaaatgccagggagactgttcacatagaattaaatgttgtttgcctgtcagggtgtccaaacagttaaagtctcacttttaacgaacattattatagtgccctcttctatataataaagtcatttttgtgtcatagcagcaaatttctatataatagaagtatttgtaaatttctgatctgttatggcaagtctgttagcgaggatgtatttacagtacattatgtattttaaataattttttttttttgagacgggagtctcactctgttgcccaggctatagtgcagtggcacaatcttggctcactgcatcctccttctcctgggttcaagtgatccttgcacctcagcttgctgagtagctaggattataggtatgcaccaccacacccagctaattttttgtatttttagtagagatggagtttcaccatgttggccaggctggtcttgaactcctgacctcaaatgatccacctgcctcagcctcccaaagtactgggattataggcctgagccatcacgcctggccagtatttaaaataattttaagtagacaaatataagtaaaaatactggcttcaagaaaacacattaaaatgtttagacccattattttggggtagttaatttttatttttattttatttttttttgcgggggagacggagtctcgctctgttgcgaggctggagtgcagtggcaccatctcagctcactgcaacctctgcctcctgggttcaagcgattctcctgcctcagcctcctgagtagctgggattacaggcacgcaccaccatgcccagctaatttttttgtatttttagtagagacggggtttcaccatgttggccaggatgatctccatctcctgacctcgtgtcccacccaccttggcctctcaaagtgctgggattacaggcgtgagccaccgcgcctggccatgaaattattatcttaatggttttctcccacctaccccatattcctctaataaatttgtgttttctactatcaaaaaaaaaatccatcagaagtttttctgtgattatttttgttggttttgttttattttgttggtgtgtttttgtgttattaagtactggaatacacaaaattgcttgatttctggttcagtgcattggtacattgtcttggaaccagaggtttcttggtgaacaggctggtgaatatagcagaagtctatggtaagactccattatgacttaagaaagtatattcaggttttagaagtgattctcatttagagatggataccccgtgaagttaatgaaatgtaagctttagatgcttcacttccaagacccatacttagttttatacaggcatacccccattttattgtgtgccactttatcgtgctccacagatactacattgtttacaagtcgcaggtttatggtaaccctgcctctagtaagtctcagtgccattttcccaacagcgcgtgttgacttcatgtctctgtttcacattttggtaattcttgcaatatttcaaactttttcactgttattctgtctgttacggtgatctgtgatcagtgatccaaatttgaggttattgtaattgttttggggtgccacaaaccatgcccatataagatgccaaacttaattgataaatgtgtgtgttctgactgttccaccaagcagctgttccctggtctccctccctctcattgggcctctctattctctgagaaccaacaatagtgaaatagcccaattaatagccctacaatagactcttaagtgttcaactgaagagtcgcacatctcttactttaaatcaaaagctagaaatgattaaacttaatgaggaaggcatattgaaagtcgagataggctgcaagctaggcctcttgcaacagttagccaagtcatgagtgcaaaggaaaagttcttgaaagaaataaagtgctgttacagagaacacatgaatgataagaaagtgaaacaaccttattgctgttacaaagaaagttttagtgatctggagagactgtcagccgcaacattcccttaagccaaagcctaatccagagcaaagctttctcttcaattctgtaaaagctgagagctatcaggaacctgcagaagaaaagaagctagcagagtttggcttatgagatttaaggaaggaagccatctccataacataaaaatgcaaggtgaaacagcaagtattgatgtacaagctcagcaatttatccagaagacatagccaagataattgatgaaggtggctacactgaacaatacgaaattcagtttagctaaaacagacttctgtaggaagaagatgtcatctaggactttcatagctagagagaagtcaatgtctggcttcaaaggacaggctgactcttgttaggaattaatgcagcttgtggctttacgttgaagccagtactcatttaccatttgaaaaaatcctaggcccttaagagttacagtaactcggttgggcatggtggctcacacctgtaatcccagcactttgggaggctgaggcaggcagatcacctgaggtcaggagttcgagaccagcctggccaacatggagaaactctgtctctattaaaaatacaaaattagccagctgtggtggcacatgcctgtaatcccagctacttgggaggctgaggcaggagaatcgcttgaacctgggaggtggaggttgcggtgagctgagattgtgcagttgcactccagcctgggcaacaagggcgaaactccatctcaaaaaaaaaaaagaattatggtaactctactctgcctgtattttataagcagaacaacaaagtctggatggcaacacatctgtttacaggatgtcactgaaaattttaagcccactgttgagatctactgctcagaaaagtaatacttctttcaaaatattactgctcattgacaatgcacctaagtcacccaaaatctctgatggagatgtacaaggagattgatgatattttcacgtctgctaacacagcatccattctgtagcccatggataaaggagtaattttgactttcaagtctgatcatttaagaaacacatttcataaggccatagctgccacagatagttattcctctgatgaatctgggcaaaatagattgaaaaccttctgaaaaggattcaccattctagatgccattaagaacgtatgtgattcatgggaggaggtcaaaataccatcattcacaggagtttggaagaagcagattccagccctcatagatgacttttagaggttccagaaatcagtgaaggaaataactacagatgtggtgaaaagagcaagagaactactagaattagaagtggagactgaattgctgcaatcgcatgataaatttgattggatgaggtgttgtatctcatgggtaagcaaaaaaaaatggtttcttgaaatggaatctactcatggtgaagatgttaagaacattgttgaaaaaacaagaaaatatttacaatattacatacactaaggtgatagggcagaggcagggtttgagaggattcactccagttttgaaaggaattctactgtgggtaaaatgttaccaaacagcatcattatgctatggagaaccatcttttgtgaaaggaggagttagtgtggcaaactttattgatgtctcactttaagaaattgccacagccattccagccttcagcagccaccaccctgctcagtcagcagcttatccacatcgaggtaagaccctccaactatcaacaagaatatgactcatgaaggtgaagatgattgttagcatttttggcaataaagcatttttaaattatggtatgtattttttttagacacgtactgttgcacacttaacagactacagtataatgtaaacataatgtatatgcactggaagaccaaaaaaatagtgtgacttgctttattttgatatttgttttattatgtggtctcaaattgaacctgcactttttccaaggtatgcctgtattctgtttcttagaggaactccacatatttgtataagattggctccaccttattctcactgtgtgtgactatgctggtcataaatgtgttcatacccactatgtgtctataagacaaaacctattctgacttgtcttttcccatgctttttcatctttctactctaggctaagagtagaggtagatcttggagaagagggacctgacaaaattttaatgtagttgtctttttctttgacaaaggaaggaaagcccatctcaagaggtcctttctttttctgtgttttgctttctccatcatttcttaagaggtcaaagtgttcctgcagccctctgcctgtccaaaatctcccattgccaccctgagtctccaagacctgctttaccctcataattctcaaattccaaatttgtgtcccacattctcattcacttcaaaaattctgttctttggctgggcatggtggcgcatgtgcctgtaatcccagctctttgggaggccaagacaggaggatcacttgagcccaggaggtcgaggctgcagtgagctatcatggtgccactacacttcagcctgggcaacagagcaagaccctgcctcaaataaataataataataataataataataataattctatttttaaaatatttactgttaggaaagcagtctgcctaagccatatgattatgccacgaggatcatgttgcatttctgctaattatcacctttgttttcttctaggctcgtttggcttcttctcacctcctccttctcacttcagctttttgggtgttgaaaatttctaaactttgctctttttccttatcttatttagataatgacaccatttgttgtgttatatggatacagttgtctatttttttcttattttcacacagcccagcatgttttgggagtccctggcagacagttttgtttacttctagatattaaaactgacatcggataagactatcactgggttgctaggtgtatatgattaaacatcatctacccccaagatcagaagaggaaaaatagcgacagattcttaccttttataaaggaagggtagtggaagcacagctccttctttttactccgtatctcatagactgaaccttgcaaaggaaatttttttattatgtaaagatagttgtaattgttatttaccacttatggtaaatcaggcttaccatttatgcctgattactgttcagggtaacagtagtagctctttaacaacaccctaagccattgaaatcaatacctggagggagagaaaatatagtattgagatactggggtggggtggctgattagtgacagaacaccaaggcaactgcagttttcttctagtaaaattatgtcctactgtgtttataatcaagtaaaagtgaaagtatattttgtaagctatgtttactttcagcatattatctgtttacataggttctgttaaatattatttactgggttatcaacacaacatttaaaaaccagaattgcatgtacttgtactatcaatgtttgtactgtcttggaggttaatagataaaaagcattttaacagtttaaagaacatcattatcgtttgtatttttattttaacagaaggggacgaactggtagtacctgaaacatcagataatagtcggaaacaaatggttagaaacattaacaataagcggcgttattccttcagagtcccagaagaggaaaggatgcagcagaggaggtagatacttaacctgtagtatcatccatgcttgcttgtaccatcaaggactactcggtgatgatactatatttgatttggaaggaaaattgggtatctgaaaatctcattgatctgtggtgtcaaaaactgcaataatgaatgtaacttctcagtgtcattttaattaatattctcaaaattgtgagtatgcactggattaaagtaccatatttttattcacttcattcacttctctatgtatatggaatagtcatatttttggattgtgtacctttctacagtataagcataggacatagctgtgcatacagtaaacctgtatatgtctaagaagttcagactaactactctcttcatgagcaaacagaagtaggactgtagtatttacagttatatataagaccgtttctaaagtgttaccaatcatttttaaagagatacataaaatgttcagaattatatgtaaaacaaagtagagcttctttgatgtcttaagtatttaaaaagatcctctactattccacctaaaatcgtaagatcttttaaaatttaaaacaaaatattctaaggaagtacatttttaattgagtatattctgttactctatattcttattaatgtgatttaacagttgtatttttattaagaaaattaagcttaatttttttcataagctacataaattaactgtggaaaaagtatctaaacagatgccttgtcttacagggaaatgctacgagatccagaaatgagaaataaattaatttctaatccaactaattttaatcacatagcacacatgggtcctggagatggaatacagatcctgaaagatctgcccatggtaagaggaattaagagtgagtgtaaaaaagggcgggtgtggggggagtataaatacataggcttaaatacatgggtagacttcaaaggatatgtgaaagtaatgaggaaatgtttctctcagtataccacaatatgtgtaagatacatgatgacatacctgatgaacaaaacataaaatttagacatctgtcaaaagccaaacaaatcaggttcttacttagttccatattggagaatttctcccatgcctaggtttactattggtggtggaagaaactaccaaattcggagaggttctataccacatttggaatgtggttaccaaatcactaaaatttcactacgtgttagcaaatcaatacagatatttggcctactctttttttttccttcttcccagagaaccacattagcttggttattaatggtcacatctctccgacagttgtgaaattaaatcgttttcctttgcccatttacaaacatgttacctggtcagttgtacgtgaaaaagtataacataggccaggtgtagtggctcatgcctgtaatcccagcactttgtgaggccaaggcatgaggattgctgaggccaggagttcaagaccagcccaggcaacataagcaagaccccatttcttttttttctttttttgagatggagtctcattctatcacccaggctggagtgcaatggtgtgatctcggctcactgcaacctttgcctcccaggttcaagtgattctcctgcttcagcctcctgagtagctgggattacaggcacccaccaccatgcccagctaattttgtatttttagtagagatggggtttcaccatgttggtcaggctggtctcgaaccctgaccttgcggtccacccaccttggcctctgaaagtgctgggattacaggcgtgagccaccaagactgaccaagaccccatttctataaaaacattgctggtcatggtaatgtgtgcctgtagtcctggttactcgggaggctaaggtgggagggttacttgagcccaggagtttgaggctgcggggagctatgattgccgttgcattgggcaactgagcaagaccctatctcaaaaaaagaaaaaaaagtacaacattggagaactattctgttttaacaatatgaagattaggctctcagaccggtcatgtttaaattaagaatattagtggaattagccaggctctttacatttcagtaccatagttctctcagcatactattaaaactttaaaaatgacccttttgattggtagtgatagctgctaaattttgcgcagaattttaagtataattcatatttcatggccatttgggagatgatttgtgaatcttgacccaaattgctctctaaggaaatctgtcttaattaaccacattatcaacactacagtgatagaagatataaacatctatcactgacagagaaattgattgataatataattcggtgtgcagtcaagtttctttaaattaaaaaaaacgccttttaaagggaggtttgcatttatttcatgggaagaaaacatatctgcattcaagtctgagttcttcattgtaaatatctcagttcatcagttggctctgcttgcttatgaatgaatgtgacattcttattaccattaccaataatatcaaaatattattatatattcatagtaatatacttttctttgtggagttcaaaccagcctcattaatcacttatgtttttggaatagggttggctgtgctgagtaaatatttaaaaagtgcaaaaactagattacagcttatcttgatatagtacttttaaaaattttcaggtcaagcacattaaatattaaaactattatacttttctatcatgtttaaaagctatactatttttttttcaacctttttctgggagtgagatctcagatttctctccatccttttcccattaggaacttttgcttaaagtcctgagtctggttttcctttaacctcagaagtaaacatagcataactatagagtccaaagcatgttgtttttccttatggaaggtgggatgtcaggcacaaaataccgtaaagaattttttaaattgtgataaacaattgaaatgtgaatatatttatgacttatatttgataagtcaagtgctgtgtagtattttaattaaactttttaaattgagttagtagttttttcaatacataatatgtagttagaattatgtaggggtagcaggatacaaatttcaaatatgtaagttgcaaagcaaaacatcagcaaatataaaattatttcagtagttacaaggttttaatagatagtcttttaaaatttaaattttttacacttttgtttcattttacacagaaaagaatgtttttatctcatacattccaacaatgtagctagaagtagtatagaaaatgtttgtagacattctattctagcccgcatactacagtgtttaacagaatgttactctgcttagtatccttctgccatcaggtgaaaaatcagtttgctaatgttccagagttctttcagaccttcatatccaataagactatgcaccttgatgccaaaatgtcagatttcaaacattataagaactttttttatcagagatccctcctctacattttcttccctcttccttccaccactttaagaaaaaaaaagagataaaaagaaaaaatctgtatcttattttttgttcaaaataaggtctatattgataagcaaaacaagtgaagatagcattttccacagaatcattagactgcttggtttttagtgtttgttgtcacctgattttgttaacacatgataatcgttctttttggcattaacacaagtgactttttgccctctcattgcatttcagtttttaatgtacctttcattgctttaatgttttcttatcacgtaagtgcttttgtatttccaggacaagaagttggtgtaaatttttgaaacctgtttcttcacctgtacatgtagtatgcatcaaagattttatctgtctgaattcctcttcttcacaaccagattttattttcacatgacttttttctatttcactaccaaaggacatataacctggggcttttttataggcatccatttaatcatctaacaaatatttattgagcacttactttgtgcccggaaccatctttatctcagagtagacactagtaaatacaataatatgcatgtctgaaaataataataatatgcatgcctgtacttgtgtatgtatctattgaaaagcttgtgatattttaatgtattctcagttgttaatatgttctttccattgaaatggaattattacaaactgtctcataaaataaagatgaattaaatttatctaaagttaacatatatgtttttaaataccaaagccacatgttgtcatctattcagagagcatgtgtcccagacatttttatattttaatttattcaaatcagtattgctttcctgtgaaatacttataagcttagaaagtttattgtatgtctttagaaatttaattctgttgcttttgtgcaagatgttcttgaaacagaattgtttgctttttgaatgtggaaggagaagtgaggctaagggagtcatattcctctatcagatgcaaacctttatttgtatctaagtgagccaagacaatataagcattaatataagatggtgtgtgtggcttataaatagatgctatattcattttcttgaatttttaaaataatttaccgttttaatacatggttgctatttgtgataaagaacaaatttttctctaatttggatgcaagaataatactttacctctagaagacttatttttagcacattacatttctcctagctttttatccattactaaaattgttaataaaagtatattttcgagcttagtgacatcctggagctttaagatgtaattttgcttcttaaagtcaaagtttcatgtacgtcagtacaacctcagagggtactgattttattcatatatctagaataaaaacaagaccttacagaagagcctttcttctaaggctaggtaatttatgcaatctttttacaaagcaggttcatctcagctcatgggaacgtgaattccatgctggattcagttagcactctctccaacacgccattcaccctctgcctctaatgaagggatcatgttttctggaaacataactgttcttcaagaactgaatcttgaaaattcccattagagaaggtgactggctactgagtgatagttcctgcgtaatcggaagtagttcttttatctctaaaaatagtccttggattcaaaagcttttttctatatctttgatattacattatttcatttttaaaaaatatacctgaacattgaagggaagatttttaccttgttaagaaaacaaacatgttctttttgggtgtctaaatggagataagcttgggctgcttaaaggtaaaccaataattagtatattccatggttaactgttataaggaaataaaatcagtaaaaacaaacacattatcagggaatgcttttacatggttttagatttctaaagaaacagtttcataacacgtctatttagaagctgttgtaggttcacttgttcatgttcttgcttgtttcttgctttttctatccttaatgtttttgatagatttttgccatattttgattgtgttattacataagaaaatactgaactaaactctgaaagtcttgtatctctgaaagtccaatttctccttacttattttaattaaagaataattttataaaaaagaatgatcttttgcaaatcaactatttaagtaactggcaagagtattacaaaatttaactttctaagacctaaaagtatttcattaaggattcagctctgatatcagatactattccatttgttctgtgtgcatgttttgtgaaatgtgaatccattttcatcaccttattgccttttttcatctttcttttgtttttgatagccaggtttcccttatccatcccctcatcatcactccggtctgatctcctctccgatcaactttgaacacatctatcacatgactgttaattctgctgaaaaatttctctctcctgattccataaaccctgaatattccccgtctctgcgctctgtccctggtacaccaagctttatgactctgagggtatgaacagctgagtcaggtgttaactttcactaaccatgtatggatttccttttgcctggcttaataagtaacaccgctgaaatggtgccatcttgtgaaccttttcctttggcctatttctcttctgccaaacttctcctcttcctcttcctccatgtatcttcctcttctttcccatttctattttgtttcttcttttgaggaagaagatggaggaatagggaagatttcaattttcatttttgaaacacagtattaaagatttatctatgtttaataaaagtttttcccattgtaagaaatagctctctattctgttaaaacaaacaggaaagggctcccagaatttctgaattttttagctccttaccctataaccagttcaattcagtgaaatgttttatatttagtgaggttaaagtctgtggtagaacccatccacagacctatctacaataaacccctttttgaaaagcttaaaaatctccaaagtgaaaagttaaaaaattaatattaatgagtaacaaaaattccaaaaataagttagattttactaatctttgagttactgaaatgcatgtaattccaaccaggatccaaggtaagaaaactaaagcaaagaaagaggaaaatttaaaaagcatgctgtattctgaaggattgtggcagctgctgttggtctcttccattcagcttgtatttttgctaccagtggctttgcagtagggtggagagctggtggccattaaagctgaatattcatatgtccatcaagtggtgtatgcttacagtaacttcaagggaacattttctgaagttagttttgcaggtaataaaatgtttatattgtatcatgcaaaccacttcccataatgtcccctaacttacaacaatagtagatcaatttttgtatttatatatgtagaaattttccaattagaaaattagctgtaggatgggaagtttattttttgttgtttgtattataaaattattctaaattataatgttttaaatgttactgacatttaaattattatatcacttaaatatcttatctgacaaaattaaattaaccacgataaaataagcctgagtttgcttagactgcagggagtggattccagaggaattttgtatatgttaatcaggaacttttcactgggcatgatggctcatacatgtaatcccagcactttgggaggctgaggcaggaggaccacttgagcctaggagtttgagacaagcctgggcaaccatagcgagaccccatttctacaaaaaataaaaataaaaaaattagttggatgtggtggcacatgcctgtggtcccagctacttgggaggctgaggggagaagatcacttgcgcctgggaggtcgaagttgcagagagccgtgattgcaccactgcactccagcttaggtgatagagaaaggctatgtctcaaaataataataataataacttttcatataaaagaccatggaaatgttgctttagttagtcatctataactagaaatactgtgccatcaaatgaaagtctcaccctaatcttatacatatttgtaccttgaaaggaccagaggcttaggttacttgtcactttataatgaagtttatttaactctagcggactgaatgataaactgttttttaacaaaacttttttttgtcattcctttattacttcagcctaagtaataaagctactaagttgttactaagtttcagatacagtttgtagccttaatttttgctatttattctaggataggtttgttttataggaaaggacaacatcaatgtatcttaaggtttacagaaaaacaataaaaaaatgtatagcctgaaacggatattcccatctcctttgagcttttctttgctgcattgttcacatgctgcttgcgtcttacagggttgctcttgtatatgtgtctgttgaattgctagcattgactatttccattttaatttttttattttcttagagaaaaaaaaagcatttgctttaaataacgcaccaagaattacatggactttgcaaatttggtagatgaaccaaattgatgtagatggcatgtagatggcagcagttaaatttggggccaactagttgccaactttcagtatgtgcgattcttcaaaatgtatgctttggagcttttcaagtggtattaatgcatgttttcctgagtgctggctttggcccttttttagttgttttgccataaccttttctcaggcatttcaggtgcatgtaggaaagctcagtgctgcaaactttctctgagctctaacctctccttggagtgagtttcacaattgcatggcaactgggaactcagtttccagttctagacttttattataacttaaaaatattttttagccacattagtacatttctttattgcttgtttcttttaacctcaaaagccttaagtatttgtgctgatttcattgtattctgtgttaaattatttaatctataccatattatagcaaagaagatagtcatcttttaagtgtttgcataaaatgttattttgtatctttaaaacacacacaggcctgtaacttcaaatcaatattaactaaacactgttttacagaaaggatcatattttgaaattacattgccaactgaattaatctcaatactcaaattagaaaattaggattgcagatataatctattcattttccatttatttattcagcaaatatctttcagcacctaactattttaggtgctggagagtttagtgttgttgtaataacagtcagtataagaaattataagatgactaaagaactgaggctagggaaccatatgcctgcttataaccaggacaaactgaactgtcagcccaaaagctgaggaccctgaggtggtgagtgtggaggagggtgtgcctaccaacagtgttcccctgggtttctatgtttaaagggcaagtatgaggacgtttagtaaaatatgtaaaatgtagtttaagcatttaggccttttctcttgctgaaacttgtaaaaccatttaggtaagatagatttctattaacgttaaatagcataagtcatgaataaacataagtcataccacaaagtttatcaatgtatggcaggtatgtggataaatttccctattgtattctctatgttaagctactagaagaaagagtaatattttctgagcccgaaatgatgagaaaagattgtgtttttgaaacagcaaaattaggcagacagttgtaacatttttttaaaaatacactagttagcttggtgttctgttgcatttgaaaatagcatgatacagtatcatcaactcttaaattatgttggaagaaaagataggaaacacatcaaattgttattagtatctggtgaattcacaggtgacttttttggtctcttataatttcattatatattctacagttcatgagtattacctttataatcagaaaaaaagcaaacaattttaatgaactagttagcactgagagaacaccaggttaataaatatatcttccagtcacatcttgttatgtcttagtggctattactatctgtgtgattttctgatgatgtagaaaagataaaggattaccctatagtaatggtagcctatagcattctatgaagagaacatctgttttcaaaggaaagttgcatgttatagtttaacttggaattaaactatttggctttacttaacaaattgatcctttggaaaagtcacaaaacttgaattttttactgcttttaatgtgtgattttttttcattgcatttccattatagagttaaggtattgtttaacacatgtaaaatttgtgatccccccaccaccaagcagaattgctggggggttgttgttgttgttgttgttgttgttgttgttgttgttgagacggagtctcactctgtcgcccaggccggagtgcagtggcgtgatcttggctcctccagcaacctccacctcccagattcaagtgattctcctgcctcagactcccgagtagctgggactacaggcatgtgccaccaaacccagctaatttttttgtatttatagtagagacggggtttcaccatattagccaggctggtctcgaattcctgacctcgtgatctgcccacctcagtctcccaaagtgctgggattacaggcctgagccaccgtgcctggccagaatggctgtttcgatgtgactaaaaggaggtttgaaagtgatactgcagatggctgtatgaggttgttactcatactcatgaggaaagaaaggcacatagtgggactgggctacatgcttgctttttgctagtgtaattagcttcatcctcagacaagccccaggaggcagttcttatcctcataatacagctgagggaactggggcacagagtttagcaaccccaagccctgctgtcagtcagctgtgcagcacagatgtgagtagtgggccaaatccctccatagcctgtgctcacagccaccatgctgcctttatggcacaccctcacaaacagccaatgaaggagaggctggctgacaataggctagtaagattaccatcatttttgtagctatgaaagaaaaatcaataaggagtagataaatcttctaagtaaatacatataaatctgtttataaattgtaagaaatcaaatccagaccatttgcatcagaagatgcttacatgaggctgggattatgtgaagaaatggagaaaagccttatgagtgttaaaagatgttaataacatggtatatgtaatttgtcttctccaggatttataaagaatattaaaatatttttccctcctctggacttgtgtcagccccaacatctcatcctcctggtctgaaaaagattggggcctttggggaggcagcatgctttctgttctcaaccgtaatcagacagctcaccctgcacaccacctatgtggctatcctttaggcgtgtgtgatttgcaaaacattctgtggcctattttcctacctcacacactaaaatcattcaataattaaatggaaatatcaataaggagtaggtaaatcttccaagtaaatgtatataaacctgtttataaactctttgaaatcaaatccagaccatttacatcagaaattagttttcaaactaatattcccaaattaactaacttcacctaggttgtgagtattgcagcgtcttatttctaatcccagtactactattggtgtgagtcagtaaagatgcctggtgactggggtgtgaacttataaatggcacatttggtatcacatcaataaaacattttcttgagaagaatataattttagtagaattttctccaaatggacgtatcagctgttaagcaaataaaaatgtcatacatagtaaaagctttcagttatcggcaaatgtccatgtaattctacaattcactgtcacatctaaccatatagcttttacttactgtgtttacaaacaaaaaaatgctgttttttactgataagtatttcagtaagtataagtacttattcctgattaggatgagatttacttcatgttagggttttattacttttgtgggtgggaaagactatataatctgccattccttactctgtgtcattctggtcaagatgattttatttcttggtaattatagtgaaaaatagacatctctgcagaaaaatcagtttctttctggctaaaggatgtctctctcgcttttgtttatagaaccctcggcctcaggaaagtcggacagtattcagtggctcagtcagtattccatctatcaccaaatcccgccctgagccaggccgctccatgagtgctagcagtggcttgtcagcaagtaagtgccggggctacaggaaggtgcctctgagacagggtgacccccagctccctcccctcctgtcccgtgggtgacatgtccttcacttacgtgtgcccattgcattctcaagtggctgcagtgtctcagaccctgctgggtaatgcctaataggcacaaatgcagttgttaagaaaatagtcccagagtccttctagagtgtacaggccatctgggagacagacaaatatgattacaaattgtgatgataaaggctctgaaggaagtaaacagcatacattaatagagaataacaaggggtagctgttagggatgaatccctacttggcagaataattaggaaaatcactccctagaggtggagtcatgtttgagtaatgtttggttaactgaaagaaggctagtatggctacatggtagtggtgaggaagtaacaaaaattagagcggggtagcaggtaagggtcagaccagcagggacttgaagaccaaggtaagacattttttactttattcaaaaggaaaaggaagacttttaagtagggaagaattttctttcaatttacattcttaaaacaatcctgcgggctgccaagtggagaatggactagaggcaggaagagtggaagccagcatccagataggagactcctagagtggtcccaatggaaaccaatgagggcttgggatgcagcaggggcagaaggagagaagatggtagattctccagatatattttcagagttaaaagcagtaagacttgatgatgaattagtcatggaaaagtaagggagagagttaaagatgactcccagacttcctgctagggccttagtatgataccatttactcccatttaccaccgtttaagaaggggctgaggcaggacattccacgcatgtccaaaggtcccgaggtagcaaaaaaaaaaaaaaatgtatctgtttgattcactgaaagaaggccaggaatggattcaagaatataccctttactacaataaagataacattcatgcagtacacatgtttgaggttccattagataccaactcttctgctgaggtgctgggggtaaaaatacatacatgacctagtacctgctttcaggtgtctcacagtctttattagtctctctctccgttttttttgtttgtttgttttttgtttttaactgtactcggaactgcagcatctctttttttacactttaaaactagaaccttgagctaagggaccatagagagaaaaaactgtgctccatggtgcacttaggttcagctactgtcgcggagcagctttttggtgggggaggaaaggtgtgtaaaaagatattgtctgcctgaataaaattatacaaagatgctaaaggctacttacctcaaaatgagagaaatgtacagatgggacatctggctgatttaaaaaagtaaattttactcccgtcaaattcactgcgatctaagatatttcctggattataagcagtgcgattgtagattatcaagtgatcattgtagcttcacttggggaagaatgttcttggctgaaggttgcttttctgtaactttctcaagtgtgtgctttcccgggagcatggtacttgcttggagatcagagagagtgtcgatcgagtgtgccagcgtaagtgcagtccccaagtgtaccaggtgcaacttagtgaaaccttttttcacctccatgcaaatgaactttaagagaaagtaactgcactaaatccaatttcacatttaaaagtcattgagtcagaaaacttacttgttcccttctattctcgaatgttacatattttgaattcaagtcttctgatgcagattgagtttcctccattcatttgctcttcattcagcaaatactgattttggcacccaatttgtgcccaggtattttttttttagattctgacgacataaagatgaatgatgctgtattagttatctcttgctgcataagaaattatcccacaaattaggagctttaaaatagcaaacatttattatctcatagcttaaaatagcaaacatttattatctcacactgcctcagggtcaggaatctgggaacatcatagactggatggctctggttcagggtttctcatcagattgcagttgagctgtctgctggggccacagtcacctcaagactcagctgtgactagggaatcagtttcctggctcacccatgtggttgtggacacgtctgagttcctcaccgactgttggttggaggcttcccgtttttgccaatgtgggcctctccatacccagtgcctgagtgtccttatggcctggctgcaggcttcctccagggcaagtgatcagagagacagaagcagcagtgttctacctaacctcaggagtgctgtatcaccacttcttcagtatgctgttgatcacacagaccagccctggtactgtgtgggaagggactacatacgagtgaatactagaagggacgatcatcttggagcctggctacagacatagagcatgctttcaaggagctttcaattgacaagaagatgttgaaatgtacagtttgcaactttcttgccattatctcctaacatcatctgtacccagtatgtagccctgaccacaatgaaaaaatcttgccaagtgggaaaatgacaaccacttttattaattatctttttattagcgatagctagatttgatgattgatgtaagctgttactaactataagttatctagaagtcagttacaaaggttttacaactttaggaattaagtgatggtccataattccttacctgaaacccttgaggccagaatgtttttatgttttagaaagggatctaccatatattatgttatattcccagcaagatccatgacaaacaaacacattaagttcccacgaacacattaatacttacacagcaaaagttatgactgtttacactgtaaatataggaataaatgtagtctttataaataaagcctaaaatcggcctccaggatttttttgggttttgttgttgttttgttttttgttttggttttgcgtttttgttttttgttttttgtttttgttttttttgagacagagtctcactctgttggccaagctcgagtgcagtggcacgatcttggctcactgaaacctctgcctcccaggttcaagcaattctcctgcctcagcctcccgagtagctgggactacaggtgggtgccatcacacccagccaatttctgtatttttagtagagacggggtttcaccatgttggccaggctgctcttgaactcccgacctcaggtaatctgcccgcctcagcctcccaaagtgctgggattacaggcgtgacccaccgcgcccagcccagttcacattaggttttgtcatcacatgaatttgcatcaagcttgtgaaaaaacttttggttttcagcacttttggatttgtggaattgcagataaagataggccctaaactgcaaagatttaaagagccaagacagcccatctccttagaagtgctgaagtgaggctcccatatcactgctgatggcatgcaaacgacagactggagctggcctggcgttcttgtcaaggcccttaacgcattcattgagtttggccttttttatgtccctggttaccagctgcttgtccagtaaagatataagaggaaagaccctctcgggaatgccaaggggcatctggtgtttagagtgcctcctgctggcttcaagactagcccagctcatataaaatgtttccagaaggacagccagccaagagaggaacaggccaactatgttcagtctctacagcaagaagcacaggatcttccacagccagactcaacgatttcatgatgcaaagtcagattgaaacagcggtgctcaccatgcttccttccctccttcctagttcacctgaggaatgagacccgctcctgtcagcagtaggggctcactgggagcctcccagaccctcctggaagtcatgcagaggttggatattgctcatccccaagagatttagggcccagtccccagagtcaccacttaaaactctctggaaggggctgtatcctgagtacagtatgtggtctcaggccagagggtccccaggctccttttgctgttcaatcaaaacagccctgccttgatttttttttttttttaaaggttgtagtgtttgaatgtaacctttctttgaaaagaaaaattcctgaagtttaaaagtgaaaaaaacatactggaaatcactggggtgggattcagttcagttatctgctctataaatgatcactaggaaggagggaggcagtgaaaagaccagaagcacagtgtgggccagcacacaccttctgtcttgcatgttactctccatgcattgagtggtgagttgttcccacaaagtcctgtaaggtagaatttatttttttacctaaaggtgaagaaaatggaaactcaggaaggtaaaataacttgcttaaggtcacaccattagtaagcagagcagtggaagtctctggcagtttgactttctgtgcagaataacaaaatgttaaagtaattttttcattattttttgaactgagcactaaactagacaaggaattggaagaaaactcaaaaaatattttatggcgttcaattagctcgtaatctaaggagacacacgttccacatcaaaaaagtaaagagtatatttcccaagaaatatgtcaacaaatgaagaccagtgcatgtggagaaacccagtggtggggatggagtgttaggggaaacgtgtctcctgaggagtgaggttgtcagtgattctcagtccgaggggccttctggaatcatcaggagagcttattattattattattattattattattattattattattatactttaagttctgggatacatgtgtagaacatgcaggtttgttacataggtatacatgtgccatggtagtttgctgcacccatcaacccgtcatctacattaggtatttctcttaatgctatccctcccctaaccccccaccccccaacaggccccagtgtgtgatattcccctccctgtgtctgggtattctcattgttcaactcccacttatgaatgagaacatgcggtgtttggttttctgttcctgtgttagtttgctgagaatgattgtttccagcttcatccatgtccctgtaaaggacatgaactcatccttttttatggttgcatagtattccatggtgtatatgtgccacattttctttatccagtctatcattgatgggtatttgggttggttccaaatctttgctatggtgaatagtgctgcaataaacatacgtgtgcatgtgtctttatagtagaatgatttataatcctttgagtatatacccaataatgggattgctgggtcaaatggtatttctggttctagatccttgaggaatcgccacactgtcttccacaatggttgaactaacttatactcccaccaacagtgtaaaagcattcctaggctgggcgcggtggctcaggcctataatcccagcactttgggaggctgaggtgggtggatcacgaggtcaggagatcgagaccatccttgctaagatggtgaaaacctgtctctactaaaaattcaaaaaattagctgggcatggtggcgggcgcctgtagtcccagccactcgagaggctgaggcaggagaatggcgtgaatctgggaggcagagcttgcagtgagctgagatcacgccactgcactccagcctgggcaacagaacaaggctgtgtctcaaaaaaaaaaaaaaagtgttcctatttctccacatcctctccagaatctgttgtttcctgattttttaatgatcaccattctaactggcatgagatgctatctcattgtggttttgatttgcatttctctaatgaccagtgatgatgagcttttttttgtatgtttcttggccgcataaatgtcttcttttgagaagtgtctgttcatattcttcacccactttttgatggggttggttttttttcttgtaaatttgtttaagttccttgtagattctggatattagccctttgtcagatggatagatggcaaaacttttctcccattctgtaggttgcctgttctctctgatggtagtttcttttgctgtgcagaagctctttagtttaattagatcccatttgccaattttggcttttgttgccattgcttttggtgttgtagtcatgaagtccttgcccatgcctgtgtcctcgatggtattgcctaggttttcttctaggatttttatggttttagatcttatgtttaaatctttaatccatcttgagttaatttttgcataaggtgtaaggaaggggtccaatttcagttttctgcatatggctagccagttttcccaacaccatttattaaatagggaatcctttccccgttgcttgtttttgtcaggtttgtcaaagatcagatggttgtagatacgtggcattacttctgaggcctctattgtgttccattggtctatatctctgttttggtaccagtaccttgttgttttggttactgtagccttgtagtatagtttgaaatcaggtagtatcatgcctccagctttgttctttttgcttaggattgtcttggctatacaggctattttttggttccatatgaaatttaaagtagttttttctaattctgtgaagaaaatcattggtagcttgatggggataacattaaatctataaattactttgggcattatggccattttcatgatattgattcttcctatccataagcatggaatgtttttccatttgtttgtgtcctctcttatttccttgaccagtggtttgtagttttccttgaagaggtccttcacatcccttgtaagttggattcctaggtattttattctgtttgtagcaattgtaatgggagttcactcatgatttggctctctattattggtgtgtaggaatgcttgtgatttttgcacactggttcaagcggacctaatacacacctacagaactctccaccccaaatccacggaatatacattcttctcagcaccacatcacacttattctaaaactgaccacataattggaagtaaaacactcctcaccaaatgcaaaggaatggaaattataagtctctcagaccacagtgcaatcaaattagaactcaggattaagaaactcactcaaaaccgcacaactacttggaaactgaacaacctgctcttgaatgactactgggtaaataacgaaatgaaggcagaaataaataagttctttaagaccaatgagaacaaagacacaacataccagaatctcttgagacacagctaaagcaatgtttagagggaaatttatagcactaaatgcccacaggagaaagcgggaaagatctaaaatcgacaccctaacatcacaattaaaagaactagagaagcaagagcaaacacattcaaaagctagcataagacaagaaataactaagatcagagcagaactgaaggagatagagacacgaaaaacccttcaaaaaatcagtgattccaggagctggttttttgaaaagattaacaaaataggccgctagccagactaataagaaaagagagaagaatcaaatagacataataaaaaaatgataaaggggagatcaccactgatcccacagaaatagaaactaccatcaaagaatactataaacacctctacagaaataaaatagaaaatctagaagaaatggataaattcctggacacatacaccttcccaagtctaaaccaggaagaagttgaatccctgaatataccaataacaagttctgaaattgaggcagtaattaatagcctaccagctaaaaaaaggcaggaccaggcagattcacagccgaattctaccagaggtacaaagaggagctgataccattccttctgaaactattccaaacaatagaaaaagagggactcctccctaactcattttatgaggccagcatcatcctgataccaaaacctagcaaagacacagcaaaaaaagaaaatttcaagccaatatccctgatgaacattgatgcgaaaatcctcaattaaatactagcaaacattccagcagcacatcaaaaagcttatccaccacgatcaagtcggcttcatccctgagatgcaagactggttccacatacgcaagtcaataaacgtaatccatcacataaacagaaccagtgacaaaaaccacatgattatctcaatagatgcagaaaaggcctttgataaaattcaacacctttatactaaaaactcaataaactaggtattgatggaacgtatctcaaaataatagctatttatgacaaacccacagccaatatcataccgaatgggcaaaagctggaagcattccctttgaaaactgcaaccagacaaggatgccctctctcaccactcctattcaacatagtattggaagttctggccagggcaaccagacaagagaaggaaataaagggtattcagataggaagagaggaagtcaaattgtctctgtctgcaggtgacatgattgtattagaaaaccccttcatctcagtccaaaatctccttaagctgacaagcaacttcagcaaagtctcaggttacaaaatcaatgtgcaaaaatgacaggaggacttttaaagttaagtcagaaactctgcaaattgttctgggatagggctgggcattggtgtttgtcaagctccatcatacgctaaacagtaaaccaccagtgttgctgatctgcagtcagtcagttccagtgtcgaggaaatgccagcgagcctgtgactgcagcccaggggcttcgcaaagaagctgaggaagggaggtggaagggggattaccaaggacagagtctgcccaggcaggctcttgttcagtttatttatgaacgcttgtgtattttggtggtcattagataaatgtgctgctttaaactatcattttttgacatttttcactgaattcttaatgtgaaaaacaattgagacatgttaaacccgcaaaaagttgaaacaagcacaacctgtaccttagagttgtgctaaaacctcaacacagtcttttggaccaatcatcctaatactgaaactcagcacgatgaaggcatgtttaagcaaaaaccttgtcatagtagggaatgctaagagctttaaataggtactgacaatagccccgagattccccagggtgttctgggtcttcccttttcttcctgtgaatgctgctggtccactgttacctaccccaactctgagaacaaaatagagagtgagtttgaagaaaacctcacttttattttacatctcaggctctttgccctgtgaagtaaaagtacttttatgttttctttatggttttatgaaaattttctagaagataagcgatatttcttcattgagtggcatggcctctctcgggatggtacaaccgagcttcctcatttagcgcatctgttttgagcctgccgtgtgccatgttctgtgctaggccctggagaggcaaagacacctcatctattgggagaatgacaggcacatgatgataacatgacttcataaatgccatgaaagagatgtgtgaaagatggctgggagccgtcagtggctgggaaagatgtgcaaggcagcaccaggcaggtggagacttgagggattctggggaattgttaacaagcagggcattggggcagatggcacagcctagagagtgctcgctcaccgggagtccccgtggctggggtggacaggaacagcgagtgagattgctggattcggggttgagctcagggcaaaggtactacagttggacttactgcgcttcaggggaggagtggtctggggcctgcaaaggatggcctgtcagctcagcagtctgtgtgctttctagagtattgtcccctctgcatatgtaaaattttgcctgcatgatgtactgtagggtcatccgcacagaatggcagcgcattaaagagggaattctctggaggaagctacagtgccaagcggcagcccatgccctccccgtcagagggctctttgtcctctggaggcatggaccaaggaagtgatgccccagcgagggactttgacggagaggtgagtgggcagggttccggagacgtcatgtatcagaaagactagggcatcttcccaggcaatcccaccaccttgggattggcctcagctgcagcagggttcggggtcaggggccagcattcactcttggccagcgggcagctaggccagtcactcgggctgcaaactgcagttttcccagaggcattcagacttctggcttttgtgtgaaaataccatcatctgtaaaggcctacagaaaggacggagcttgtatgttccgtgcctcttcctcagctcatagaaggggctttataccgacagtgaggccgccccatggggttatttggtcagggtggctggagggggcggtggtaatctgagccccttctgcctccctttctccccctccttctcattaatgttttactttgggccttacaggactctgactctccgaggcattccacagcttccaacagttccaacctaagcagccccccaagcccagtttcaccccgaaaaaccaagagcctctccctggagagcactgaccgcgggagctgggacccgtga

SEQ ID NO: 4 - DNA, 634 bp, Homo sapiens
agaaaaacaaattcagagaccgctcagtctatgttcccaaagaggcttatgacagcactctacccctcattaaaacaacccaggcagccgcaatcatagatcatgaaagaattgctttgggaaacgaagaagggttatttgttgtacatgtcaccaaagatgaaattattagagttggtgacaataagaagattcatcagattgaactcattccaaatgatcagcttgttgctgtgatctcaggacgaaatcgtcatgtacgactttttcctatgtcagcattggatgggcgagagaccgatttttacaagctgtcagaaactaaagggtgtcaaaccgtaacttctggaaaggtgcgccatggagctctcacatgcctgtgtgtggctatgaaaaggcaggtcctctgttatgaactatttcagagcaagacccgtcacagaaaatttaaagaaattcaagtcccatataatgtccagtggatggcaatcttcagtgaacaactctgtgtgggattccagtcaggatttctaagataccccttgaatggagaaggaaatccatacagtatgctccattcaaatgaccatacactatcatttattgcacatcaaccaatggatgctatctgcgc

SEQ ID NO: 5 - DNA, 27 bp, Homo sapiens
agaaaaacaaattcagagaccgctcag

SEQ ID NO: 6 - DNA, 23 bp, Homo sapiens
gcgcagatagcatccattggttg

SEQ ID NO: 7 - DNA, 21 bp, Homo sapiens
aagatggaacggttcagtcct

SEQ ID NO: 8 - RNA, 21 nt, Homo sapiens
gauggaacgguucaguccuuu

SEQ ID NO: 9 - RNA, 21 nt, Homo sapiens
uucuaccuugccaagucagga

SEQ ID NO: 10 - DNA, 21 bp, Homo sapiens
aaggatcttattcgaaggctc

SEQ ID NO: 11 - RNA, 21 nt, Homo sapiens
ggaucuuauucgaaggcucuu

SEQ ID NO: 12 - RNA, 21 nt, Homo sapiens
uuccuagaauaagcuuccgag

SEQ ID NO: 13 - DNA, 21 bp, Homo sapiens
aatgttcagaggactctagac

SEQ ID NO: 14 - RNA, 21 nt, Homo sapiens
uguucagaggacucuagacuu

SEQ ID NO: 15 - RNA, 21 nt, Homo sapiens
uuacaagucuccugagaucug

SEQ ID NO: 16 - DNA, 21 bp, Homo sapiens
aatgagcggctaacagaattg

SEQ ID NO: 17 - RNA, 21 nt, Homo sapiens
ugagcggcuaacagaauuguu

SEQ ID NO: 18 - RNA, 21 nt, Homo sapiens
uuacucgccgauugucuuaac

SEQ ID NO: 19 - DNA, 21 bp, Homo sapiens
aagacaggaagctacgtgaac

SEQ ID NO: 20 - RNA, 21 nt, Homo sapiens
gacaggaagcuacgugaacuu

SEQ ID NO: 21 - RNA, 21 nt, Homo sapiens
uucuguccuucgaugcacuug

SEQ ID NO: 22 - DNA, 21 bp, Homo sapiens
aagctacgtgaacagagtgag

SEQ ID NO: 23 - RNA, 21 nt, Homo sapiens
gcuacgugaacagagugaguu

SEQ ID NO: 24 - RNA, 21 nt, Homo sapiens
uucgaugcacuugucucacuc

SEQ ID NO: 25 - DNA, 21 bp, Homo sapiens
aagctgacgagtgaacttgat

SEQ ID NO: 26 - RNA, 21 nt, Homo sapiens
gcugacgagugaacuugauuu

SEQ ID NO: 27 - RNA, 21 nt, Homo sapiens
uucgacugcucacuugaacua

SEQ ID NO: 28 - DNA, 21 bp, Homo sapiens
aaggcagacgtactgtagact

SEQ ID NO: 29 - RNA, 21 nt, Homo sapiens
ggcagacguacuguagacuuu

SEQ ID NO: 30 - RNA, 21 nt, Homo sapiens
uuccgucugcaugacaucuga

SEQ ID NO: 31 - DNA, 21 bp, Homo sapiens
aaggatgtcctggttcaactg

SEQ ID NO: 32 - RNA, 21 nt, Homo sapiens
ggauguccugguucaacuguu

SEQ ID NO: 33 - RNA, 21 nt, Homo sapiens
uuccuacaggaccaaguugac

SEQ ID NO: 34 - DNA, 21 bp, Homo sapiens
aaggtcatgtcaggattccta

SEQ ID NO: 35 - RNA, 21 nt, Homo sapiens
ggucaugucaggauuccuauu

SEQ ID NO: 36 - RNA, 21 nt, Homo sapiens
uuccaguacaguccuaaggau

SEQ ID NO: 37 - DNA, 21 bp, Homo sapiens
aagaggcttatgacagcactc

SEQ ID NO: 38 - RNA, 21 nt, Homo sapiens
gaggcuuaugacagcacucuu

SEQ ID NO: 39 - RNA, 21 nt, Homo sapiens
uucuccgaauacugucgugag

SEQ ID NO: 40 - DNA, 21 bp, Homo sapiens
aatgcaccatatctctcggtg

SEQ ID NO: 41 - RNA, 21 nt, Homo sapiens
ugcaccauaucucucgguguu

SEQ ID NO: 42 - RNA, 21 nt, Homo sapiens
uuacgugguauagagagccac

SEQ ID NO: 43 - DNA, 21 bp, Homo sapiens
aataagcggcgttattccttc

SEQ ID NO: 44 - RNA, 21 nt, Homo sapiens
uaagcggcguuauuccuucuu

SEQ ID NO: 45 - RNA, 21 nt, Homo sapiens
uuauucgccgcaauaaggaag

SEQ ID NO: 46 - DNA, 21 bp, Homo sapiens
aagcggcgttattccttcaga

SEQ ID NO: 47 - RNA, 21 nt, Homo sapiens
gcggcguuauuccuucagauu

SEQ ID NO: 48 - RNA, 21 nt, Homo sapiens
uucgccgcaauaaggaagucu

SEQ ID NO: 49 - DNA, 21 bp, Homo sapiens
aacagttccaacctaagcagc

SEQ ID NO: 50 - RNA, 21 nt, Homo sapiens
caguuccaaccuaagcagcuu

SEQ ID NO: 51 - RNA, 21 nt, Homo sapiens
uugucaagguuggauucgucg

SEQ ID NO: 52 - DNA, 21 bp, Homo sapiens
cagaatcgctggtggagacat

SEQ ID NO: 53 - RNA, 21 nt, Homo sapiens
gaaucgcugguggagacauuu

SEQ ID NO: 54 - RNA, 21 nt, Homo sapiens
uucuuagcgaccaccucugua

SEQ ID NO: 55 - DNA, 21 bp, Homo sapiens
cagagaacatcgacttggtca

SEQ ID NO: 56 - RNA, 21 nt, Homo sapiens
gagaacaucgacuuggucauu

SEQ ID NO: 57 - RNA, 21 nt, Homo sapiens
uucucuuguagcugaaccagu

SEQ ID NO: 58 - DNA, 21 bp, Homo sapiens
caagaactgcgcagaacagaa

SEQ ID NO: 59 - RNA, 21 nt, Homo sapiens
agaacugcgcagaacagaauu

SEQ ID NO: 60 - RNA, 21 nt, Homo sapiens
uuucuugacgcgucuugucuu

SEQ ID NO: 61 - DNA, 21 bp, Homo sapiens
caggaagctacgtgaacagag

SEQ ID NO: 62 - RNA, 21 nt, Homo sapiens
ggaagcuacgugaacagaguu

SEQ ID NO: 63 - RNA, 21 nt, Homo sapiens
uuccuucgaugcacuugucuc

SEQ ID NO: 64 - DNA, 21 bp, Homo sapiens
cagctagactggagttgcagt

SEQ ID NO: 65 - RNA, 21 nt, Homo sapiens
gcuagacuggaguugcaguuu

SEQ ID NO: 66 - RNA, 21 nt, Homo sapiens
uucgaucugaccucaacguca

SEQ ID NO: 67 - DNA, 21 bp, Homo sapiens
cagacgtactgtagactccac

SEQ ID NO: 68 - RNA, 21 nt, Homo sapiens
gacguacuguagacuccacuu

SEQ ID NO: 69 - RNA, 21 nt, Homo sapiens
uucugcaugacaucugaggug

SEQ ID NO: 70 - DNA, 21 bp, Homo sapiens
caagtgtcatcagtgtacctc

SEQ ID NO: 71 - RNA, 21 nt, Homo sapiens
agugucaucaguguaccucuu

SEQ ID NO: 72 - RNA, 21 nt, Homo sapiens
uuucacaguagucacauggag

SEQ ID NO: 73 - DNA, 21 bp, Homo sapiens
cactggctatagtgtgtgact

SEQ ID NO: 74 - RNA, 21 nt, Homo sapiens
cuggcuauagugugugacuuu

SEQ ID NO: 75 - RNA, 21 nt, Homo sapiens
uugaccgauaucacacacuga

SEQ ID NO: 76 - DNA, 21 bp, Homo sapiens
caggcagccgcaatcatagat

SEQ ID NO: 77 - RNA, 21 nt, Homo sapiens
ggcagccgcaaucauagauuu

SEQ ID NO: 78 - RNA, 21 nt, Homo sapiens
uuccgucggcguuaguaucua

SEQ ID NO: 79 - DNA, 21 bp, Homo sapiens
cagccgcaatcatagatcatg

SEQ ID NO: 80 - RNA, 21 nt, Homo sapiens
gccgcaaucauagaucauguu

SEQ ID NO: 81 - RNA, 21 nt, Homo sapiens
uucggcguuaguaucuaguac

SEQ ID NO: 82 - DNA, 21 bp, Homo sapiens
caatgcaccatatctctcggt

SEQ ID NO: 83 - RNA, 21 nt, Homo sapiens
augcaccauaucucucgguuu

SEQ ID NO: 84 - RNA, 21 nt, Homo sapiens
uuuacgugguauagagagcca

SEQ ID NO: 85 - DNA, 21 bp, Homo sapiens
caccatatctctcggtgtaca

SEQ ID NO: 86 - RNA, 21 nt, Homo sapiens
ccauaucucucgguguacauu

SEQ ID NO: 87 - RNA, 21 nt, Homo sapiens
uugguauagagagccacaugu

SEQ ID NO: 88 - DNA, 21 bp, Homo sapiens
catatctctcggtgtacagtg

SEQ ID NO: 89 - RNA, 21 nt, Homo sapiens
uaucucucgguguacaguguu

SEQ ID NO: 90 - RNA, 21 nt, Homo sapiens
uuauagagagccacaugucac

SEQ ID NO: 91 - DNA, 21 bp, Homo sapiens
gatggaacggttcagtcctca

SEQ ID NO: 92 - RNA, 21 nt, Homo sapiens
uggaacgguucaguccucauu

SEQ ID NO: 93 - RNA, 21 nt, Homo sapiens
uuaccuugccaagucaggagu

SEQ ID NO: 94 - DNA, 21 bp, Homo sapiens
gaattaagcgccttgagcaag

SEQ ID NO: 95 - RNA, 21 nt, Homo sapiens
auuaagcgccuugagcaaguu

SEQ ID NO: 96 - RNA, 21 nt, Homo sapiens
uuuaauucgcggaacucguuc

SEQ ID NO: 97 - DNA, 21 bp, Homo sapiens
gatcaatgagcggctaacaga

SEQ ID NO: 98 - RNA, 21 nt, Homo sapiens
ucaaugagcggcuaacagauu

SEQ ID NO: 99 - RNA, 21 nt, Homo sapiens
uuaguuacucgccgauugucu

SEQ ID NO: 100 - DNA, 21 bp, Homo sapiens
gacaggaagctacgtgaacag

SEQ ID NO: 101 - RNA, 21 nt, Homo sapiens
caggaagcuacgugaacaguu

SEQ ID NO: 102 - RNA, 21 nt, Homo sapiens
uuguccuucgaugcacuuguc

SEQ ID NO: 103 - DNA, 21 bp, Homo sapiens
gaagctacgtgaacagagtga

SEQ ID NO: 104 - RNA, 21 nt, Homo sapiens
agcuacgugaacagagugauu

SEQ ID NO: 105 - RNA, 21 nt, Homo sapiens
uuucgaugcacuugucucacu

SEQ ID NO: 106 - DNA, 21 bp, Homo sapiens
gaatacgcctaccgatgctct

SEQ ID NO: 107 - RNA, 21 nt, Homo sapiens
auacgccuaccgaugcucuuu

SEQ ID NO: 108 - RNA, 21 nt, Homo sapiens
uuuaugcggauggcuacgaga

SEQ ID NO: 109 - DNA, 21 bp, Homo sapiens
gaaggtcatgtcaggattcct

SEQ ID NO: 110 - RNA, 21 nt, Homo sapiens
aggucaugucaggauuccuuu

SEQ ID NO: 111 - RNA, 21 nt, Homo sapiens
uuuccaguacaguccuaagga

SEQ ID NO: 112 - DNA, 21 bp, Homo sapiens
gagagcactggctatagtgtg

SEQ ID NO: 113 - RNA, 21 nt, Homo sapiens
gagcacuggcuauaguguguu

SEQ ID NO: 114 - RNA, 21 nt, Homo sapiens
uucucgugaccgauaucacac

SEQ ID NO: 115 - DNA, 21 bp, Homo sapiens
gagcactggctatagtgtgtg

SEQ ID NO: 116 - RNA, 21 nt, Homo sapiens
gcacuggcuauaguguguguu

SEQ ID NO: 117 - RNA, 21 nt, Homo sapiens
uucgugaccgauaucacacac

SEQ ID NO: 118 - DNA, 21 bp, Homo sapiens
gatgttatccatgcaagtcgg

SEQ ID NO: 119 - RNA, 21 nt, Homo sapiens
uguuauccaugcaagucgguu

SEQ ID NO: 120 - RNA, 21 nt, Homo sapiens
uuacaauagguacguucagcc

SEQ ID NO: 121 - DNA, 21 bp, Homo sapiens
gatgctagcagacactgagaa

SEQ ID NO: 122 - RNA, 21 nt, Homo sapiens
ugcuagcagacacugagaauu

SEQ ID NO: 123 - RNA, 21 nt, Homo sapiens
uuacgaucgucugugacucuu

SEQ ID NO: 124 - DNA, 21 bp, Homo sapiens
gaggcttatgacagcactcta

SEQ ID NO: 125 - RNA, 21 nt, Homo sapiens
ggcuuaugacagcacucuauu

SEQ ID NO: 126 - RNA, 21 nt, Homo sapiens
uuccgaauacugucgugagau

SEQ ID NO: 127 - DNA, 21 bp, Homo sapiens
gatgctatctgcgcagttgag

SEQ ID NO: 128 - RNA, 21 nt, Homo sapiens
ugcuaucugcgcaguugaguu

SEQ ID NO: 129 - RNA, 21 nt, Homo sapiens
uuacgauagacgcgucaacuc

SEQ ID NO: 130 - DNA, 21 bp, Homo sapiens
gatgtgaactccatggaatgg

SEQ ID NO: 131 - RNA, 21 nt, Homo sapiens
ugugaacuccauggaaugguu

SEQ ID NO: 132 - RNA, 21 nt, Homo sapiens
uuacacuugagguaccuuacc

SEQ ID NO: 133 - DNA, 21 bp, Homo sapiens
gacgaactggtagtacctgaa

SEQ ID NO: 134 - RNA, 21 nt, Homo sapiens
cgaacugguaguaccugaauu

SEQ ID NO: 135 - RNA, 21 nt, Homo sapiens
uugcuugaccaucauggacuu

SEQ ID NO: 136 - DNA, 21 bp, Homo sapiens
tagcagagaacatcgacttgg

SEQ ID NO: 137 - RNA, 21 nt, Homo sapiens
gcagagaacaucgacuugguu

SEQ ID NO: 138 - RNA, 21 nt, Homo sapiens
uucgucucuuguagcugaacc

SEQ ID NO: 139 - DNA, 21 bp, Homo sapiens
tattcggaactgtgaagcacc

SEQ ID NO: 140 - RNA, 21 nt, Homo sapiens
uucggaacugugaagcaccuu

SEQ ID NO: 141 - RNA, 21 nt, Homo sapiens
uuaagccuugacacuucgugg

SEQ ID NO: 142 - DNA, 21 bp, Homo sapiens
taaggcaagaactgcgcagaa

SEQ ID NO: 143 - RNA, 21 nt, Homo sapiens
aggcaagaacugcgcagaauu

SEQ ID NO: 144 - RNA, 21 nt, Homo sapiens
uuuccguucuugacgcgucuu

SEQ ID NO: 145 - DNA, 21 bp, Homo sapiens
tatgcagcatagaacatcagc

SEQ ID NO: 146 - RNA, 21 nt, Homo sapiens
ugcagcauagaacaucagcuu

SEQ ID NO: 147 - RNA, 21 nt, Homo sapiens
uuacgucguaucuuguagucg

SEQ ID NO: 148 - DNA, 21 bp, Homo sapiens
tacacaaccagcagttagaag

SEQ ID NO: 149 - RNA, 21 nt, Homo sapiens
cacaaccagcaguuagaaguu

SEQ ID NO: 150 - RNA, 21 nt, Homo sapiens
uuguguuggucgucaaucuuc

SEQ ID NO: 151 - DNA, 21 bp, Homo sapiens
tatagagcaccaagactcaca

SEQ ID NO: 152 - RNA, 21 nt, Homo sapiens
uagagcaccaagacucacauu

SEQ ID NO: 153 - RNA, 21 nt, Homo sapiens
uuaucucgugguucugagugu

SEQ ID NO: 154 - DNA, 21 bp, Homo sapiens
tactactcctaccaagtgtca

SEQ ID NO: 155 - RNA, 21 nt, Homo sapiens
cuacuccuaccaagugucauu

SEQ ID NO: 156 - RNA, 21 nt, Homo sapiens
uugaugaggaugguucacagu

SEQ ID NO: 157 - DNA, 21 bp, Homo sapiens
tactcctaccaagtgtcatca

SEQ ID NO: 158 - RNA, 21 nt, Homo sapiens
cuccuaccaagugucaucauu

SEQ ID NO: 159 - RNA, 21 nt, Homo sapiens
uugaggaugguucacaguagu

SEQ ID NO: 160 - DNA, 21 bp, Homo sapiens
tatctgcgcagttgagatctc

SEQ ID NO: 161 - RNA, 21 nt, Homo sapiens
ucugcgcaguugagaucucuu

SEQ ID NO: 162 - RNA, 21 nt, Homo sapiens
uuagacgcgucaacucuagag

SEQ ID NO: 163 - DNA, 21 bp, Homo sapiens
tatctctcggtgtacagtgaa

SEQ ID NO: 164 - RNA, 21 nt, Homo sapiens
ucucucgguguacagugaauu

SEQ ID NO: 165 - RNA, 21 nt, Homo sapiens
uuagagagccacaugucacuu

SEQ ID NO: 166 - DNA, 21 bp, Homo sapiens
taagcggcgttattccttcag

SEQ ID NO: 167 - RNA, 21 nt, Homo sapiens
agcggcguuauuccuucaguu

SEQ ID NO: 168 - RNA, 21 nt, Homo sapiens
uuucgccgcaauaaggaaguc

(19) United States
(12) Patent Application Publication
(10) Pub. No.: US 2005/0059025 A1
(43) Pub. Date: Mar. 17, 2005

(54) Compositions, organisms and methodologies employing a novel human kinase

(75) Inventors: Wei Liu, Sudbury, MA (US); Leeying Wu, Lexington, MA (US)

Correspondence Address: NIXON PEABODY, LLP, 401 9TH STREET, NW, SUITE 900, WASHINGTON, DC, 20004-2128, US

(73) Assignee: Five Giralda Farms, Madison, NJ (US)

(21) Appl. No.: 10/679,366

(22) Filed: Oct. 7, 2003

Related U.S. Application Data
(60) Provisional application No. 60/416,257, filed on Oct. 7, 2002.

(51) Int. Cl. C12Q 1/68; G01N 33/53; C12N 9/12; C07H 21/04

(52) U.S. Cl. 435/6; 435/7.1; 435/69.1; 435/194; 435/320.1; 435/325; 536/23.2

(57) ABSTRACT

This invention provides compositions, organisms and methodologies employing a novel human protein kinase, MCRK2. The novel human kinase has sequence homology to rat myotonic dystrophy kinase-related Cdc42 binding kinase (MRCK) alpha. The gene encoding the novel kinase is localized in locus 1q42 of human chromosome 1. The novel protein kinase comprises multiple functional/structural domains that include a kinase domain, a pkinase_C domain, a DAG-PE binding domain, and a CNH domain. The sequence and structure similarity between the novel human protein and rat MRCK alpha indicates that the novel human protein may function as a downstream effector of Cdc42 in cytoskeleton reorganization.

The present application incorporates by reference U.S. Provisional Application Ser. No. 60/416,257, filed Oct. 7, 2002 and entitled “Composition, Organisms and Methodologies Employing a Novel Human Kinase.”

### FIELD OF THE INVENTION

The present invention relates to compositions, organisms and methodologies employing a novel human protein kinase, MRCK2, which has 95% sequence homology to rat myotonic dystrophy kinase-related Cdc42 binding kinase alpha (MRCK alpha). This invention can be used for diagnosing, prognosing, and treating kinase-related diseases and, in particular, diseases associated with aberrant expression of MRCK2.

### BACKGROUND OF THE INVENTION

Protein kinases regulate many different cell proliferation, differentiation, and signaling processes by adding phosphate groups to proteins. Uncontrolled signaling has been implicated in a variety of disease conditions including inflammation, cancer, arteriosclerosis, and psoriasis. Reversible protein phosphorylation is the main strategy for controlling activities of eukaryotic cells. It is estimated that more than 1,000 of the 10,000 proteins active in a typical mammalian cell are phosphorylated. The high energy phosphate, which drives activation, is generally transferred from adenosine triphosphate molecules (ATP) to a particular protein by protein kinases and removed from that protein by protein phosphatases. Phosphorylation occurs in response to extracellular signals (hormones, neurotransmitters, growth and differentiation factors, etc.), cell cycle checkpoints, and environmental or nutritional stresses. The phosphorylation process is roughly analogous to turning on a molecular switch. When the switch goes on, the appropriate protein kinase activates a metabolic enzyme, regulatory protein, receptor, cytoskeletal protein, ion channel or pump, or transcription factor.

The kinases comprise the largest known protein group, a superfamily of enzymes with widely varied functions and specificities. They are usually named after their substrate, their regulatory molecules, or some aspect of a mutant phenotype. With regard to substrates, the protein kinases may be roughly divided into two groups: those that phosphorylate tyrosine residues (protein tyrosine kinases, PTK) and those that phosphorylate serine or threonine residues (serine/threonine kinases, STK). A few protein kinases have dual specificity and phosphorylate threonine and tyrosine residues. Almost all kinases contain a similar 250-300 amino acid catalytic domain. The primary structure of the kinase domains is conserved and can be further subdivided into 11 subdomains. The N-terminal of the kinase domain, which contains subdomains I-IV, generally folds into a lobe-like structure that binds and orients the ATP (or GTP) donor molecule. The C terminal of the kinase domain forms a larger lobe, which contains subdomains VI-XI, binds the protein substrate and carries out the transfer of the gamma phosphate from ATP to the hydroxyl group of a serine, threonine, or tyrosine residue. Subdomain V spans the two lobes. Each of the 11 subdomains contains specific residues and motifs or patterns of amino acids that are characteristic of that subdomain and are highly conserved.

The kinases may be categorized into families by the different amino acid sequences (generally between 5 and 100 residues) located on either side of, or inserted into loops of, the kinase domain. These added amino acid sequences allow the regulation of each kinase as it recognizes and interacts with its target protein.

The presence of a phosphate moiety modulates protein function in multiple ways. A common mechanism involves changes in the catalytic properties (Vmax and Km) of an enzyme, leading to its activation or inactivation.

A second widely recognized mechanism involves promoting protein-protein interactions. An example of this is the tyrosine autophosphorylation of the ligand-activated EGF receptor tyrosine kinase. This event triggers the high-affinity binding to the phosphotyrosine residue on the receptor's C-terminal intracellular domain to the SH2 motif of an adaptor molecule Grb2. Grb2, in turn, binds through its SH3 motif to a second adaptor molecule, such as SHC. The formation of this complex activates the signaling events that are responsible for the biological effects of EGF. Serine and threonine phosphorylation events also have been recently recognized to exert their biological function through protein-protein interaction events that are mediated by the high-affinity binding of phosphoserine and phosphothreonine to the WW motifs present in a large variety of proteins.

A third important outcome of protein phosphorylation is changes in the subcellular localization of the substrate. As an example, nuclear import and export events in a large diversity of proteins are regulated by protein phosphorylation.

Many kinases are involved in regulatory cascades wherein their substrates may include other kinases whose activities are regulated by their phosphorylation state. Ultimately the activities of some downstream effectors are modulated by phosphorylation resulting from activation of such a pathway.

Myotonic dystrophy kinase-related Cdc42 binding kinases (MRCKs) are serine/threonine kinases. MRCKs have been implicated in the morphological activities of Cdc42 in nonneural cells and are suggest to be downstream effectors of Cdc42 in cytoskeletal reorganization. At least two types of MRCKs, MRCK alpha and MRCK beta, have been identified. MRCKs interact with the GTP-bound form of Cdc42 and, to a lesser extent, the GTP-bound form of Rac. The catalytic domain of MRCKs phosphorylates nonmuscle myosin light chain 2 at serine 19. The phosphorylation is believed to be involved in myosin contractile activity and associated changes in the organization of actin microfilaments in intact cells.

MRCK alpha and Rho-binding kinase (ROK) alpha are believed to have contrasting roles in regulating neurite morphology. ROK alpha acts downstream of RhoA in inducing neurite retraction, while MRCK alpha acts downstream of Cdc42/Rac1 in promoting neurite outgrowth. The neurite outgrowth induced by either kinase-dead ROK alpha or nerve growth factor can be effectively blocked by a kinase-dead and p21-binding deficient MRCK alpha mutant. In addition, expression of kinase-dead MRCK alpha blocks Cdc42^{V12}-dependent formation of focal complexes and peripheral microspikes. Microinjection of a plasmid encoding MRCK alpha results in actin and myosin reorganization.

MRCKs have multiple functional domains. These domains include three coiled-coil alpha-helix domains, a cysteine-rich motif resembling those of protein kinase C and n-chimaerin, and a Pleckstrin homology domain. Native MRCK kinases tend to form high-molecular-weight multimers. The intermolecular interactions among the three coiled-coil domains and the N-terminal region preceding the kinase domain in MRCK alpha are believed to be responsible for the multimerization.

MRCK alpha can be activated by the N-terminus-mediated dimerization. The dimerization leads to trans-autophosphorylation of MRCK kinases. In addition, MRCK alpha kinases can be negatively regulated through intramolecular interactions between the two distal coiled-coil domains. Deletion of these coiled-coil domains leads to a more active kinase, showing the negative autoregulatory role of these domains. The N-terminus-mediated dimerization and the intramolecular interaction between the distal coiled-coil domains are considered to be two mutually exclusive events, which regulate the catalytic state of MRCK kinases.

Two MRCK alpha substrates, p130 and p85, have been identified. P130 is identical to myosin binding subunit p130. P85 contains various ankyrin repeats at its N-terminus, an alpha-helical C terminus with leucine repeats, and a conserved motif with an MRCK alpha-kinase phosphorylation site.

### SUMMARY OF THE INVENTION

The present invention discloses compositions, organisms and methodologies employing a novel human protein kinase. The new human protein kinase shares sequence homology with rat MRCK alpha. The gene encoding this new protein kinase is localized between loci 1q41 and 1q42 of human chromosome 1. This new gene is hereinafter referred to as the MRCK2 gene, and its encoded protein(s) is referred to as MRCK2 or MRCK2 kinase. MRCK2 has multiple domains including at least a kinase domain, a protein kinase C terminal domain, a myosin tail domain, a small phorbol esters/diacylglycerol binding domain (DAG_PE binding domain), a Pleckstrin homology domain, a CNH domain, and a P21-Rho-binding domain. The sequence and structure similarity between MRCK2 and rat MRCK alpha indicates that MRCK2 may be involved in Cdc42-mediated cytoskeleton reorganization in human cells.

MRCK2 shows 95% sequence identities to rat MRCK alpha. The kinase domain in MRCK2 also shows 100% sequence alignment with the consensus sequences of the catalytic domains of at least two subfamilies of protein kinases. The utilities of various kinase domains are known in the art. The utilities of other domains, such as protein kinase C terminal domains, Phorbol esters/diacylglycerol binding domains, Pleckstrin homology domains, CNH domains, and P21-Rho-binding domains are also known in the art. The unique peptide sequence of MRCK2, and nucleic acid sequences that encode the peptide, can be used as models for the development of human therapeutic targets, aid in the identification of therapeutic proteins, and serve as targets for the development of human therapeutic agents that modulate kinase activity in cells and tissues that express the kinase.

In one aspect, the invention provides isolated polynucleotides comprising a nucleotide sequence encoding MRCK2 or a variant of MRCK2.

In another aspect, the invention provides isolated polypeptides comprising the amino acid sequence of MRCK2 or a variant of MRCK2.

In yet another aspect, the invention provides agents that modulate expression levels of the MRCK2 gene or an activity of MRCK2.

The invention also provides methods for (a) detecting polynucleotides comprising a nucleotide sequence encoding MRCK2 or a variant of MRCK2 and (b) detecting polypeptides comprising an amino acid sequence of MRCK2 or a variant of MRCK2 in a biological sample.

The invention further provides methods for screening agents that modulate expression level of the MRCK2 gene or an activity of MRCK2.

The invention further provides cell lines harboring the MRCK2 gene, animals transgenic for the MRCK2 gene, and animals with an interrupted MRCK2 gene (MRCK2 knockout animals). These cell lines and animals can be used to study the functions of MRCK2.

In still another aspect, the invention provides polynucleotides capable of inhibiting MRCK2 gene expression by RNA interference.

The invention further provide methods of inhibiting MRCK2 gene expression by introducing siRNAs or other RNAi sequences into target cells.

The preferred embodiments of the inventions are described below in the Detailed Description of the Invention. Unless specifically noted, it is intended that the words and phrases in the specification and claims be given the ordinary and accustomed meaning to those of ordinary skill in the applicable art or arts. If any other meaning is intended, the specification will specifically state that a special meaning is being applied to a word or phrase.

It is further intended that the inventions not be limited only to the specific structure, material or methods that are described in the preferred embodiments, but include any and all structures, materials or methods that perform the claimed function, along with any and all known or later-developed equivalent structures, materials or methods for performing the claimed function.

Further examples exist throughout the disclosure, and it is not applicant's intention to exclude from the scope of his invention the use of structures, materials, or methods that are not expressly identified in the specification, but nonetheless are capable of performing a claimed function.

### BRIEF DESCRIPTION OF THE DRAWINGS

The inventions of this application are better understood in conjunction with the following drawings, in which:

FIG. 1 is the sequence alignment between MRCK2 and rat MRCK alpha.

FIG. 2 compares amino acid residues 77 to 343 of MRCK2 to the catalytic domain of a family of serine/threonine protein kinases.

FIG. 3 shows the sequence alignment between amino acid residues 77 to 343 of MRCK2 and the protein kinase domain of pkinases.

FIG. 4 shows the sequence alignment between amino acid residues 78 to 309 of MRCK2 and the catalytic domain of a family of tyrosine kinases.

FIG. 5 illustrates the sequence alignment between amino acid residues 344 to 404 of MRCK2 and the extension to serine/threonine-type protein kinases

FIG. 6 compares amino acid residues 344 to 406 of MRCK2 to a protein kinase C terminal domain.

FIG. 7 compares the amino acid residues 424 to 942 of MRCK2 to a myosin tail domain.

FIG. 8 shows the sequence alignment between amino acid residues 1013 to 1062 of MRCK2 to a DAG_PE binding (C1 domain). This domain is also known as the Protein kinase C conserved region 1 (C₁) domain.

FIG. 9 depicts the sequence alignment between the amino acid residues 1085 to 1201 of MRCK2 and a Pleckstrin homology domain.

FIG. 10 illustrates the sequence alignment between the amino acid residues 1235 to 1490 of MRCK2 and a consensus sequence of CNH domains.

FIG. 11 shows the sequence alignment between the amino acid residues 1571 to 1606 of MRCK2 and the consensus sequence of P21-Rho-binding domains.

FIG. 12 shows the hydrophobicity profile of MRCK2.

FIG. 13 shows the position and nucleotide sequence of human MRCK2 probe and PCR primers used for the amplification of the probe sequences.

### DETAILED DESCRIPTION OF THE INVENTION

The following detailed description is presented to enable any person skilled in the art to make and use the invention. For purposes of explanation, specific nomenclature is set forth to provide a thorough understanding of the present invention. However, it will be apparent to one skilled in the art that these specific details are not required to practice the invention. Descriptions of specific applications are provided only as representative examples. Various modifications to the preferred embodiments will be readily apparent to one skilled in the art, and the general principles defined herein may be applied to other embodiments and applications without departing from the scope of the invention. The present invention is not intended to be limited to the embodiments shown, but is to be accorded the widest possible scope consistent with the principles and features disclosed herein.

The present invention is based on the sequence information obtained from a newly developed genomic prediction pipeline. Briefly, the X-ray crystal structures of the catalytic domains of protein kinases were collected and aligned together according to their structural identity/similarities. The alignment was converted into a “scoring matrix” which carried the structural profile of the kinase catalytic domains. This scoring matrix was then used to search the Celera Human Genome database and pull out sequences that have kinase catalytic domains.

Based on this analysis, the present invention provides the amino acid sequence of a human kinase peptide that is highly homologous to rat myotonic dystrophy kinase-related Cdc42 binding kinase (MRCK), cDNA sequences and genomic sequences that encode the kinase peptide, and information about the closest art known protein/peptide/domain that has structural or sequence homology to the kinase of the present invention.

In addition to being previously unknown, the peptide of the present invention is selected based on its ability to be used for the development of commercially important products and services. Specifically, the present peptides are selected based on homology to a known kinase protein of rat MRCK alpha.

Various aspects of the invention are described in detail in the following subsections. The use of subsections is not meant to limit the invention. Each subsection applies to any aspect of the invention.

Definitions and Terms

To facilitate the understanding of the present invention, a number of terms and phrases are defined below:

As used herein, a polynucleotide or a polypeptide is “isolated” if it is removed from its native environment. For instance, a polynucleotide or a polypeptide is isolated through a purification process such that the polynucleotide or polypeptide is substantially free of cellular material or free of chemical precursors. The polynucleotide/polypeptide of the present invention can be purified to homogeneity or other degrees of purity. The level of purification will be based on the intended use. As appreciated by one of ordinary skill in the art, a polynucleotide/polypeptide can perform its desired function(s) even in the presence of considerable amounts of other components or molecules.

In some uses, a polynucleotide/polypeptide that is “substantially free of cellular material” includes preparations which have less than about 30% (by weight) other polynucleotides/polypeptides including contaminating polynucleotides/polypeptides. For instance, the preparations can have less than about 20%, less than about 10%, or less than about 5% other polynucleotides/polypeptides. If a polynucleotide/polypeptide preparation is recombinantly produced, it can be substantially free of culture medium, i.e., culture medium components representing less than about 20% by weight of the polynucleotide/polypeptide preparation.

The language “substantially free of chemical precursors” includes preparations in which the polynucleotide/polypeptide is separated from chemical precursors or other chemicals that are involved in the synthesis of the polynucleotide/polypeptide. In one embodiment, the language “substantially free of chemical precursors” includes kinase preparations having less than about 30% (by weight), less than about 20% (by weight), less than about 10% (by weight), or less than about 5% (by weight) chemical precursors or other chemicals used in the synthesis.

A “polynucleotide” can include any number of nuceloetides. For instance, a polynucleotide can have at least 10, 20, 25, 30, 40, 50, 100 or more nucleotides. A polynucleotide can be DNA or RNA, double-stranded or single-stranded. A polynucleotide encodes a polypeptide if the polypeptide is capable of being transcribed and/or translated from the polynucleotide. Transcriptional and/or translational regulatory sequences, such as promoter and/or enhancer(s), can be added to the polynucleotide before said transcription and/or translation occurs. Moreover, if the polynucleotide is singled-stranded, the corresponding double-stranded DNA containing the original polynucleotide and its complementary sequence can be prepared before said transcription and/or translation.

As used herein, “a variant of a polynucleotide” refers to a polynucleotide that differs from the original polynucleotide by one or more substitutions, additions, and/or deletions. For instance, a variant of a polynucleotide can have 1, 2, 5, 10, 15, 20, 25 or more nucleotide substitutions, additions or deletions. Preferably, the modification(s) is in-frame, i.e., the modified polynucleotide can be transcribed and translated to the original or intended stop codon. If the original polynucleotide encodes a polypeptide with a biological activity, the polypeptide encoded by a variant of the original polynucleotide variants substantially retains such activity.

Preferably, the biological activity is reduced/enhanced by less than 50%, or more preferably, less than 20%, relative to the original activity.

A variant of a polynucleotide can be a polynucleotide that is capable of hybridizing to the original polynucleotide, or the complementary sequence thereof, under reduced stringent conditions, preferably stringent conditions, or more preferably, highly stringent conditions. Examples of conditions of different stringency are listed in Table 1. Highly stringent conditions are those that are at least as stringent as conditions A-F; stringent conditions are at least as stringent as conditions G-L; and reduced stringency conditions are at least as stringent as conditions M-R. As used in Table 1, hybridization is carried out under a given hybridization condition for about 2 hours, followed by two 15-minute washes under the corresponding washing condition(s).

TABLE 1
Stringency Conditions

| Stringency | Poly-nucleotide | Hybrid | Hybridization | Wash Temp. |
|---|---|---|---|---|
| Condition | Hybrid | Length (bp)¹ | Temperature and Buffer^{H} | and Buffer^{H} |
| A | DNA:DNA | >50 | 65° C.; 1xSSC -or- | 65° C.; 0.3xSSC |
| | | | 42° C.; 1xSSC, 50% formamide | |
| B | DNA:DNA | <50 | T_{B}*; 1xSSC | T_{B}*; 1xSSC |
| C | DNA:RNA | >50 | 67° C.; 1xSSC -or- | 67° C.; 0.3xSSC |
| | | | 45° C.; 1xSSC, 50% formamide | |
| D | DNA:RNA | <50 | T_{D}*; 1xSSC | T_{D}*; 1xSSC |
| E | RNA:RNA | >50 | 70° C.; 1xSSC -or- | 70° C.; 0.3xSSC |
| | | | 50° C.; 1xSSC, 50% formamide | |
| F | RNA:RNA | <50 | T_{F}*; 1xSSC | T_{f}*; 1xSSC |
| G | DNA:DNA | >50 | 65° C.; 4xSSC -or- | 65° C.; 1xSSC |
| | | | 42° C.; 4xSSC, 50% formamide | |
| H | DNA:DNA | <50 | T_{H}*; 4xSSC | T_{H}*; 4xSSC |
| I | DNA:RNA | >50 | 67° C.; 4xSSC -or- | 67° C.; 1xSSC |
| | | | 45° C.; 4xSSC, 50% formamide | |
| J | DNA:RNA | <50 | T_{J}*; 4xSSC | T_{J}*; 4xSSC |
| K | RNA:RNA | >50 | 70° C.; 4xSSC -or- | 67° C.; 1xSSC |
| | | | 50° C.; 4xSSC, 50% formamide | |
| L | RNA:RNA | <50 | T_{L}*; 2xSSC | T_{L}*; 2xSSC |
| M | DNA:DNA | >50 | 50° C.; 4xSSC -or- | 50° C.; 2xSSC |
| | | | 40° C.; 6xSSC, 50% formamide | |
| N | DNA:DNA | <50 | T_{N}*; 6XSSC | T_{N}*; 6xSSC |
| O | DNA:RNA | >50 | 55° C.; 4xSSC -or- | 55° C.; 2xSSC |
| | | | 42° C.; 6xSSC, 50% formamide | |
| P | DNA:RNA | <50 | T_{P}*; 6xSSC | T_{P}*; 6xSSC |
| Q | RNA:RNA | >50 | 60° C.; 4xSSC -or- | 60° C.; 2xSSC |
| | | | 45° C.; 6xSSC, 50% formamide | |
| R | RNA:RNA | <50 | T_{R}*; 4xSSC | T_{R}*; 4xSSC |
| ¹The hybrid length is that anticipated for the hybridized region(s) of the hybridizing polynucleotides. When hybridizing a polynucleotide to a target polynucleotide of unknown sequence, the hybrid length is assumed to be that of the hybridizing polynucleotide. When polynucleotides of known sequence are hybridized, the hybrid length can be determined by aligning the sequences of the polynucleotides and identifying the region or regions of optimal sequence complementarity. | | | | |
| ^{H}SSPE (1xSSPE is 0.15 M NaCl, 10 mM NaH₂PO₄, and 1.25 mM EDTA, pH 7.4) can be substituted for SSC (1 × SSC is 0.15 M NaCl and 15 mM sodium citrate) in the hybridization and wash buffers. | | | | |
| T_{B}*-T_{R}* The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.) = 2(# of A + T bases) + 4(# of G + C bases). For hybrids between 18 and 49 base pairs in | | | | |
| # length, Tₘ(° C.) = 81.5 + 16.6(log₁₀Na⁺) + 0.41(% G + C) − (600/N), where N is the number of bases in the hybrid, and Na⁺ is the molar concentration of sodium ions in the hybridization buffer (Na⁺ for 1xSSC = 0.165 M). | | | | |

It will be appreciated by those of ordinary skill in the art that, as a result of the degeneracy of the genetic code, there are many polynucleotide variants that encode the same polypeptide. Some of these polynucleotide variants bear minimal sequence homology to the original polynucleotide. Nonetheless, polynucleotides that vary due to differences in codon usage are specifically contemplated by the present invention.

As used herein, a “polypeptide” can include any number of amino acid residues. For instance, a polypeptide can have at least 5, 10, 15, 20, 30, 40, 50 or more amino acid residues.

As used herein, a “variant of a polypeptide” is a polypeptide that differs from the original polypeptide by one or more substitutions, deletions, and/or insertions. Preferably, these modifications do not substantially change (e.g. reduce or enhance) the original biological function of the polypeptide. For instance, a variant can reduce or enhance or maintain the biological activities of the original polypeptide. Preferably, the biological activities of the variant is reduced or enhanced by less than 50%, or more preferably, less than 20%, relative to the original polypeptide.

Similarly, the ability of a variant to react with antigen-specific antisera can be enhanced or reduced by less than 50%, preferably less than 20%, relative to the original polypeptide. These variants can be prepared and evaluated by modifying the original polypeptide sequence and then determining the reactivity of the modified polypeptide with the antigen-specific antibodies or antisera.

Preferably, a variant polypeptide contains one or more conservative substitutions. A “conservative substitution” is one in which an amino acid is substituted for another amino acid which has similar properties, such that one skilled in the art would expect that the secondary structure and hydropathic nature of the substituted polypeptide will not be substantially changed. Conservative amino acid substitutions can be made on the basis of similarity in polarity, charge, solubility, hydrophobicity, hydrophilicity and/or the amphipathic nature of the residues. Negatively charged amino acids include aspartic acid and glutamic acid, and positively charged amino acids include lysine and arginine. Amino acids having uncharged polar head groups and similar hydrophilicity values include leucine, isoleucine and valine, or glycine and alanine, or asparagine and glutamine, or serine, threonine, phenylalanine and tyrosine. Other groups of amino acids that can produce conservative changes include: (1) ala, pro, gly, glu, asp, gln, asn, ser, thr; (2) cys, ser, tyr, thr; (3) val, ile, leu, met, ala, phe; (4) lys, arg, his; and (5) phe, tyr, trp, his. A polypeptide variant can also contain nonconservative changes.

Polypeptide variants can be prepared by the deletion and/or addition of amino acids that have minimal influence on the biological activity, immunogenicity, secondary structure and/or hydropathic nature of the polypeptide. Variants can be prepared, for instance, by substituting, modifying, deleting or adding one or more amino acids residues in the original sequence. Polypeptide variants preferably exhibit at least 96%, more preferably at least 97%, and most preferably at least 98% sequence homology to the original polypeptide.

Polypeptide variants include polypeptides that are modified from the original polypeptides either by a natural process, such as a post-translational modification, or by a chemical modification. These modifications are well known in the art. Modifications can occur anywhere in the polypeptide, including the backbone, the amino acid side-chains and the amino or carboxyl termini. It will be appreciated that the same type of modification can be present in the same or varying degrees at several sites in a given polypeptide. Also, a given polypeptide can contain many types of modifications. Polypeptides may be branched, for example, as a result of ubiquitination, and they may be cyclic, with or without branching. Cyclic, branched, and branched cyclic polypeptides can result from natural post-translational processes or be made through synthetic methods. Suitable modifications for this invention include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination.

As used herein, the term “modulation” includes up-regulation, induction, stimulation, potentiation, inhibition, down-regulation or suppression, or relief of inhibition.

A nucleotide sequence is “operably linked” to another nucleotide sequence if the two sequences are placed into a functional relationship. For example, a coding sequence is operably linked to a 5′ regulatory sequence if the 5′ regulatory sequence can initiate transcription of the coding sequence in an in vitro transcription/translation system or in a host cell. “Operably linked” does not require that the DNA sequences being linked are contiguous to each other. Intervening sequences may exist between two operably linked sequences.

As used herein, a “disease-free” human refers to a human who does not have MRCK2-related diseases. Disease-free cells, tissues or samples refer to cells, tissues or samples obtained from such disease-free human(s).

A polynucleotide is “capable of hybridizing” to a gene if the polynucleotide can hybridize to at least one of the following sequences: (1) the sequence of a RNA transcript of the gene, (2) the complementary sequence of a RNA transcript of the gene, (3) the cDNA sequence of a RNA transcript of the gene, (4) the complementary sequence of the cDNA sequence of a RNA transcript of the gene, (5) a genomic sequence of the gene, and (6) the complementary sequence of a genomic sequence of the gene.

As used herein, sequence identity or percentage alignment can be determined by the standard protein-protein or nucleotide-nucleotide BLAST programs (i.e., blastp or blastn, respectively). Sequence identity or percentage alignment can also be determined by the BLAST2 program. Suitable BLAST programs can be found at the BLAST web site maintained by the National Center of Biotechnology Information (NCBI) (National Library of Medicine, USA).

The Human MRCK2 Gene and MRCK2 Kinase

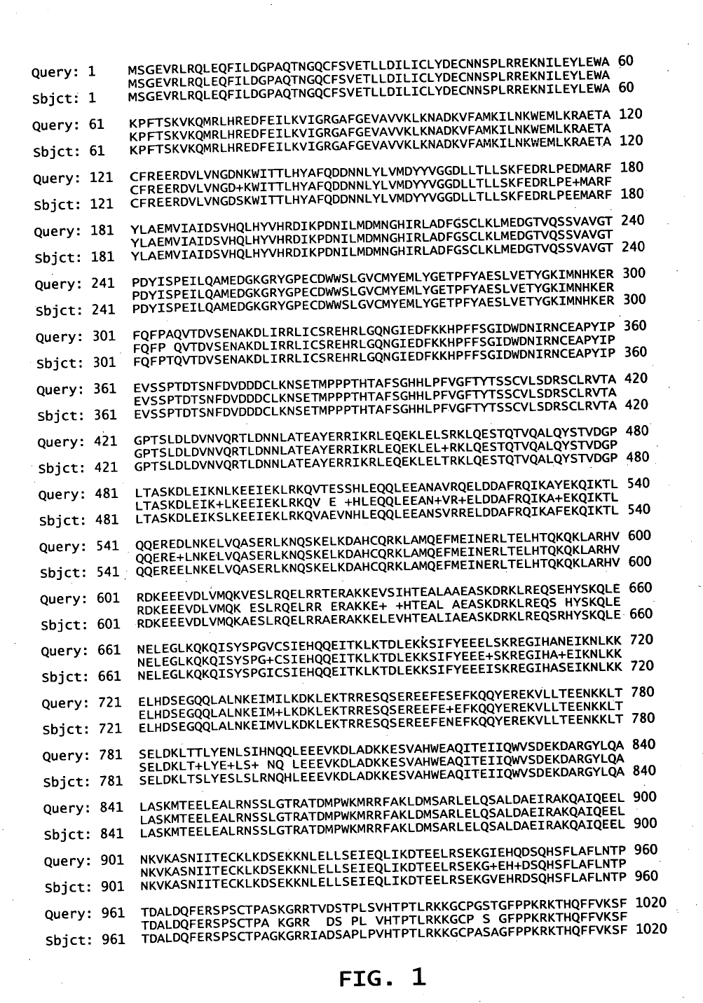

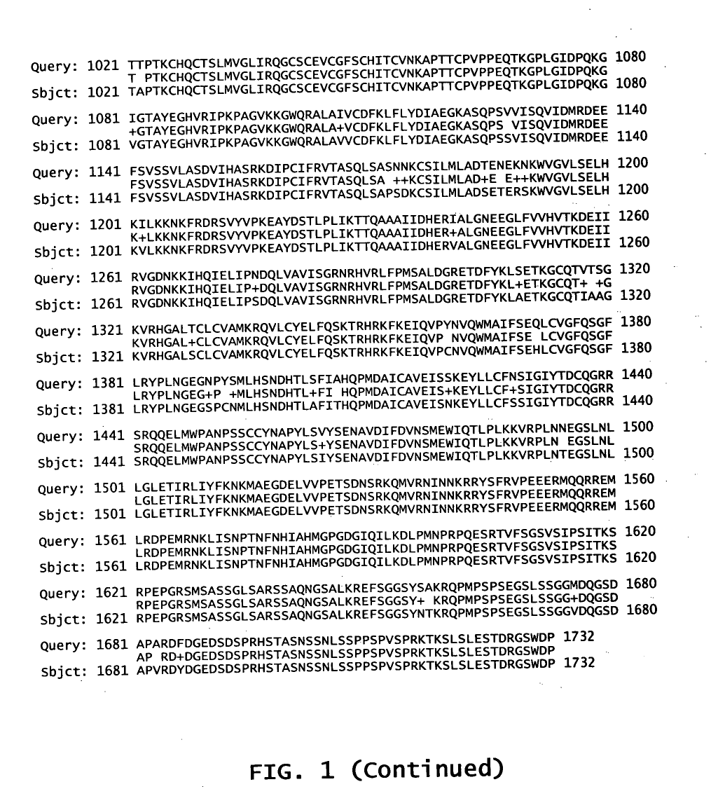

The present invention identifies a new human gene (the MRCK2 gene) that encodes a protein highly homologous to rat MRCK alpha. The nucleotide sequence encoding MRCK2 and the amino acid sequence of MRCK2 are depicted in SEQ ID NOS:1 and 2, respectively. FIG. 1 shows the amino acid sequence alignment between MRCK2 (denoted as “Query”) and rat MRCK alpha (denoted as “Sbjct’) using standard protein-protein BLAST algorithm with no filter chosen, “Expect” setting at 10.0, “Word Size” setting at 3, “Matrix” setting at BLOSUM62, “Gap costs” setting at Existence: 11 and Extension: 1. The two proteins show 95% sequences identities with a score of 3269 bits and an E value of 0.0. Further analysis using standard nucleotide-nucleotide BLAST search in database nr with no filter chosen, “Expect” setting at 10.0, “Word Size” setting at 3, revealed 90% sequence identities at the nucleic acid level with a score of 6383 and an E value of 0.0.

The standard protein-protein BLAST search also identifies sequence similarity between MRCK2 and other proteins. These proteins include, but are not limited to, a human KIAA1124 protein (Entrez accession number: AB032950), rat MRCK beta (Entrez accession number: AF021936), and human Cdc42-binding protein kinase beta (Entrez accession number: AF128625_{—}1).

The MRCK2 gene is localized in locus 1q42 of human chromosome 1. Specifically, the MRCK2 gene is located between loci LOC163851 and LOC200096, and overlaps with loci LOC205022, LOC163855, PRO2012 and KIAA0451.

Human chromosome locus 1q42 and the neighboring regions harbor multiple disease-related genes. These diseases include breast cancer, prostate cancer, lung cancer, Type I diabetes mellitus, systemic lupus erythematosus, schizophrenia, congenital myopathic dystrophies, and childhood acute lymphoblastic leukemia.

The human MRCK2 gene has at least 37 exons. Exons 1-3, 7, and 11-37 are mapped between nucleotides 3700505 to 3973937 of human chromosome 1 in the Entrez Human Genome Sequence Database maintained by NCBI. All the exons are mapped to chromosome 1 in Celera genomic database. The genomic sequence of human MRCK2 is shown in SEQ ID NO:3. Table 2 lists the location of each of these 37 exons in the genomic sequence SEQ ID NO:3.

TABLE 2
Exons in Human MRCK2 Gene

| | Corresponding Sequence | Corresponding Sequence |
|---|---|---|
| Exon Numbers | in SEQ ID NO: 3 | in SEQ ID NO: 1 |
| 1 | 1-178 | 1-178 |
| 2 | 62,974-63,065 | 179-270 |
| 3 | 103,941-104,024 | 271-354 |
| 4 | 117,513-117,608 | 355-450 |
| 5 | 123,240-123,388 | 451-599 |
| 6 | 156,548-156,641 | 600-693 |
| 7 | 169,586-169,786 | 694-894 |
| 8 | 171,410-171,658 | 895-1,143 |
| 9 | 174,124-174,203 | 1,144-1,223 |
| 10 | 177,440-177,606 | 1,224-1,390 |
| 11 | 187,923-188,045 | 1,391-1,513 |
| 12 | 197,217-197,350 | 1,514-1,647 |
| 13 | 204,241-204,490 | 1,648-1,897 |
| 14 | 204,739-204,842 | 1,898-2,001 |
| 15 | 215,916-216,163 | 2,002-2,249 |
| 16 | 225,163-225,168 | 2,250-2,355 |
| 17 | 236,136-236,260 | 2,356-2,480 |
| 18 | 242,789-242,894 | 2,481-2,586 |
| 19 | 243,141-243,289 | 2,587-2,735 |
| 20 | 244,854-244,945 | 2,736-2,827 |
| 21 | 247,297-247,373 | 2,828-2,904 |
| 22 | 257,745-257,783 | 2,905-2,943 |
| 23 | 269,149-269,232 | 2,944-3,027 |
| 24 | 276,918-277,023 | 3,028-3,133 |
| 25 | 281,550-281,686 | 3,134-3,270 |
| 26 | 282,367-282,506 | 3,271-3,410 |
| 27 | 283,746-283,827 | 3,411-3,492 |
| 28 | 285,727-285,863 | 3,493-3,709 |
| 29 | 286,691-286,753 | 3,710-3,772 |
| 30 | 287,913-288,506 | 3,773-4,366 |
| 31 | 291,012-291,109 | 4,367-4,464 |
| 32 | 293,817-293,901 | 4,465-4,549 |
| 33 | 300,116-300,239 | 4,550-4,673 |
| 34 | 300,969-301,086 | 4,674-4,791 |
| 35 | 312,040-312,157 | 4,792-4,909 |
| 36 | 322,173-322,330 | 4,910-5,067 |
| 37 | 322,754-322,885 | 5,068-5,199 |

A conserved domain search using RPS-BLAST program (RPS-BLAST 2.2.3 [Apr. 24, 2000], available the BLAST web site maintained by NCBI), showed that MRCK2 contained sequences homologous to at least three kinase domains, a protein kinase C terminal domain, a myosin tail domain, a small phorbol esters/diacylglycerol binding domain (DAG_PE binding domain), a Pleckstrin homology domain, a CNH domain, and a P21-Rho-binding domain.

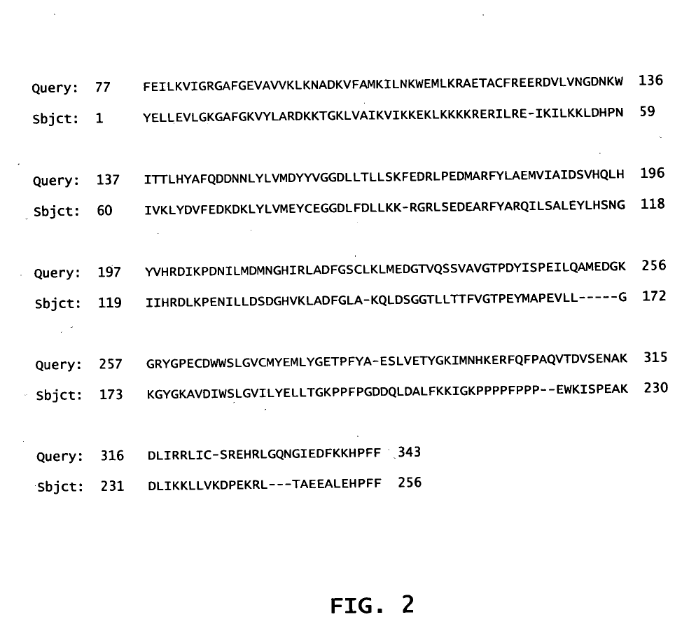

Specifically, the amino acid residues 77 to 343 of MRCK2 are highly homologous to a catalytic domain of a family of serine/threonine protein kinases (smart00220). This kinase family includes C-Jun N-terminal kinase (JNK3), abelson tyrosine kinase, a calmodulin-binding, vesicle-associated, protein kinase-like protein (1G5), serine/threonine-protein kinase prp4, Cdc2/Cdc28 subfamily of serine/threonine protein kinases in C. elegans, and ribosomal S6 kinase of C. elegans. FIG. 2 shows that the two sequences share 100% alignment with a score of 252 and an E value of 1×10⁻⁶⁷. As used in other figures of this invention, “Query” denotes to the sequence of MRCK2, and “Sbjct” refers to the sequence being compared to the MRCK2 sequence.

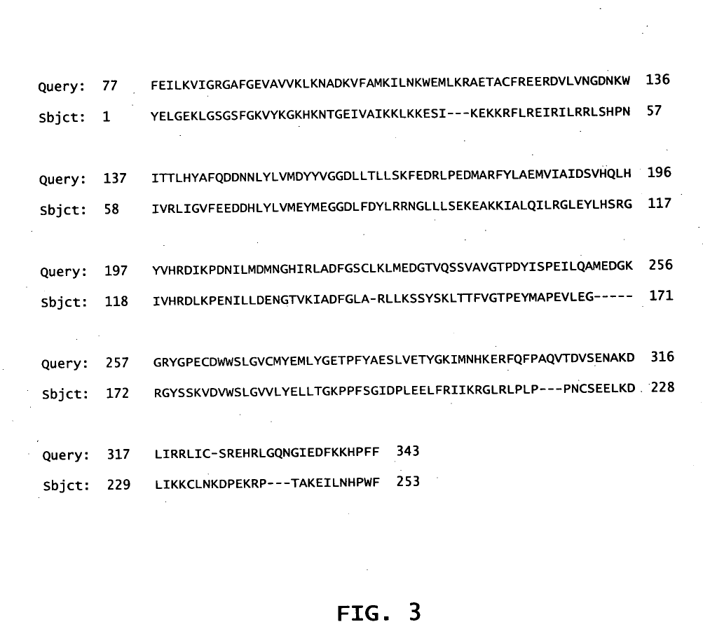

The amino acid residues 77 to 343 of MRCK2 also aligned 100% with the protein kinase domain of pkinases (pfam00069) (FIG. 3). The alignment has a score of 205 bit, and an E value of 1×10⁻⁵³. This pkinase family includes protein kinase Ck2, wee1-like protein kinase (WEE1hu), and tyrosine-protein kinase RYK.

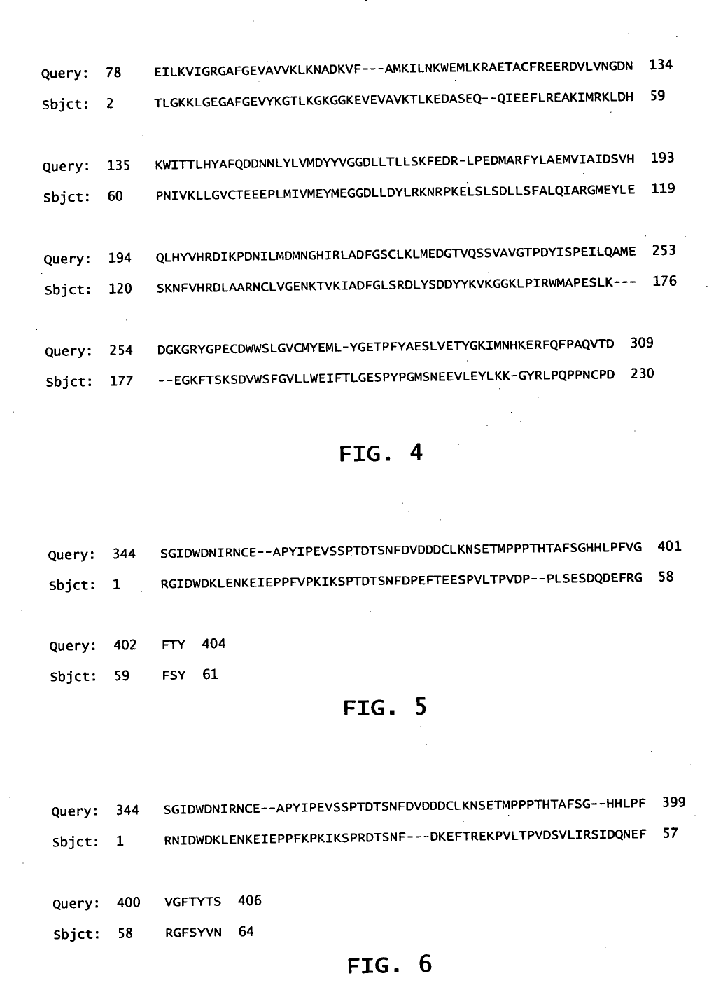

FIG. 4 shows the sequence alignment between amino acid residues 78 to 309 of MRCK2 and the catalytic domain of a family of tyrosine kinases (smart00219). This family includes the tyrosine kinase domain of fibroblast growth factor receptor 1, tyrosine-protein kinase (KIN15/KIN16 subfamily), and a Drosophila receptor protein-tyrosine kinase family member (drl-P1). The amino acid residues 78-309 in MRCK2's kinase domain has 89.1% sequence identities to smart00219, with a score of 110 bits and an E value of 4×10⁻²⁵.

FIG. 5 illustrates the sequence alignment between amino acid residues 344 to 404 of MRCK2 and S_TK X (smart00133), the consensus sequence for the extension to a family of serine/threonine-type protein kinases. This family of protein kinases includes cAMP-dependent protein kinase, protein kinase cek1, and cell cycle protein kinase DBF2. The two sequences share 96.8% sequence identities with a score of 65.4 bits and an E value of 1×10⁻¹¹.

FIG. 6 illustrates the sequence alignment between amino acid residues 344 to 406 of MRCK2 and a consensus sequence (CD NO: pfam00433) of the protein kinase C terminal domain. The two sequences are 95.5% aligned with a score of 43.7 bits and an E value of 6×10⁻⁵. Ribosomal protein S6 kinase (S6K), serine/threonine-protein kinase YPK1, and protein kinase C, zeta type (NPKC-ZETA) share the consensus sequence pfam00433.

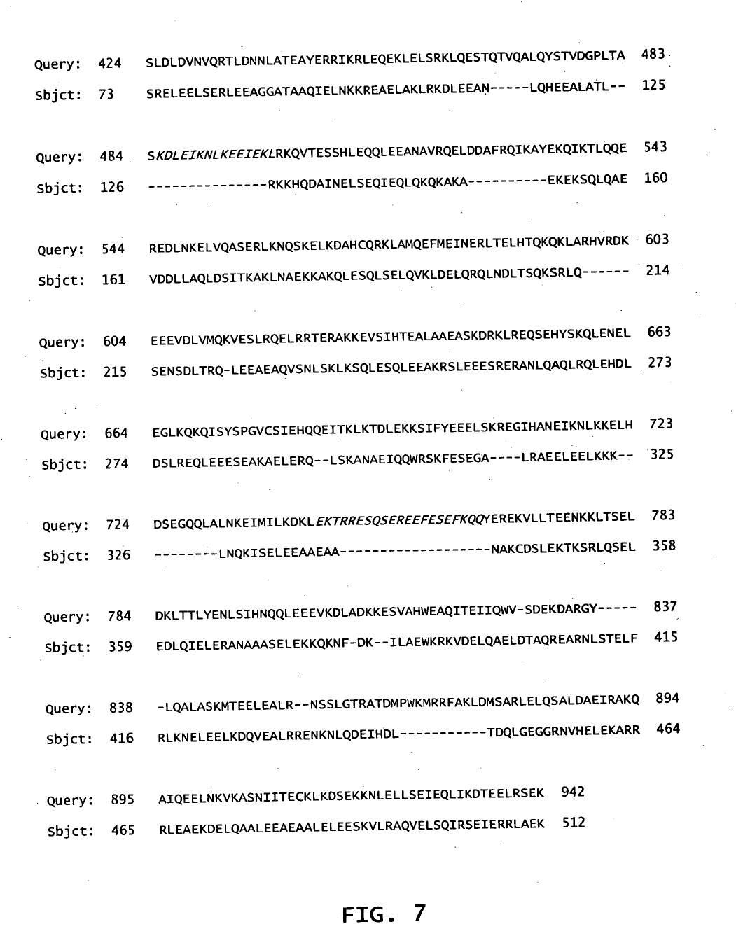

FIG. 7 compares the amino acid residues 424 to 942 of MRCK2 to a myosin tail domain (CD NO: pfam01576). The myosin molecule is a multi-subunit complex made up of two heavy chains and four light chains. It is a fundamental contractile protein found in all eukaryotic cell types. The myosin tail domain consists of the coiled-coil myosin heavy chain tail region. The coiled-coil is composed of the tail from two molecules of myosin. These can then assemble into the macromolecular thick filament. The coiled-coil region provides the structural backbone the thick filament. The alignment shows 51.2% sequence identities with a score of 58.0 bits and an E value of 3×10⁻⁹.

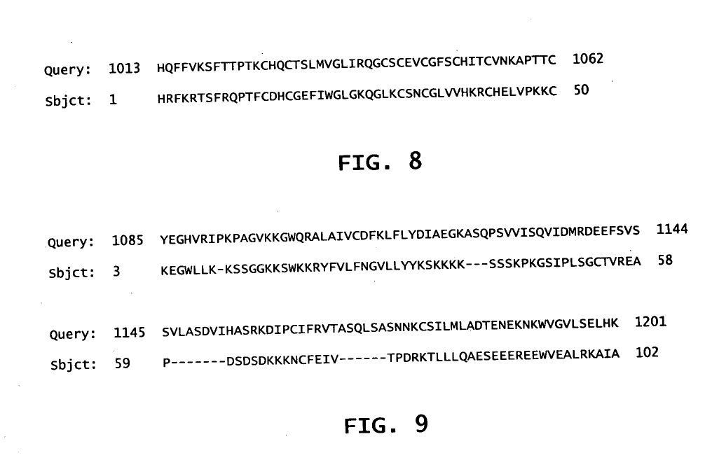

FIG. 8 shows the sequence alignment between amino acid residues 1013 to 1062 of MRCK2 to a DAG_PE binding (C1 domain). This domain is also known as the Protein kinase C conserved region 1 (C1 or cysteine-rich) domain. The two sequences show 100% sequence identities with a score of 61.9 bits and an E value of 2×10⁻¹⁰. The CD number for the consensus sequence is pfam00130.

FIG. 9 depicts the sequence alignment between the amino acid residues 1085 to 1201 of MRCK2 and a consensus sequence (CD NO: smart00233.4) of the Pleckstrin homology (PH) domains of a family of proteins which include Rac1 and GTPase activating protein BEM2/IPL2. The comparison indicates 96.2% sequence identities with a score of 41.3 bits and an E value of 2×10⁻⁴. PH domains are commonly found in eukaryotic signaling proteins. The domain family possesses multiple functions including the abilities to bind inositol phosphates and various other proteins. PH domains have been found to possess inserted domains (such as in PLC gamma, syntrophins) and/or to be inserted within other domains. Mutations in Brutons tyrosine kinase (Btk) within its PH domain cause X-linked agammaglobulinaemia (XLA) in patients.

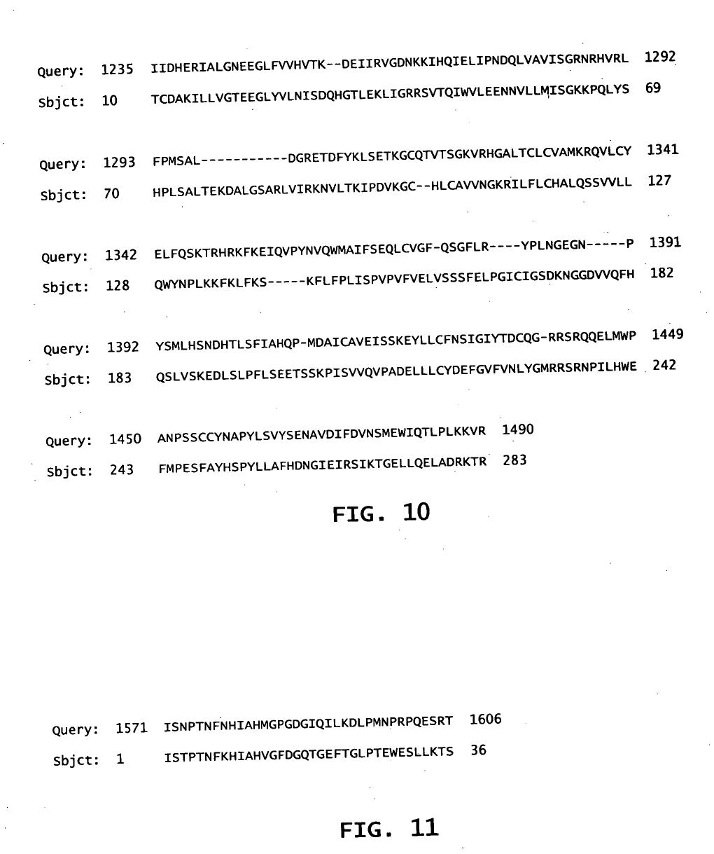

FIG. 10 illustrates the sequence alignment between the amino acid residues 1235 to 1490 of MRCK2 and a consensus CNH domain sequence (CD NO: smart00036). The alignment shows 91.0% sequence identities with a score of 98.8 bits and an E value of 1×10⁻²¹. The consensus CNH domain sequence is found in NIK1-like kinase, mouse citron (Rho-interacting, serine/threonine kinase 21), and yeast ROM1 and ROM2.

FIG. 11 shows the sequence alignment between the amino acid residues 1571 to 1606 of MRCK2 and the consensus sequence of P21-Rho-binding domains (CD NO: smart00285). The two sequences have 100.0% sequence identities with a score of 45.6 bits and an E value of 2×10⁻⁵. P21-Rho-binding domain is a domain that binds Cdc42p- and/or Rho-like small GTPases. The domain also knows as the Cdc42/Rac interactive binding domain (CRIB domain).

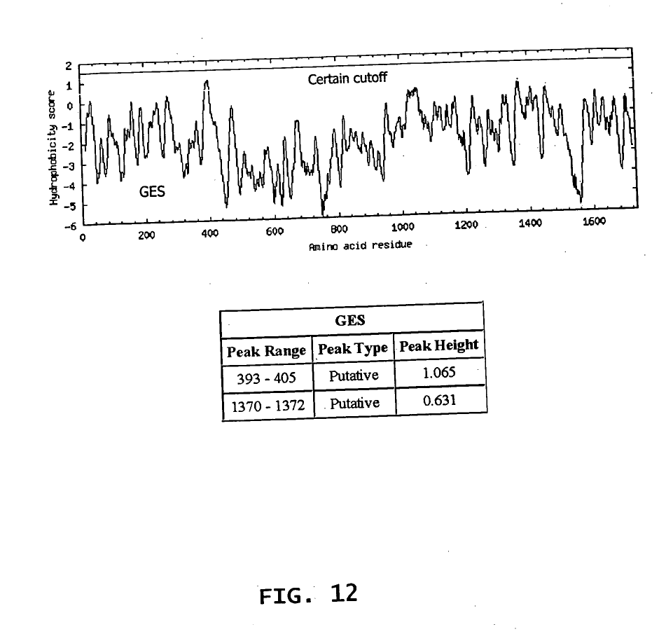

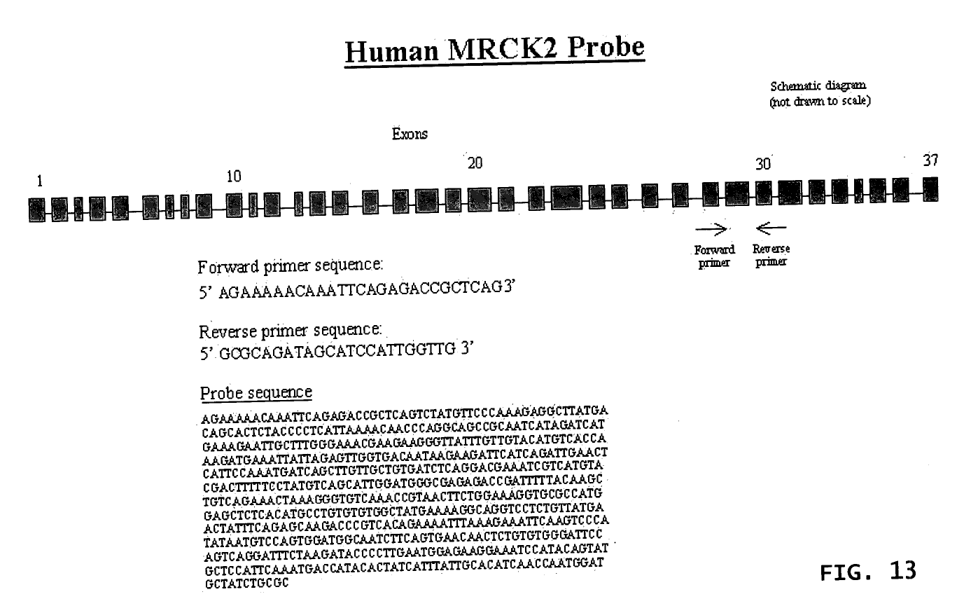

FIG. 12 shows the hydrophobicity profile of MRCK2. The hydrophobicity analysis indicates that MRCK2 kinase is not likely a membrane or transmembrane protein.

The existence and expression of the MRCK2 gene in humans are supported by various EST sequences. For instance, nucleotides 1-758 of SEQ ID NO:1 are supported by the EST sequences disclosed under GenBank accession numbers BQ232716 and BG545697; nucleotides 844-1467 of SEQ ID NO:1 are supported by the EST sequence disclosed under GenBank accession number A1693000; nucleotides 1899-2423 of SEQ ID NO:1 are supported by the EST sequences disclosed under GenBank accession numbers BG285359; nucleotides 3012-5194 of SEQ ID NO:1 are supported by the EST sequences disclosed under GenBank accession numbers BM806560, BM549190, BM453497, BM053102, BG117325, and BM794603.

A multiple tissue Northern analysis (MTN) using the human MRCK2 probe depicted in FIG. 13 revealed two transcripts of MRCK2, a 7.5 kb smear band and a 11 kb sharp band. Both bands were detected in human brain, heart, skeletal muscle, colon, spleen, kidney, liver, small intestine, placenta, and lung; but not in thymus and peripheral blood leukocyte. In addition, a 4 kb band was detected only in brain and skeletal muscle. The MRCK2 expression was further confirmed by a multiple tissue expression array (MTA), in which MRCK2 expression was found in most tissues except thymus, peripheral blood leukocyte, ovary and two Burkitt's lymphomas.

Utility of Protein Kinases

Protein kinases are involved in the regulation of many critical biological processes such as signal transduction pathways. Malfunctions of cellular signaling have been associated with many diseases. Regulation of signal transduction by cytokines and association of signal molecules with protooncogenes and tumor suppressor genes have been the subjects of intense research. Many therapeutic strategies can now be developed through the synthesis of compounds which activate or inactivate protein kinases.

The importance of kinases in the etiology of diseases has been well established. Kinase proteins are a major target for drug action and development. A January 2002 survey of ongoing clinical trials in the USA revealed more than 100 clinical trials involving the modulation of kinases. Trials are ongoing in a wide variety of therapeutic indications including asthma, Parkinson's, inflammation, psoriasis, rheumatoid arthritis, spinal cord injuries, muscle conditions, osteoporosis, graft versus host disease, cardiovascular disorders, autoimmune disorders, retinal detachment, stroke, epilepsy, ischemia/reperfusion, breast cancer, ovarian cancer, glioblastoma, non-Hodgkin's lymphoma, colorectal cancer, non-small cell lung cancer, brain cancer, Kaposi's sarcoma, pancreatic cancer, liver cancer, and other tumors. Numerous kinds of modulators of kinase activity are currently in clinical trials including antisense molecules, antibodies, small molecules, and even gene therapy. Accordingly, it is valuable to the field of pharmaceutical development to identify and characterize previously unknown members of the kinase family proteins. The present invention advances the state of the art by providing previously unidentified human kinase proteins which are structurally related to MRCKs.

Many therapeutic strategies are aimed at critical components in signal transduction pathways. Approaches for regulating kinase gene expression include specific antisense oligonucleotides for inhibiting post-transcriptional processing of the messenger RNA, naturally occurring products and their chemical derivatives to inhibit kinase activity and monoclonal antibodies to inhibit receptor linked kinases. In some cases, kinase inhibitors also allow other therapeutic agents additional time to become effective and act synergistically with current treatments.

Among the areas of pharmaceutical research that are currently receiving a great deal of attention are the role of phosphorylation in transcriptional control, apoptosis, protein degradation, nuclear import and export, cytoskeletal regulation, and checkpoint signaling. The accumulating knowledge about signaling networks and the proteins involved will be put to practical use in the development of potent and specific pharmacological modulators of phosphorylation-dependent signaling. The rational structure-based design and development of highly specific kinase modulators is becoming routine and drugs that intercede in signaling pathways are becoming a major class of drug. The functions of some of the kinases are described below.

The second messenger dependent protein kinases primarily mediate the effects of second messengers such as cyclic AMP (cAMP), cyclic GMP, inositol triphosphate, phosphatidylinositol, 3,4,5-triphosphate, cyclic-ADPribose, arachidonic acid, diacylglycerol and calcium-calmodulin. The cyclic-AMP dependent protein kinases (PKA) are important members of the STK family. Cyclic-AMP is an intracellular mediator of hormone action in all prokaryotic and animal cells that have been studied. Such hormone-induced cellular responses include thyroid hormone secretion, cortisol secretion, progesterone secretion, glycogen breakdown, bone resorption, and regulation of heart rate and force of heart muscle contraction. PKA is found in all animal cells and is thought to account for the effects of cyclic-AMP in most of these cells. Altered PKA expression is implicated in a variety of disorders and diseases including cancer, thyroid disorders, diabetes, atherosclerosis, and cardiovascular disease.

Calcium-calmodulin (CaM) dependent protein kinases are also members of the STK family. Calmodulin is a calcium receptor that mediates many calcium regulated processes by binding to target proteins in response to the binding of calcium. The principle target protein in these processes is CaM dependent protein kinases. CaM-kinases are involved in regulation of smooth muscle contraction (MLC kinase), glycogen breakdown (phosphorylase kinase), and neurotransmission (CaM kinase I and CaM kinase II). CaM kinase I phosphorylates a variety of substrates including the neurotransmitter related proteins synapsin I and II, the gene transcription regulator, CREB, and the cystic fibrosis conductance regulator protein, CFTR. CaM II kinase also phosphorylates synapsin at different sites, and controls the synthesis of catecholamines in the brain through phosphorylation and activation of tyrosine hydroxylase. Many of the CaM kinases are activated by phosphorylation in addition to binding to CaM. The kinase may autophosphorylate itself, or be phosphorylated by another kinase as part of a “kinase cascade”.

Another ligand-activated protein kinase is 5′-AMP-activated protein kinase (AMPK). Mammalian AMPK is a regulator of fatty acid and sterol synthesis through phosphorylation of the enzymes acetyl-CoA carboxylase and hydroxymethylglutaryl-CoA reductase and mediates responses of these pathways to cellular stresses such as heat shock and depletion of glucose and ATP. AMPK is a heterotrimeric complex comprised of a catalytic alpha subunit and two non-catalytic beta and gamma subunits that are believed to regulate the activity of the alpha subunit. Subunits of AMPK have a much wider distribution in non-lipogenic tissues, such as brain, heart, spleen, and lung, than expected. This distribution suggests that AMPK's functions may extend beyond regulation of lipid metabolism alone.

The mitogen-activated protein kinases (MAP) are also members of the STK family. MAP kinases also regulate intracellular signaling pathways. They mediate signal transduction from the cell surface to the nucleus via phosphorylation cascades. Several subgroups have been identified, and each manifests different substrate specificities and responds to distinct extracellular stimuli. MAP kinase signaling pathways are present in mammalian cells as well as in yeast. The extracellular stimuli that activate mammalian pathways include epidermal growth factor (EGF), ultraviolet light, hyperosmolar medium, heat shock, endotoxic lipopolysaccharide (LPS), and pro-inflammatory cytokines, such as tumor necrosis factor (TNF) and interleukin-1 (IL-1).

EGF receptor is found in over half of breast tumors unresponsive to hormone. EGF is found in many tumors, and EGF may be required for tumor cell growth. Antibodies to EGF blocked the growth of tumor xenografts in mice. An antisense oligonucleotide for amphiregulin inhibited growth of a pancreatic cancer cell line.

Tamoxifen, a protein kinase C inhibitor with anti-estrogen activity, is currently a standard treatment for hormone-dependent breast cancer. The use of this compound may increase the risk of developing cancer in other tissues such as the endometrium. Raloxifene, a related compound, has been shown to protect against osteoporosis. The tissue specificity of inhibitors must be considered when identifying therapeutic targets.

Signal transduction to the nucleus in response to extracellular stimulus by a growth factor involves the mitogen activated protein (MAP) kinases. MAP kinases are a family of protein serine/threonine kinases which mediate signal transduction from extracellular receptors or heat shock, or UV radiation. Cell proliferation and differentiation in normal cells are under the regulation and control of multiple MAP kinase cascades. Aberrant and deregulated functioning of MAP kinases can initiate and support carcinogenesis. Insulin and IGF1 also activate a mitogenic MAP kinase pathway that may be important in acquired insulin resistance occurring in type 2 diabetes.

Many cancers become refractory to chemotherapy by developing a survival strategy involving the constitutive activation of the phosphatidylinositol 3-kinase-protein kinase B/Akt signaling cascade. This survival signaling pathway thus becomes an important target for the development of specific inhibitors that would block its function. PI-3 kinase/Akt signaling is equally important in diabetes. The pathway activated by RTKs subsequently regulates glycogen synthase 3 (GSK3) and glucose uptake. Since AKT has decreased activity in type 2 diabetes, it provides a therapeutic target.

Protein kinase inhibitors provide much of our knowledge about in vivo regulation and coordination of physiological functions of endogenous peptide inhibitors. A pseudosubstrate sequence within PKC acts to inhibit the kinase in the absence of its lipid activator. A PKC inhibitor, such as chelerythrine, acts on the catalytic domain to block substrate interaction, while calphostin acts on the regulatory domain to mimic the pseudosubstrate sequence and block ATPase activity, or to inhibit cofactor binding.

Although some protein kinases have, to date, no known system of physiological regulation, many are activated or inactivated by autophosphorylation or phosphorylation by upstream protein kinases. The regulation of protein kinases also occurs during the transcription, post-transcription, and post-translation processes. The mechanism of post-transcriptional regulation is alternative splicing of precursor mRNA. For example, protein kinase C βI and βII are two isoforms of a single PKCβ gene derived from differences in the splicing of the exon encoding the C-terminal 50-52 amino acids. Splicing can be regulated by a kinase cascade in response to peptide hormones, such as insulin and IGF-1. PKC βI and βII have different specificities for phosphorylating members of the mitogen activated protein (MAP) kinase family, for glycogen synthase 3β, for nuclear transcription factors, such as TLS/Fus, and for other nuclear kinases. By inhibiting the post-transcriptional alternative splicing of PKC βII mRNA, PKC βII-dependent processes are inhibited.

The development of antisense oligonucleotides to inhibit the expression of various protein kinases has been successful. Antisense oligonucleotides are short lengths of synthetically manufactured, chemically modified DNA or RNA designed to specifically interact with mRNA transcripts encoding target proteins. The interaction of the antisense moiety with mRNA inhibits protein translation and, in some cases, post-transcriptional processing (e.g., alternative splicing and stability) of mRNA. Antisense oligonucleotides have been developed to alter alternative splicing of mRNA forms for inhibiting the translation of PKCα.

Protein kinase C isoforms have been implicated in cellular changes observed in the vascular complications of diabetes. Hyperglycemia is associated with increased levels of PKCα and β isoforms in renal glomeruli of diabetic rats. Oral administration of a PKCβ inhibitor prevented the increased mRNA expression of TGF-β1 and extracellular matrix component genes. Administration of the specific PKCβ inhibitor (LY333531) also normalized levels of cytokines, caldesmon, and hemodynamics of retinal and renal blood flow. Overexpression of the PKCβ isoform in the myocardium resulted in cardiac hypertrophy and failure. The use of LY333531 to prevent adverse effects of cardiac PKCβ overexpression in diabetic subjects is under investigation. The compound is also in Phase I/II clinical trials for diabetic retinopathy and diabetic macular edema indicating that it may be pharmacodynamically active.

PRK (proliferation-related kinase) is a serum/cytokine inducible STK that is involved in regulation of the cell cycle and cell proliferation in human megakaroytic cells. PRK is related to the polo (derived from human polo gene) family of STKs implicated in cell division. PRK is down-regulated in lung tumor tissue and may be a proto-oncogene whose deregulated expression in normal tissue leads to oncogenic transformation. Altered MAP kinase expression is implicated in a variety of disease conditions including cancer, inflammation, immune disorders, and disorders affecting growth and development.

DNA-dependent protein kinase (DNA-PK) is involved in the repair of double-strand breaks in mammalian cells. This enzyme requires ends of double stranded DNA or transitions from single-stranded to double-stranded DNA in order to act as a serine/threonine kinase. Cells with defective or deficient DNA-PK activity are unable to repair radiation induced DNA double-strand breaks and are consequently very sensitive to the lethal effects of ionizing radiation. Inhibition of DNA-PK has the potential to increase the efficacy of anti-tumor treatment with radiation or chemotherapeutic agents.

The cyclin-dependent protein kinases (CDKs) are another group of STKs that control the progression of cells through the cell cycle. Cyclins are small regulatory proteins that act by binding to and activating CDKs that then trigger various phases of the cell cycle by phosphorylating and activating selected proteins involved in the mitotic process. CDKs are unique in that they require multiple inputs to become activated. In addition to the binding of cyclin, CDK activation requires the phosphorylation of a specific threonine residue and the dephosphorylation of a specific tyrosine residue.

Cellular inhibitors of CDKs also play a major role in cell cycle progression. Alterations in the expression, function, and structure of cyclin and CDK are encountered in the cancer phenotype. Therefore, CDKs may be important targets for new cancer therapeutic agents.

Chemotherapy resistant cells tend to escape apoptosis. Under certain circumstances, inappropriate CDK activation may even promote apoptosis by encouraging the progression of the cell cycle under unfavorable conditions, i.e., attempting mitosis while DNA damage is largely unrepaired.

Purines and purine analogs act as CDK inhibitors. Flavopiridol is a flavonoid that causes 50% growth inhibition of tumor cells at 60 nM. It also inhibits EGFR and protein kinase A. Flavopiridel induces apoptosis and inhibits lymphoid, myeloid, colon, and prostate cancer cells grown in vivo as tumor xenografts in nude mice.

Staurosporine and its derivative, UCN-01, in addition to inhibiting protein kinase C, inhibit cyclin B/CDK (IC₅₀=3 to 6 nM). Staurosporine is toxic, but its derivative 7-hydroxystaurosporine (UCN-01) has anti-tumor properties and is in clinical trials. UCN-01 affects the phosphorylation of CDKs and alters the cell cycle checkpoint functioning. These compounds illustrate that multiple intracellular targets may be affected as the concentration of an inhibitor is increased within cells.

Protein tyrosine kinases, PTKs, specifically phosphorylate tyrosine residues on their target proteins and may be divided into transmembrane, receptor PTKs and non-transmembrane, non-receptor PTKs. Transmembrane protein tyrosine kinases are receptors for most growth factors. Binding of a growth factor to the receptor activates the transfer of a phosphate group from ATP to selected tyrosine side chains of the receptor and other specific proteins. Growth factors (GF) associated with receptor PTKs include; epidermal GF, platelet-derived GF, fibroblast GF, hepatocyte GF, insulin and insulin-like GFs, nerve GF, vascular endothelial GF, and macrophage colony stimulating factor.

Since RTKs stimulate tumor cell proliferation, inhibitors of RTKs may inhibit the growth and proliferation of such cancers. Inhibitors of RTKs are also useful in preventing tumor angiogenesis and can eliminate support from the host tissue by targeting RTKs located on vascular cells, such as blood vessel endothelial cells and stromal fibroblasts. For example, VEGF stimulates endothelial cell growth during angiogenesis, and increases the permeability of tumor vasculature so that proteins and other growth factors become accessible to the tumor. Broad-spectrum antitumor efficacy of an oral dosage form of an inhibitor of VEGF signaling has been reported. Thus, inhibition of VEGF receptor signaling presents an important therapeutic target. An extracellular receptor can also be a target for inhibition. For example, the EGF receptor family and its ligands are overexpressed and exist as an autocrine loop in many tumor types.

Increasing knowledge of the structure and activation mechanism of RTKs and the signaling pathways controlled by tyrosine kinases provided the possibility for the development of target specific drugs and new anti-cancer therapies. Approaches towards the prevention or interception of deregulated RTK signaling include the development of selective components that target either the extracellular ligand-binding domain or the intracellular substrate binding region.

The most successful strategy to selectively kill tumor cells is the use of monoclonal antibodies (mAbs) that are directed against the extracellular domain of RTKs, which are critically involved in cancer and are expressed at the surface of tumor cells. In the past years, recombinant antibody technology has made an enormous progress in the design, selection and production of newly engineered antibodies. It is also possible to generate humanized antibodies, human-mouse chimeric or bispecific antibodies for targeted cancer therapy. Mechanistically, anti-RTK mAbs might work by blocking the ligand-receptor interaction and therefore inhibiting ligand-induced RTK signaling and increasing RTK down-regulation and internalization. In addition, binding of mAbs to certain epitopes on the cancer cells may induce immune-mediated responses, such as opsonization and complement-mediated lysis, and trigger antibody-dependent cellular cytotoxicity by macrophages or natural killer cells. In recent years, it became evident that mAbs control tumor growth by altering the intracellular signaling pattern inside the targeted tumor cell, leading to growth inhibition and/or apoptosis. In addition, bispecific antibodies can bridge selected surface molecules on a target cell with receptors on an effector cell, thus triggering cytotoxic responses against the target cell. Despite the toxicity that has been seen in clinical trials of bispecific antibodies, advances in antibody engineering, characterization of tumor antigens and immunology might help to produce rationally designed bispecific antibodies for anti-cancer therapy.

Another promising approach to inhibiting aberrant RTK signaling is to develop small molecule drugs that selectively interfere with the intrinsic tyrosine kinase activity and thereby block receptor autophosphorylation and activation of downstream signal transducers. The tyrphostins, which belong to the quinazolines, are one important group of such inhibitors that compete with ATP for the ATP binding site at the receptor's tyrosine kinase domain and some members of the group have been shown to specifically inhibit the EGFR. Potent and selective inhibitors of receptors involved in neovascularization have been developed and are now undergoing clinical evaluation. New classes of tyrosine kinase inhibitors (TKIs) with increased potency and selectivity, higher in vitro and in vivo efficacy and decreased toxicity have been developed using the advantages of structure-based drug design, crystallographic structure information, combinatorial chemistry and high-throughput screening.

Recombinant immunotoxins provide another possibility of target-selective drug design. Recombinant immunotoxins are composed of a bacterial or plant toxin either fused or chemically conjugated to a specific ligand, such as the variable domains of the heavy and light chains of mAbs or to a growth factor. Immunotoxins may contain bacterial toxins, such as Pseudomouas exotoxin A or diphtheria toxin, or plant toxins, such as ricin A or clavin. These recombinant molecules can selectively kill their target cells when internalized after binding to cell surface receptors of the target cells.

The use of antisense oligonucleotides represents another strategy to inhibit the activation of RTKs. Antisense oligonucleotides are short pieces of synthetic DNA or RNA that are designed to interact with the mRNA to block the transcription and thus the expression of the target proteins. Antisense oligonucleotides interact with the mRNA by Watson-Crick base-pairing and are therefore highly specific to the target protein. Several preclinical and clinical studies suggest that antisense therapy might be therapeutically useful for the treatment of solid tumors.

The potential of RTKs and their relevant signaling as selective anti-cancer targets for therapeutic intervention has been recognized. As a consequence, a variety of successful target specific drugs such as mAbs and RTK inhibitors have been developed and are currently being evaluated in clinical trials. Table 3 summarizes the most successful drugs against receptor tyrosine kinase signaling which are currently evaluated in clinical phases or have already been approved by the FDA.

TABLE 3
RTK drugs currently under clinical evaluation

| RTK | Drug | Company | Description | Status |
|---|---|---|---|---|
| EGFR | ZA18539 | AstraZeneca | TKI that inhibits | Phase |
| | Iressa | | EGFR signaling | III |
| EGFR | Cetuximab | ImClone | Mab directed | Phase |
| | C225 | Systems | against EGFR | III |
| EGFR | EGF | Seragen | Recombinant | Phase |
| | fusion protein | | diphtheria | II |
| | | | toxin-hEGF | |
| | | | fusion protein | |
| HER2 | Trastuzumab | Genetech | Mab directed | Approved |
| | Herceptin | | against HER2 | by the |
| | | | | FDA in |
| | | | | 1998 |
| IGF-IR | INX-4437 | INEX USA | Antisense | Phase |
| | | | oligonucleotides | I |
| | | | targeting IGR-IR | |
| VEGFR | SU5416 | SUGEN | TKI that | Phase |
| | | | inhibits VEGFR2 | II |
| VEGFR/ | SU6668 | SUGEN | RTK inhibition | Phase |
| FGFR/ | | | of VEGFR, | I |
| PDGFR | | | FGFR, and | |
| | | | PDGFR | |

Non-Receptor PTKs Lack transmembrane regions and, instead, form complexes with the intracellular regions of cell surface receptors. Receptors that function through non-receptor PTKs include those for cytokines, hormones (growth hormone and prolactin) and antigen-specific receptors on T and B lymphocytes.

Many of the PTKs were first identified as the products of mutant oncogenes in cancer cells where their activation was no longer subject to normal cellular controls. In fact, about one third of the known oncogenes encode PTKs, and it is well known that cellular transformation (oncogenesis) is often accompanied by increased tyrosine phosphorylation activity.

Many tyrosine kinase inhibitors, such as flavopiridol, genistem, erbstatin, lavendustin A, staurosporine, and UCN-01, are derived from natural products. Inhibitors directed to the ATP binding site are also available. Signals from RTKs can also be inhibited at other target sites such as nuclear tyrosine kinases, membrane anchors (inhibition of farnesylation) and transcription factors.

Targeting the signaling potential of growth promoting tyrosine kinases such as EGFR, HER2, PDGFR, src, and abl, will block tumor growth while blocking IGF-I and TRK will interfere with tumor cell survival. Inhibition of these kinases will lead to tumor shrinkage and apoptosis. FklI/KDR and src are kinases necessary for neovascularization (angiogenesis) of tumors. Inhibition of these kinases will slow tumor growth and decrease metastases.

Inhibitors of RTKs suppress tumor development by preventing cell migration, invasion and metastases. These drugs are likely to increase the time required for tumor progression, and may inhibit or attenuate the aggressiveness of the disease but may not initially result in measurable tumor regression.

An example of cancer arising from a defective tyrosine kinase is a class of ALK positive lymphomas referred to as “ALKomas” which display inappropriate expression of a neural-specific tyrosine kinase, anaplastic lymphoma kinase (ALK).

Iressa (ZD1839) is an orally active selective EGF-R inhibitor. This compound disrupts signaling involved in cancer cell proliferation. The clinical efficacy of this agent shows that it is well tolerated by patients undergoing Phase I/II clinical trials. The compound has shown promising cytotoxicity towards several cancer cell lines.

Since the majority of protein kinases are expressed in the brain, often in a neuron-specific fashion, protein phosphorylation must play a key role in the development and function of the vertebrate central nervous system. Thus neuron-specific kinases are well established as targets for the development of pharmacologically active modulators.

In summary, kinase proteins are a major target for drug action and development. Accordingly, it is valuable to the field of pharmaceutical development to identify and characterize previously unknown members of kinase proteins. The present invention advances the state of the art by providing a previously unidentified human kinase protein that has homology to rat MRCK.

Utility of the MRCK2 Gene and MRCK2 Kinase

MRCKs are serine/threonine kinases with multiple functional domains. MRCKs are thought to act as a downstream effector of Cdc42 in cytoskeletal reorganization. Cdc42 is an essential GTPase that belongs to the Rho/Rac subfamily of Ras-like GTPases. These proteins act as molecular switches by responding to exogenous and/or endogenous signals and relaying those signals to activate downstream components of a biological pathway. The 11 current members of the Cdc42 family display between 75 and 100% amino acid identity and are functional, as well as structural, homologs. Cdc42 transduces signals to the actin cytoskeleton to initiate and maintain polarized growth and to mitogen-activated protein morphogenesis. In the budding yeast Saccharomyces cerevisiae, Cdc42 plays an important role in multiple actin-dependent morphogenetic events such as bud emergence, mating-projection formation, and pseudohyphal growth. In mammalian cells, Cdc42 regulates a variety of actin-dependent events and induces the JNK/SAPK protein kinase cascade, which leads to the activation of transcription factors within the nucleus. Cdc42 mediates these processes through interactions with a myriad of downstream effectors. In addition, Cdc42 has been implicated in a number of human diseases through interactions with its regulators and downstream effectors.

The MRCK family includes at least two related protein kinases: MRCK alpha and MRCK beta, which were isolated from a human brain cDNA library using a monoclonal antibody directed against myotonic dystrophy protein kinase (DMPK). The epitope shared by DMPK and MRCKs was located at the catalytic site of DMPK using a phage-displayed random peptide library (Lam, Hum Mol Genet 9:2167-2173, 2000).

MRCKs are involved in Cdc42-mediated myosin light chain phosphorylation (Dong et al., Eur J Cell Biol 81:231-42, 2002). Specifically, MRCK alpha was implicated in Cdc42-mediated peripheral actin formation and neurite outgrowth in HeLa and PC12 cells, respectively. It was suggest that MRCK alpha may act as a downstream effector of Cdc42 in cytoskeletal reorganization (Leung et al., Mol Cell Biol 18:130-40, 1998).

The native MRCK exists in high-molecular-weight complexes. The three independent coiled-coil (CC) domains and the N-terminal region preceding the kinase domain are responsible for intermolecular interactions leading to MRCK alpha multimerization. N-terminus-mediated dimerization and consequent trans-autophosphorylation are critical processes regulating MRCK alpha catalytic activities. A region containing the two distal CC domains (CC2 and CC3; residues 658 to 930) was found to interact intramolecularly with the kinase domain and negatively regulates its activity. Its deletion also resulted in an active kinase, confirming a negative auto-regulatory role. The N-terminus-mediated dimerization and activation of MRCK and the negative kinase-distal CC domain interaction are two mutually exclusive events that tightly regulate the catalytic state of the kinase. Disruption of this interaction by a mutant kinase domain resulted in increased kinase activity. MRCK kinase activity was also elevated when cells were treated with phorbol ester, which can interact directly with a cysteine-rich domain next to the distal CC domain. It therefore appears that binding of phorbol ester to MRCK releases its auto-inhibition, allowing N-terminal dimerization and subsequent kinase activation (Tan et al., Mol Cell Biol 21:2767-78, 2001).

The approximately 190-kD MRCK kinases preferentially phosphorylate non-muscle myosin light chain at serine 19, which is known to be crucial for activating actin-myosin contractility. The p21-binding domain binds GTP-Cdc42 but not GDP-Cdc42. The multidomain structure includes a cysteine-rich motif resembling those of protein kinase C and n-chimaerin and a putative Pleckstrin homology domain. MRCK alpha and Cdc42^{V12} co-localize, particularly at the cell periphery in transfected HeLa cells. Microinjection of a plasmid encoding MRCK alpha resulted in actin and myosin reorganization. Expression of kinase-dead MRCK alpha blocked Cdc42^{V12}-dependent formation of focal complexes and peripheral microspikes. This was not due to possible sequestration of the p21, as a kinase-dead MRCK alpha mutant defective in Cdc42 binding was an equally effective blocker. Coinjection of an MRCK alpha plasmid with a Cdc42 plasmid, at concentrations where Cdc42 plasmid by itself elicited no effect, led to the formation of the peripheral structures associated with a Cdc42-induced morphological phenotype. These Cdc42-type effects were not promoted upon coinjection with plasmids of kinase-dead or Cdc42-binding-deficient MRCK alpha mutants. These results suggest that MRCK alpha may act as a downstream effector of Cdc42 in cytoskeletal reorganization (Leung et al., Mol Cell Biol 18:130-40, 1998).

Two major substrates, p130 and p85, for MRCK alpha-kinase have been identified. P130 is identified as the myosin binding subunit p130, whereas p85 is a novel related protein. P85 contains N-terminal ankyrin repeats, an alpha-helical C terminus with leucine repeats, and a centrally located conserved motif with the MRCK alpha kinase phosphorylation site. Like MBS 130, p85 is specifically associated with protein phosphatase 1delta (PP1delta), and this requires the N-terminus, including the ankyrin repeats. This association is required for the regulation of both the catalytic activities and the assembly of actin cytoskeleton. The N-terminus, in association with PP1delta, is essential for actin depolymerization, whereas the C-terminus antagonizes this action. The C-terminal effects consist of two independent events that involved both a conserved phosphorylation inhibitory motif and an alpha-helical leucine repeats. The former was able to interact with PP1delta only in the phosphorylated state and result in inactivation of PP1delta activity. This provides further evidence that phosphorylation of a myosin binding subunit protein by specific kinases confers conformational changes in a highly conserved region that plays an essential role in the regulation of its catalytic subunit activities (Tan et al., J Biol Chem 276:21209-16, 2001).

Taken together, MRCKs appear to be an important player in cytoskeletal reorganization, neuronal differentiation, and myotonic dystrophy. MRCKs may serve as a major target for drug action and development.

The present invention provides a new human kinase (MRCK2) which has sequence and structure similarities to rat MRCK alpha and other kinases. The multiple domains in MRCK2 share high sequence identities with the corresponding domains in other kinases. Each of these domains, either in its native form or in a mutant form, can be used to affect the function of the corresponding domain in other kinases. The kinase domain in MRCK2 can be used to phosphorylate suitable substrates, including p130 and p85 or substrate peptides containing MRCK alpha phosphorylation sites. The substrate peptides can be conjugated to antibodies, and the phosphate groups added to the substrate peptides can be radioactively or fluorescently labeled. Antibodies thus labeled can be used in various detection assays, as appreciated by one of skill in the art.

The MRCK2 gene and gene products can be used as molecular markers for diagnosing, prognosing, and monitoring the treatment of disorders related to the aberrant expression of MRCK2. In addition, the MRCK2 gene can be used to screen for potential agents or drugs capable of enhancing or inhibiting the MRCK2 gene expression in human cells. The MRCK2 gene products (polynucleotide and polypeptide) can be used to screen for potential agents or drugs capable of enhancing or inhibiting MRCK2 activity. Furthermore, various therapeutic methods for treating disorders related to the aberrant expression of MRCK2 can be designed based on the MRCK2 gene, its variants, or the agents/drugs that affect the expression of the MRCK2 gene or the activity of the MRCK2 gene products.

The following subsections illustrate examples of the utilities of human MRCK2 gene and MRCK2 kinase. Various changes and modifications within the scope of the present invention will become apparent to those skilled in the art from the present description.

Polynucleotides and Variants Thereof

One aspect of the invention pertains to isolated polynucleotide probes capable of hybridizing to the MRCK2 gene or its transcripts, such as MRCK2 mRNAs. These probes can be used to detect the expression level of the MRCK2 gene in human tissue or cells. The present invention also contemplates polynucleotide fragments for use as PCR primers for the amplification or mutation of the MRCK2 gene or the MRCK2 kinase-coding sequences. Another aspect of the invention pertains to isolated polynucleotides that encode MRCK2, or a fragment or mutant thereof. These polynucleotides can be used for expressing MRCK2, or a fragment or mutant thereof. The protein products thus expressed can be used to screen for agents/drugs that modulate an activity of MRCK2. In addition, these polynucleotides can be used to designing gene therapy vectors which target the expression of the MRCK2 gene or an activity of MRCK2 in humans.

A polynucleotide comprising SEQ ID NO:1 or SEQ ID NO:3 can be prepared using standard molecular biology techniques as appreciated by one of ordinary skill in the art. For instance, primers derived from the 5′ and 3′ ends of SEQ ID NO:1 can be used to amplify mRNAs isolated from human tissues. The cDNA thus produced contains SEQ ID NO:1. Likewise, primers for amplifying the human genomic sequence containing SEQ ID NO:3 can be designed and used to prepare the genomic sequence of the MRCK2 gene. A variant (such as a homolog) or a fragment of SEQ ID NO:1 or SEQ ID NO:3 can be similarly prepared. Alternatively, probes can be designed to screen for cDNA or genomic sequence libraries in order to identify polynucleotide molecules comprising the full-length or fragments of SEQ ID NO:1 or SEQ ID NO:3. The molecules thus identified can be used to create suitable vectors comprising the full-length SEQ ID NO:1 or SEQ ID NO:3.

Polynucleotides capable of hybridizing to the MRCK2 gene can be prepared by standard synthetic techniques, e.g., using an automated DNA synthesizer. Preferably, the polynucleotide probes can hybridize to the MRCK2 gene under reduced stringent conditions, stringent conditions, or highly stringent conditions. In one embodiment, the polynucleotides comprise at least 15, 20, 25, 30, 50, 100, 150, 200, 250, 300, 350, 400, 450, 500, 550, 600, 650, 700, 750, 800, 850, 900, 950, 1000 or more consecutive nucleotides of SEQ ID NO:1. Any fragments of SEQ ID NO:1 and SEQ ID NO:3 may be used as hybridization probes or PCR primers for the MRCK2 gene or its transcripts. The probes/primers can be substantially purified.

In a preferred embodiment, the hybridization probes for the MRCK2 gene comprise a label group. The label group can be a radioisotope, a fluorescent compound, an enzyme, or an enzyme co-factor. Probes thus labeled can be used as part of a diagnostic kit for determining the expression level of the MRCK2 gene in human tissues.

This invention encompasses human MRCK2 gene homologs in other species. These homologs can be determined by search different sequence databases, such as the Entrez/GenBank sequence databases maintained by the NCBI. The invention also encompasses polynucleotide molecules which are structurally different from the molecules described above, but have the substantially same properties as the molecules described above. Such molecules include allelic variants, which will be described below in greater detail.

DNA sequence polymorphism in human MRCK2 gene exists among different individuals due to natural allelic variations. An allele is one of a group of genes which occur alternatively at a given genetic locus. DNA polymorphisms that affect the RNA expression level of the MRCK2 gene can also exist, e.g. through affecting the regulation or degradation of expression of the gene. The present invention contemplates all allelic variants of human MRCK2 gene. Allelic variants and other homologs of the MRCK2 gene can be isolated using probes/primers derived from SEQ ID NO:1 or SEQ ID NO:3.

It should, of course, be understood that SEQ ID NO:1 and SEQ ID NO:3 can be modified. The modified polynucleotides can comprise one or more mutations. These mutations can be substitutions, additions or deletions of 1, 2, 3, 5, 10, 15, 20 or more nucleotide residues in SEQ ID NO:1 or SEQ ID NO:3. Standard techniques can be used, such as site-directed mutagenesis or PCR-mediated mutagenesis. Preferably, these mutations create conservative amino acid substitutions. Alternatively, mutations can be introduced randomly along all or part of the MRCK2 gene or its cDNA, such as by saturation mutagenesis. Following mutagenesis, the encoded proteins can be expressed recombinantly and their activities can be determined.

In one embodiment, nucleotide substitutions leading to amino acid substitutions at “non-essential” amino acid residues can be introduced. A “non-essential” amino acid residue is a residue that can be altered without changing the biological activity of the protein. In contrast, an “essential” amino acid residue is required for the biological activity of the protein. Amino acid residues that are conserved among allelic variants or homologs of the MRCK2 gene from different species preferably are not changed in the present invention.

Accordingly, another aspect of the invention pertains to MRCK2 proteins that contain changes in amino acid residues that are not essential for the biological activity of MRCK2. These proteins differ in amino acid sequence from the original human MRCK2 kinase, but retain its biological activity. In one embodiment, the modified protein comprises an amino acid sequence at least about 96%, 97%, 98%, 99% or more homologous to SEQ ID NO:2.

In another embodiment, MRCK2 proteins contain mutations in amino acid residues which result in inhibition of MRCK2 activity. These mutated MRCK2 proteins can be used to inhibit MRCK2 activity in patients with disorders related to the aberrant expression of MRCK2.

A polynucleotide of this invention can be further modified to increase its stability in vivo. Possible modifications include, but are not limited to, the addition of flanking sequences at the 5′ and/or 3′ ends; the use of phosphorothioate or 2-o-methyl rather than phosphodiester linkages in the backbone; and/or the inclusion of nontraditional bases such as inosine, queosine and wybutosine, as well as acetyl-methyl-, thio- and other modified forms of adenine, cytidine, guanine, thymine and uridine.

Polynucleotide molecules which are antisense to the MRCK2 gene can be prepared. An “antisense” polynucleotide comprises a nucleotide sequence which is complementary to a “sense” polynucleotide which encodes a protein. An antisense polynucleotide can bind via hydrogen bonds to the sense polynucleotide.

Antisense polynucleotides of the invention can be designed according to the rules of Watson and Crick base pairing. The antisense polynucleotide molecule can be complementary to the entire coding region or part of the coding region of the MRCK2 gene. The antisense polynucleotide molecule can also be complementary to a “noncoding region” in the coding strand of the MRCK2 gene. Preferably, the antisense polynucleotide is an oligonucleotide which is antisense to only a portion of the MRCK2 gene. An antisense polynucleotide can be, for example, about 5, 10, 15, 20, 25, 30, 35, 40, 45 or 50 nucleotides in length. An antisense polynucleotide of the invention can be constructed using chemical synthesis and enzymatic ligation reactions as appreciated by one of ordinary skill in the art. For example, an antisense polynucleotide can be chemically synthesized using naturally occurring nucleotides or variously modified nucleotides designed to increase the biological stability of the molecules or to increase the physical stability of the duplex formed between the antisense and sense polynucleotides. Examples of modified nucleotides which can be used to generate the antisense polynucleotide include 5-fluorouracil, 5-bromouracil, 5-chlorouracil, 5-iodouracil, hypoxanthine, xantine, 4-acetylcytosine, 5-(carboxyhydroxymethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyl adenosine, unacil-5-oxyacetic acid, wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, (acp3)w, and 2,6-diaminopurine. Phosphorothioate derivatives and acridine substituted nucleotides can also be used. Alternatively, the antisense polynucleotide can be produced biologically using an expression vector into which a polynucleotide has been subcloned in an antisense orientation (i.e., RNA transcribed from the inserted polynucleotide will be of an antisense orientation to the target polynucleotide of interest).

The antisense polynucleotides of the invention can be administered to a subject or applied in situ such that they hybridize or bind to cellular mRNAs and/or genomic DNAs that encode MRCK2 kinase, thereby inhibiting the expression of MRCK2 kinase. The hybridization can result in a stable duplex via conventional nucleotide complementarity. An example route for administering antisense polynucleotides includes direct injection at a tissue site. Antisense polynucleotides can also be modified first, and then administered systemically. For example, for systemic administration, antisense molecules can be modified such that they specifically bind to receptors or antigens expressed on a selected cell surface. Suitable modifications include linking the antisense polynucleotides to peptides or antibodies which bind to the cell surface receptors or antigens. In addition, the antisense polynucleotides can be delivered to cells using vectors. To achieve sufficient intracellular concentrations of the antisense molecules, strong pol II or pol III promoters may be used in the vectors.

In one embodiment, the antisense polynucleotides are α-anomeric polynucleotides. An α-anomeric polynucleotide molecule forms specific double-stranded hybrid with a complementary RNA in which, contrary to the usual β-units, the strands run parallel to each other. The antisense polynucleotide molecule can also comprise a 2-o-methylribonucleotide or a chimeric RNA-DNA analogue.

In another embodiment, the antisense polynucleotide is a ribozyme. Ribozymes are catalytic RNA molecules with ribonuclease activity which are capable of cleaving a single-stranded polynucleotide, such as an mRNA, to which they have a complementary region. Thus, ribozymes (e.g., hammerhead ribozymes described in Haselhoif and Gerlach Nature 334:585-591, 1988) can be used to catalytically cleave mRNA transcripts of MRCK2 in order to inhibit its expression. A ribozyme having specificity for the MRCK2 gene or its transcripts can be designed based upon SEQ ID NO:1 or 3. mRNAs transcribed from the MRCK2 gene can be used to select from a pool of RNA molecules a catalytic RNA having a specific ribonuclease activity.

Alternatively, the expression of the MRCK2 gene can be inhibited by using nucleotide sequences complementary to the regulatory region (e.g., the promoter and/or enhancers). These nucleotide sequences can form triple helical structures that prevent transcription of the gene in the target cells.

Expression of the MRCK2 gene can also be inhibited using RNA interference (“RNAi”). RNAi is a phenomenon in which the introduction of double-stranded RNA (dsRNA) into certain organisms or cell types causes degradation of the homologous mRNA. First discovered in the nematode Caenorhabditis elegans, RNAi has since been found to operate in a wide range of organisms. For example, in mammalian cells, introduction of long dsRNA (>30 nt) can initiate a potent antiviral response, exemplified by nonspecific inhibition of protein synthesis and RNA degradation. RNA interference provides a mechanism of gene silencing at the mRNA level. In recent years, RNAi has become an endogenous and potent gene-specific silencing technique that uses double-stranded RNAs (dsRNA) to mark a particular transcript for degradation in vivo. It also offers an efficient and broadly applicable approach for gene knock-out. In addition, RNAi technology can be used for therapeutic purposes. For example, RNAi targeting Fas-mediated apoptosis has been shown to protect mice from fulminant hepatitis. RNAi technology has been disclosed in numerous publications, such as U.S. Pat. Nos. 5,919,619, 6,506,559 and PCT Publication Nos. WO99/14346, WO01/70949, WO01/36646, WO00/63364, WO00/44895, WO01/75164, WO01/92513, WO01/68836 and WO01/29058.

In a preferred embodiment, short interfering RNAs (siRNA) are used. siRNAs are dsRNAs having 19-25 nucleotides. siRNAs can be produced endogenously by degradation of longer dsRNA molecules by an RNase III-related nuclease called Dicer. siRNAs can also be introduced into a cell exogenously or by transcription of an expression construct. Once formed, the siRNAs assemble with protein components into endoribonuclease-containing complexes known as RNA-induced silencing complexes (RISCs). An ATP-generated unwinding of the siRNA activates the RISCs, which in turn target the complementary mRNA transcript by Watson-Crick base-pairing, thereby cleaving and destroying the mRNA. Cleavage of the mRNA takes place near the middle of the region bound by the siRNA strand. This sequence-specific mRNA degradation results in gene silencing.

At least two ways can be employed to achieve siRNA-mediated gene silencing. First, siRNAs can be synthesized in vitro and introduced into cells to transiently suppress gene expression. Synthetic siRNA provides an easy and efficient way to achieve RNAi. siRNA are duplexes of short mixed oligonucleotides which can include, for example, 19 nucleotides with symmetric dinucleotide 3′ overhangs. Using synthetic 21 bp siRNA duplexes (e.g., 19 RNA bases followed by a UU or dTdT 3′ overhang), sequence-specific gene silencing can be achieved in mammalian cells. These siRNAs can specifically suppress targeted gene translation in mammalian cells without activation of DNA-dependent protein kinase (PKR) by longer dsRNA, which may result in non-specific repression of translation of many proteins.

Second, siRNAs can be expressed in vivo from vectors. This approach can be used to stably express siRNAs in cells or transgenic animals. In one embodiment, siRNA expression vectors are engineered to drive siRNA transcription from polymerase III (pol III) transcription units. Pol III transcription units are suitable for hairpin siRNA expression, since they deploy a short AT rich transcription termination site that leads to the addition of 2 bp overhangs (e.g., UU) to hairpin siRNAs—a feature that is helpful for siRNA function. The Pol III expression vectors can also be used to create transgenic mice that express siRNA.

In another embodiment, siRNAs can be expressed in a tissue-specific manner. Under this approach, long double-stranded RNAs (dsRNAs) are first expressed from a promoter (such as CMV (pol II)) in the nuclei of selected cell lines or transgenic mice. The long dsRNAs are processed into siRNAs in the nuclei (e.g., by Dicer). The siRNAs exit from the nuclei and mediate gene-specific silencing. A similar approach can be used in conjunction with tissue-specific promoters to create tissue-specific knockdown mice.

Any 3′ dinucleotide overhang, such as UU, can be used for siRNA design. In some cases, G residues in the overhang are avoided because of the potential for the siRNA to be cleaved by RNase at single-stranded G residues.

With regard to the siRNA sequence itself, it has been found that siRNAs with 30-50% GC content can be more active than those with a higher G/C content in certain cases. Moreover, since a 4-6 nucleotide poly(T) tract may act as a termination signal for RNA pol III, stretches of ≧4 Ts or As in the target sequence may be avoided in certain cases when designing sequences to be expressed from an RNA pol III promoter. In addition, some regions of mRNA may be either highly structured or bound by regulatory proteins. Thus, it may be helpful to select siRNA target sites at different positions along the length of the gene sequence. Finally, the potential target sites can be compared to the appropriate genome database (human, mouse, rat, etc.). Any target sequences with more than 16-17 contiguous base pairs of homology to other coding sequences may be eliminated from consideration in certain cases.

In one embodiment, siRNA can be designed to have two inverted repeats separated by a short spacer sequence and end with a string of Ts that serve as a transcription termination site. This design produces an RNA transcript that is predicted to fold into a short hairpin siRNA. The selection of siRNA target sequence, the length of the inverted repeats that encode the stem of a putative hairpin, the order of the inverted repeats, the length and composition of the spacer sequence that encodes the loop of the hairpin, and the presence or absence of 5′-overhangs, can vary to achieve desirable results.

The siRNA targets can be selected by scanning an mRNA sequence for AA dinucleotides and recording the 19 nucleotides immediately downstream of the AA. Other methods can also been used to select the siRNA targets. In one example, the selection of the siRNA target sequence is purely empirically determined (see e.g., Sui et al., Proc. Natl. Acad. Sci. USA 99: 5515-5520, 2002), as long as the target sequence starts with GG and does not share significant sequence homology with other genes as analyzed by BLAST search. In another example, a more elaborate method is employed to select the siRNA target sequences. This procedure exploits an observation that any accessible site in endogenous mRNA can be targeted for degradation by synthetic oligodeoxyribonucleotide/RNase H method (Lee et al., Nature Biotechnology 20:500-505, 2002).

In another embodiment, the hairpin siRNA expression cassette is constructed to contain the sense strand of the target, followed by a short spacer, the antisense strand of the target, and 5-6 Ts as transcription terminator. The order of the sense and antisense strands within the siRNA expression constructs can be altered without affecting the gene silencing activities of the hairpin siRNA. In certain instances, the reversal of the order may cause partial reduction in gene silencing activities.

The length of nucleotide sequence being used as the stem of siRNA expression cassette can range, for instance, from 19 to 29. The loop size can range from 3 to 23 nucleotides. Other lengths and/or loop sizes can also be used.

In yet another embodiment, a 5′ overhang in the hairpin siRNA construct can be used, provided that the hairpin siRNA is functional in gene silencing. In one specific example, the 5′ overhang includes about 6 nucleotide residues.

In a preferred embodiment, the target sequence for RNAi is a 21-mer sequence fragment selected from SEQ ID NO:1. The 5′ end of the target sequence has dinucleotide “NA,” where “N” can be any base and “A” represents adenine. The remaining 19-mer sequence has a GC content of between 45% and 55%. In addition, the remaining 19-mer sequence does not include (1) any three consecutive identical bases (i.e., GGG, CCC, TTT, or AAA); (2) seven “GC” in a role; and (3) any palindrome sequence with 5 or more bases. Furthermore, the target sequence has low sequence homology to other human genes. In one specific example, potential target sequences are searched by BLASTN against NCBI's human UniGene cluster sequence database. The human UniGene database contains non-redundant sets of gene-oriented clusters. Each UniGene cluster includes sequences that represent a unique gene. Fragments of SEQ ID NO:1 that produce no hit to other human genes under BLASTN search are selected as the preferred candidate sequences for RNAi. During the search, the e-value may be set at a stringent value (such as “1”). Table 4 lists exemplary MRCK2 gene target sequences for RNAi prepared using the above-described criteria. The siRNA sequences for each target sequence (the sense strand and the antisense strand) are also disclosed. In addition, the 5′ end location of each target sequence in SEQ ID NO:1 is identified (“5 End”).

TABLE 4

Exemplary RNAi Target Sequences of the MRCK2 Gene and the
Corresponding siRNAs

| | | | |
|---|---|---|---|
| Target Sequence | | siRNA Sense Strand | siRNA Antisense Strand |
| (SEQ ID NO) | 5′ End | (SEQ ID NO) | (SEQ ID NO) |

| | | | |
|---|---|---|---|
| AAGATGGAACGGTTCAGTCCT | 682 | GAUGGAACGGUUCAGUCCUUU | UUCUACCUUGCCAAGUCAGGA |
| (SEQ ID NO: 7) | | (SEQ ID NO: 8) | (SEQ ID NO: 9) |
| AAGGATCTTATTCGAAGGCTC | 942 | GGAUCUUAUUCGAAGGCUCUU | UUCCUAGAAUAAGCUUCCGAG |
| (SEQ ID NO: 10) | | (SEQ ID NO: 11) | (SEQ ID NO: 12) |
| AATGTTCAGAGGACTCTAGAC | 1287 | UGUUCAGAGGACUCUAGACUU | UUACAAGUCUCCUGAGAUCUG |
| (SEQ ID NO: 13) | | (SEQ ID NO: 14) | (SEQ ID NO: 15) |
| AATGAGCGGCTAACAGAATTG | 1746 | UGAGCGGCUAACAGAAUUGUU | UUACUCGCCGAUUGUCUUAAC |
| (SEQ ID NO: 16) | | (SEQ ID NO: 17) | (SEQ ID NO: 18) |
| AAGACAGGAAGCTACGTGAAC | 1930 | GACAGGAAGCUACGUGAACUU | UUCUGUCCUUCGAUGCACUUG |
| (SEQ ID NO: 19) | | (SEQ ID NO: 20) | (SEQ ID NO: 21) |
| AAGCTACGTGAACAGAGTGAG | 1938 | GCUACGUGAACAGAGUGAGUU | UUCGAUGCACUUGUCUCACUC |
| (SEQ ID NO: 22) | | (SEQ ID NO: 23) | (SEQ ID NO: 24) |
| AAGCTGACGAGTGAACTTGAT | 2331 | GCUGACGAGUGAACUUGAUUU | UUCGACUGCUCACUUGAACUA |
| (SEQ ID NO: 25) | | (SEQ ID NO: 26) | (SEQ ID NO: 27) |
| AAGGCAGACGTACTGTAGACT | 2932 | GGCAGACGUACUGUAGACUUU | UUCCGUCUGCAUGACAUCUGA |
| (SEQ ID NO: 28) | | (SEQ ID NO: 29) | (SEQ ID NO: 30) |
| AAGGATGTCCTGGTTCAACTG | 2992 | GGAUGUCCUGGUUCAACUGUU | UUCCUACAGGACCAAGUUGAC |
| (SEQ ID NO: 31) | | (SEQ ID NO: 32) | (SEQ ID NO: 33) |
| AAGGTCATGTCAGGATTCCTA | 3256 | GGUCAUGUCAGGAUUCCUAUU | UUCCAGUACAGUCCUAAGGAU |
| (SEQ ID NO: 34) | | (SEQ ID NO: 35) | (SEQ ID NO: 36) |
| AAGAGGCTTATGACAGCACTC | 3649 | GAGGCUUAUGACAGCACUCUU | UUCUCCGAAUACUGUCGUGAG |
| (SEQ ID NO: 37) | | (SEQ ID NO: 38) | (SEQ ID NO: 39) |
| AATGCACCATATCTCTCGGTG | 4371 | UGCACCAUAUCUCUCGGUGUU | UUACGUGGUAUAGAGAGCCAC |
| (SEQ ID NO: 40) | | (SEQ ID NO: 41) | (SEQ ID NO: 42) |
| AATAAGCGGCGTTATTCCTTC | 4617 | UAAGCGGCGUUAUUCCUUCUU | UUAUUCGCCGCAAUAAGGAAG |
| (SEQ ID NO: 43) | | (SEQ ID NO: 44) | (SEQ ID NO: 45) |
| AAGCGGCGTTATTCCTTCAGA | 4620 | GCGGCGUUAUUCCUUCAGAUU | UUCGCCGCAAUAAGGAAGUCU |
| (SEQ ID NO: 46) | | (SEQ ID NO: 47) | (SEQ ID NO: 48) |
| AACAGTTCCAACCTAAGCAGC | 5100 | CAGUUCCAACCUAAGCAGCUU | UUGUCAAGGUUGGAUUCGUC |
| (SEQ ID NO: 49) | | (SEQ ID NO: 50) | G |
| | | | (SEQ ID NO: 51) |
| CAGAATCGCTGGTGGAGACAT | 850 | GAAUCGCUGGUGGAGACAUUU | UUCUUAGCGACCACCUCUGUA |
| (SEQ ID NO: 52) | | (SEQ ID NO: 53) | (SEQ ID NO: 54) |
| CAGAGAACATCGACTTGGTCA | 971 | GAGAACAUCGACUUGGUCAUU | UUCUCUUGUAGCUGAACCAGU |
| (SEQ ID NO: 55) | | (SEQ ID NO: 56) | (SEQ ID NO: 57) |
| CAAGAACTGCGCAGAACAGAA | 1854 | AGAACUGCGCAGAACAGAAUU | UUUCUUGACGCGUCUUGUCUU |
| (SEQ ID NO: 58) | | (SEQ ID NO: 59) | (SEQ ID NO: 60) |
| CAGGAAGCTACGTGAACAGAG | 1934 | GGAAGCUACGUGAACAGAGUU | UUCCUUCGAUGCACUUGUCUC |
| (SEQ ID NO: 61) | | (SEQ ID NO: 62) | (SEQ ID NO: 63) |
| CAGCTAGACTGGAGTTGCAGT | 2629 | GCUAGACUGGAGUUGCAGUUU | UUCGAUCUGACCUCAACGUCA |
| (SEQ ID NO: 64) | | (SEQ ID NO: 65) | (SEQ ID NO: 66) |
| CAGACGTACTGTAGACTCCAC | 2936 | GACGUACUGUAGACUCCACUU | UUCUGCAUGACAUCUGAGGUG |
| (SEQ ID NO: 67) | | (SEQ ID NO: 68) | (SEQ ID NO: 69) |
| CAAGTGTCATCAGTGTACCTC | 3071 | AGUGUCAUCAGUGUACCUCUU | UUUCACAGUAGUCACAUGGAG |
| (SEQ ID NO: 70) | | (SEQ ID NO: 71) | (SEQ ID NO: 72) |
| CACTGGCTATAGTGTGTGACT | 3310 | CUGGCUAUAGUGUGUGACUUU | UUGACCGAUAUCACACACUGA |
| (SEQ ID NO: 73) | | (SEQ ID NO: 74) | (SEQ ID NO: 75) |
| CAGGCAGCCGCAATCATAGAT | 3690 | GGCAGCCGCAAUCAUAGAUUU | UUCCGUCGGCGUUAGUAUCUA |
| (SEQ ID NO: 76) | | (SEQ ID NO: 77) | (SEQ ID NO: 78) |
| CAGCCGCAATCATAGATCATG | 3694 | GCCGCAAUCAUAGAUCAUGUU | UUCGGCGUUAGUAUCUAGUAC |
| (SEQ ID NO: 79) | | (SEQ ID NO: 80) | (SEQ ID NO: 81) |
| CAATGCACCATATCTCTCGGT | 4370 | AUGCACCAUAUCUCUCGGUUU | UUUACGUGGUAUAGAGAGCC |
| (SEQ ID NO: 82) | | (SEQ ID NO: 83) | A |
| | | | (SEQ ID NO: 84) |
| CACCATATCTCTCGGTGTACA | 4375 | CCAUAUCUCUCGGUGUACAUU | UUGGUAUAGAGAGCCACAUG |
| (SEQ ID NO: 85) | | (SEQ ID NO: 86) | U |
| | | | (SEQ ID NO: 87) |
| CATATCTCTCGGTGTACAGTG | 4378 | UAUCUCUCGGUGUACAGUGUU | UUAUAGAGAGCCACAUGUCAC |
| (SEQ ID NO: 88) | | (SEQ ID NO: 89) | (SEQ ID NO: 90) |
| GATGGAACGGTTCAGTCCTCA | 684 | UGGAACGGUUCAGUCCUCAUU | UUACCUUGCCAAGUCAGGAGU |
| (SEQ ID NO: 91) | | (SEQ ID NO: 92) | (SEQ ID NO: 93) |
| GAATTAAGCGCCTTGAGCAAG | 1339 | AUUAAGCGCCUUGAGCAAGUU | UUUAAUUCGCGGAACUCGUUC |
| (SEQ ID NO: 94) | | (SEQ ID NO: 95) | (SEQ ID NO: 96) |
| GATCAATGAGCGGCTAACAGA | 1742 | UCAAUGAGCGGCUAACAGAUU | UUAGUUACUCGCCGAUUGUCU |
| (SEQ ID NO: 97) | | (SEQ ID NO: 98) | (SEQ ID NO: 99) |
| GACAGGAAGCTACGTGAACAG | 1932 | CAGGAAGCUACGUGAACAGUU | UUGUCCUUCGAUGCACUUGUC |
| (SEQ ID NO: 100) | | (SEQ ID NO: 101) | (SEQ ID NO: 102) |
| GAAGCTACGTGAACAGAGTGA | 1937 | AGCUACGUGAACAGAGUGAUU | UUUCGAUGCACUUGUCUCACU |
| (SEQ ID NO: 103) | | (SEQ ID NO: 104) | (SEQ ID NO: 105) |
| GAATACGCCTACCGATGCTCT | 2870 | AUACGCCUACCGAUGCUCUUU | UUUAUGCGGAUGGCUACGAG |
| (SEQ ID NO: 106) | | (SEQ ID NO: 107) | A |
| | | | (SEQ ID NO: 108) |
| GAAGGTCATGTCAGGATTCCT | 3255 | AGGUCAUGUCAGGAUUCCUUU | UUUCCAGUACAGUCCUAAGGA |
| (SEQ ID NO: 109) | | (SEQ ID NO: 110) | (SEQ ID NO: 111) |
| GAGAGCACTGGCTATAGTGTG | 3305 | GAGCACUGGCUAUAGUGUGUU | UUCUCGUGACCGAUAUCACAC |
| (SEQ ID NO: 112) | | (SEQ ID NO: 113) | (SEQ ID NO: 114) |
| GAGCACTGGCTATAGTGTGTG | 3307 | GCACUGGCUAUAGUGUGUGUU | UUCGUGACCGAUAUCACACAC |
| (SEQ ID NO: 115) | | (SEQ ID NO: 116) | (SEQ ID NO: 117) |
| GATGTTATCCATGCAAGTCGG | 3447 | UGUUAUCCAUGCAAGUCGGUU | UUACAAUAGGUACGUUCAGCC |
| (SEQ ID NO: 118) | | (SEQ ID NO: 119) | (SEQ ID NO: 120) |
| GATGCTAGCAGACACTGAGAA | 3539 | UGCUAGCAGACACUGAGAAUU | UUACGAUCGUCUGUGACUCUU |
| (SEQ ID NO: 121) | | (SEQ ID NO: 122) | (SEQ ID NO: 123) |
| GAGGCTTATGACAGCACTCTA | 3651 | GGCUUAUGACAGCACUCUAUU | UUCCGAAUACUGUCGUGAGAU |
| (SEQ ID NO: 124) | | (SEQ ID NO: 125) | (SEQ ID NO: 126) |
| GATGCTATCTGCGCAGTTGAG | 4230 | UGCUAUCUGCGCAGUUGAGUU | UUACGAUAGACGCGUCAACUC |
| (SEQ ID NO: 127) | | (SEQ ID NO: 128) | (SEQ ID NO: 129) |
| GATGTGAACTCCATGGAATGG | 4419 | UGUGAACUCCAUGGAAUGGUU | UUACACUUGAGGUACCUUACC |
| (SEQ ID NO: 130) | | (SEQ ID NO: 131) | (SEQ ID NO: 132) |
| GACGAACTGGTAGTACCTGAA | 4554 | CGAACUGGUAGUACCUGAAUU | UUGCUUGACCAUCAUGGACUU |
| (SEQ ID NO: 133) | | (SEQ ID NO: 134) | (SEQ ID NO: 135) |
| TAGCAGAGAACATCGACTTGG | 968 | GCAGAGAACAUCGACUUGGUU | UUCGUCUCUUGUAGCUGAACC |
| (SEQ ID NO: 136) | | (SEQ ID NO: 137) | (SEQ ID NO: 138) |
| TATTCGGAACTGTGAAGCACC | 1049 | UUCGGAACUGUGAAGCACCUU | UUAAGCCUUGACACUUCGUGG |
| (SEQ ID NO: 139) | | (SEQ ID NO: 140) | (SEQ ID NO: 141) |
| TAAGGCAAGAACTGCGCAGAA | 1849 | AGGCAAGAACUGCGCAGAAUU | UUUCCGUUCUUGACGCGUCUU |
| (SEQ ID NO: 142) | | (SEQ ID NO: 143) | (SEQ ID NO: 144) |
| TATGCAGCATAGAACATCAGC | 2029 | UGCAGCAUAGAACAUCAGCUU | UUACGUCGUAUCUUGUAGUCG |
| (SEQ ID NO: 145) | | (SEQ ID NO: 146) | (SEQ ID NO: 147) |
| TACACAACCAGCAGTTAGAAG | 2383 | CACAACCAGCAGUUAGAAGUU | UUGUGUUGGUCGUCAAUCUUC |
| (SEQ ID NO: 148) | | (SEQ ID NO: 149) | (SEQ ID NO: 150) |
| TATAGAGCACCAAGACTCACA | 2828 | UAGAGCACCAAGACUCACAUU | UUAUCUCGUGGUUCUGAGUG |
| (SEQ ID NO: 151) | | (SEQ ID NO: 152) | U |
| | | | (SEQ ID NO: 153) |
| TACTACTCCTACCAAGTGTCA | 3059 | CUACUCCUACCAAGUGUCAUU | UUGAUGAGGAUGGUUCACAG |
| (SEQ ID NO: 154) | | (SEQ ID NO: 155) | U |
| | | | (SEQ ID NO: 156) |
| TACTCCTACCAAGTGTCATCA | 3062 | CUCCUACCAAGUGUCAUCAUU | UUGAGGAUGGUUCACAGUAG |
| (SEQ ID NO: 157) | | (SEQ ID NO: 158) | U |
| | | | (SEQ ID NO: 159) |
| TATCTGCGCAGTTGAGATCTC | 4235 | UCUGCGCAGUUGAGAUCUCUU | UUAGACGCGUCAACUCUAGAG |
| (SEQ ID NO: 160) | | (SEQ ID NO: 161) | (SEQ ID NO: 162) |
| TATCTCTCGGTGTACAGTGAA | 4380 | UCUCUCGGUGUACAGUGAAUU | UUAGAGAGCCACAUGUCACUU |
| (SEQ ID NO: 163) | | (SEQ ID NO: 164) | (SEQ ID NO: 165) |
| TAAGCGGCGTTATTCCTTCAG | 4619 | AGCGGCGUUAUUCCUUCAGUU | UUUCGCCGCAAUAAGGAAGUC |
| (SEQ ID NO: 166) | | (SEQ ID NO: 167) | (SEQ ID NO: 168) |

In yet another embodiment, the polynucleotides of the present invention can be modified at the base moiety, sugar moiety or phosphate backbone to improve the stability, hybridization, or solubility of the molecules. For instance, the deoxyribose phosphate backbone of the polynucleotide molecules can be modified to generate peptide polynucleotides (see Hyrup et al., Bioorganic & Medicinal Chemistry 4:523, 1996). As used herein, the terms “peptide polynucleotides” or “PNAs” refer to polynucleotide mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. PNA oligomers can be synthesized using standard solid phase peptide synthesis protocols.

PNAs can be used in therapeutic and diagnostic applications. For example, PNAs can be used as antisense agents for sequence-specific modulation of the MRCK2 gene expression. PNAs can also be used in the analysis of single base pair mutations in a gene, (e.g., by PNA-directed PCR clamping); as artificial restriction enzymes when used in combination with other enzymes, (e.g., S1 nucleases); or as probes or primers for DNA sequencing or hybridization.

In one embodiment, PNAs can be modified to enhance their stability or cellular uptake by attaching lipophilic or other helper groups to PNA, by the formation of PNA-DNA chimeras, or by the use of liposomes or other drug delivery techniques known in the art. For example, PNA-DNA chimeras of the polynucleotides of the invention can be generated. These chimeras allow DNA recognition enzymes, such as RNase H and DNA polymerases, to interact with the DNA portion while the PNA portion provides high binding affinity and specificity. PNA-DNA chimeras can be linked using linkers of appropriate lengths which are selected based on base stacking, number of bonds between the nucleobases, and orientations. The PNA-DNA chimeras can be synthesized as follows. A DNA chain is synthesized on a solid support using standard phosphoramidite coupling chemistry and modified nucleoside analogs. PNA monomers are then coupled in a stepwise manner to produce a chimeric molecule with a 5′ PNA segment and a 3′ DNA segment. Alternatively, chimeric molecules can be synthesized with a 5′ DNA segment and a 3′ PNA segment.

In other embodiments, the polynucleotides of this invention may include other appended groups such as peptides (e.g., for targeting host cell receptors in vivo), or agents facilitating transportation across the cell membrane or the blood-kidney barrier (see, e.g., PCT Publication No. WO89/10134). In addition, polynucleotides can be modified using hybridization-triggered cleavage agents or intercalating agents. To this end, the polynucleotides can be conjugated to another molecule (e.g., a peptide, hybridization triggered cross-linking agent, transport agent, or hybridization-triggered cleavage agent). Furthermore, the polynucleotide can be detectably labeled.

Polypeptides and Variants Thereof

Several aspects of the invention pertain to isolated MRCK2 polypeptides and mutated MRCK2 polypeptides capable of inhibiting normal MRCK2 activity. The present invention also contemplates immunogenic polypeptide fragments suitable for raising anti-MRCK2 antibodies.

In one embodiment, native MRCK2 polypeptides can be isolated from cells or tissue sources by using standard protein purification techniques. Standard purification methods include electrophoresis, molecular, immunological and chromatographic techniques. Specific examples include ion exchange, hydrophobic, affinity or reverse-phase HPLC chromatography, and chromatofocusing. In one embodiment, MRCK2 polypeptides are purified using a standard affinity column coupled with anti-MRCK2 antibodies. Ultrafiltration and diafiltration techniques can also be used. The degree of purification depends on the purpose of the use of the MRCK2 polypeptides. In some instances, purification is not necessary.

In another embodiment, MRCK2 polypeptides or mutated MRCK2 polypeptides capable of inhibiting normal MRCK2 activity are produced by recombinant DNA techniques. Alternative to recombinant expression, MRCK2 polypeptides or mutated MRCK2 polypeptides can be synthesized chemically using standard peptide synthesis techniques.

The invention provides MRCK2 polypeptides encoded by the human MRCK2 gene, or homologs thereof. The polypeptides of this invention can be substantially homologous to human MRCK2 kinase (SEQ ID NO:2). Preferably, these polypeptides retain the biological activity of the native MRCK2 kinase. In one embodiment, the polypeptides comprise an amino acid sequence which is at least about 96%, 97%, 98%, 99% or more homologous to SEQ ID NO:2.

Comparison of sequences and determination of percent identity between two sequences can be accomplished using a mathematical algorithm. The percent identity between two amino acid sequences can be determined using the Needleman and Wunsch (J. Mol. Biol. 48:444-453, 1970) algorithm, or the GAP program in the GCG software package which uses either a Blossom 62 matrix or a PAM250 matrix, and a gap weight of 16, 14, 12, 10, 8, 6, or 4 and a length weight of 1, 2, 3, 4, 5, or 6. The percent identity between two nucleotide sequences can be determined using the GAP program in the GCG software package, which uses a NWSgapdna.CMP matrix and a gap weight of 40, 50, 60, 70, or 80 and a length weight of 1, 2, 3, 4, 5, or 6. The percent identity between two amino acid or nucleotide sequences can also be determined using the algorithm of E. Meyers and W. Miller (CABIOS, 4:11-17, 1989) which has been incorporated into the ALIGN program (version 2.0), or the pairwise BLAST program available at NCBI's BLAST web site.

The polypeptide and polynucleotide sequences of the present invention can be used as query sequences for searching public databases in order to identify similar sequences. The search can be conducted using BLAST programs, such as the protein BLAST, nucleotide BLAST, pairwise BLAST, and genomic BLAST that are available at the BLAST web site maintained by the NCBI. When using BLAST programs, the default parameters of the respective programs can also be used.

The invention further provides chimeric or fusion MRCK2 polypeptides. A fusion MRCK2 polypeptide contains an MRCK2-related polypeptide and a non-MRCK2 polypeptide. The MRCK2-related polypeptides include all or a portion of SEQ ID NO:2 or its variant. A peptide linker sequence can be employed to separate the MRCK2-related polypeptide from the non-MRCK2 polypeptide components by a distance sufficient to ensure that each polypeptide folds into its native secondary and tertiary structures. Such a peptide linker sequence is incorporated into the fusion protein using standard techniques well known in the art. Suitable peptide linker sequences can be chosen based on the following factors: (1) their ability to adopt a flexible extended conformation; (2) their inability to adopt a secondary structure that could interact with functional epitopes on the MRCK2-related polypeptide and non-MRCK2 polypeptide; and (3) the lack of hydrophobic or charged residues that might react with the polypeptide functional epitopes. Preferred peptide linker sequences contain Gly, Asn and Ser residues. Other near neutral amino acids, such as Thr and Ala can also be used in the linker sequence. Amino acid sequences suitable as linkers include those disclosed in Maratea et al., Gene 40:39-46, 1985; Murphy et al., Proc. Natl. Acad. Sci. USA 83:8258-8262, 1986; and U.S. Pat. Nos. 4,935,233 and 4,751,180. The linker sequences may be from 1 to about 50 amino acids in length. Linker sequences are not required when the MRCK2-related polypeptide or the non-MRCK2 polypeptide has non-essential N-terminal amino acid regions that can be used to separate the respective functional domains and thereby prevent steric interference.

In one embodiment, the fusion protein is a GST-MRCK2 fusion protein in which an MRCK2-related sequence, such as SEQ ID NO:2, is fused to the C-terminus of the GST sequence. This fusion protein can facilitate the purification of the recombinant MRCK2.

The MRCK2-fusion proteins of the invention can be incorporated into pharmaceutical compositions and administered to a subject. The MRCK2-fusion proteins can be used to affect the bioavailability of an MRCK2 substrate. The MRCK2-fusion proteins can also be used for the treatment or prevention of damages caused by (i) aberrant modification or mutation of MRCK2, or (ii) aberrant post-translational modification of MRCK2. It is also conceivable that a fusion protein containing a normal or mutated MRCK2 polypeptide, or a fragment thereof, can be used to inhibit MRCK2 activity in a human subject.

Moreover, the MRCK2-fusion proteins can be used as immunogens to produce anti-MRCK2 antibodies. They can also be used to purify MRCK2 ligands and to screen for molecules capable of inhibiting the interaction between MRCK2 and its substrates.

Preferably, the MRCK2-chimeric or fusion proteins of the invention are produced using standard recombinant DNA techniques. Commercially available expression vectors which encode a fusion moiety (e.g., a GST polypeptide) can be used.

A signal sequence can be used to facilitate secretion and isolation of the secreted protein or other proteins of interest. Signal sequences are typically characterized by a core of hydrophobic amino acids which are generally cleaved from the mature protein. Such signal peptides contain processing sites that allow cleavage of the signal sequence from the mature proteins as they pass through the secretory pathway. The present invention encompasses MRCK2 polypeptides having a signal sequence, or the polynucleotide sequences encoding the same.

The present invention also pertains to MRCK2 mutants which function as antagonists to MRCK2. In one embodiment, antagonists of MRCK2 are used as therapeutic agents. For example, a mutant of MRCK2 that forms a non-functional dimer with a wide-type MRCK2 (the so-called dominant negative mutant) can decrease the activity of MRCK2 and may ameliorate diseases in a subject wherein MRCK2 are abnormally increased in level or activity. Dominant negative MRCK2 mutants can be generated by mutagenesis, as appreciated by one skilled in the art.

MRCK2 mutants which function as either MRCK2 agonists or antagonists can be identified by screening combinatorial libraries of mutants. A variegated library of MRCK2 mutants can be produced by, for example, enzymatically ligating a mixture of synthetic oligonucleotides into gene sequences such that a degenerate set of potential MRCK2 sequences is expressible as individual polypeptides, or alternatively, as a set of larger fusion proteins containing the set of MRCK2 sequences therein. There are a variety of methods which can be used to produce libraries of potential MRCK2 mutants from a degenerate oligonucleotide sequence. A degenerate gene sequence can be chemically synthesized using an automatic DNA synthesizer. The synthetic gene can then be ligated into an appropriate expression vector.

In one embodiment, a library of coding sequences can be generated using nucleases. For instance, double stranded PCR fragments of the MRCK2 coding sequence can be treated by a nuclease which produces about one nick per molecule. The double-stranded DNAs then are subject to a cycle of denaturing and re-naturing. The newly reformed DNAs, which may include sense/antisense pairs from different nicked products, are treated with S1 nuclease to remove single stranded portions. Using this method, an expression library which encodes N-terminal, C-terminal or internal fragments of MRCK2 can be derived.

In addition, recursive ensemble mutagenesis (REM), a technique which enhances the frequency of functional mutants in the libraries, can be used to prepare MRCK2 mutants (Delgrave et al., Protein Engineering 6:327-331, 1993).

MRCK2 fragments, or variants thereof, can also be generated using synthetic means, such as solid-phase synthesis methods. Preferably, the synthesized fragment has less than about 100 amino acids, or preferably, less than about 50 amino acids.

Antibodies

In accordance with another aspect of the present invention, antibodies specific to MRCK2 or its variants are prepared. An antibody is considered to bind “specifically” to an antigen if the binding affinity between the antibody and the antigen is equal to, or greater than 10⁵ M⁻¹. The antibodies can be monoclonal or polyclonal. Preferably, the antibodies are monoclonal. More preferably, the antibodies are humanized antibodies.

Polyclonal anti-MRCK2 antibodies can be prepared by immunizing a suitable subject with MRCK2 or fragments thereof. The anti-MRCK2 antibody titer in the immunized subject can be monitored over the time using standard techniques, such as ELISA. The anti-MRCK2 antibody can be isolated from the immunized subject using well known techniques.

In one embodiment, hybridomas capable of producing anti-MRCK2 antibodies are prepared. Purified MRCK2 or its variants, or fragments thereof, are used to immunize a vertebrate, such as a mammal. Suitable mammals include mice, rabbits and sheep. Preferably, the fragment used for immunization comprises at least 8 amino acid residues, more preferably at least 12 amino acid residues, highly preferably at least 16 amino acid residues, and most preferably at least 20 amino acid residues.

Immunogenic fragments (epitopes) of MRCK2 can be identified using well known techniques. In general, any fragment of SEQ ID NO:2 can be used to raise antibodies specific to MRCK2. Preferred epitopes are regions that are located on the surface of MRCK2. These regions are usually hydrophilic.

Splenocytes are isolated from the immunized vertebrate and fused with an immortalized cell line (such as a myeloma) to form hybridomas. Preferably, the immortal cell line is derived from the same mammalian species as the lymphocytes. For example, murine hybridomas can be made by fusing an immortalized mouse cell line with lymphocytes isolated from a mouse that is immunized with an immunogenic preparation of the present invention. Preferred immortalized cell lines include mouse myeloma cell lines that are sensitive to culture medium containing hypoxanthine, aminopterin and thymidine (“HAT medium”). Suitable myeloma cell lines include, but are not limited to, the P3-NS/1-Ag4-1, P3-x63-Ag8.653 or Sp210-Ag14 myeloma lines, all of which are available from ATCC. In one embodiment, HAT-sensitive mouse myeloma cells are fused to mouse splenocytes using polyethylene glycol (“PEG”). Hybridoma cells thus produced are selected against HAT medium, which kills unfused or unproductively fused myeloma cells. Hybridoma cells which produce monoclonal anti-MRCK2 antibodies are then detected by screening the hybridoma culture supernatants.

A monoclonal anti-MRCK2 antibody can also be prepared by screening a recombinant combinatorial immunoglobulin library (e.g., an antibody phase display library). Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612).

The anti-MRCK2 antibodies of the present invention also include “single-chain Fv” or “scFv.” The scFv fragments comprise the V_{H} and V_{L} domains of an antibody. Generally, the scFv polypeptide further comprises a polypeptide linker between the V_{H} and V_{L} domains. The polypeptide linker enables the scFv to form the desired structure for antigen binding. Additionally, recombinant anti-MRCK2 antibodies, such as chimeric and humanized monoclonal antibodies, can be prepared, as appreciated by one of ordinary skill in the art.

Humanized antibodies are particularly desirable for therapeutic treatment of human subjects. Humanized forms of non-human (e.g., murine) antibodies are chimeric immunoglobulins, immunoglobulin chains, or fragments thereof (such as Fv, Fab, Fab′, F(ab′)₂ or other antigen-binding subsequences of antibodies) which contain minimal sequence derived from non-human immunoglobulin. Humanized antibodies are derived from human immunoglobulins in which the residues forming the complementary determining regions (CDRs) are replaced by the residues from CDRs of a non-human antibody, such as a mouse, rat or rabbit antibody having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. The humanized antibody can comprise at least one or two variable domains, in which all or substantially all of the CDR regions correspond to those of a non-human immunoglobulin and all or substantially all of the constant regions are those of a human immunoglobulin consensus sequence. The humanized antibody preferably comprises at least a portion of an immunoglobulin constant region (Fc) of a human immunoglobulin.

Humanized antibodies can be produced using transgenic mice which are incapable of expressing endogenous immunoglobulin heavy and light chains but can express human heavy and light chains. The transgenic mice are immunized in the normal fashion with a selected antigen. Monoclonal antibodies directed against the antigen can be obtained using conventional hybridoma technology. The human immunoglobulin transgenes harbored in the transgenic mice rearrange during B cell differentiation, and subsequently undergo class switching and somatic mutation. Using this technique, therapeutically useful IgG, IgA and IgE antibodies can be prepared.

In addition, humanized antibodies which recognize a selected epitope can be generated using a technique referred to as “guided selection.” In this approach a selected non-human monoclonal antibody, e.g., a murine antibody, is used to guide the selection of a humanized antibody recognizing the same epitope.

In a preferred embodiment, the antibodies to MRCK2 are capable of reducing or eliminating the biological function of MRCK2. Preferably, the antibodies reduce at least 25% of MCRK2 activity. More preferably, the antibodies reduce at least about 50% of the activity. Highly preferably, the antibodies reduce about 95-100% of MRCK2 activity.

Anti-MRCK2 antibodies can be used to isolate MRCK2. Suitable methods include affinity chromatography and immunoprecipitation. Moreover, anti-MRCK2 antibodies can be used to evaluate the expression level of MRCK2. Anti-MRCK2 antibodies can also be used to monitor MRCK2 level as part of a clinical testing procedure, or to determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive materials include ¹²⁵I, ¹³¹I, ³⁵S or ³H.

Anti-MRCK2 antibodies are also useful for targeting a therapeutic agent/drug to a particular cell or tissue. The therapeutic agent/drug may be coupled to an antibody, either covalently or non-covalently. For instance, a therapeutic agent can be coupled to an antibody via a linker group. A linker group can function as a spacer to separate the antibody from the agent so as to avoid interference with antibody's binding capabilities. The linker group can also serve to increase the chemical reactivity of a substituent on the agent or the antibody, and thus increase the coupling efficiency. A variety of bifunctional or polyfunctional reagents, either homo- or hetero-functional (such as those described in the catalog of the Pierce Chemical Co., Rockford, Ill.), can be employed as the linker group. Coupling may be effected, for example, through amino groups, carboxyl groups, sulfhydryl groups or oxidized carbohydrate residues. There are numerous references describing this methodology. See e.g., U.S. Pat. No. 4,671,958.

Where a therapeutic agent is more potent when free from the antibody, it may be desirable to use a linker group which is cleavable during or upon internalization into the target cell. A number of different cleavable linker groups have been described. The mechanisms for the intracellular release of an agent from these linker groups include cleavage by reduction of a disulfide bond (e.g., U.S. Pat. No. 4,489,710), by irradiation of a photolabile bond (e.g., U.S. Pat. No. 4,625,014), by hydrolysis of derivatized amino acid side chains (e.g., U.S. Pat. No. 4,638,045), by serum complement-mediated hydrolysis (e.g., U.S. Pat. No. 4,671,958), or by acid-catalyzed hydrolysis (e.g., U.S. Pat. No. 4,569,789).

It may also be desirable to couple more than one agent to an antibody. In one embodiment, multiple agents are coupled to one antibody molecule. In another embodiment, at least two different types of agents are coupled to one antibody. Regardless of the particular embodiment, immunoconjugates coupled with more than one agent can be prepared in a variety of ways, as appreciated by one of ordinary skill in the art.

Vectors, Expression Vectors and Gene Delivery Vectors

Another aspect of the invention pertains to vectors containing a polynucleotide encoding MRCK2 or a portion thereof. One type of vector is a “plasmid,” which includes a circular double stranded DNA into which additional DNA segments can be introduced. Vectors also include expression vectors and gene delivery vectors.

The expression vectors of the present invention comprise a polynucleotide encoding MRCK2 or a portion thereof. The expression vectors also include one or more regulatory sequences operably linked to the polynucleotide being expressed. These regulatory sequences are selected based on the type of host cells. It will be appreciated by those skilled in the art that the design of the expression vector depends on such factors as the choice of the host cells and the desired expression levels. MRCK2 can be expressed in bacterial cells such as E. coli, insect cells (using baculovirus expression vectors), yeast cells or mammalian cells. The expression vector can also be transcribed and translated in vitro, for example, by using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of the recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Suitable cleavage enzymes include Factor Xa, thrombin and enterokinase. Examples of fusion expression vectors include pGEX (Pharmacia Piscataway, N.J.), pMAL (New England Biolabs, Beverly, Mass.) and pRITS (Pharmacia, Piscataway, N.J.). Purified fusion proteins can be utilized in MRCK2 activity assays, or to generate antibodies specific for MRCK2.

Examples of suitable inducible non-fusion E. coli expression vectors include pTrc and pET 11d. Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21(DE3) or HSLE174(DE3) from a resident prophage harboring a T7 gn1 gene under the transcriptional control of the lacUV 5 promoter.

One strategy to maximize recombinant protein expression in E. coli is to express the protein in host bacteria that have an impaired capacity to proteolytically cleave the recombinant protein. Another strategy is to alter the polynucleotide sequence encoding the protein so that the individual codons for each amino acid are those preferentially utilized in E. coli.

In another embodiment, the MRCK2 expression vector is a yeast expression vector. Examples of yeast expression vectors include pYepSec1, pMFa, pJRY88, pYES2 (Invitrogen Corporation, San Diego, Calif.), and picZ (Invitrogen Corp, San Diego, Calif.).

Alternatively, MRCK2 or its variant can be expressed in insect cells using baculovirus expression vectors. Suitable baculovirus vectors include the pAc series and the pVL series.

In yet another embodiment, MRCK2 or its variant is expressed in mammalian cells using a mammalian expression vector. Examples of mammalian expression vectors include pCDM8 and pMT2PC. When used in mammalian cells, the expression vector's control functions are often provided by viral regulatory elements. For example, commonly used promoters are derived from polyoma, adenovirus 2, cytomegalovirus and Simian Virus 40.

In another embodiment, the mammalian expression vector contains tissue-specific regulatory elements. Examples of suitable tissue-specific promoters include the liver-specific albumin promoter, lymphoid-specific promoters, promoters of T cell receptors and immunoglobulins, neuron-specific promoters (e.g., the neurofilament promoter), pancreas-specific promoters, and mammary gland-specific promoters (e.g., milk whey promoter). Developmentally-regulated promoters are also contemplated, which include, for example, the α-fetoprotein promoter.

The present invention also provides a recombinant expression vector comprising a polynucleotide which encodes MRCK2 but is cloned into the expression vector in an antisense orientation. Regulatory sequences that are operatively linked to the antisense-oriented polynucleotide can be chosen to direct continuous expression of the antisense RNA molecule in a variety of cell types. Suitable regulatory sequences include viral promoters and/or enhancers. Regulatory sequences can also be chosen to direct constitutive, tissue specific or cell type specific expression of the antisense RNA. The antisense expression vector can be in the form of a recombinant plasmid, phagemid, or attenuated virus in which antisense polynucleotides are produced under the control of a highly efficient regulatory region.

The present invention further provides gene delivery vehicles for delivering polynucleotides to mammals. A polynucleotide sequence of the invention can be administered either locally or systemically via a gene delivery vehicle. Expression of the polynucleotide can be induced using endogenous mammalian or heterologous promoters. Expression of the polynucleotide in vivo can be either constituted or regulated. The gene delivery vehicles preferably are viral vectors, including retroviral, lentiviral, adenoviral, adeno-associated viral (AAV), herpes viral, or alphavirus vectors. The viral vectors can also be astrovirus, coronavirus, orthomyxovirus, papovavirus, paramyxovirus, parvovirus, picomavirus, poxvirus, or togavirus vectors.

Delivery of gene therapy constructs is not limited to the above mentioned viral vectors. Other delivery methods can also be employed. These methods include nucleic acid expression vectors, polycationic condensed DNA linked or unlinked to killed adenovirus, ligand linked DNA, liposome-DNA conjugates, gene guns, ionizing radiation, nucleic charge neutralization, or fusion with cell membranes. Naked DNA can also be employed. Uptake efficiency of the naked DNA may be improved using biodegradable latex beads. This method can be further improved by treating the beads to increase their hydrophobicity.

Regulatable Expression Systems

Another aspect of the present invention pertains to the use of regulatable expression systems to express desirable polynucleotides or polypeptides in cells. Systems suitable for this invention are briefly described below:

Tet-on/off system. The Tet-system is based on two regulatory elements derived from the tetracycline-resistance operon of the E. coli Tn10 transposon: the tet repressor protein (TetR) and the Tet operator DNA sequence (tetO) to which TetR binds (Gossen et al., Science 268: 1766-1769, 1995). The system consists of two components, a “regulator” and a “reporter” plasmid. The “regulator” plasmid encodes a hybrid protein containing a mutated Tet repressor (rtetR) fused to the VP 16 activation domain of herpes simplex virus. The “reporter” plasmid contains a tet-responsive element (TRE), which controls the “reporter” gene of choice. The rtetR-VP16 fusion protein can only bind to the TRE, therefore activating the transcription of the “reporter” gene in the presence of tetracycline. The system has been incorporated into a number of viral vectors including retrovirus, adenovirus and AAV.

Ecdysone system. The Ecdysone system is based on the molting induction system found in Drosophila, but modified for inducible expression in mammalian cells. The system uses an analog of the Drosophila steroid hormone ecdysone, muristerone A, to activate expression of the gene of interest via a heterodimeric nuclear receptor. Expression levels have been reported to exceed 200-fold over basal levels with no effect on mammalian cell physiology (No et al., Proc. Natl. Acad. Sci. USA 93: 3346-3351, 1996).

Progesterone-system. The progesterone receptor is normally stimulated to bind to a specific DNA sequence and to activate transcription through an interaction with its hormone ligand. Conversely, the progesterone antagonist mifepristone (RU486) is able to block hormone-induced nuclear transport and subsequent DNA binding. A mutant form of the progesterone receptor that can be stimulated to bind through an interaction with RU486 has been generated. To generate a specific, regulatable transcription factor, the RU486-binding domain of the progesterone receptor has been fused to the DNA-binding domain of the yeast transcription factor GAL4 and the transactivation domain of the HSV protein VP16. The chimeric factor is inactive in the absence of RU486. The addition of hormone, however, induces a conformational change in the chimeric protein, and this change allows binding to a GAL4-binding site and the activation of transcription from promoters containing the GAL4-binding site (Wang et al., Nat. Biotech 15: 239-243, 1997).

Rapamycin-system. Immunosuppressive agents, such as FK506 and rapamycin, act by binding to specific cellular proteins and facilitating their dimerization. For example, the binding of rapamycin to FK506-binding protein (FKBP) results in its heterodimerization with another rapamycin binding protein FRAP, which can be reversed by removal of the drug. The ability to bring two proteins together by addition of a drug potentiates the regulation of a number of biological processes, including transcription. A chimeric DNA-binding domain has been fused to the FKBP, which enables binding of the fusion protein to a specific DNA-binding sequence. A transcriptional activation domain also has been fused to FRAP. When these two fusion proteins are co-expressed in the same cell, a fully functional transcription factor can be formed by heterodimerization mediated by addition of rapamycin. The dimerized chimeric transcription factor can then bind to a synthetic promoter sequence containing copies of the synthetic DNA-binding sequence. This system has been successfully integrated into adenoviral and AAV vectors. Long term regulatable gene expression has been achieved in both mice and baboons (Ye et al., Science 283: 88-91, 1999).

Detection Methods

In patients with disorders related to the aberrant expression of MRCK2. The expression level of MRCK2 can be used as an indicator for detecting the presence of MRCK2-related diseases in humans. Detection and measurement of the relative amount of the MRCK2 gene product can be carried out using various methods known in the art.

Typical methodologies for detecting the transcription level of a gene include extracting RNA from a cell or tissue sample, hybridizing a labeled probe to the extracted RNA or derivative thereof (such as cDNA or cRNA), and detecting the probe. Suitable methods include Northern Blot and quantitative RCR or RT-PCR. In situ hybridization can also be used to detect the transcription level of the MRCK2 gene in human tissues.

Typical methodologies for detecting a polypeptide include extracting proteins from a cell or tissue sample, binding an antibody to the target polypeptide and detecting the antibody. Suitable methods include enzyme linked immunosorbent assays (ELISAs), Western blots, immunoprecipitations, and immunofluorescence. The antibody can be polyclonal, or preferably, monoclonal. The antibody can be an intact antibody, or a fragment thereof (e.g. Fab or F(ab′)₂). The antibody can be labeled with a radioisotope, a fluorescent compound, an enzyme, an enzyme co-factor, or a detectable ligand. The term “labeled,” with regard to a probe or antibody, is intended to encompass direct labeling such as through covalent coupling, as well as indirect labeling such as being mediated by another reagent which is directly labeled. Examples of indirect labeling include labeling a primary antibody using a fluorescently labeled secondary antibody, or attaching a DNA probe with a biotin which can be detected, for example, by a fluorescence-labeled streptavidin.

Preferably, the binding affinity of the antibody to MRCK2 is at least 10⁵ M⁻¹. More preferably, the binding affinity is at least 10⁶ M⁻¹. Other methods such as electrophoresis, chromatography or direct sequencing can also be used to detect the amount of a polypeptide in a biological sample. Anti-MRCK2 antibodies can also be directly introduced into a subject. The antibody can be labeled with a radioactive marker whose presence and location in the subject can be detected using standard imaging techniques.

In one embodiment, the genomic copies of the MRCK2 gene in the genome of a human subject may indicate the presence or predisposition of a disease. Detection of the presence or number of copies of the MRCK2 gene in the genome can be performed using methods known in the art. For instance, it can be assessed using Southern Blot. The probes for Southern Blot can be labeled with a radioisotope, a fluorescent compound, an enzyme, or an enzyme co-factor.

In the field of diagnostic assays, the above-described detection methods can be used to determine the severity of MRCK2-related diseases. A biological sample is isolated from a test subject, and the presence, quantity and/or activity of MRCK2 in the sample relative to a disease-free or control sample is evaluated. The expression level of MRCK2 in the biological sample can indicate the presence or severity of MRCK2-related diseases in the test subject. The term “biological sample” is intended to include tissues, cells or biological fluids isolated from the subject. A preferred biological sample is a serum sample isolated from the subject using conventional means.

Screening Methods

The present invention also provides methods for identifying MRCK2 modulators. The activity of MRCK2 can be evaluated using various methods, such as those disclosed in Leung et al., Mol. Cell Biol. 18:130-140, 1998, and Chen et al., J. Biol. Chem. 274: 19901-19905, 1999, both of which are incorporated herein by reference.

Suitable modulators include compounds or agents comprising therapeutic moieties, such as peptides, peptidomimetics, peptoids, polynucleotides, small molecules or other drugs. These moieties can either bind to MRCK2, or have a modulatory (e.g., stimulatory or inhibitory) effect on the activity of MRCK2. In one embodiment, the moieties have a modulatory effect on the interactions of MRCK2 with one or more of its natural substrates. These moieties can also exert a modulatory effect on the expression of MRCK2. The screen assays of the present invention comprise detecting the interactions between MRCK2 and test components.

The test compounds of the present invention can be either small molecules or bioactive agents. In a preferred embodiment, the test compound is a small organic or inorganic molecule. In another preferred embodiment, the test compounds are polypeptides, oligopeptides, polysaccharides, nucleotides or polynucleotides.

In accordance with one aspect of this invention, methods for screening for compounds that inhibit the biological activities of MRCK2 are provided. Pharmaceutical compositions comprising these compounds can subsequently be prepared. The screening method comprises (1) contacting a sample with a compound, and (2) comparing expression profile or biological activity of MRCK2 in the sample to determine whether the compound substantially decreases the expression level or activities of MRCK2. The screening method can be carried out either in vivo or in vitro.

The present invention further includes a method for screening for compounds capable of modulating the binding between MRCK2 and a binding partner. As used herein, the term “binding partner” refers to a bioactive agent which serves as either a substrate for MRCK2, or a ligand having a binding affinity to MRCK2. The bioactive agent may be selected from a variety of naturally-occurring or synthetic compounds, proteins, peptides, polysaccharides, nucleotides or polynucleotides.

Inhibitors of the expression, activity or binding ability of MRCK2 may be used as therapeutic compositions. These inhibitors can be formulated in suitable pharmaceutical compositions, as described herein below.

The present invention also provides methods for conducting high-throughput screening for compounds capable of inhibiting activity or expression of MRCK2. In one embodiment, the high-throughput screening method involves contacting test compounds with MRCK2, and then detecting the effect of the test compounds on MRCK2. Functional assays, such as cytosensor microphysiometer-based assays, calcium flux assays (e.g. FLIPR®, Molecular Devices Corp, Sunnyvale, Calif.), or the TUNEL assay, can be employed to measure MRCK2 cellular activity. Fluorescence-based techniques can be used for high-throughput and ultra high-throughput screening. They include, but are not limited to, BRET® and FRET® (both by Packard Instrument Co., Meriden, Conn.).

In a preferred embodiment, the high-throughput screening assay uses label-free plasmon resonance technology as provided by BIACORE® systems (Biacore International AB, Uppsala, Sweden). Plasmon free resonance occurs when surface plasmon waves are excited at a metal/liquid interface. By reflecting directed light from the surface as a result of contact with a sample, the surface plasmon resonance causes a change in the refractive index at the surface layer. The refractive index change for a given change of mass concentration at the surface layer is similar for many bioactive agents (including proteins, peptides, lipids and polynucleotides), and since the BIACORE® sensor surface can be functionalized to bind a variety of these bioactive agents, detection of a wide selection of test compounds can thus be accomplished.

Monitoring Efficacy of a Drug During Clinical Trials

Using the MRCK2 detection methods of this invention, the efficacy of a therapeutic agent for MRCK2-related diseases can be monitored during clinical trials. The therapeutic agent may be a drug, small molecule, agonist, antagonist, peptidomimetic, protein, peptide, or polynucleotide. The changes in the expression or activity of the MRCK2 gene in response to the treatment of the agent can be used to evaluate the therapeutic effect of the agent on patients with MRCK2-related diseases. In addition, the expression or activity of MRCK2 in response to the agent can be measured at various points during the clinical trial.

In a preferred embodiment, the method for monitoring the effectiveness of the therapeutic agent includes the steps of (i) obtaining a pre-administration sample from a subject; (ii) detecting the level of expression or activity of MRCK2 in the pre-administration sample; (iii) obtaining one or more post-administration samples from the subject; (iv) detecting the level of expression or activity of MRCK2 in the post-administration samples; (v) comparing the level of expression or activity of MRCK2 in the pre-administration sample to the level of expression or activity of MRCK2 in the post administration samples. The dose or frequency of the administration of the agent may be adjusted based on the effectiveness of the agent in a particular patient. Therefore, MRCK2 expression or activity can be used as an indicator of the effectiveness of a therapeutic agent for MRCK2-related diseases, even if the agent does not produce an observable phenotypic response.

Prognostic Assays

The detection methods described herein can be used to identify subjects having or at risk of developing MRCK2-related diseases. In addition, the detection methods can be used to determine whether an agent (e.g., an agonist, antagonist, peptidomimetic, protein, peptide, polynucleotide, small molecule, or other drug candidate) can be administered to a subject for effectively treating or preventing MRCK2-related diseases.

MRCK2 expression profiles at different progression stages of MRCK2-related diseases can be established. In addition, MRCK2 expression profiles in different patients who have different responses to a drug treatment are determined. A pattern may emerge such that a particular expression profile may be correlated to an increased likelihood of a poor prognosis. Therefore, the prognostic assay of the present invention may be used to determine whether a subject undergoing a treatment for an MRCK2-related disease has a poor outlook for long term survival or disease progression. Preferably, prognosis is performed shortly after diagnosis, such as within a few days after diagnosis. The result of prognosis can then be used to devise individualized treatment program, thereby enhancing the effectiveness of the treatment as well as the likelihood of long-term survival and well being.

The method of the invention can also be used to detect genetic alterations in the MRCK2 gene, thereby determining if a subject with the altered gene is at risk for damages characterized by aberrant regulation in MRCK2 activity or expression. In a preferred embodiment, the method includes detecting the presence or absence of a genetic alteration that affects the integrity of the MRCK2 gene, or detecting the aberrant expression of the MRCK2 gene. The genetic alteration can be detected by ascertaining the existence of at least one of the following: 1) deletion of one or more nucleotides from the MRCK2 gene; 2) addition of one or more nucleotides to the MRCK2 gene; 3) substitution of one or more nucleotides of the MRCK2 gene, 4) a chromosomal rearrangement in the MRCK2 gene; 5) alteration in the level of a messenger RNA transcript of the MRCK2 gene, 6) aberrant modification of the MRCK2 gene, 7) the presence of a non-wild type splicing pattern of a messenger RNA transcript of the MRCK2 gene, 8) non-wild type level MRCK2, 9) allelic loss of an MRCK2 gene, and 10) inappropriate post-translational modification of MRCK2.

In one embodiment, detection of the alteration involves the use of a probe/primer in a polymerase chain reaction (such as anchor PCR or RACE PCR) or alternatively, in a ligation chain reaction (LCR). LCR can be particularly useful for detecting point mutations in the MRCK2 gene. This method includes the steps of collecting a sample from a subject, isolating polynucleotides (e.g., genomic DNA, mRNA, or both) from the sample, contacting the polynucleotide with one or more primers which specifically hybridize to the MRCK2 gene or gene product, and detecting the presence or absence of an amplification product, or detecting the size of the amplification product and comparing its length to a control. It is understood that PCR and/or LCR can be used as a preliminary amplification step in conjunction with any other techniques described herein.

Alternative amplification methods include: self sustained sequence replication (Guatelli et al., Proc. Natl. Acad. Sci. USA 87:1874-1878, 1990), transcriptional amplification system (Kwoh et al., Proc. Natl. Acad. Sci. USA 86:1173-1177, 1989), and Q-Beta Replicase (Lizardi et al., Bio-Technology 6:1197, 1988).

In another embodiment, mutations in the MRCK2 gene can be identified using restriction enzymes. Differences in restriction enzyme digestion patterns indicates mutation(s) in the MRCK2 gene or its transcripts. Moreover, sequence specific ribozymes can be used to detect the presence of specific mutations. See, for example, U.S. Pat. No. 5,498,531.

In yet another embodiment, genetic mutations in the MRCK2 gene can be identified using high density arrays which contain a large number of oligonucleotides probes. For example, genetic mutations in the MRCK2 gene can be identified in two dimensional arrays. In this example, a first hybridization array of probes is used to scan through long stretches of DNA in a sample and a control in order to identify base changes between the two sequences. This step allows the identification of point mutations. This step is followed by a second hybridization array that allows the characterization of specific mutations by using smaller and specialized probe arrays which are complementary to all variants or mutations detected. Each mutation array is composed of parallel probe sets, one complementary to the wild-type gene and the other complementary to the mutant gene.

In still another embodiment, any sequencing reactions known in the art can be used to directly sequence the MRCK2 gene in order to detect mutations. It is contemplated that any automated sequencing procedures can be utilized, including sequencing by mass spectrometry.

In one embodiment, protection from cleavage agents is used to detect mismatched bases in RNA/RNA or RNA/DNA heteroduplexes. In general, the “mismatch cleavage” technique involves forming heteroduplexes by hybridizing a RNA or DNA (labeled) containing the wild-type MRCK2 gene sequence to a potentially mutant RNA or DNA obtained from a tissue sample. The double-stranded duplexes are treated with an agent which cleaves single-stranded regions of the duplex. The agent may be RNase (for RNA/DNA duplexes), or S1 nuclease (for DNA/DNA hybrids). In one case, either DNA/DNA or RNA/DNA duplexes are treated with piperidine and hydroxylamine, or piperidine and osmium tetroxide, in order to digest mismatched regions. After the digestion, the resulting material is separated by size on a denaturing polyacrylamide gel from which the site(s) of mutation may be determined.

In a preferred embodiment, the mismatch cleavage reaction employs one or more proteins that recognize mismatched base pairs in double-stranded DNA. Examples of these proteins include “DNA mismatch repair” enzymes. For instance, the mutY enzyme of E. coli cleaves A at G/A mismatches, and the thymidine DNA glycosylase from HeLa cells cleaves T at G/T mismatches. In one case, cDNAs are prepared from mRNAs isolated from test cells. The cDNAs are then hybridized to a probe derived from the MRCK2 gene. The duplex thus formed is treated with a DNA mismatch repair enzyme, and the cleavage products, if any, can be detected from electrophoresis protocols or the like. See, for example, U.S. Pat. No. 5,459,039.

In another embodiment, alterations in electrophoretic mobility are used to identify mutations in the MRCK2 gene. Differences in electrophoretic mobility between mutant and wild type polynucleotides can be detected using single strand conformation polymorphism (SSCP). The resulting alteration in electrophoretic mobility enables the detection of a single base change. The DNA fragments can be labeled or detected with probes. In one case, the sensitivity of the assay is enhanced by using RNA, in which the secondary structure is more sensitive to a change in sequence. In a preferred embodiment, the assay utilizes heteroduplex analysis to separate double stranded heteroduplex molecules on the basis of changes in electrophoretic mobility (Keen et al., Trends Genet 7:5, 1991).

In yet another embodiment, the movement of mutant or wild-type fragments is evaluated using denaturing gradient gel electrophoresis (DGGE). For this purpose, DNA fragments can be modified to insure that they do not completely denature. For instance, a GC clamp of approximately 40 GC-rich base pairs can be added to the DNA fragment using PCR. In a further embodiment, a temperature gradient is used in place of a denaturing gradient (Rosenbaum and Reissner, Biophys Chem 265:12753, 1987).

Examples of other techniques for detecting point mutations include, but are not limited to, selective oligonucleotide hybridization, selective amplification, or selective primer extension. In one embodiment, oligonucleotide primers for specific amplification carry the mutation of interest in the center of the molecule (so that amplification depends on differential hybridization) or at the extreme 3′ end of one primer where, under appropriate conditions, mismatch can prevent or reduce polymerase extension. See, for example, Saiki et al., Proc. Natl. Acad. Sci USA 86:6230, 1989. In addition, it may be desirable to introduce a novel restriction site in the region of the mutation to create cleavage-based detection.

The methods described herein can be performed using prepackaged diagnostic kits which comprise at least one polynucleotide probe or one antibody of the present invention. These kits can be used in clinical settings to diagnose subjects exhibiting symptoms or family history of an MRCK2-related disease. Any cell type or tissue in which MRCK2 is expressed can be used for prognostic or diagnostic purposes.

Prophylactic Methods

This invention also provides methods for preventing diseases associated with aberrant MRCK2 expression or activity. The methods comprise administering to a target subject an agent which modulates MRCK2 expression or activity.

Subjects at risk of diseases which are caused by or attributed to aberrant MRCK2 expression or activity can be identified using the diagnostic or prognostic assays described herein. A prophylactic agent can be administered prior to the manifestation of MRCK2-related disease symptoms in order to prevent or delay MRCK2-related diseases. Suitable prophylactic agents include mutant MRCK2 proteins, MRCK2 antagonist agents, or MRCK2 antisense polynucleotides.

The prophylactic methods of this invention can be specifically tailored or modified, based on knowledge obtained from the study of pharmacogenomics. Pharmacogenomics includes the application of genomics technologies, such as gene sequencing, statistical genetics, and gene expression analysis, to drugs which are either in clinical development or on the market. Pharmacogenomics can be used to determine a subject's response to a drug (e.g., a subject's “drug response phenotype” or “drug response genotype”). Thus, another aspect of this invention is to provide methods for tailoring an individual's prophylactic or therapeutic treatment using MRCK2 modulators according to the individual's drug response genotype. Pharmacogenomics allows a clinician or physician to target prophylactic or therapeutic treatments to subjects who will most benefit from the treatment and to avoid treatment of subjects who will experience toxic drug-related side effects.

One pharmacogenomics approach to identify genes that predict drug response, known as “a genome-wide association,” relies primarily on a high-resolution map of the human genome consisting of already known gene-related sites (e.g., a “bi-allelic” gene marker map which consists of 60,000-100,000 polymorphic or variable sites on the human genome, each of which has two variants). Such a high-resolution genetic map can be compared to a map of the genome of each of a statistically substantial number of subjects taking part in a Phase II/III drug trial in order to identify genes associated with a particular observed drug response or side effect. Alternatively, such a high resolution map can be generated from a combination of some ten-million known single nucleotide polymorphisms (SNPs) in the human genome. A “SNP” is a common alteration that occurs in a single nucleotide base in a stretch of DNA. For example, a SNP may occur once per every 1000 bases of DNA. A SNP may be involved in a disease process. However, the vast majority of SNPs may be not related to diseases. Given a genetic map based on the occurrence of SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals. Thus, mapping of the MRCK2 gene to SNP maps of patients with MRCK2-related diseases may facilitate the identification of drug-response-prediction genes.

Alternatively, the “candidate gene approach” can be utilized to identify genes that predict drug response. According to this method, if a gene that encodes a drug target is known, all common variants of that gene can be easily identified in the population. It then can be determined if a particular drug response is associated with one version of the gene versus another.

The activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYPZC19) has provided an explanation as to why some subjects do not obtain the expected drug effects or show exaggerated drug response and serious toxicity after taking the standard and safe dose of a drug. These polymorphisms are expressed in two phenotypes in the population, extensive metabolizer and poor metabolizer. The prevalence of poor metabolizer phenotypes is different among different populations. For example, the gene coding for CYP2D6 is highly polymorphic and several mutations have been identified in poor metabolizers, which all lead to the absence of functional CYP2D6. Poor metabolizers of CYP2D6 and CYP2C19 quite frequently experience exaggerated drug response and side effects when they receive standard doses. If a metabolite is the active therapeutic moiety, poor metabolizers show no therapeutic response. The other extreme are the so called ultra-rapid metabolizers who do not respond to standard doses. Recently, the molecular basis of ultra-rapid metabolism has been identified to be due to CYP2D6 gene amplification.

In one embodiment, the “gene expression profiling” method can be utilized to identify genes that predict drug response. In this regard, the gene expression profile of an animal dosed with a drug can give an indication of whether the gene pathways related to toxicity have been turned on.

Information generated from the above pharmacogenomics approaches can be used to determine the appropriate dosage or treatment regimen suitable for a particular individual. This knowledge can avoid adverse reactions or therapeutic failure, and therefore enhance therapeutic or prophylactic efficiency when treating a subject with an MRCK2 modulator.

Therapeutic Methods

As described above, the present invention includes therapeutic methods for treating a subject at risk for, susceptible to, or diagnosed with MRCK2-related diseases. The therapeutic methods can be individually tailored based on the subject's drug response genotype. Typically, the therapeutic methods comprise modulating the expression or activity of MRCK2 in the subject. In one embodiment, the method comprises contacting a plurality of cells in the subject with an agent that inhibits the expression or activity of MRCK2. Suitable agents include polynucleotides (e.g., an antisense oligonucleotides of MRCK2), polypeptides (e.g., a dominant negative mutant of MRCK2), or polysaccharides, naturally-occurring target molecules of MRCK2 protein (e.g., an MRCK2 protein substrate or receptor), anti-MRCK2 antibodies, MRCK2 antagonists, or other small organic and inorganic molecule. They may also include vectors comprising polynucleotides encoding MRCK2 inhibitors or antisense sequences. Moreover, the agents can be anti-MRCK2 antibodies conjugated with therapeutic moieties. Suitable agents can be identified using the screening assays of the present invention.

Pharmaceutical Compositions

The present invention is further directed to pharmaceutical compositions comprising an MRCK2 modulator and a pharmaceutically acceptable carrier. As used herein, a “pharmaceutically acceptable carrier” is intended to include any and all solvents, solubilizers, fillers, stabilizers, binders, absorbents, bases, buffering agents, lubricants, controlled release vehicles, diluents, emulsifying agents, humectants, lubricants, dispersion media, coatings, antibacterial or antifungal agents, isotonic and absorption delaying agents, and the like, compatible with pharmaceutical administration. The use of such media and agents for pharmaceutically active substances is well-known in the art. Except insofar as any conventional media or agent is incompatible with the active compound, use thereof in the compositions is contemplated. Supplementary agents can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine; propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the injectable composition should be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active modulator (e.g., an anti-MRCK2 antibody, an MRCK2 activity inhibitor, or a gene therapy vector expressing antisense nucleotide to MRCK2) in the required amount in an appropriate solvent, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active, ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Stertes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the bioactive compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the therapeutic moieties, which may contain a bioactive compound, are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from e.g. Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein includes physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The pharmaceutical compositions can be included in a container, pack, or dispenser together with instructions for administration.

Kits

The invention also encompasses kits for detecting the presence of an MRCK2 gene product in a biological sample. An example kit comprises reagents for assessing expression of MRCK2 at mRNA or protein level. Preferably, the reagents include an antibody or fragment thereof, wherein the antibody or fragment specifically binds to MRCK2. Optionally, the kits may comprise a polynucleotide probe capable of specifically binding to a transcript of the MRCK2 gene. The kit may also contain means for determining the amount of MRCK2 protein or mRNA in the test sample, and/or means for comparing the amount of MRCK2 protein or mRNA in the test sample to a control or standard. The compound or agent can be packaged in a suitable container.

The invention further provides kits for assessing the suitability of each of a plurality of compounds for inhibiting MRCK2-related diseases in cells or human subjects. Such kits include a plurality of compounds to be tested, and a reagent (such as an antibody specific to MRCK2 proteins, or a polynucleotide probe or primer capable of hybridizing to the MRCK2 gene) for assessing expression of MRCK2.

It should be understood that the above-described embodiments are given by way of illustration, not limitation. Various changes and modifications within the scope of the present invention will become apparent to those skilled in the art from the present description.

Host Cells

Another aspect of the invention pertains to host cells into which a polynucleotide molecule of the invention is introduced, e.g., an MRCK2 gene or homolog thereof, within an expression vector, a gene delivery vector, or a polynucleotide molecule of the invention containing sequences which allow it to homologously recombine into a specific site of the host cell's genome. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, an MRCK2 gene can be expressed in bacterial cells such as E. coli, insect cells, yeast or mammalian cells (such as Chinese hamster ovary cells (CHO), COS cells, Fischer 344 rat cells, HLA-B27 rat cells, HeLa cells, A549 cells, or 293 cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign polynucleotide (e.g., DNA) into a host cell, including calcium phosphate or calcium chloride co-precipitation, DAKD-dextran-mediated transfection, lipofection, or electoporation.

For stable transfection of mammalian cells, it is known that, depending upon the expression vector and transfection technique used, only a small fraction of cells may integrate the foreign DNA into their genome. In order to identify and select these integrants, a gene that encodes a selectable flag (e.g., resistance to antibiotics) is generally introduced into the host cells along with the gene of interest. Preferred selectable flags include those which confer resistance to drugs, such as G418, hygromycin and methotrexate. Polynucleotides encoding a selectable flag can be introduced into a host cell by the same vector as that encoding MRCK2 or can be introduced by a separate vector. Cells stably transfected with the introduced polynucleotide can be identified by drug selection (e.g., cells that have incorporated the selectable flag gene will survive, while the other cells die).

A host cell of the invention, such as a prokaryotic or eukaryotic host cell in culture, can be used to produce (i.e., express) MRCK2. Accordingly, the invention further provides methods for producing MRCK2 using the host cells of the invention. In one embodiment, the method comprises culturing the host cell of invention (into which a recombinant expression vector containing an MRCK2 gene has been introduced) in a suitable medium such that MRCK2 is produced. In another embodiment, the method further comprises isolating MRCK2 from the medium or the host cell.

Transgenic and Knockout Animals

The host cells of the invention can also be used to produce non-human transgenic animals. For example, in one embodiment, a host cell of the invention is a fertilized oocyte or an embryonic stem cell into which MRCK2-coding sequences have been introduced. Such host cells can then be used to create non-human transgenic animals in which exogenous sequences encoding MRCK2 have been introduced into their genome or homologous recombinant animals in which endogenous sequences encoding MRCK2 have been altered. Such animals are useful for studying the function and/or activity of MRCK2 and for identifying and/or evaluating modulators of MRCK2 activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, and the like. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, a “homologous recombinant animal” or “knockout animal” is a non-human animal, preferably a mammal, more preferably a mouse, in which an endogenous MRCK2 gene has been altered by homologous recombination between the endogenous gene and an exogenous DNA molecule introduced into a cell of the animal, e.g., an embryonic cell of the animal, prior to development of the animal.

A transgenic animal of the invention can be created by introducing an MRCK2-encoding polynucleotide into the mate pronuclei of a fertilized oocyte, e.g., by microinjection or retroviral infection, and allowing the oocyte to develop in a pseudopregnant female foster animal. Intronic sequences and polyadenylation signals can also be included in the transgene to increase the efficiency of expression of the transgene. A tissue-specific regulatory sequence(s) can be operably linked to a transgene to direct expression of MRCK2 to particular cells. Methods for generating transgenic animals via embryo manipulation and microinjection, particularly animals such as mice, have become conventional in the art. Similar methods are used for production of other transgenic animals. A transgenic founder animal can be identified based upon the presence of a transgene of the invention in its genome and/or expression of mRNA corresponding to a gene of the invention in tissues or cells of the animals. A transgenic founder animal can then be used to breed additional animals carrying the transgene. Moreover, transgenic animals carrying a transgene encoding MRCK2 can further be bred to other transgenic animals carrying other transgenes.

To create a homologous recombinant animal (knockout animal), a vector is prepared which contains at least a portion of a gene of the invention into which a deletion, addition or substitution has been introduced to thereby alter, e.g., functionally disrupt, the gene. The gene can be a human gene, but more preferably, is a non-human homolog of a human gene of the invention (e.g., a homolog of the MRCK2 gene). For example, a mouse gene can be used to construct a homologous recombination polynucleotide molecule, e.g., a vector, suitable for altering an endogenous gene of the invention in the mouse genome. In a preferred embodiment, the homologous recombination polynucleotide molecule is designed such that, upon homologous recombination, the endogenous gene of the invention is functionally disrupted (i.e., no longer encodes a functional protein; also referred to as a “knockout” vector). Alternatively, the homologous recombination polynucleotide molecule can be designed such that, upon homologous recombination, the endogenous gene is mutated or otherwise altered but still encodes functional protein (e.g., the upstream regulatory region can be altered to thereby alter the expression of the endogenous MRCK2 gene). In the homologous recombination polynucleotide molecule, the altered portion of the gene of the invention is flanked at its 5′ and 3′ ends by additional polynucleotide sequence of the gene of the invention to allow for homologous recombination to occur between the exogenous gene carried by the homologous recombination polynucleotide molecule and an endogenous gene in a cell, e.g., an embryonic stem cell. The additional flanking polynucleotide sequence is of sufficient length for successful homologous recombination with the endogenous gene.

Typically, several kilobases of flanking DNA (both at the 5′ and 3′ ends) are included in the homologous recombination polynucleotide molecule. The homologous recombination polynucleotide molecule is introduced into embryonic stem cells by electroporation. The cells in which the introduced gene has homologously recombined with the endogenous gene are selected. The selected cells can then be injected into a blastocyst of an animal (e.g., a mouse) to form aggregation chimeras. A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the homologously recombined DNA. Methods for constructing homologous recombination polynucleotide molecules, e.g., vectors, or homologous recombinant animals are well known in the art.

In another embodiment, transgenic non-human animals can be produced which contain selected systems which allow for regulated expression of the transgene. One example of such a system is the cre/loxP recombinase system of bacteriophage P1. Another example of a recombinase system is the FLP recombinase system of Saccharomyces cerevisiae (see e.g., O'Gorman et al., Science 251:1351-1355, 1991). If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, I. et al., Nature 385:810-813, 1997, and PCT International Publication Nos. WO97/07668 and WO97/07669. In brief, a cell, e.g., a somatic cell, from the transgenic animal can be isolated and induced to exit the growth cycle and enter Go phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell, e.g., the somatic cell, is isolated.

### EXAMPLES

### Example 1

### Identification of the MRCK2 Sequence in Human Genome Database

The nucleic acid sequence of MRCK2 is obtained from a newly developed genomic prediction pipeline. Briefly, the X-ray crystal structures of the catalytic domains of protein kinases were collected and aligned together according to their structural identity/similarities. The alignment was converted into a “scoring matrix” which carried the structural profile of the kinase catalytic domains. This scoring matrix was then used to search the Celera Human Genome database for sequences that have kinase catalytic domains.

### Example 2

### BLAST and Hydrophobicity Analysis

Sequence alignments between MRCK2 and other sequences in GenBank database were performed using the standard protein-protein BLAST (blastp), standard nucleotide-nucleotide BLAST (blastn), BLAST2 Sequences, and human genome BLAST programs that are available at NCBI's BLAST website.

A standard protein-protein BLAST search in database nr with no filter chosen, “Expect” setting at 10.0, “Word Size” setting at 3, “Matrix” setting at BLOSUM62, “Gap costs” setting at Existence:11 and Extension:1, identified amino acid sequence similarities between MRCK2 and other proteins. These proteins include, but are not limited to, rat MRCK alpha (Entrez accession numbers: AF021935 or NM_{—}053657, 95% sequence identities), rat MRCK beta (Entrez accession number: AF021936, 60% sequence identities), a human KIAA1124 protein (Entrez accession number: AB032950, 60% sequence identities), and human Cdc42-binding protein kinase beta (Entrez accession number: AF128625 or NM_{—}006035, 60% sequence identities).

Further analysis using the standard nucleotide-nucleotide BLAST search in database nr with no filter chosen, “Expect” setting at 10.0, “Word Size” setting at 3, identified nucleotide sequence similarities between MRCK2 and other proteins. These proteins include, but are not limited to, rat MRCK alpha (Entrez accession number: AF021935, 90% sequence identities); human KIAA gene product (Entrez accession number XM037220.4, 100% sequence identities to nucleotides 3228-4910 of MRCK2); human clone IMAGE:3868557 (Entrez accession number BC008677, 99% sequence identities to nucleotides 504-2150 of MRCK2; and human cDNA FLJ25525 fis, clone CBR07120 (Entrez accession number AK098391, 99% sequence identities to nucleotides 2944-4546 of MRCK2).

A conserved domain search was performed within the standard protein-protein BLAST search with the RPS-BLAST 2.2.3 [Apr. 24, 2002] program. Multiple domains, including at least a kinase domain, a protein kinase C terminal domain, a myosin tail domain, a small phorbol esters/diacylglycerol binding domain (DAG_PE binding domain), a Pleckstrin homology domain, a CNH domain, and a P21-Rho-binding domain, were identified in MRCK2.

A human genome search was carried out using blastn program with Expect setting at 0.01, Filter setting at default, Descriptions setting at 100, and Alignment settings at 100. The alignment revealed 31 alignments between the MRCK2 gene and nucleotides 3700505 to 3973937 of human chromosome 1, indicating that the MRCK2 gene is localized in locus 1q42 of human chromosome 1. Specifically, the MRCK2 gene is located between LOC163851 and LOC200096, and overlaps with LOC205022, LOC163855, PRO2012 and KIAA0451. The exons/introns in the MRCK2 gene were determined using the program “sim4” described by Florea et al. in “A computer program for aligning a cDNA sequence with a genomic DNA sequence,” Genome Res. 8:967-974, 1998.

### Example 3

### Hydrophobicity Analysis

The hydrophobicity profile of MRCK2 sequence (FIG. 12) was generated using the GES (Goldman, Engelman and Steitz) hydrophobicity scale (Engelman et al., Ann. Rev. Biophys. Biophys. Chem. 15:321-353, 1986). Briefly, the GES scale is used to identify nonpolar transbilayer helices. The curve is the average of a residue-specific hydrophobicity scale over a window of 20 residues. When the line is in the upper half of the frame (positive), it indicates a hydrophobic region and when it is in the lower half (negative), a hydrophilic region.

In FIG. 12, the X-axis represents the length of the protein in amino acids (aa), while the Y-axis represents the GES score. The curve line shows the GES pattern of the entire protein, while the strait line represent certain cutoff for potential membrane spanning domains. The hydrophobicity profile indicates that MRCK2 is probably not a membrane protein.

### Example 4

### MTN and MTA Analysis

The DNA probe for the MTN and MTA analysis is a PCR amplified 634 nucleotide fragment (SEQ ID NO:4) from human cDNA library. The probe located at exon 28-30 of the MRCK2 gene and nucleotide 3611-4244 of the MRCK2 cDNA. The PCR primers were designed based on the cDNA prediction. The Forward primer sequence is:

5′ AGAAAAACAAATTCAGAGACCGCTCAG 3′ (SEQ ID NO:5). The reverse primer sequence is: 5′ GCGCAAGATAGCATCCATTGGTTG 3′ (SEQ ID NO:6). The PCR amplified probe was sequence verified. The positions of the probe and the primers relative to the MRCK2 gene are shown in FIG. 13.

The MTN analysis was performed using the Multiple Tissue Northern (MTN) Blot Kit (Clontech Laboratoryies, Inc., Palo Alto, Calif.) under conditions specified in the User Manual. Briefly, the human MRCK2 probe was labeled with ³²P The MTN Blot was prehybridized in ExpressHyb solution at 68° C. for 30 min, and then hybridized with the labeled probe at 68° C. for 1 hr. The blot was washed two times with Wash Solution 1 for 30 min at room temperature and two times with Wash Solution 2 for 30 min at 50° C., and was then exposed to X-ray film. Two transcripts of MRCK2, a 11 kb sharp band and a 7.5 kb smear band, were detected in human brain, heart, skeletal muscle, colon, spleen, kidney, liver, small intestine, placenta, and lung; but not in thymus and peripheral blood leukocyte. A 4 kb band was detected only in brain and skeletal muscle.

The MTA analysis was performed using the Multiple Tissue Expression (MTE®) Array Kit (Clontech Laboratoryies, Inc., Palo Alto, Calif.) under conditions specified in the User Manual. Briefly, the MTE array was hybridized to ³²P-labeled human MRCK2 probe in ExpressHyb solution at 65° C. overnight with continuous agitation. The array was washed four times with Wash Solution 1 for 20 min at 65° C. and two times with Wash Solution 2 for 20 min at 55° C., and was then exposed to X-ray film. MRCK2 expression was found in most tissues except thymus, peripheral blood leukocyte, ovary and two Burkitt's lymphomas.

Having described preferred embodiments of compositions, organisms and methodologies employing a novel human gene MRCK2 (which are intended to be illustrative and not limiting), it is noted that modifications and variations can be made by persons skilled in the art in light of the above teachings. Therefore, it is understood that changes may be made in the particular embodiments disclosed which are within the scope of what is described as defined by the appended claims.

1. An isolated polynucleotide comprising a nucleic acid sequence which encodes the amino acid sequence depicted in SEQ ID NO:2.

2. The polynucleotide according to claim 1, wherein the nucleic acid sequence is selected from the group consisting of:
(a) the nucleic acid sequence as shown in SEQ ID NO:1;
(b) the complement of (a); and
(c) a nucleic acid sequence that differs from (a) or (b) due to the degeneracy of the genetic code.

3. The polynucleotide according to claim 1, wherein the nucleic acid sequence is selected from the group consisting of:
(a) the nucleic acid sequence as shown in SEQ ID NO:3;
(b) the complement of (a); and
(c) a nucleic acid sequence that differs from (a) or (b) due to the degeneracy of the genetic code.

4. An isolated polynucleotide comprising a variant of a nucleic acid sequence, wherein said nucleic acid sequence encodes the amino acid sequence depicted in SEQ ID NO:2, and wherein the variant and said nucleic acid sequence have at least 96% sequence identity.

5. The polynucleotide according to claim 4, wherein the variant and said nucleic acid sequence have at least 98% sequence identity.

6. An isolated polynucleotide that is capable of hybridizing under highly stringent conditions to the sequence set forth in SEQ ID NO:1, or the complement thereof, wherein said polynucleotide encodes a protein having MRCK activity.

7. The polynucleotide of claim 6, wherein said polynucleotide and the sequence set forth in SEQ ID NO:1 have a sequence identity of at least 96%.

8. An isolated polypeptide comprising a fragment of SEQ ID NO:2, wherein said fragment comprises at least 500 consecutive amino acid residues of SEQ ID NO:2.

9. The polypeptide according to claim 8, wherein the fragment consists of SEQ ID NO:2.

10. An isolated polypeptide comprising a variant of a fragment of SEQ ID NO:2, wherein said fragment includes at least 500 consecutive amino acid residues of SEQ ID NO:2.

11. The polypeptide according to claim 10, wherein the variant and said fragment have at least 91% sequence identity.

12. An antibody capable of binding to the amino acid sequence depicted in SEQ ID NO:2 with a binding affinity of no less than 10⁵ M-1.

13. An MRCK2 detection kit comprising at least one of:
(a) the antibody of claim 12; and
(b) a probe that is capable of hybridizing under stringent conditions to the nucleotide sequence of SEQ ID NO:1 or the complement thereof.

14. A host cell containing the polynucleotide of claim 1 or a variant thereof.

15. A transgenic non-human animal comprising the polynucleotide of claim 1 or a variant thereof.

16. A non-human animal, wherein at least one allele of a gene in the genome of said animal is functionally disrupted, and wherein said gene encodes a polypeptide that has at least 70% sequence identity to SEQ ID NO:2.

17. A method for identifying an agent capable of binding to MRCK2 kinase, said method comprising:
contacting a candidate agent with a polypeptide comprising:
(a) an amino acid sequence recited in SEQ ID NO:2,
(b) a fragment of SEQ ID NO:2, or
(c) a variant of (a) or (b); and
detecting the binding between said candidate agent and said polypeptide.

18. A method for identifying an agent capable of modulating an activity of an MRCK2 kinase, comprising:
contacting a candidate agent with an polypeptide comprising:
(a) an amino acid sequence recited in SEQ ID NO:2, or
(b) a biologically active portion of SEQ ID NO:2; and
detecting a change in an activity of said polypeptide.

19. A pharmaceutical composition for preventing or treating MRCK2-related diseases, comprising a pharmaceutically acceptable carrier and an agent that modulates an MRCK2 activity or the MRCK2 gene expression.

20. A method for preventing or treating an MRCK2-related disease in a subject, said method comprising the step of:
introducing into the subject an effective amount of the pharmaceutical composition of claim 19.

21. A polynucleotide capable of inhibiting human MRCK2 gene expression by RNA interference.

22. The polynucleotide according to claim 21, comprising a siRNA sense strand or a siRNA antisense strand selected from Table 4.

23. A method, comprising introducing a polynucleotide of claim 21 into a cell which expresses human MRCK2 gene, thereby inhibiting the expression of said gene in said cell by RNA interference.
